(19) United States
(12) Patent Application Publication
(10) Pub. No.: US 2023/0265094 A1
(43) Pub. Date: Aug. 24, 2023

(54) RIPK1 INHIBITORS AND METHODS OF USE

(71) Applicants: Jenny Lorena RICO DUQUE, Boston, MA (US); Zachary G. BRILL, Boston, MA (US); Xavier FRADERA, Boston, MA (US); Phieng SILIPHAIVANH, Boston, MA (US); Merck Sharp & Dohme LLC, Rahway, NJ (US)

(72) Inventors: Jenny Lorena Rico Duque, Canton, MA (US); Phieng Siliphaivanh, Newton, MA (US); Xavier Fradera, Boston, MA (US); Zachary G. Brill, Boston, MA (US); Jing SU, Scotch Plains, NJ (US)

(73) Assignee: Merck Sharp & Dohme LLC, Rahway, NJ (US)

(21) Appl. No.: 18/007,776

(22) PCT Filed: Jun. 7, 2021
(86) PCT No.: PCT/US2021/036080

§ 371 Date: Dec. 2, 2022

Related U.S. Application Data
(60) Provisional application No. 63/038,467, filed on Jun. 12, 2020.

(51) Int. Cl. C07D 471/10; C07D 403/14; C07D 401/14; C07D 417/14; C07D 403/06; C07D 491/107; C07D 413/14; C07D 487/10; C07D 513/14

(52) U.S. Cl. CPC C07D 471/10; C07D 403/14; C07D 401/14; C07D 417/14; C07D 403/06; C07D 491/107; C07D 413/14; C07D 487/10; C07D 513/14

(57) ABSTRACT

Described herein are compounds of Formula (I) or a pharmaceutically acceptable salt thereof. The compounds of Formula (I) act as RIPK1 inhibitors and can be useful in preventing, treating or acting as a remedial agent for RIPK1-related diseases.

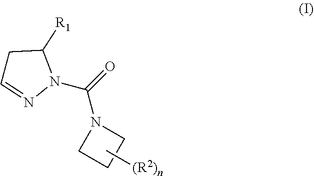

### FIELD OF THE INVENTION

The present invention is directed to RIPK1 inhibitors. Specifically, the RIPK1 inhibitors described herein can be useful in preventing, treating or acting as a remedial agent for RIPK1-related diseases.

### BACKGROUND OF THE INVENTION

Receptor-interacting protein-1 kinase (RIPK1) belongs to the family serine/threonine protein kinase involved in innate immune signaling. RIPK1 has emerged as a promising therapeutic target for the treatment of a wide range of human neurodegenerative, autoimmune, and inflammatory diseases. This is supported by extensive studies which have demonstrated that RIPK1 is a key mediator of apoptotic and necrotic cell death as well as inflammatory pathways.

For example, RIPK1 inhibition has been found to be useful as a treatment of acute kidney injury (AKI), a destructive clinical condition induced by multiple insults including ischemic reperfusion, nephrotoxic drugs and sepsis. It has been found that RIPK1-mediated necroptosis plays an important role in AKI and a RIPK1 inhibitor may serve as a promising clinical candidate for AKI treatment. Wang J N, Liu M M, Wang F, Wei B, Yang Q, Cai Y T, Chen X, Liu X Q, Jiang L, Li C, Hu X W, Yu J T, Ma T T, Jin J, Wu Y G, Li J, Meng X M, RIPK1 Inhibitor Cpd-71 Attenuates Renal Dysfunction in Cisplatin-Treated Mice via Attenuating Necroptosis, Inflammation and Oxidative Stress. Clin Sci (Lond). 2019 Jul. 25; 133(14):1609-1627.

Additionally, human genetic evidence has linked the dysregulation of RIPK1 to the pathogenesis of amyotrophic lateral sclerosis (ALS), Alzheimer's disease and multiple sclerosis as well as other inflammatory and neurodegenerative diseases. Alexei Degterev, Dimitry Ofengeim, and Junying Yuan, Targeting RIPK1 for the treatment of human diseases, PNAS, May 14, 2019, 116 (20), 9714-9722; Ito Y, Ofengeim D, Najafov A, Das S, Saberi S, Li Y, et al., RIPK1 mediates axonal degeneration by promoting inflammation and necroptosis in ALS, Science, 2016, 353:603-8; Caccamo A, Branca C, Piras I S, Ferreira E, Huentelman M J, Liang W S, et al., Necroptosis activation in Alzheimer's disease, Nat Neurosci, 2017, 20:1236-46; Ofengeim D, Ito Y, Najafov A, Zhang Y, Shan B, DeWitt J P, et al., Activation of necroptosis in multiple sclerosis, Cell Rep., 2015, 10:1836-49.

It also has been demonstrated that necroptosis is a delayed component of ischemic neuronal injury, thus RIPK1 inhibition may also play a promising role as a treatment for stroke. Degterev A, et al., Chemical inhibitor of nonapoptotic cell death with therapeutic potential for ischemic brain injury, Nat Chem Biol 2005, 1(2):112-119.

Therefore, there is a need for inhibitors of RIPK1 that offer high selectivity which can penetrate the blood-brain barrier, thus offering the possibility to target neuroinflammation and cell death which drive various neurologic conditions including Alzheimer's disease, ALS, and multiple sclerosis as well as acute neurological diseases such as stroke and traumatic brain injuries.

### BRIEF SUMMARY OF THE INVENTION

Described herein are compounds of Formula I:

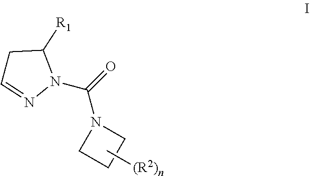

and pharmaceutically acceptable salts thereof, wherein R¹ and R² are described below.

The compounds described herein are RIPK1 inhibitors, which can be useful in the prevention, treatment or amelioration of neurodegenerative, autoimmune, inflammatory diseases and other RIPK1-related diseases.

Also described herein are methods of treating neurodegenerative, autoimmune, and inflammatory diseases comprising administering to a patient in need thereof a compound described herein, or a pharmaceutically acceptable salt thereof.

Also described herein are uses of a compound described herein, or a pharmaceutically acceptable salt thereof, to treat neurodegenerative, autoimmune, and inflammatory diseases in a patient in need thereof.

Also described herein are pharmaceutical compositions comprising a compound described herein, or a pharmaceutically acceptable salt thereof and a pharmaceutically acceptable carrier.

Also described herein are pharmaceutical compositions comprising a compound described herein and a pharmaceutically acceptable carrier.

Also described herein are methods of treating neurodegenerative, autoimmune, and inflammatory diseases comprising administering to a patient in need thereof a compound described herein, or a pharmaceutically acceptable salt thereof, and at least one additional therapeutic agent.

Also described herein are uses of a compound described herein, or a pharmaceutically acceptable salt thereof, in combination with at least one additional agent, to treat neurodegenerative, autoimmune, and inflammatory diseases in a patient in need thereof.

Also described herein are pharmaceutical compositions comprising a compound described herein, or a pharmaceutically acceptable salt thereof, at least one additional therapeutic agent and a pharmaceutically acceptable carrier.

Also described herein are pharmaceutical compositions comprising a compound described herein, at least one additional therapeutic agent and a pharmaceutically acceptable carrier.

### DETAILED DESCRIPTION OF THE INVENTION

Described herein are compounds of Formula I:

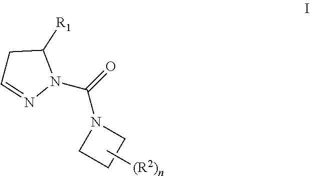

or a pharmaceutically acceptable salt thereof, wherein:

R¹ is aryl, C₃-C₁₀cycloalkyl or heteroaryl, wherein the aryl, C₃-C₁₀cycloalkyl or 20 heteroaryl is unsubstituted or substituted with one to three substituents selected from the group consisting of halogen, C₁-C₆alkyl, CN, OH, alkoxy, —N(R³)₂, —SC₁-C₆alkyl and C₃-C₆cycloalkyl;

Each occurrence of R² is selected from the group consisting of hydrogen, OH, C₁-C₆alkylOH, CN, C₁-C₆alkylCN, C₁-C₆alkyl, haloC₁-C₆alkyl, halogen, alkoxy, C₁-C₆alkylOC₁-C₆alkyl, aryl, heteroaryl, cycloheteroalkyl, C₃-C₁₀cycloalkyl, —O-aryl, —O-heteroaryl, —O— cycloheteroalkyl, —OC₃-C₁₀cycloalkyl, C₁-C₆alkylaryl, C₁-C₆alkylheteroaryl, C₁-C₆alkyl-cycloheteroalkyl, C₁-C₆alkylC₃-C₁₀cycloalkyl, haloC₁-C₆alkylaryl, haloC₁-C₆alkylheteroaryl, haloC₁-C₆alkyl-cycloheteroalkyl, haloC₁-C₆alkylC₃-C₁₀cycloalkyl, —CO-aryl, —OC₁-C₆alkylaryl, —OC₁-C₆alkylheteroaryl, —OC₁-C₆alkyl-cycloheteroalkyl, —OC₁-C₆alkylC₃-C₆cycloalkyl, —SO₂C₁-C₆alkyl, —SO₂aryl, —S-aryl, —SC₁-C₆alkyl, —N(R³)₂, and C₁-C₆alkylN(R³)₂, wherein any aryl, heteroaryl, cycloalkyl or cycloheteroalkyl is unsubstituted or substituted with one to three substituents selected from the group consisting of OH, haloC₁-C₆alkyl, aryl, heteroaryl, N(R³)₂, SO₂C₁-C₆alkyl, alkoxy, CN, halogen, C₁-C₆alkyl, —SC₁-C₆alkyl, C₁-C₆alkyl-cycloheteroalkyl, C₁-C₆alkylheteroaryl and C₁-C₆alkylOH;

R³ is hydrogen, C₁-C₆alkyl, aryl or heteroaryl, wherein the aryl or heteroaryl is unsubstituted or substituted with 1-3 substituents selected from the group consisting of CN, C₁-C₆alkyl, haloC₁-C₆alkyl and alkoxy; and

n is 1, 2, 3, 4, 5 or 6, wherein, when n is 2, 3, 4, 5 or 6, two R² substituents can be taken together to form a cycloheteroalkyl or C₃-C₁₀cycloalkyl, wherein the cycloheteroalkyl or C₃-C₁₀cycloalkyl is unsubstituted or substituted with one to four substituents selected from the group consisting of halogen, C₁-C₆alkyl, aryl, alkoxy, —O-aryl, —O-heteroaryl, N(R³)₂, CN, —SO₂C₁-C₆alkyl, C₁-C₆alkylOH, heteroaryl, C(O)OC₁-C₆alkyl, and C₁-C₆alkyl-cycloheteroalkyl.

Also described herein are compounds of Formula I:

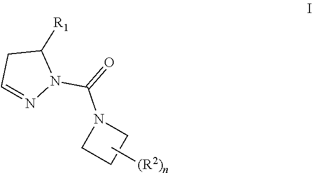

or a pharmaceutically acceptable salt thereof, wherein:

R¹ is aryl, C₃-C₁₀cycloalkyl or heteroaryl, wherein the aryl, C₃-C₁₀cycloalkyl or heteroaryl is unsubstituted or substituted with one to three substituents selected from the group consisting of halogen, C₁-C₆alkyl, CN, OH, alkoxy, —N(R³)₂, —SC₁-C₆alkyl and C₃-C₆cycloalkyl;

Each occurrence of R² is selected from the group consisting of hydrogen, OH, C₁-C₆alkylOH, CN, C₁-C₆alkylCN, C₁-C₆alkyl, haloC₁-C₆alkyl, halogen, alkoxy, C₁-C₆alkylOC₁-C₆alkyl, aryl, heteroaryl, cycloheteroalkyl, C₃-C₁₀cycloalkyl, —O-aryl, —O-heteroaryl, —O— cycloheteroalkyl, —OC₃-C₁₀cycloalkyl, C₁-C₆alkylaryl, C₁-C₆alkylheteroaryl, C₁-C₆alkyl-cycloheteroalkyl, C₁-C₆alkylC₃-C₁₀cycloalkyl, —CO-aryl, —OC₁-C₆alkylaryl, —OC₁-C₆alkylheteroaryl, —OC₁-C₆alkyl-cycloheteroalkyl, —OC₁-C₆alkylC₃-C₆cycloalkyl, —SO₂C₁-C₆alkyl, —SO₂aryl, —S-aryl, —SC₁-C₆alkyl, —N(R³)₂, and C₁-C₆alkylN(R³)₂, wherein any aryl, heteroaryl, cycloalkyl or cycloheteroalkyl is unsubstituted or substituted with one to three substituents selected from the group consisting of OH, haloC₁-C₆alkyl, aryl, heteroaryl, N(R³)₂, SO₂C₁-C₆alkyl, alkoxy, CN, halogen, C₁-C₆alkyl, —SC₁-C₆alkyl, C₁-C₆alkyl-cycloheteroalkyl, C₁-C₆alkylheteroaryl and C₁-C₆alkylOH;

R³ is hydrogen, C₁-C₆alkyl, aryl or heteroaryl, wherein the aryl or heteroaryl is unsubstituted or substituted with 1-3 substituents selected from the group consisting of CN, C₁-C₆alkyl, haloC₁-C₆alkyl and alkoxy; and

n is 1, 2, 3, 4, 5 or 6, wherein, when n is 2, 3, 4, 5 or 6, two R² substituents can be taken together to form a cycloheteroalkyl or C₃-C₁₀cycloalkyl, wherein the cycloheteroalkyl or C₃-C₁₀cycloalkyl is unsubstituted or substituted with one to four substituents selected from the group consisting of halogen, C₁-C₆alkyl, aryl, alkoxy, —O-aryl, —O-heteroaryl, N(R³)₂, CN, —SO₂C₁-C₆alkyl, C₁-C₆alkylOH, heteroaryl, C(O)OC₁-C₆alkyl, and C₁-C₆alkyl-cycloheteroalkyl.

With regard to the compounds described herein, R¹ is aryl, C₃-C₁₀cycloalkyl or heteroaryl, wherein the aryl, C₃-C₁₀cycloalkyl or heteroaryl is unsubstituted or substituted with one to three substituents selected from the group consisting of halogen, C₁-C₆alkyl, CN, OH, alkoxy, —N(R³)₂, —SC₁-C₆alkyl and C₃-C₆cycloalkyl.

In certain embodiments, R¹ is aryl. In certain embodiments, R¹ is a monocyclic aryl. In other embodiments, R¹ is a bicyclic aryl. In other embodiments, R¹ is a multicyclic aryl. Suitable aryls include, but are not limited to, phenyl and naphthyl. In certain embodiments, R¹ is aryl, wherein the aryl is phenyl. In certain embodiments, R¹ is aryl, wherein the aryl is naphthyl. In certain embodiments, the aryl is

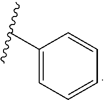 [SMILES: *c1ccccc1]

In certain embodiments, R¹ is C₃-C₁₀cycloalkyl. In certain embodiments, R¹ is a monocyclic cycloalkyl. In other embodiments, R¹ is a bicyclic cycloalkyl. In other embodiments, R¹ is a multicyclic cycloalkyl. Suitable cycloalkyls include, but are not limited to, cyclopropyl, cyclobutyl, cyclopentyl, cyclohexyl, cycloheptyl, tetrahydronaphthyl, decahydronaphthyl, indanyl. In certain embodiments, R¹ is C₃-C₁₀cycloalkyl, wherein the C₃-C₁₀cycloalkyl is:

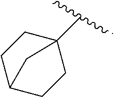 [SMILES: *C12CCC(CC1)C2]

In certain embodiments, R¹ is heteroaryl. In certain embodiments, R′ is a nitrogen-containing heteroaryl. In certain embodiments, R¹ is a monocyclic heteroaryl. In other embodiments, R¹ is a bicyclic heteroaryl. In other embodiments, R¹ is a multicyclic heteroaryl. Suitable heteroaryls include, but are not limited to, pyridyl (pyridinyl), oxazolyl, imidazolyl, triazolyl, furyl, triazinyl, thienyl, pyrimidyl, pyridazinyl, indolizinyl, cinnolinyl, phthalazinyl, quinazolinyl, naphthyridinyl, quinoxalinyl, purinyl, benzimidazolyl, quinolyl, and isoquinolyl. In certain embodiments, R¹ is pyridyl. In certain embodiments, R¹ is heteroaryl, wherein the heteroaryl is:

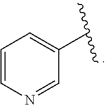 [SMILES: *c1cccnc1]

In certain embodiments, R¹ is unsubstituted.

In certain embodiments, R¹ is substituted with one to three substituents selected from the group consisting of halogen, C₁-C₆alkyl, CN, OH, alkoxy, —N(R³)₂, —SC₁-C₆alkyl and C₃-C₆cycloalkyl. In certain embodiments, R¹ is substituted with one substituent selected from the group consisting of halogen, C₁-C₆alkyl, CN, OH, alkoxy, —N(R³)₂, —SC₁-C₆alkyl and C₃-C₆cycloalkyl. In certain embodiments, R¹ is substituted with two substituents selected from the group consisting of halogen, C₁-C₆alkyl, CN, OH, alkoxy, —N(R³)₂, —SC₁-C₆alkyl and C₃-C₆cycloalkyl. In certain embodiments, R¹ is substituted with three substituents selected from the 15 group consisting of halogen, C₁-C₆alkyl, CN, OH, alkoxy, —N(R³)₂, —SC₁-C₆alkyl and C₃-C₆cycloalkyl.

In certain embodiments, R¹ is substituted with halogen. Suitable halogens include, but are not limited to, fluorine, chlorine, bromine or iodine. In certain embodiments, R¹ is substituted with fluorine or chlorine. In certain embodiments, R¹ is substituted with two fluorines. In certain embodiments, R¹ is substituted with chlorine. In certain embodiments, R¹ is phenyl substituted with fluorine or chlorine.

In certain embodiments, R¹ is substituted with C₁-C₆alkyl. Suitable alkyls include, but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl and 1-ethyl-1-methylpropyl. In certain embodiments, R¹ is substituted with methyl or ethyl.

In certain embodiments, R¹ is substituted with CN.

In certain embodiments, R¹ is substituted with OH.

In certain embodiments, R¹ is substituted with alkoxy. Suitable alkoxys include, but are not limited to, methoxy, ethoxy, n-propoxy, isopropoxy and n-butoxy. In certain embodiments, R¹ is substituted with methoxy.

In certain embodiments, R¹ is substituted with —N(R³)₂. R³ is discussed in further detail below.

In certain embodiments, R¹ is substituted with —SC₁-C₆alkyl. Suitable —SC₁-C₆alkyl substituents include, but are not limited to, —SCH₂CH₃, and —SCH₃.

In certain embodiments, R¹ is substituted with C₃-C₆cycloalkyl. In certain embodiments, R¹ is substituted with a monocyclic cycloalkyl. In other embodiments, R¹ is substituted with a bicyclic cycloalkyl. In other embodiments, R¹ is substituted with a multicyclic cycloalkyl. Suitable cycloalkyls include, but are not limited to, cyclopropyl, cyclobutyl, cyclopentyl, cyclohexyl, cycloheptyl, tetrahydronaphthyl, decahydronaphthyl, indanyl. In certain embodiments, R¹ is substituted with

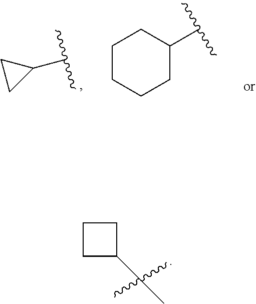

In certain embodiments, R¹ is

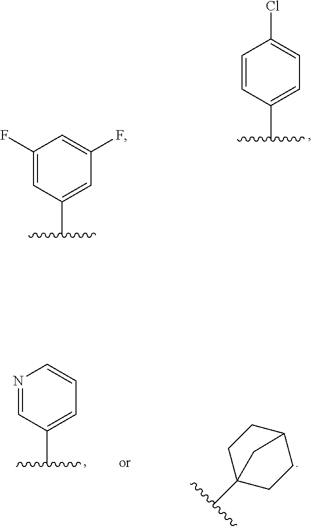 [SMILES: *c1cc(F)cc(F)c1 ; *c1ccc(Cl)cc1 ; *c1cccnc1 ; *C12CCC(CC1)C2]

In certain embodiments, R¹ is

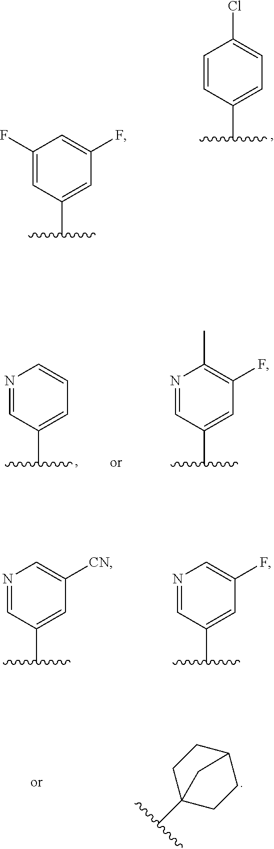

With regard to the compounds described herein, R³ is hydrogen, C₁-C₆alkyl, aryl or heteroaryl, wherein the aryl or heteroaryl is unsubstituted or substituted with 1-3 substituents selected from the group consisting of CN, C₁-C₆alkyl, haloC₁-C₆alkyl and alkoxy. In certain embodiments, R³ is hydrogen. In certain embodiments, R³ is C₁-C₆alkyl. Suitable alkyls include, but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl and 1-ethyl-1-methylpropyl. In certain embodiments, R³ is substituted with methyl or ethyl.

In certain embodiments, R³ is aryl. In certain embodiments, R³ is a monocyclic cycloalkyl. In other embodiments, R³ is a bicyclic cycloalkyl. In other embodiments, R³ is a multicyclic cycloalkyl. Suitable cycloalkyls include, but are not limited to, cyclopropyl, cyclobutyl, cyclopentyl, cyclohexyl, cycloheptyl, tetrahydronaphthyl, decahydronaphthyl, indanyl. In certain embodiments, R³ is or

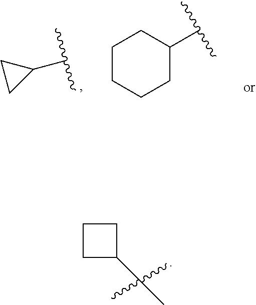

In certain embodiments, R³ is heteroaryl. In certain embodiments, R³ is a nitrogen-containing heteroaryl. In certain embodiments, R³ is a monocyclic heteroaryl. In other embodiments, R³ is a bicyclic heteroaryl. In other embodiments, R³ is a multicyclic heteroaryl. Suitable heteroaryls include, but are not limited to, pyridyl (pyridinyl), oxazolyl, imidazolyl, triazolyl, furyl, triazinyl, thienyl, pyrimidyl, pyridazinyl, indolizinyl, cinnolinyl, phthalazinyl, quinazolinyl, naphthyridinyl, quinoxalinyl, purinyl, benzimidazolyl, quinolyl, and isoquinolyl.

In certain embodiments, R³ is heteroaryl, wherein the heteroaryl is:

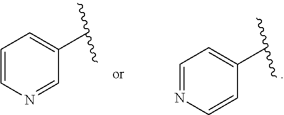 [SMILES: *c1cccnc1 ; *c1ccncc1]

In certain embodiments, wherein R³ is aryl or heteroaryl, the aryl or heteroaryl is unsubstituted or substituted with one, two or three substituents selected from the group consisting of CN, C₁-C₆alkyl, haloC₁-C₆alkyl and alkoxy. In certain embodiments, R³ is aryl, wherein the aryl is unsubstituted. In certain embodiments, R³ is aryl, wherein the aryl is substituted with CN. In certain embodiments, R³ is aryl, wherein the aryl is substituted with C₁-C₆alkyl. In certain embodiments, R³ is aryl, wherein the aryl is substituted with haloC₁-C₆alkyl. In certain embodiments, R³ is aryl, wherein the aryl is substituted with alkoxy. In certain embodiments, R³ is heteroaryl, wherein the heteroaryl is substituted with CN. In certain embodiments, R³ is heteroaryl, wherein the heteroaryl is unsubstituted. In certain embodiments, R³ is heteroaryl, wherein the heteroaryl is substituted with C₁-C₆alkyl. In certain embodiments, R³ is heteroaryl, wherein the heteroaryl is substituted with haloC₁-C₆alkyl. In certain embodiments, R³ is heteroaryl, wherein the heteroaryl is substituted with alkoxy.

In certain embodiments, R³ is hydrogen, methyl or phenyl.

In certain embodiments, —N(R³)₂ is

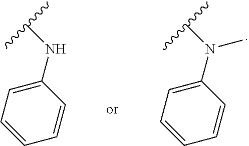

With regard to the compounds described herein, n is 1, 2, 3, 4, 5 or 6. In certain embodiments, n is 1. In certain embodiments, n is 2. In certain embodiments, n is 3. In certain embodiments, n is 4. In certain embodiments, n is 5. In certain embodiments, n is 6. In certain embodiments, n is 1, 2 or 3. In certain embodiments, n is 2 or 3.

With regard to the compounds described herein, each occurrence of R² is selected from the group consisting of hydrogen, OH, C₁-C₆alkylOH, CN, C₁-C₆alkylCN, C₁-C₆alkyl, haloC₁-C₆alkyl, halogen, alkoxy, C₁-C₆alkylOC₁-C₆alkyl, aryl, heteroaryl, cycloheteroalkyl, C₃-C₁₀cycloalkyl, —O-aryl, —O-heteroaryl, —O-cycloheteroalkyl, —OC₃-C₁₀cycloalkyl, C₁-C₆alkylaryl, C₁-C₆alkylheteroaryl, C₁-C₆alkyl-cycloheteroalkyl, C₁-C₆alkylC₃-C₁₀cycloalkyl, haloC₁-C₆alkylaryl, haloC₁-C₆alkylheteroaryl, haloC₁-C₆alkyl-cycloheteroalkyl, haloC₁-C₆alkylC₃-C₁₀cycloalkyl, —CO-aryl, —OC₁-C₆alkylaryl, —OC₁-C₆alkylheteroaryl, —OC₁-C₆alkyl-cycloheteroalkyl, —OC₁-C₆alkylC₃-C₆cycloalkyl, —SO₂C₁-C₆alkyl, —SO₂aryl, —S-aryl, —S—C₁-C₆alkyl, —N(R³)₂, and C₁-C₆alkylN(R³)₂, wherein any aryl, heteroaryl, cycloalkyl or cycloheteroalkyl is unsubstituted or substituted with one to three substituents selected from the group consisting of OH, haloC₁-C₆alkyl, aryl, heteroaryl, N(R³)₂, SO₂C₁-C₆alkyl, alkoxy, CN, halogen, C₁-C₆alkyl, —SC₁-C₆alkyl, C₁-C₆alkyl-cycloheteroalkyl, C₁-C₆alkylheteroaryl and C₁-C₆alkylOH.

In certain embodiments described herein, each occurrence of R² is selected from the group consisting of hydrogen, OH, C₁-C₆alkylOH, CN, C₁-C₆alkylCN, C₁-C₆alkyl, haloC₁-C₆alkyl, halogen, alkoxy, C₁-C₆alkylOC₁-C₆alkyl, aryl, heteroaryl, cycloheteroalkyl, C₃-C₁₀cycloalkyl, —O-aryl, —O-heteroaryl, —O-cycloheteroalkyl, —OC₃-C₁₀cycloalkyl, C₁-C₆alkylaryl, C₁-C₆alkylheteroaryl, C₁-C₆alkyl-cycloheteroalkyl, C₁-C₆alkylC₃-C₁₀cycloalkyl, —CO-aryl, —OC₁-C₆alkylaryl, —OC₁-C₆alkylheteroaryl, —OC₁-C₆alkyl-cycloheteroalkyl, —OC₁-C₆alkylC₃-C₆cycloalkyl, —SO₂C₁-C₆alkyl, —SO₂aryl, —S-aryl, —S—C₁-C₆alkyl, —N(R³)₂, and C₁-C₆alkylN(R³)₂, wherein any aryl, heteroaryl, cycloalkyl or cycloheteroalkyl is unsubstituted or substituted with one to three substituents selected from the group consisting of OH, haloC₁-C₆alkyl, aryl, heteroaryl, N(R³)₂, SO₂C₁-C₆alkyl, alkoxy, CN, halogen, C₁-C₆alkyl, —SC₁-C₆alkyl, C₁-C₆alkyl-cycloheteroalkyl, C₁-C₆alkylheteroaryl and C₁-C₆alkylOH.

In certain embodiments, one or more R² substituents are hydrogen.

In certain embodiments, one or more R² substituents are OH.

In certain embodiments, one or more R² substituents are C₁-C₆alkylOH. Suitable alcohols include, but are not limited to, methanol, ethanol, propanol and butanol. In certain embodiments, R² is

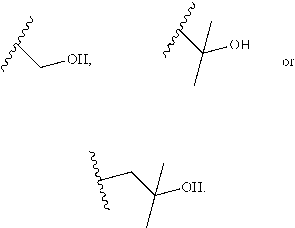

In certain embodiments, R² is

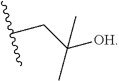 [SMILES: *CC(C)(C)O]

In certain embodiments, one or more R² substituents are CN.

In certain embodiments, one or more R² substituents are C₁-C₆alkyl-CN. In certain embodiments, R² is

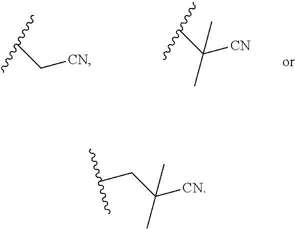

In certain embodiments, one or more R² substituents are C₁-C₆alkyl. Suitable alkyls include, but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl and 1-ethyl-1-methylpropyl. In certain embodiments, R² is methyl, ethyl or

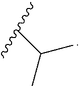 [SMILES: *C(C)C]

In certain embodiments, one or more R² substituents are halo-C₁-C₆alkyl. Suitable examples of haloalkyls include, but are not limited to, fluoromethyl, difluoromethyl, trifluoromethyl, 2-fluoroethyl, 1,2-difluoroethyl and 2,2-difluoroethyl. In certain embodiments, R² is difluoromethyl. In certain embodiments, R² is trifluoromethyl. In certain embodiments, R² is difluoromethyl or trifluoromethyl.

In certain embodiments, one or more R² substituents are halogen. Suitable halogens include, but are not limited to, fluorine, chlorine, bromine or iodine. In certain embodiments, R² is fluorine or chlorine.

In certain embodiments, one or more R² substituents are alkoxy. Suitable alkoxys include, but are not limited to, methoxy, ethoxy, n-propoxy, isopropoxy and n-butoxy. In certain embodiments, R² is methoxy, ethoxy or

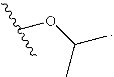 [SMILES: *OC(C)C]

In certain embodiments, one or more R² substituents are C₁-C₆alkylOC₁-C₆alkyl. In certain embodiments, R² is CH₂OCH₃ or CH₂CH₂OCH₃. In certain embodiments, R² is CH₂CH₂OCH₃.

In certain embodiments, one or more R² substituents are aryl. Suitable aryls include, but are not limited to, phenyl and naphthyl. In certain embodiments, R² is phenyl.

In certain embodiments, one or more R² substituents are heteroaryl. In certain embodiments, R² is a nitrogen-containing heteroaryl. In certain embodiments, R² is a monocyclic heteroaryl. In other embodiments, R² is a bicyclic heteroaryl. In other embodiments, R² is a multicyclic heteroaryl. Suitable heteroaryls include, but are not limited to, pyridyl (pyridinyl), oxazolyl, imidazolyl, triazolyl, furyl, triazinyl, thienyl, pyrimidyl, pyridazinyl, indolizinyl, cinnolinyl, phthalazinyl, quinazolinyl, naphthyridinyl, quinoxalinyl, purinyl, benzimidazolyl, quinolyl, and isoquinolyl. In certain embodiments, R² is pyridyl. In certain embodiments, R² is

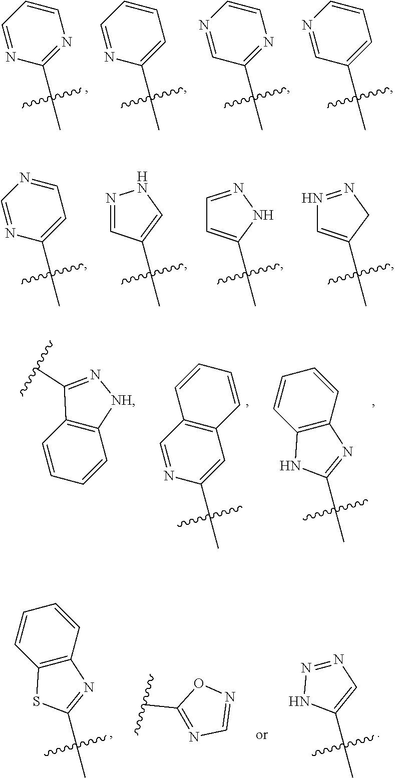

In certain embodiments, R² is

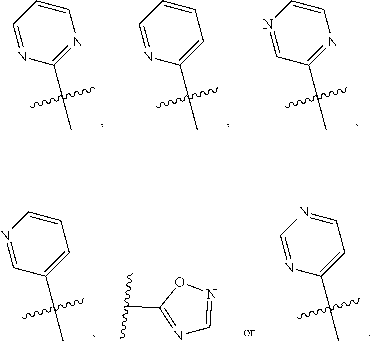 [SMILES: *c1ncccn1 ; *c1ccccn1 ; *c1cnccn1 ; *c1cccnc1 ; *c1ncno1 ; *c1ccncn1]

In certain embodiments, one or more R² substituents are cycloheteroalkyl. In certain embodiments, R² is

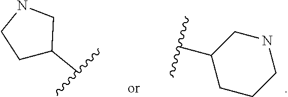 [SMILES: *C1CCNC1 ; *C1CCCNC1]

In certain embodiments, one or more R² substituents are C₃-C₁₀cycloalkyl. In certain embodiments, R² is a monocyclic cycloalkyl. In other embodiments, R² is a bicyclic cycloalkyl. In other embodiments, R² is a multicyclic cycloalkyl. Suitable cycloalkyls include, but are not limited to, cyclopropyl, cyclobutyl, cyclopentyl, cyclohexyl, cycloheptyl, tetrahydronaphthyl, decahydronaphthyl, indanyl. In certain embodiments, R² is

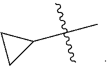 [SMILES: *C1CC1]

In certain embodiments, one or more R² substituents are —O-aryl. In certain embodiments, R² is

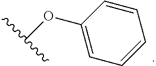 [SMILES: *Oc1ccccc1]

In certain embodiments, one or more R² substituents are —O-heteroaryl. In certain embodiments, R² is

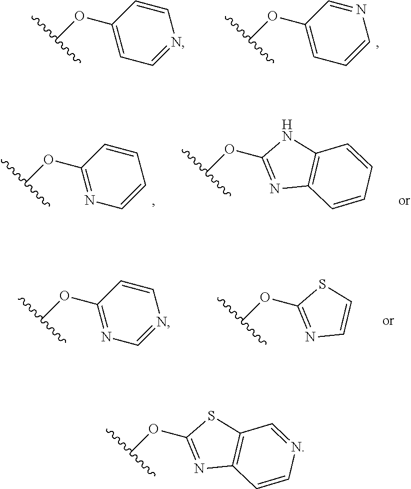

In certain embodiments, one or more R² substituents are —O-cycloheteroalkyl. In certain embodiments, R² is

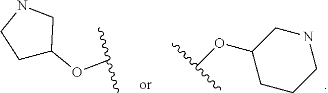 [SMILES: *OC1CCNC1 ; *OC1CCCNC1]

In certain embodiments, one or more R² substituents are —OC₃-C₁₀cycloalkyl. In certain embodiments, R² is

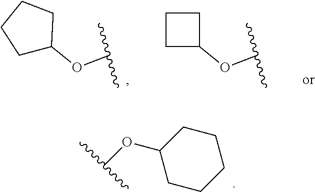 [SMILES: *OC1CCCC1 ; *OC1CCC1 ; *OC1CCCCC1]

In certain embodiments, one or more R² substituents are C₁-C₆alkyl-aryl. In certain embodiments, R² is

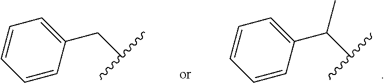

In certain embodiments, one or more R² substituents are C₁-C₆alkyl-heteroaryl. In certain embodiments, R² is

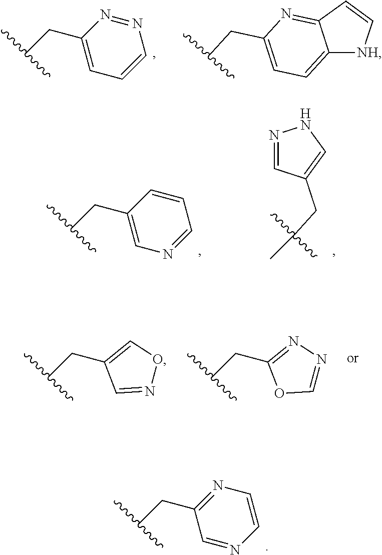

In certain embodiments, one or more R² substituents are C₁-C₆alkyl-cycloheteroalkyl. In certain embodiments, R² is

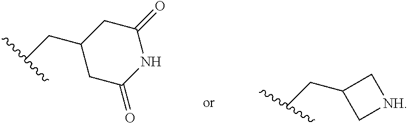 [SMILES: *CC1CC(=O)NC(=O)C1 ; *CC1CNC1]

In certain embodiments, one or more R² substituents are haloC₁-C₆alkylC₃-C₁₀cycloalkyl. In certain embodiments, R² is

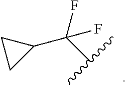 [SMILES: *C(F)(F)C1CC1]

In certain embodiments, one or more R² substituents are haloC₁-C₆alkylaryl. In certain embodiments, R² is

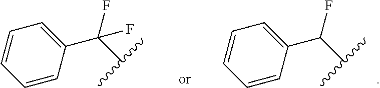 [SMILES: *C(F)(F)c1ccccc1 ; *C(F)c1ccccc1]

In certain embodiments, one or more R² substituents are haloC₁-C₆alkyl-heteroaryl. In certain embodiments, R² is

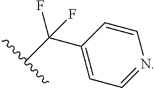 [SMILES: *C(F)(F)c1ccncc1]

In certain embodiments, one or more R² substituents are haloC₁-C₆alkyl-cycloheteroalkyl. In certain embodiments, R² is

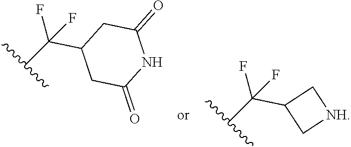 [SMILES: *C(F)(F)C1CC(=O)NC(=O)C1 ; *C(F)(F)C1CNC1]

In certain embodiments, one or more R² substituents are haloC₁-C₆alkyl-C₃-C₁₀cycloalkyl. In certain embodiments, R² is

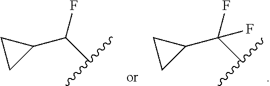 [SMILES: *C(F)C1CC1 ; *C(F)(F)C1CC1]

In certain embodiments, one or more R² substituents are —CO-aryl. In certain embodiments, R² is

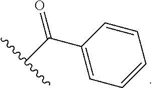 [SMILES: *C(=O)c1ccccc1]

In certain embodiments, one or more R² substituents are —OC₁-C₆alkylaryl. In certain embodiments, R² is

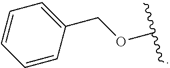 [SMILES: *OCc1ccccc1]

In certain embodiments, one or more R² substituents are —OC₁-C₆alkylheteroaryl. In certain embodiments, R² is

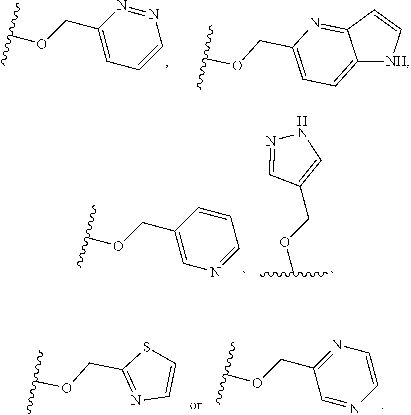

In certain embodiments, one or more R² substituents are —OC₁-C₆alkyl-cycloheteroalkyl. In certain embodiments, R² is

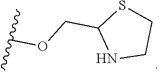 [SMILES: *OCC1NCCS1]

In certain embodiments, one or more R² substituents are —OC₁-C₆alkylC₃-C₆cycloalkyl. In certain embodiments, R² is

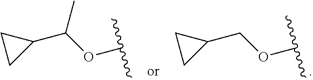

In certain embodiments, one or more R² substituents are —SO₂C₁-C₆alkyl. In certain embodiments, R² substituents are —SO₂CH₃, —SO₂CH₂CH₃, or —SO₂CH₂CH₃.

In certain embodiments, one or more R² substituents are —SO₂aryl. In certain embodiments, R² is

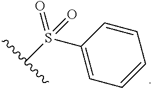 [SMILES: *S(=O)(=O)c1ccccc1]

In certain embodiments, one or more R² substituents are —S-aryl. In certain embodiments, R² is

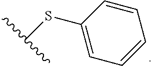 [SMILES: *Sc1ccccc1]

In certain embodiments, one or more R² substituents are —S—C₁-C₆alkyl. In certain embodiments, R² is —SCH₃, —SCH₂CH₃, or —SCH₂CH₃.

In certain embodiments, one or more R² substituents are —N(R³)₂. In certain embodiments, —N(R³)₂ is

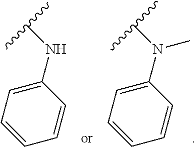

In certain embodiments, one or more R² substituents are C₁-C₆alkylN(R³)₂. In certain embodiments, C₁-C₆alkylN(R³)₂ is

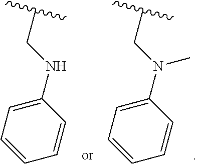

In certain embodiments R² is unsubstituted. In certain embodiments, wherein the R² substituent includes an aryl, heteroaryl, cycloalkyl or cycloheteroalkyl, the aryl, heteroaryl, cycloalkyl or cycloheteroalkyl is substituted with one, two or three substituents selected from the group consisting of OH, haloC₁-C₆alkyl, aryl, heteroaryl, N(R³)₂, SO₂C₁-C₆alkyl, alkoxy, CN, halogen, C₁-C₆alkyl, —SC₁-C₆alkyl, C₁-C₆alkyl-cycloheteroalkyl, C₁-C₆alkylheteroaryl and C₁-C₆alkylOH.

In certain embodiments, wherein R² is aryl, —O-aryl, C₁-C₆alkylaryl, —CO-aryl or —OC₁-C₆alkylaryl, the aryl, —O-aryl, C₁-C₆alkylaryl, —CO-aryl or —OC₁-C₆alkylaryl is substituted with one to three substituents selected from the group consisting of OH, haloC₁-C₆alkyl, aryl, heteroaryl, N(R³)₂, SO₂C₁-C₆alkyl, alkoxy, CN, halogen, C₁-C₆alkyl, —SC₁-C₆alkyl, C₁-C₆alkyl-cycloheteroalkyl, C₁-C₆alkylheteroaryl and C₁-C₆alkylOH.

In certain embodiments, wherein R² is heteroaryl, —O-heteroaryl, C₁-C₆alkyl-heteroaryl, or —OC₁-C₆alkyl-heteroaryl, the heteroaryl, —O-heteroaryl, C₁-C₆alkylheteroaryl or —OC₁-C₆alkylheteroaryl is substituted with one to three substituents selected from the group consisting of OH, haloC₁-C₆alkyl, aryl, heteroaryl, N(R³)₂, SO₂C₁-C₆alkyl, alkoxy, CN, halogen, C₁-C₆alkyl, —SC₁-C₆alkyl, C₁-C₆alkyl-cycloheteroalkyl, C₁-C₆alkylheteroaryl and C₁-C₆alkylOH.

In certain embodiments, wherein R² is cycloheteroalkyl, —O-cycloheteroalkyl, C₁-C₆alkyl-cycloheteroalkyl or —OC₁-C₆alkyl-cycloheteroalkyl, the cycloheteroalkyl, —O— cycloheteroalkyl, C₁-C₆alkyl-cycloheteroalkyl or —OC₁-C₆alkyl-cycloheteroalkyl is substituted with one to three substituents selected from the group consisting of OH, haloC₁-C₆alkyl, aryl, heteroaryl, N(R³)₂, SO₂C₁-C₆alkyl, alkoxy, CN, halogen, C₁-C₆alkyl, —SC₁-C₆alkyl, C₁-C₆alkyl-cycloheteroalkyl, C₁-C₆alkylheteroaryl and C₁-C₆alkylOH.

In certain embodiments, wherein R² is C₃-C₁₀cycloalkyl, —OC₃-C₁₀cycloalkyl, C₁-C₆alkylC₃-C₁₀cycloalkyl or —OC₁-C₆alkylC₃-C₆cycloalkyl, the C₃-C₁₀cycloalkyl, —OC₃-C₁₀cycloalkyl, C₁-C₆alkylC₃-C₁₀cycloalkyl or —OC₁-C₆alkylC₃-C₆cycloalkyl is substituted with one to three substituents selected from the group consisting of OH, haloC₁-C₆alkyl, aryl, heteroaryl, N(R³)₂, SO₂C₁-C₆alkyl, alkoxy, CN, halogen, C₁-C₆alkyl, —SC₁-C₆alkyl, C₁-C₆alkyl-cycloheteroalkyl, C₁-C₆alkylheteroaryl and C₁-C₆alkylOH.

In certain embodiments, wherein n is 2, 3, 4, 5 or 6 more, two R² substituents can be taken together to form a cycloheteroalkyl or C₃-C₁₀cycloalkyl, wherein the cycloheteroalkyl or C₃-C₁₀cycloalkyl is unsubstituted or substituted with one to four substituents selected from the group consisting of halogen, C₁-C₆alkyl, aryl, alkoxy, —O-aryl, —O-heteroaryl, N(R³)₂, CN, —SO₂C₁-C₆alkyl, C₁-C₆alkylOH, heteroaryl, —C(O)OC₁-C₆alkyl, and C₁-C₆alkyl-cycloheteroalkyl.

In certain embodiments, wherein n is two or more, two R² substituents can be taken together to form a cycloheteroalkyl. In certain embodiments, wherein n is two or more, two R² substituents can be taken together to form a cycloheteroalkyl or C₃-C₁₀cycloalkyl. In certain embodiments, the cycloheteroalkyl or C₃-C₁₀cycloalkyl is unsubstituted. In certain embodiments, the cycloheteroalkyl or C₃-C₁₀cycloalkyl is substituted with one, two, three or four substituents selected from the group consisting of halogen, C₁-C₆alkyl, aryl, alkoxy, —O-aryl, —O-heteroaryl, N(R³)₂, CN, —SO₂C₁-C₆alkyl, C₁-C₆alkylOH, heteroaryl, C(O)OC₁-C₆alkyl, and C₁-C₆alkyl-cycloheteroalkyl.

In certain embodiments, two R² substituents can be taken together to form:

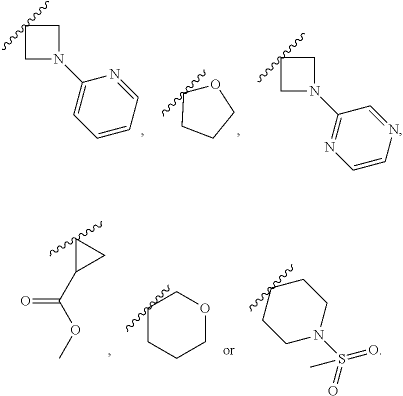

In certain embodiments, each occurrence of R² is independently selected from the group consisting of methyl, OH, fluorine, CN, methoxy, chlorine, ethoxy, difluoromethyl, trifluoromethyl, —SO₂CH₃, isopropyl, cyclopropyl,

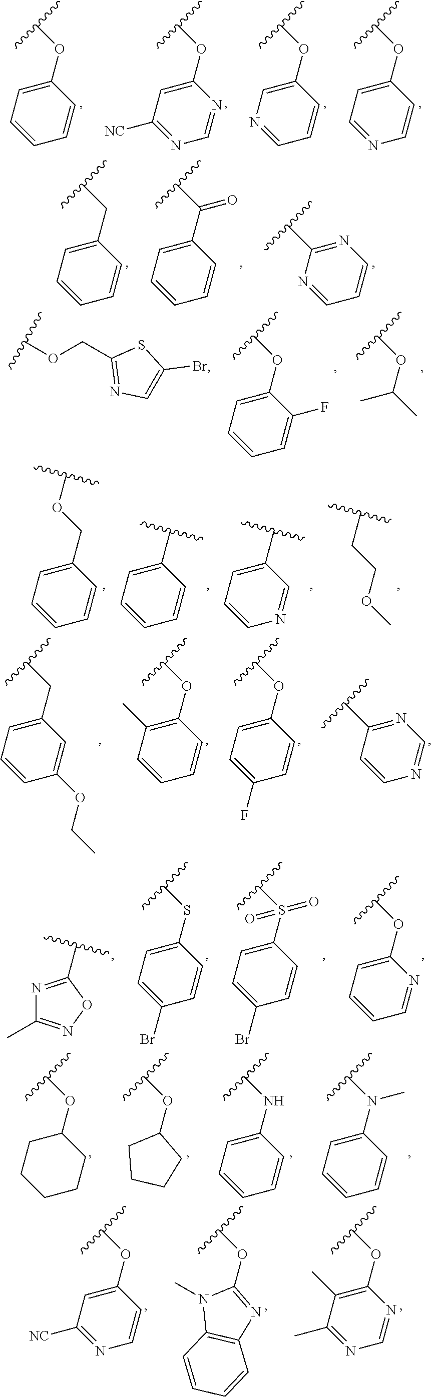

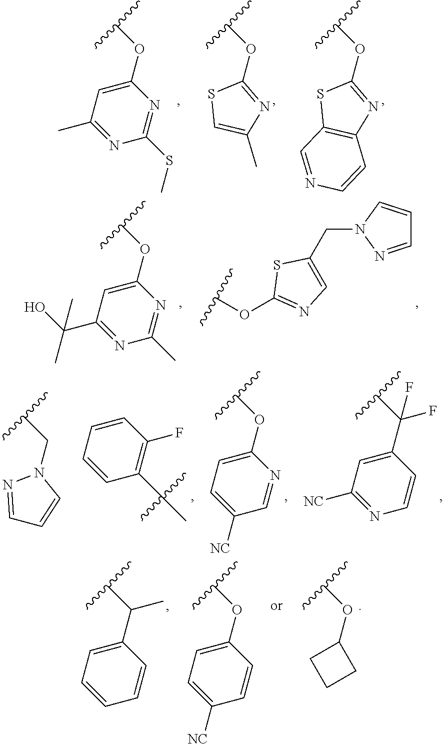

In certain embodiments, each occurrence of R² is independently selected from the group consisting of methyl, OH, fluorine, CN, methoxy, chlorine, CN, methoxy, ethoxy, difluoromethyl, trifluoromethyl, —SO₂CH₃, isopropyl, cyclopropyl,

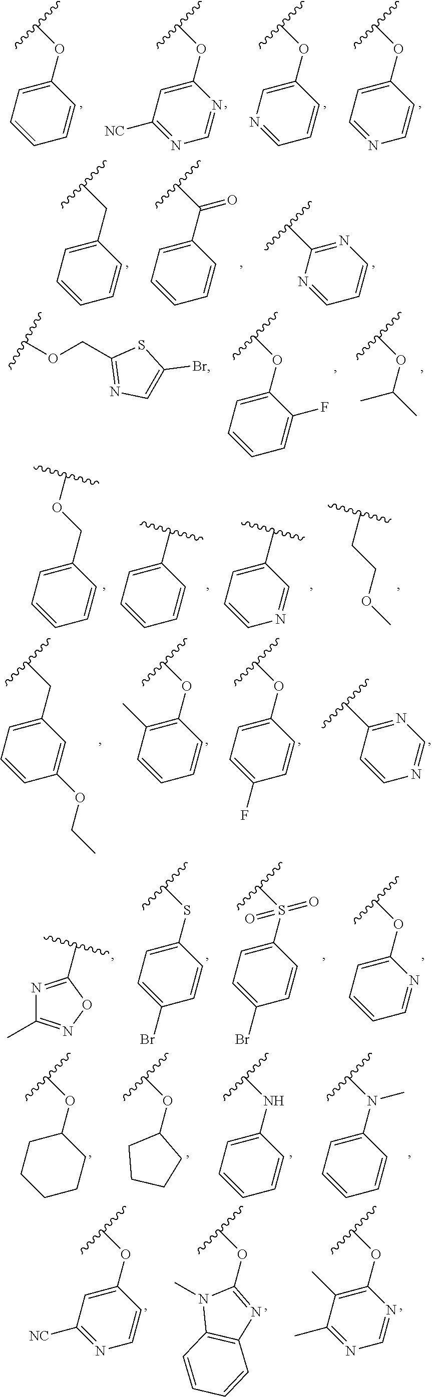

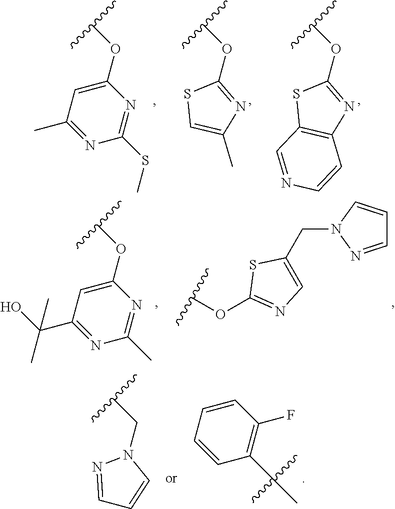

Also described herein are compounds of Formula Ia and Ib:

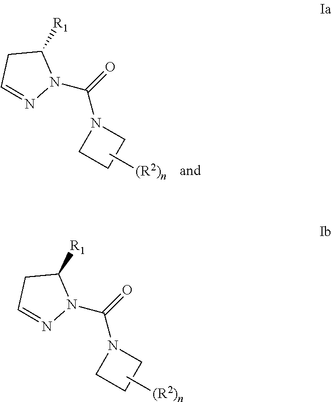

or a pharmaceutically acceptable salt thereof, wherein R¹, R² and n are described above.

Also described herein are compounds of Formula II:

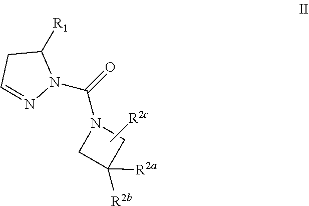

or a pharmaceutically acceptable salt thereof, wherein:

R¹ is aryl, C₃-C₁₀cycloalkyl or heteroaryl, wherein the aryl, C₃-C₁₀cycloalkyl or heteroaryl is unsubstituted or substituted with one to three substituents selected from the group consisting of halogen, C₁-C₆alkyl, CN, OH, alkoxy, —N(R³)₂, —SC₁-C₆alkyl and C₃-C₆cycloalkyl;

R^{2a} is hydrogen, halogen, CN, OH, C₁-C₆alkyl, alkoxy, C₃-C₆cycloalkyl, C₁-C₆alkylOH, C₁-C₆alkylOC₁-C₆alkyl, N(R³)₂ or haloC₁-C₆alkyl, or taken with R^{2b} forms a cycloheteroalkyl or C₃-C₁₀cycloalkyl, wherein the cycloheteroalkyl or C₃-C₁₀cycloalkyl is unsubstituted or substituted with one to four substituents selected from the group consisting of —SO₂C₁-C₆alkyl, C₁-C₆alkylOH, heteroaryl, C(O)OC₁-C₆alkyl, halogen, C₁-C₆alkyl, aryl, alkoxy, —O-aryl, —O— heteroaryl, N(R³)₂, CN and C₁-C₆alkyl-cycloheteroalkyl;

R^{2b} is hydrogen, OH, C₁-C₆alkylOH, CN, C₁-C₆alkylCN, C₁-C₆alkyl, haloC₁-C₆alkyl, halogen, alkoxy, C₁-C₆alkylOC₁-C₆alkyl, aryl, heteroaryl, cycloheteroalkyl, C₃-C₁₀cycloalkyl, —O-aryl, —O-heteroaryl, —O-cycloheteroalkyl, —OC₃-C₁₀cycloalkyl, C₁-C₆alkylaryl, C₁-C₆alkylheteroaryl, C₁-C₆alkyl-cycloheteroalkyl, C₁-C₆alkylC₃-C₁₀cycloalkyl, haloC₁-C₆alkylaryl, haloC₁-C₆alkylheteroaryl, haloC₁-C₆alkyl-cycloheteroalkyl, haloC₁-C₆alkylC₃-C₁₀cycloalkyl, —CO-aryl, —OC₁-C₆alkylaryl, —OC₁-C₆alkylheteroaryl, —OC₁-C₆alkyl-cycloheteroalkyl, —OC₁-C₆alkylC₃-C₆cycloalkyl, —SO₂C₁-C₆alkyl, —SO₂aryl, —S-aryl, —SC₁-C₆alkyl, —N(R³)₂ or C₁-C₆alkylN(R³)₂, wherein any aryl, heteroaryl, cycloalkyl or cycloheteroalkyl is unsubstituted or substituted with one to three substituents selected from the group consisting of OH, haloC₁-C₆alkyl, aryl, heteroaryl, N(R³)₂, SO₂C₁-C₆alkyl, alkoxy, CN, halogen, C₁-C₆alkyl, —SC₁-C₆alkyl, C₁-C₆alkyl-cycloheteroalkyl, C₁-C₆alkylheteroaryl and C₁-C₆alkylOH or, when taken with R^{2a} forms a cycloheteroalkyl or C₃-C₁₀cycloalkyl, wherein the cycloheteroalkyl or C₃-C₁₀cycloalkyl is unsubstituted or substituted with one to four substituents selected from the group consisting of halogen, C₁-C₆alkyl, aryl, alkoxy, —O-aryl, —O-heteroaryl, N(R³)₂, CN, —SO₂C₁-C₆alkyl, C₁-C₆alkylOH, heteroaryl, C(O)OC₁-C₆alkyl, and C₁-C₆alkyl-cycloheteroalkyl;

R^{2c} is hydrogen, C₁-C₆alkyl, C₁-C₆alkylOH, C₃-C₁₀cycloalkyl, heteroaryl, C₁-C₆alkylOC₁-C₆alkyl, C₁-C₆alkylO-heteroaryl, C₁-C₆alkylheteroaryl, aryl or C₁-C₆alkylaryl, wherein the aryl, heteroaryl, C₃-C₆cycloalkyl, C₁-C₆alkylheteroaryl or C₁-C₆alkylaryl is unsubstituted or substituted with one to four substituents selected from the group consisting of halogen, C₁-C₆alkyl, CN, OH and alkoxy; and

R³ is hydrogen, C₁-C₆alkyl, aryl or heteroaryl, wherein the aryl and heteroaryl is substituted with 1-3 substituents selected from the group consisting of CN, C₁-C₆alkyl, haloC₁-C₆alkyl and alkoxy.

Also described herein are compounds of Formula III:

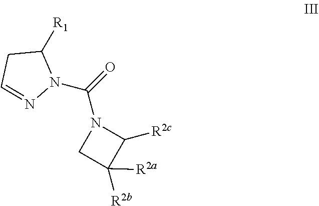

or a pharmaceutically acceptable salt thereof, wherein:

R¹ is aryl, C₃-C₁₀cycloalkyl or heteroaryl, wherein the aryl, C₃-C₁₀cycloalkyl or heteroaryl is unsubstituted or substituted with one to three substituents selected from the group consisting of halogen, C₁-C₆alkyl, CN, OH, alkoxy, —N(R³)₂, —SC₁-C₆alkyl and C₃-C₆cycloalkyl;

R^{2a} is hydrogen, halogen, CN, OH, C₁-C₆alkyl, alkoxy, C₃-C₆cycloalkyl, C₁-C₆alkylOH, C₁-C₆alkylOC₁-C₆alkyl, N(R³)₂ or haloC₁-C₆alkyl, or taken with R^{2b} forms a cycloheteroalkyl or C₃-C₁₀cycloalkyl, wherein the cycloheteroalkyl or C₃-C₁₀cycloalkyl is unsubstituted or substituted with one to four substituents selected from the group consisting of —SO₂C₁-C₆alkyl, C₁-C₆alkylOH, heteroaryl, C(O)OC₁-C₆alkyl, halogen, C₁-C₆alkyl, aryl, alkoxy, —O-aryl, —O— heteroaryl, N(R³)₂, CN and C₁-C₆alkyl-cycloheteroalkyl;

R^{2b} is hydrogen, OH, C₁-C₆alkylOH, CN, C₁-C₆alkylCN, C₁-C₆alkyl, haloC₁-C₆alkyl, halogen, alkoxy, C₁-C₆alkylOC₁-C₆alkyl, aryl, heteroaryl, cycloheteroalkyl, C₃-C₁₀cycloalkyl, —O-aryl, —O-heteroaryl, —O-cycloheteroalkyl, —OC₃-C₁₀cycloalkyl, C₁-C₆alkylaryl, C₁-C₆alkylheteroaryl, C₁-C₆alkyl-cycloheteroalkyl, C₁-C₆alkylC₃-C₁₀cycloalkyl, haloC₁-C₆alkylaryl, haloC₁-C₆alkylheteroaryl, haloC₁-C₆alkyl-cycloheteroalkyl, haloC₁-C₆alkylC₃-C₁₀cycloalkyl, —CO-aryl, —OC₁-C₆alkylaryl, —OC₁-C₆alkylheteroaryl, —OC₁-C₆alkyl-cycloheteroalkyl, —OC₁-C₆alkylC₃-C₆cycloalkyl, —SO₂C₁-C₆alkyl, —SO₂aryl, —S-aryl, —SC₁-C₆alkyl, —N(R³)₂ or C₁-C₆alkylN(R³)₂, wherein any aryl, heteroaryl, cycloalkyl or cycloheteroalkyl is unsubstituted or substituted with one to three substituents selected from the group consisting of OH, haloC₁-C₆alkyl, aryl, heteroaryl, N(R³)₂, SO₂C₁-C₆alkyl, alkoxy, CN, halogen, C₁-C₆alkyl, —SC₁-C₆alkyl, C₁-C₆alkyl-cycloheteroalkyl, C₁-C₆alkylheteroaryl and C₁-C₆alkylOH or, when taken with R^{2a} forms a cycloheteroalkyl or C₃-C₁₀cycloalkyl, wherein the cycloheteroalkyl or C₃-C₁₀cycloalkyl is unsubstituted or substituted with one to four substituents selected from the group consisting of halogen, C₁-C₆alkyl, aryl, alkoxy, —O-aryl, —O-heteroaryl, N(R³)₂, CN, —SO₂C₁-C₆alkyl, C₁-C₆alkylOH, heteroaryl, C(O)OC₁-C₆alkyl, and C₁-C₆alkyl-cycloheteroalkyl;

R^{2c} is hydrogen, C₁-C₆alkyl, C₁-C₆alkylOH, C₃-C₁₀cycloalkyl, heteroaryl, C₁-C₆alkylOC₁-C₆alkyl, C₁-C₆alkyl-O-heteroaryl, C₁-C₆alkylheteroaryl, aryl or C₁-C₆alkylaryl, wherein the aryl, heteroaryl, C₃-C₆cycloalkyl, C₁-C₆alkylheteroaryl or C₁-C₆alkylaryl is unsubstituted or substituted with one to four substituents selected from the group consisting of halogen C₁-C₆alkyl, CN, OH and alkoxy; and

R³ is hydrogen, C₁-C₆alkyl, aryl or heteroaryl, wherein the aryl and heteroaryl are unsubstituted or substituted with 1-3 substituents selected from the group consisting of CN, C₁-C₆alkyl, haloC₁-C₆alkyl and alkoxy.

With regard to the compounds of Formula II and Formula III, R¹ is as described above.

With regard to the compounds described herein, R^{2a} is hydrogen, halogen, CN, OH, C₁-C₆alkyl, alkoxy, C₃-C₆cycloalkyl, C₁-C₆alkylOH, C₁-C₆alkylOC₁-C₆alkyl, N(R³)₂ or haloC₁-C₆alkyl, or taken with R^{2b} forms a cycloheteroalkyl or C₃-C₁₀cycloalkyl, wherein the cycloheteroalkyl or C₃-C₁₀cycloalkyl is unsubstituted or substituted with one to four substituents selected from the group consisting —SO₂C₁-C₆alkyl, C₁-C₆alkylOH, heteroaryl, C(O)OC₁-C₆alkyl, halogen, C₁-C₆alkyl, aryl, alkoxy, —O-aryl, —O-heteroaryl, N(R³)₂, CN and C₁-C₆alkyl-cycloheteroalkyl.

In certain embodiments, R^{2a} is hydrogen.

In certain embodiments, R^{2a} is halogen. Suitable halogens include, but are not limited to, fluorine, chlorine, bromine or iodine. In certain embodiments, R^{2a} is fluorine, or chlorine.

In certain embodiments, R^{2a} is CN.

In certain embodiments, R^{2a} is OH.

In certain embodiments, R^{2a} is C₁-C₆alkyl. Suitable alkyls include, but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl and 1-ethyl-1-methylpropyl. In certain embodiments, R^{2a} is methyl. In certain embodiments, R^{2a} is ethyl.

In certain embodiments, R^{2a} is alkoxy. Suitable alkoxys include, but are not limited to, methoxy, ethoxy, n-propoxy, isopropoxy and n-butoxy. In certain embodiments, R^{2a} is methoxy.

In certain embodiments, R^{2a} is C₃-C₆cycloalkyl. In certain embodiments, R^{2a} is a monocyclic cycloalkyl. In other embodiments, R^{2a} is a bicyclic cycloalkyl. In other embodiments, R^{2a} is a multicyclic cycloalkyl. Suitable cycloalkyls include, but are not limited to, cyclopropyl, cyclobutyl, cyclopentyl, cyclohexyl, cycloheptyl, tetrahydronaphthyl, decahydronaphthyl, indanyl. In certain embodiments, R^{2a} is

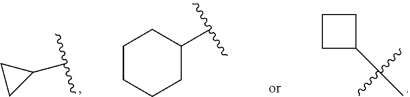

In certain embodiments, R^{2a} is C₁-C₆alkylOH. Suitable alcohols include, but are not limited to, methanol, ethanol, propanol and butanol. In certain embodiments, R^{2a} is

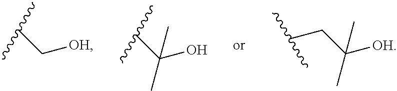

In certain embodiments, R^{2a} is C₁-C₆alkylOC₁-C₆alkyl.

In certain embodiments, R^{2a} is N(R³)₂. In certain embodiments, —N(R³)₂ is

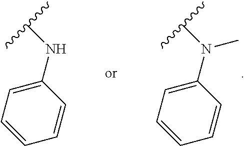

In certain embodiments, R^{2a} is haloC₁-C₆alkyl. Suitable examples of haloalkyls include, but are not limited to, fluoromethyl, difluoromethyl, trifluoromethyl, 2-fluoroethyl, 1,2-difluoroethyl and 2,2-difluoroethyl. In certain embodiments, R^{2a} is difluoromethyl. In certain embodiments, R^{2a} is trifluoromethyl. In certain embodiments, R^{2a} is difluoromethyl or trifluoromethyl.

In certain embodiments, R^{2a} is hydrogen, methyl, ethyl, OH, fluorine, CN or methoxy.

In certain embodiments, R^{2a} is hydrogen, methyl, OH, fluorine, CN or methoxy.

In certain embodiments, R^{2a} is hydrogen, methyl, ethyl, OH, fluorine, CN or methoxy.

With regard to the compounds described herein, R^{2b} is hydrogen, OH, C₁-C₆alkylOH, CN, C₁-C₆alkylCN, C₁-C₆alkyl, haloC₁-C₆alkyl, halogen, alkoxy, C₁-C₆alkylOC₁-C₆alkyl, aryl, heteroaryl, cycloheteroalkyl, C₃-C₁₀cycloalkyl, —O-aryl, —O-heteroaryl, —O-cycloheteroalkyl, —OC₃-C₁₀cycloalkyl, C₁-C₆alkylaryl, C₁-C₆alkylheteroaryl, C₁-C₆alkyl-cycloheteroalkyl, C₁-C₆alkylC₃-C₁₀cycloalkyl, haloC₁-C₆alkylaryl, haloC₁-C₆alkylheteroaryl, haloC₁-C₆alkyl-cycloheteroalkyl, haloC₁-C₆alkylC₃-C₁₀cycloalkyl, —CO-aryl, —OC₁-C₆alkylaryl, —OC₁-C₆alkylheteroaryl, —OC₁-C₆alkyl-cycloheteroalkyl, —OC₁-C₆alkylC₃-C₆cycloalkyl, —SO₂C₁-C₆alkyl, —SO₂aryl, —S-aryl, —SC₁-C₆alkyl, —N(R³)₂ or C₁-C₆alkylN(R³)₂, wherein any aryl, heteroaryl, cycloalkyl or cycloheteroalkyl is unsubstituted or substituted with one to three substituents selected from the group consisting of OH, haloC₁-C₆alkyl, aryl, heteroaryl, N(R³)₂, SO₂C₁-C₆alkyl, alkoxy, CN, halogen, C₁-C₆alkyl, —SC₁-C₆alkyl, C₁-C₆alkyl-cycloheteroalkyl, C₁-C₆alkylheteroaryl and C₁-C₆alkylOH or, when taken with R^{2a} forms a cycloheteroalkyl or C₃-C₁₀cycloalkyl, wherein the cycloheteroalkyl or C₃-C₁₀cycloalkyl is unsubstituted or substituted with one to four substituents selected from the group consisting of halogen, C₁-C₆alkyl, aryl, alkoxy, —O-aryl, —O-heteroaryl, N(R³)₂, CN, —SO₂C₁-C₆alkyl, C₁-C₆alkylOH, heteroaryl, C(O)OC₁-C₆alkyl, and C₁-C₆alkyl-cycloheteroalkyl.

In certain embodiments, R^{2b} is hydrogen, OH, C₁-C₆alkylOH, CN, C₁-C₆alkylCN, C₁-C₆alkyl, haloC₁-C₆alkyl, halogen, alkoxy, C₁-C₆alkylOC₁-C₆alkyl, aryl, heteroaryl, cycloheteroalkyl, C₃-C₁₀cycloalkyl, —O-aryl, —O-heteroaryl, —O-cycloheteroalkyl, —OC₃-C₁₀cycloalkyl, C₁-C₆alkylaryl, C₁-C₆alkylheteroaryl, C₁-C₆alkyl-cycloheteroalkyl, C₁-C₆alkylC₃-C₁₀cycloalkyl, —CO-aryl, —OC₁-C₆alkylaryl, —OC₁-C₆alkylheteroaryl, —OC₁-C₆alkyl-cycloheteroalkyl, —OC₁-C₆alkylC₃-C₆cycloalkyl, —SO₂C₁-C₆alkyl, —SO₂aryl, —S-aryl, —SC₁-C₆alkyl, —N(R³)₂ or C₁-C₆alkylN(R³)₂, wherein any aryl, heteroaryl, cycloalkyl or cycloheteroalkyl is unsubstituted or substituted with one to three substituents selected from the group consisting of OH, haloC₁-C₆alkyl, aryl, heteroaryl, N(R³)₂, SO₂C₁-C₆alkyl, alkoxy, CN, halogen, C₁-C₆alkyl, —SC₁-C₆alkyl, C₁-C₆alkyl-cycloheteroalkyl, C₁-C₆alkylheteroaryl and C₁-C₆alkylOH.

In certain embodiments, R^{2b} is hydrogen.

In certain embodiments, R^{2b} is OH.

In certain embodiments, R^{2b} is C₁-C₆alkylOH. Suitable alcohols include, but are not limited to, methanol, ethanol, propanol and butanol. In certain embodiments, R^{2b} is

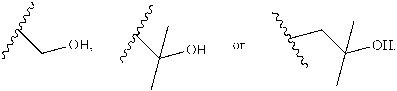

In certain embodiments, R^{2b} is

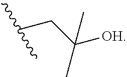 [SMILES: *CC(C)(C)O]

In certain embodiments, R^{2b} is CN.

In certain embodiments, R^{2b} is C₁-C₆alkylCN. In certain embodiments, R^{2b} is

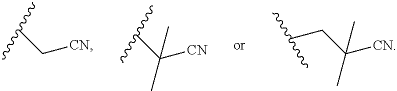

In certain embodiments, R^{2b} is C₁-C₆alkyl. Suitable alkyls include, but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl and 1-ethyl-1-methylpropyl. In certain embodiments, R^{2b} is methyl, ethyl or

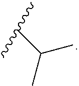 [SMILES: *C(C)C]

In certain embodiments, R^{2b} is haloC₁-C₆alkyl. Suitable examples of haloalkyl include, but are not limited to, fluoromethyl, difluoromethyl, trifluoromethyl, 2-fluoroethyl, 1,2-difluoroethyl and 2,2-difluoroethyl. In certain embodiments, R^{2b} is difluoromethyl. In certain embodiments, R^{2b} is trifluoromethyl. In certain embodiments, R^{2b} is difluoromethyl or trifluoromethyl.

In certain embodiments, R^{2b} is halogen. Suitable halogens include, but are not limited to, fluorine, chlorine, bromine or iodine. In certain embodiments, R^{2b} is fluorine or chlorine.

In certain embodiments, R^{2b} is alkoxy. Suitable alkoxys include, but are not limited to, methoxy, ethoxy, n-propoxy, isopropoxy and n-butoxy. In certain embodiments, R^{2b} is methoxy, ethoxy or

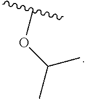 [SMILES: *OC(C)C]

In certain embodiments, R^{2b} is C₁-C₆alkylOC₁-C₆alkyl. In certain embodiments, R^{2b} is CH₂OCH₃ or CH₂CH₂OCH₃. In certain embodiments, R^{2b} is CH₂CH₂OCH₃.

In certain embodiments, R^{2b} is aryl. Suitable aryls include, but are not limited to, phenyl and naphthyl. In certain embodiments, R^{2b} is phenyl.

In certain embodiments, R^{2b} is heteroaryl. In certain embodiments, R^{2b} is a nitrogen-containing heteroaryl. In certain embodiments, R^{2b} is a monocyclic heteroaryl. In other embodiments, R^{2b} is a bicyclic heteroaryl. In other embodiments, R^{2b} is a multicyclic heteroaryl. Suitable heteroaryls include, but are not limited to, pyridyl (pyridinyl), oxazolyl, imidazolyl, triazolyl, furyl, triazinyl, thienyl, pyrimidyl, pyridazinyl, indolizinyl, cinnolinyl, phthalazinyl, quinazolinyl, naphthyridinyl, quinoxalinyl, purinyl, benzimidazolyl, quinolyl, and isoquinolyl. In certain embodiments, R^{2b} is pyridyl. In certain embodiments, R^{2b} is

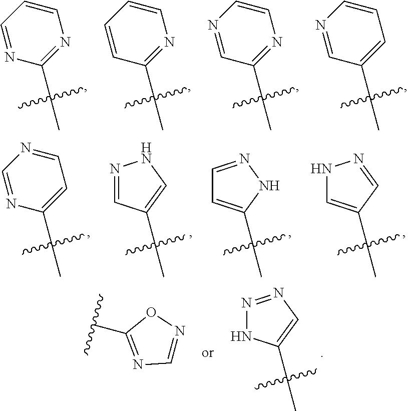

In certain embodiments, R^{2b} is

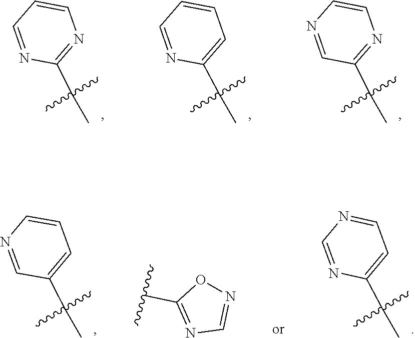 [SMILES: *c1ncccn1 ; *c1ccccn1 ; *c1cnccn1 ; *c1cccnc1 ; *c1ncno1 ; *c1ccncn1]

In certain embodiments, one or more R^{2b} substituents are cycloheteroalkyl. In certain embodiments, R^{2b} is

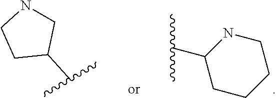 [SMILES: *C1CCNC1 ; *C1CCCCN1]

In certain embodiments, R^{2b} is C₃-C₁₀cycloalkyl. In certain embodiments, R^{2b} is a monocyclic cycloalkyl. In other embodiments, R^{2b} is a bicyclic cycloalkyl. In other embodiments, R^{2b} is a multicyclic cycloalkyl. Suitable cycloalkyls include, but are not limited to, cyclopropyl, cyclobutyl, cyclopentyl cyclohexyl, cycloheptyl, tetrahydronaphthyl, decahydronaphthyl, indanyl. In certain embodiments, R^{2b} is

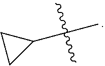 [SMILES: *C1CC1]

In certain embodiments, R^{2b} is —O-aryl. In certain embodiments, R^{2b} is

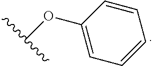 [SMILES: *Oc1ccccc1]

In certain embodiments, R^{2b} is —O-heteroaryl. In certain embodiments, R^{2b} is

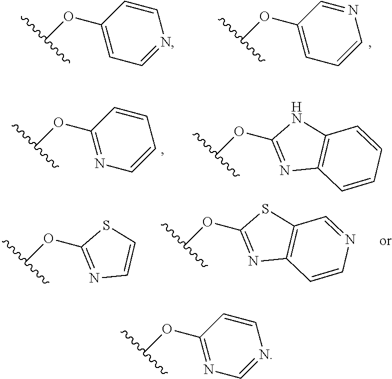

In certain embodiments, R^{2b} is —O-cycloheteroalkyl. In certain embodiments, R^{2b} is

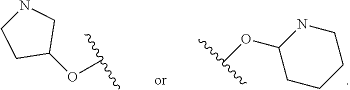 [SMILES: *OC1CCNC1 ; *OC1CCCCN1]

In certain embodiments, R^{2b} is —OC₃-C₁₀cycloalkyl. In certain embodiments, R^{2b} is

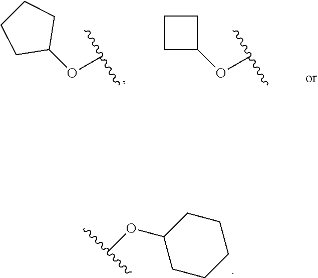 [SMILES: *OC1CCCC1 ; *OC1CCC1 ; *OC1CCCCC1]

In certain embodiments, R^{2b} is C₁-C₆alkylaryl. In certain embodiments, R^{2b} is

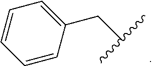 [SMILES: *Cc1ccccc1]

In certain embodiments, R^{2b} is C₁-C₆alkylheteroaryl. In certain embodiments, R^{2b} is

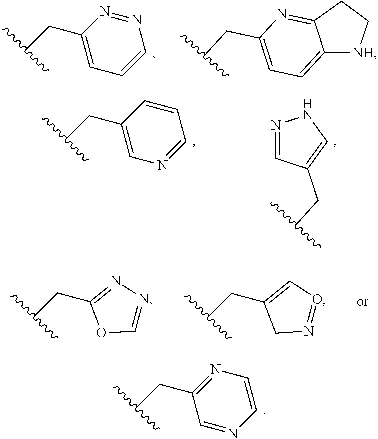

In certain embodiments, R^{2b} is C₁-C₆alkyl-cycloheteroalkyl. In certain embodiments, R^{2b} is

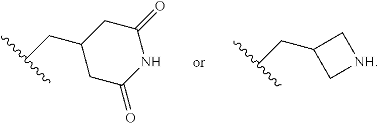 [SMILES: *CC1CC(=O)NC(=O)C1 ; *CC1CNC1]

In certain embodiments, R^{2b} is C₁-C₆alkylC₃-C₁₀cycloalkyl. In certain embodiments, R^{2b} is

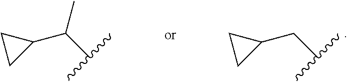

In certain embodiments, one or more R^{2b} substituents are haloC₁-C₆alkylaryl. In certain embodiments, R^{2b} is

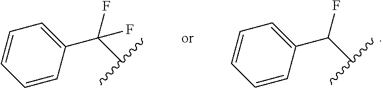 [SMILES: *C(F)(F)c1ccccc1 ; *C(F)c1ccccc1]

In certain embodiments, one or more R^{2b} substituents are haloC₁-C₆alkyl-heteroaryl. In certain embodiments, R^{2b} is.

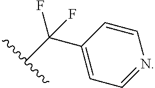 [SMILES: *C(F)(F)c1ccncc1]

In certain embodiments, one or more R^{2b} substituents are haloC₁-C₆alkyl-cycloheteroalkyl. In certain embodiments, R^{2b} is

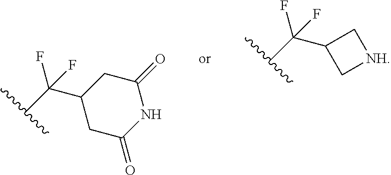 [SMILES: *C(F)(F)C1CC(=O)NC(=O)C1 ; *C(F)(F)C1CNC1]

In certain embodiments, one or more R^{2b} substituents are haloC₁-C₆alkyl-C₃-C₁₀cycloalkyl. In certain embodiments, R^{2b} is

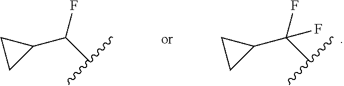 [SMILES: *C(F)C1CC1 ; *C(F)(F)C1CC1]

In certain embodiments, R^{2b} is —CO-aryl. In certain embodiments, R^{2b} is

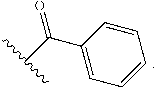 [SMILES: *C(=O)c1ccccc1]

In certain embodiments, R^{2b} is —OC₁-C₆alkylaryl. In certain embodiments, R^{2b} is

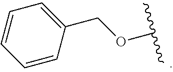 [SMILES: *OCc1ccccc1]

In certain embodiments, R^{2b} is —OC₁-C₆alkylheteroaryl. In certain embodiments, R^{2b} is

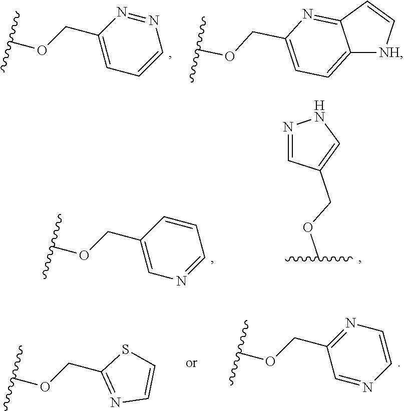

In certain embodiments, R^{2b} is —OC₁-C₆alkyl-cycloheteroalkyl. In certain embodiments, R^{2b} is

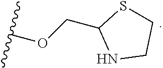 [SMILES: *OCC1NCCS1]

In certain embodiments, R^{2b} is —OC₁-C₆alkylC₃-C₆cycloalkyl. In certain embodiments, R^{2b} is

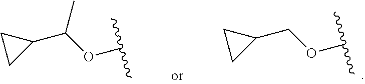

In certain embodiments, R^{2b} is —SO₂C₁-C₆alkyl. In certain embodiments, R^{2b} substituents are —SO₂CH₃, —SO₂CH₂CH₃, or —SO₂CH₂CH₃.

In certain embodiments, R^{2b} is —SO₂aryl. In certain embodiments, R^{2b} is

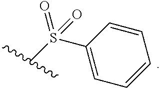 [SMILES: *S(=O)(=O)c1ccccc1]

In certain embodiments, R^{2b} is —S-aryl. In certain embodiments, R^{2b} is

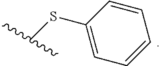 [SMILES: *Sc1ccccc1]

In certain embodiments, R^{2b} is —SC₁-C₆alkyl. In certain embodiments, R^{2b} is —SCH₃, —SCH₂CH₃, or —SCH₂CH₃.

In certain embodiments, R^{2b} is —N(R³)₂. In certain embodiments, —N(R³)₂ is

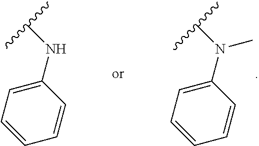

In certain embodiments, R^{2b} is C₁-C₆alkylN(R³)₂. In certain embodiments, R^{2b} is

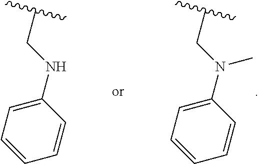

In certain embodiments R^{2b} is unsubstituted. In certain embodiments, wherein R^{2b} is an aryl, heteroaryl, cycloalkyl or cycloheteroalkyl, the aryl, heteroaryl, cycloalkyl or cycloheteroalkyl is substituted with one, two or three substituents selected from the group consisting of OH, haloC₁-C₆alkyl, aryl, heteroaryl, N(R³)₂, SO₂C₁-C₆alkyl, alkoxy, CN, halogen, C₁-C₆alkyl, —SC₁-C₆alkyl, C₁-C₆alkyl-cycloheteroalkyl, C₁-C₆alkylheteroaryl and C₁-C₆alkylOH.

In certain embodiments, wherein R^{2b} is aryl, —O-aryl, C₁-C₆alkylaryl, —CO-aryl or —OC₁-C₆alkylaryl, the aryl, —O-aryl, C₁-C₆alkylaryl, —CO-aryl or —OC₁-C₆alkylaryl is substituted with one to three substituents selected from the group consisting of OH, haloC₁-C₆alkyl, aryl, heteroaryl, N(R³)₂, SO₂C₁-C₆alkyl, alkoxy, CN, halogen, C₁-C₆alkyl, —SC₁-C₆alkyl, C₁-C₆alkyl-cycloheteroalkyl, C₁-C₆alkylheteroaryl and C₁-C₆alkylOH.

In certain embodiments, wherein R^{2b} is heteroaryl, —O-heteroaryl, C₁-C₆alkylheteroaryl, or —OC₁-C₆alkylheteroaryl, the heteroaryl, —O-heteroaryl, C₁-C₆alkylheteroaryl or —OC₁-C₆alkylheteroaryl is substituted with one to three substituents selected from the group consisting of OH, haloC₁-C₆alkyl, aryl, heteroaryl, N(R³)₂, SO₂C₁-C₆alkyl, alkoxy, CN, halogen, C₁-C₆alkyl, —SC₁-C₆alkyl, C₁-C₆alkyl-cycloheteroalkyl, C₁-C₆alkylheteroaryl and C₁-C₆alkylOH.

In certain embodiments, wherein R^{2b} is cycloheteroalkyl, —O-cycloheteroalkyl, C₁-C₆alkyl-cycloheteroalkyl or —OC₁-C₆alkyl-cycloheteroalkyl, the cycloheteroalkyl, —O— cycloheteroalkyl, C₁-C₆alkyl-cycloheteroalkyl or —OC₁-C₆alkyl-cycloheteroalkyl is substituted with one to three substituents selected from the group consisting of OH, haloC₁-C₆alkyl, aryl, heteroaryl, N(R³)₂, SO₂C₁-C₆alkyl, alkoxy, CN, halogen, C₁-C₆alkyl, —SC₁-C₆alkyl, C₁-C₆alkyl-cycloheteroalkyl, C₁-C₆alkylheteroaryl and C₁-C₆alkylOH.

In certain embodiments, wherein R^{2b} is C₃-C₁₀cycloalkyl, —OC₃-C₁₀cycloalkyl, C₁-C₆alkylC₃-C₁₀cycloalkyl or —OC₁-C₆alkylC₃-C₆cycloalkyl, the C₃-C₁₀cycloalkyl, —OC₃-C₁₀cycloalkyl, C₁-C₆alkylC₃-C₁₀cycloalkyl or —OC₁-C₆alkylC₃-C₆cycloalkyl is substituted with one to three substituents selected from the group consisting of OH, haloC₁-C₆alkyl, aryl, heteroaryl, N(R³)₂, SO₂C₁-C₆alkyl, alkoxy, CN, halogen, C₁-C₆alkyl, —SC₁-C₆alkyl, C₁-C₆alkyl-cycloheteroalkyl, C₁-C₆alkylheteroaryl and C₁-C₆alkylOH.

In certain embodiments, R^{2b} is hydrogen, OH, chlorine, fluorine, CN, methoxy, ethoxy, methyl, difluoromethyl, trifluoromethyl, —SO₂CH₃, isopropyl, cyclopropyl,

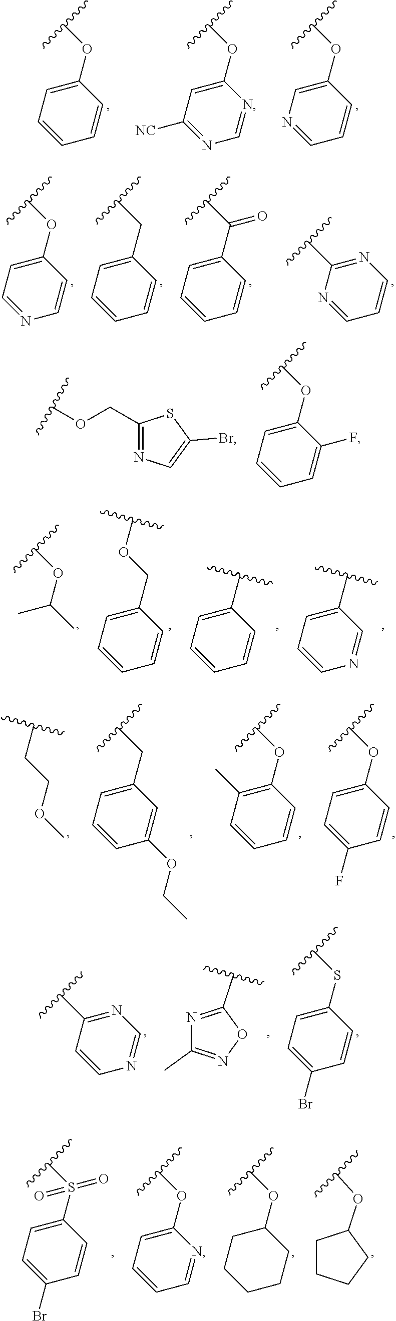

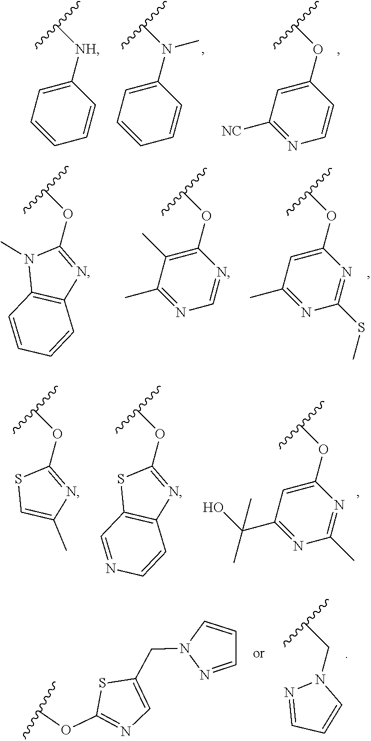

In certain embodiments, R^{2b} is hydrogen, OH, chlorine, fluorine, CN, methoxy, ethoxy, methyl, difluoromethyl, trifluoromethyl, —SO₂CH₃, isopropyl, cyclopropyl,

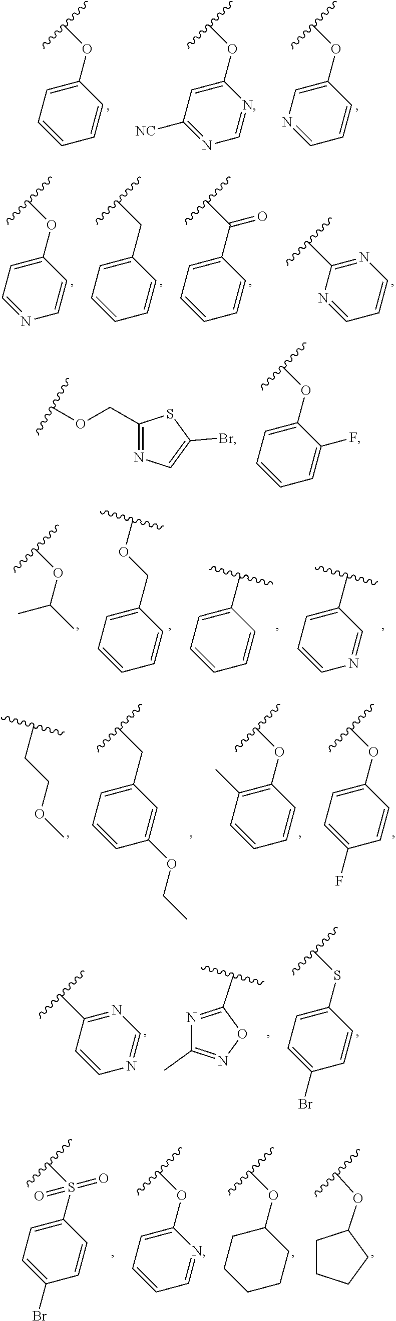

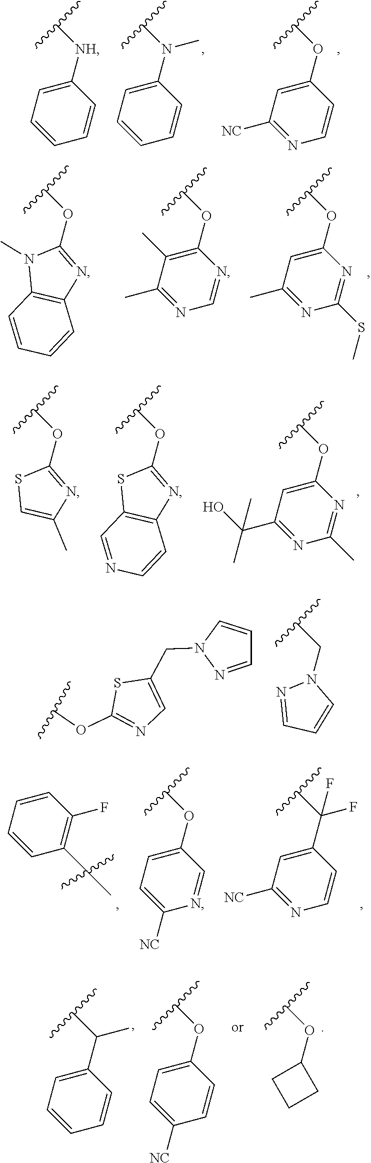

In certain embodiments, R^{2a} is taken with R^{2b} and forms a cycloheteroalkyl or C₃-C₁₀cycloalkyl, wherein the cycloheteroalkyl or C₃-C₁₀cycloalkyl is unsubstituted or substituted with one to four substituents selected from the group consisting —SO₂C₁-C₆alkyl, C₁-C₆alkylOH, heteroaryl, C(O)OC₁-C₆alkyl, halogen, C₁-C₆alkyl, aryl, alkoxy, —O-aryl, —O-heteroaryl, N(R³)₂, CN and C₁-C₆alkyl-cycloheteroalkyl.

In certain embodiments, R^{2a} is taken with R^{2b} to form a cycloheteroalkyl. In certain embodiments, R^{2a} is taken with R^{2b} to form a cycloheteroalkyl or C₃-C₁₀cycloalkyl. In certain embodiments, the cycloheteroalkyl or C₃-C₁₀cycloalkyl is unsubstituted. In certain embodiments, the cycloheteroalkyl or C₃-C₁₀cycloalkyl is substituted with one, two, three or four substituents selected from the group consisting of halogen, C₁-C₆alkyl, aryl, alkoxy, —O-aryl, -, N(R³)₂, CN—SOCN, —SO₂C₁-C₆alkyl, C₁-C₆alkylOH, heteroaryl, C(O)OC₁-C₆alkyl, and C₁-C₆alkyl-cycloheteroalkyl.

In certain embodiments, R^{2a} is taken with R^{2b} and forms:

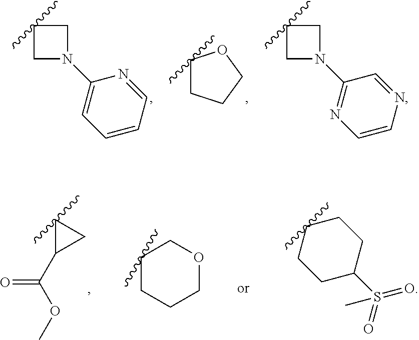

With regards to the compounds described herein, R^{2c} is hydrogen, C₁-C₆alkyl, C₁-C₆alkylOH, C₃-C₁₀cycloalkyl, heteroaryl, C₁-C₆alkylOC₁-C₆alkyl, C₁-C₆alkylO-heteroaryl, C₁-C₆alkylheteroaryl, aryl or C₁-C₆alkylaryl, wherein the aryl, heteroaryl, C₃-C₆cycloalkyl, C₁-C₆alkylheteroaryl or C₁-C₆alkylaryl is unsubstituted or substituted with one to four substituents selected from the group consisting of halogen, C₁-C₆alkyl, CN, OH and alkoxy.

In certain embodiments, R^{2c} is C₁-C₆alkyl, C₁-C₆alkylOH, C₃-C₁₀cycloalkyl, heteroaryl, C₁-C₆alkylOC₁-C₆alkyl, C₁-C₆alkylO-heteroaryl, C₁-C₆alkylheteroaryl, aryl or C₁-C₆alkylaryl, wherein the aryl, heteroaryl, C₃-C₆cycloalkyl, C₁-C₆alkylheteroaryl or C₁-C₆alkylaryl is unsubstituted or substituted with one to four substituents selected from the group consisting of halogen, C₁-C₆alkyl, CN, OH and alkoxy.

In certain embodiments, R^{2c}, is hydrogen.

In certain embodiments, R^{2c}, is not hydrogen.

In certain embodiments, R^{2c}, is C₁-C₆alkyl. Suitable alkyls include, but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl and 1-ethyl-1-methylpropyl. In certain embodiments, R^{2c} is methyl.

In certain embodiments, R^{2c} is C₁-C₆alkylOH. Suitable alcohols include, but are not limited to, methanol, ethanol, propanol and butanol. In certain embodiments, R^{2c} is

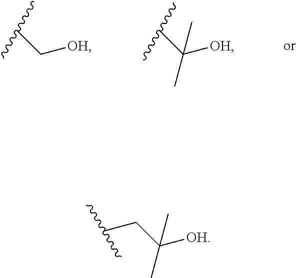

In certain embodiments, R^{2c} is C₁-C₆alkylOC₁-C₆alkyl.

In certain embodiments, R^{2c} is aryl. Suitable aryls include, but are not limited to, phenyl and naphthyl. In certain embodiments, R^{2c} is phenyl. In certain embodiments, R^{2c} is phenyl substituted with fluorine.

In certain embodiments, R^{2c} is heteroaryl. In certain embodiments, R^{2c} is

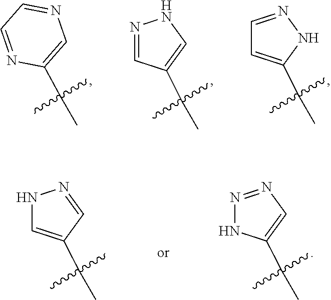 [SMILES: *c1cnccn1 ; *c1cn[nH]c1 ; *c1ccn[nH]1 ; *c1cn[nH]c1 ; *c1cnn[nH]1]

In certain embodiments, R^{2c} is C₃-C₁₀cycloalkyl. In certain embodiments, R^{2c} is a monocyclic cycloalkyl. In other embodiments, R^{2c} is a bicyclic cycloalkyl. In other embodiments, R^{2c} is a multicyclic cycloalkyl. Suitable cycloalkyls include, but are not limited to, cyclopropyl, cyclobutyl, cyclopentyl, cyclohexyl, cycloheptyl, tetrahydronaphthyl, decahydronaphthyl, indanyl. In certain embodiments, R^{2c} is

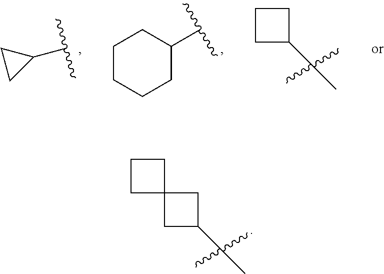

In certain embodiments, R^{2c} is C₁-C₆alkylO-heteroaryl. In certain embodiments, R^{2c} is

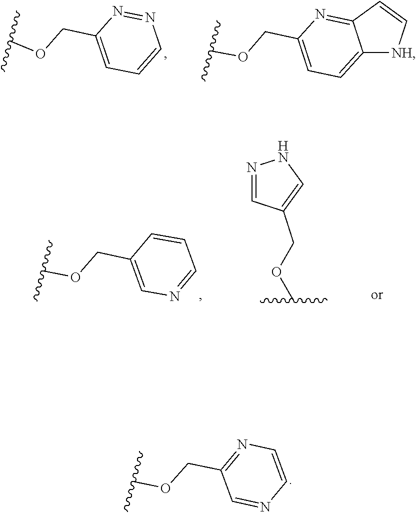 [SMILES: *OCc1cccnn1 ; *OCc1ccc2[nH]ccc2n1 ; *OCc1cccnc1 ; *OCc1cn[nH]c1 ; *OCc1cnccn1]

In certain embodiments, R^{2c} is C₁-C₆alkylheteroaryl. In certain embodiments, R^{2c} is

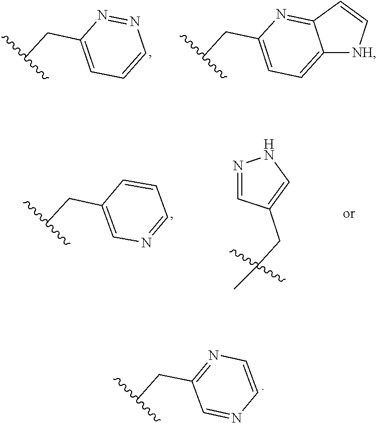 [SMILES: *Cc1cccnn1 ; *Cc1ccc2[nH]ccc2n1 ; *Cc1cccnc1 ; *Cc1cn[nH]c1 ; *Cc1cnccn1]

In certain embodiments, R^{2c} is C₁-C₆alkylaryl. In certain embodiments, R^{2c} is

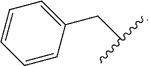 [SMILES: *Cc1ccccc1]

In certain embodiments, R^{2c} is hydrogen, methyl,

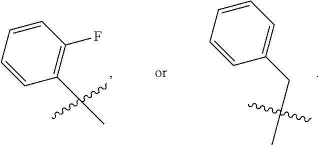 [SMILES: *c1ccccc1F ; *Cc1ccccc1]

In certain embodiments, R^{2c} is hydrogen, methyl, ethyl,

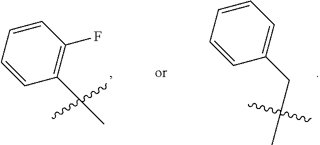 [SMILES: *c1ccccc1F ; *Cc1ccccc1]

With regard to the compounds of Formula II and III, R³ is as described above.

In certain embodiments, the compounds described herein can be described as having the following formula:

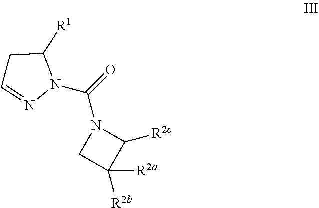

or a pharmaceutically acceptable salt thereof, wherein:

R¹ is

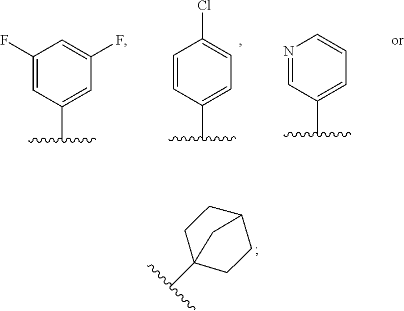 [SMILES: *c1cc(F)cc(F)c1 ; *c1ccc(Cl)cc1 ; *c1cccnc1 ; *C12CCC(CC1)C2]

R^{2a} is hydrogen, halogen, CN, OH, C₁-C₆alkyl, alkoxy or haloC₁-C₆alkyl, or taken with R^{2b} forms a cycloheteroalkyl or C₃-C₁₀cycloalkyl, wherein the cycloheteroalkyl or C₃-C₁₀cycloalkyl is unsubstituted or substituted with one to four substituents selected from the group consisting of —SO₂C₁-C₆alkyl, C₁-C₆alkylOH, heteroaryl, C(O)OC₁-C₆alkyl, and C₁-C₆alkyl-cycloheteroalkyl;

R^{2b} is hydrogen, —O-aryl, —O-heteroaryl, C₁-C₆alkylaryl, OH, —CO-aryl, —OC₃-C₁₀cycloalkyl, heteroaryl, alkoxy, —OC₁-C₆alkylaryl, haloC₁-C₆alkyl, C₁-C₆alkyl, halogen, C₃-C₁₀cycloalkyl, C₁-C₆alkylOC₁-C₆alkyl, aryl, —OC₁-C₆alkyl-cycloheteroalkyl, —SO₂C₁-C₆alkyl, cycloheteroalkyl, —S-aryl, —SO₂aryl, —N(R³)₂, C₁-C₆alkylheteroaryl, C₁-C₆alkylC₃-C₁₀cycloalkyl, —O-cycloheteroalkyl, C₁-C₆alkyl-cycloheteroalkyl, —OC₁-C₆alkylheteroaryl or CN, wherein the —O-heteroaryl, —O-aryl, C₁-C₆alkylaryl, —OC₁-C₆alkyl-cycloheteroalkyl, cycloheteroalkyl, —S-aryl, —SO₂aryl, heteroaryl, —OC₁-C₆alkylheteroaryl or —O-cycloheteroalkyl is unsubstituted or substituted with one to three substituents selected from the group consisting of alkoxy, CN, halogen, C₁-C₆alkyl, —SC₁-C₆alkyl, C₁-C₆alkyl-cycloheteroalkyl, C₁-C₆alkylheteroaryl and C₁-C₆alkylOH or, when taken with R^{2a} forms a cycloheteroalkyl or C₃-C₁₀cycloalkyl, wherein the cycloheteroalkyl or C₃-C₁₀cycloalkyl is unsubstituted or substituted with one to four substituents selected from the group consisting of —SO₂C₁-C₆alkyl, C₁-C₆alkylOH, heteroaryl, C(O)OC₁-C₆alkyl, and C₁-C₆alkyl-cycloheteroalkyl;

R^{2c} is hydrogen, C₁-C₆alkyl, aryl or C₁-C₆alkylaryl, wherein the aryl or C₁-C₆alkylaryl is unsubstituted or substituted with one to four halogen substituents; and

R³ is hydrogen, C₁-C₆alkyl or aryl.

In certain embodiments, the compounds described herein can have the following formula;

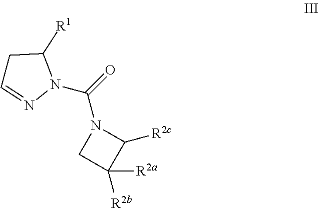

or a pharmaceutically acceptable salt thereof, wherein: R¹ is

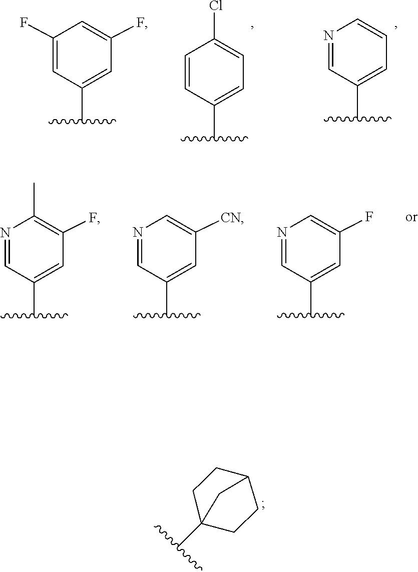

R^{2a} is hydrogen, halogen, CN, OH, C₁-C₆alkyl, alkoxy or haloC₁-C₆alkyl, or taken with R^{2b} forms a cycloheteroalkyl or C₃-C₁₀cycloalkyl, wherein the cycloheteroalkyl or C₃-C₁₀cycloalkyl is unsubstituted or substituted with one to four substituents selected from the group consisting —SO₂C₁-C₆alkyl, C₁-C₆alkylOH, heteroaryl, C(O)OC₁-C₆alkyl, and C₁-C₆alkyl-cycloheteroalkyl;

R^{2b} is hydrogen, —O-aryl, —O-heteroaryl, C₁-C₆alkylaryl, OH, —CO-aryl, —OC₃-C₁₀cycloalkyl, heteroaryl, alkoxy, —OC₁-C₆alkylaryl, haloC₁-C₆alkyl, C₁-C₆alkyl, halogen, C₃-C₁₀cycloalkyl, C₁-C₆alkylOC₁-C₆alkyl, aryl, —OC₁-C₆alkyl-cycloheteroalkyl, —SO₂C₁-C₆alkyl, cycloheteroalkyl, —S-aryl, —SO₂aryl, —N(R³)₂, C₁-C₆alkylheteroaryl, haloC₁-C₆alkylheteroaryl, C₁-C₆alkylC₃-C₁₀cycloalkyl, —O-cycloheteroalkyl, C₁-C₆alkyl-cycloheteroalkyl, —OC₁-C₆alkylheteroaryl or CN, wherein the —O-heteroaryl, —O-aryl, C₁-C₆alkylaryl, —OC₁-C₆alkyl-cycloheteroalkyl, cycloheteroalkyl, —S-aryl, —SO₂aryl, heteroaryl, —OC₁-C₆alkylheteroaryl or —O— cycloheteroalkyl is unsubstituted or substituted with one to three substituents selected from the group consisting of alkoxy, CN, halogen, C₁-C₆alkyl, —SC₁-C₆alkyl, C₁-C₆alkyl-cycloheteroalkyl, C₁-C₆alkylheteroaryl and C₁-C₆alkylOH or, when taken with R^{2a} forms a cycloheteroalkyl or C₃-C₁₀cycloalkyl, wherein the cycloheteroalkyl or C₃-C₁₀cycloalkyl is unsubstituted or substituted with one to four substituents selected from the group consisting —SO₂C₁-C₆alkyl, C₁-C₆alkylOH, heteroaryl, C(O)OC₁-C₆alkyl, and C₁-C₆alkyl-cycloheteroalkyl;

R^{2c} is hydrogen, C₁-C₆alkyl, aryl or C₁-C₆alkylaryl, wherein the aryl or C₁-C₆alkylaryl is unsubstituted or substituted with one to four halogen substituents; and

R³ is hydrogen, C₁-C₆alkyl or aryl.

Also described are the following compounds, or a pharmaceutically acceptable salt thereof:

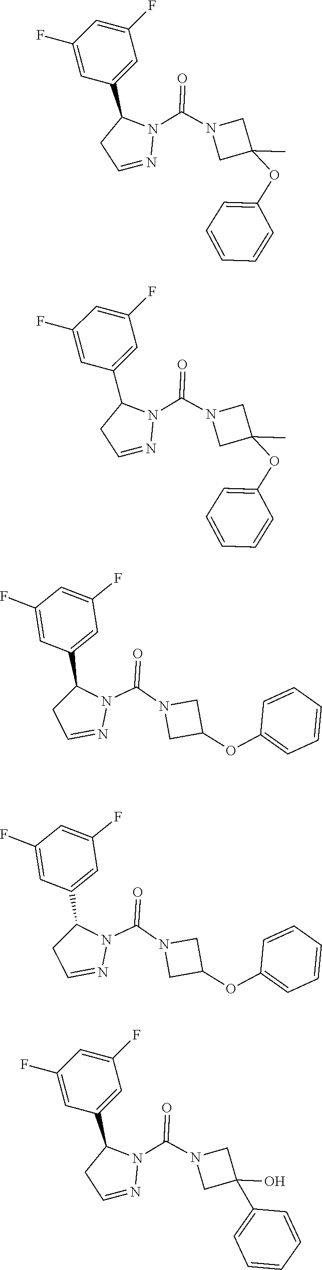 [SMILES: CC1(Oc2ccccc2)CN(C(=O)N2N=CC[C@H]2c2cc(F)cc(F)c2)C1 ; CC1(Oc2ccccc2)CN(C(=O)N2N=CCC2c2cc(F)cc(F)c2)C1 ; O=C(N1CC(Oc2ccccc2)C1)N1N=CC[C@H]1c1cc(F)cc(F)c1 ; O=C(N1CC(Oc2ccccc2)C1)N1N=CC[C@@H]1c1cc(F)cc(F)c1 ; O=C(N1CC(O)(c2ccccc2)C1)N1N=CC[C@H]1c1cc(F)cc(F)c1]

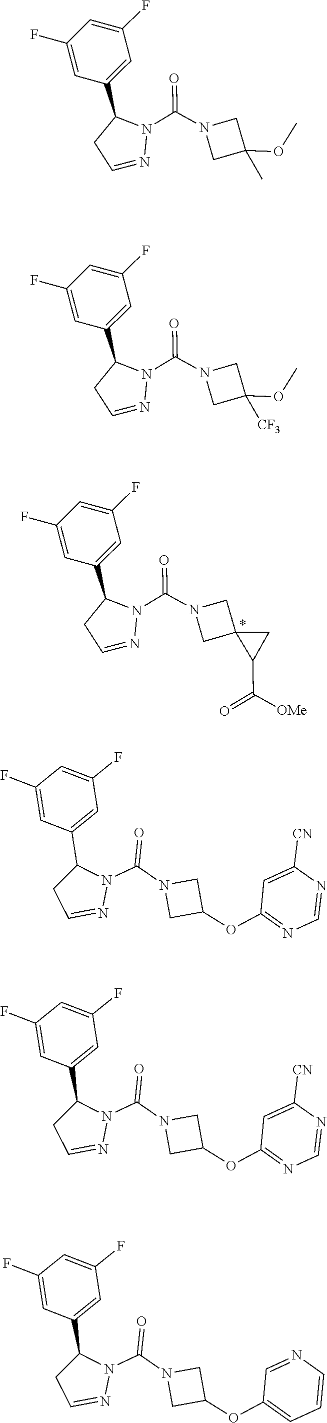 [SMILES: COC1(C)CN(C(=O)N2N=CC[C@H]2c2cc(F)cc(F)c2)C1 ; COC1(C(F)(F)F)CN(C(=O)N2N=CC[C@H]2c2cc(F)cc(F)c2)C1 ; COC(=O)C1CC12CN(C(=O)N1N=CC[C@H]1c1cc(F)cc(F)c1)C2 ; N#Cc1cc(OC2CN(C(=O)N3N=CCC3c3cc(F)cc(F)c3)C2)ncn1 ; N#Cc1cc(OC2CN(C(=O)N3N=CC[C@H]3c3cc(F)cc(F)c3)C2)ncn1 ; O=C(N1CC(Oc2cccnc2)C1)N1N=CC[C@H]1c1cc(F)cc(F)c1]

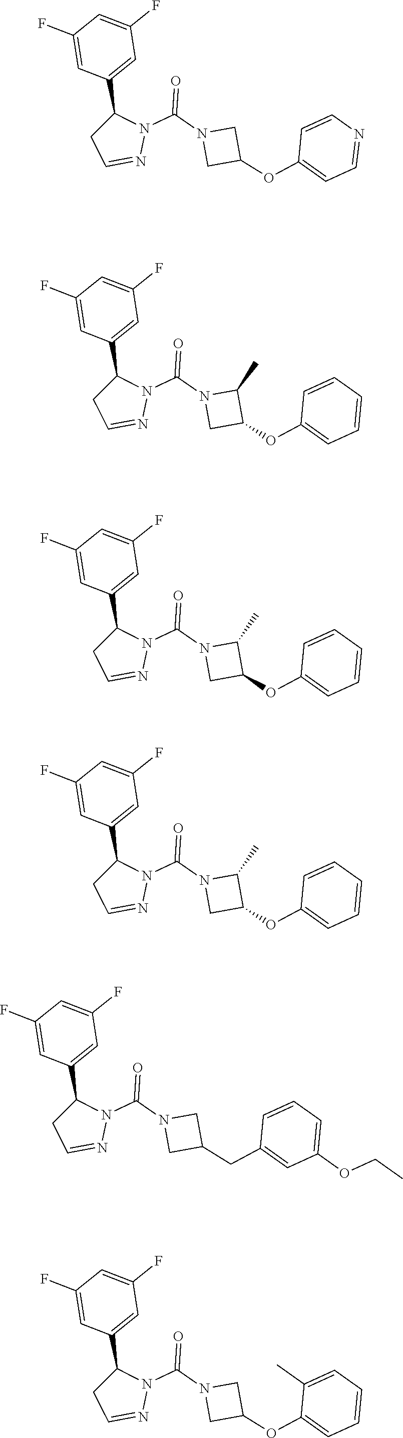 [SMILES: O=C(N1CC(Oc2ccncc2)C1)N1N=CC[C@H]1c1cc(F)cc(F)c1 ; C[C@H]1[C@H](Oc2ccccc2)CN1C(=O)N1N=CC[C@H]1c1cc(F)cc(F)c1 ; C[C@@H]1[C@@H](Oc2ccccc2)CN1C(=O)N1N=CC[C@H]1c1cc(F)cc(F)c1 ; C[C@@H]1[C@H](Oc2ccccc2)CN1C(=O)N1N=CC[C@H]1c1cc(F)cc(F)c1 ; CCOc1cccc(CC2CN(C(=O)N3N=CC[C@H]3c3cc(F)cc(F)c3)C2)c1 ; Cc1ccccc1OC1CN(C(=O)N2N=CC[C@H]2c2cc(F)cc(F)c2)C1]

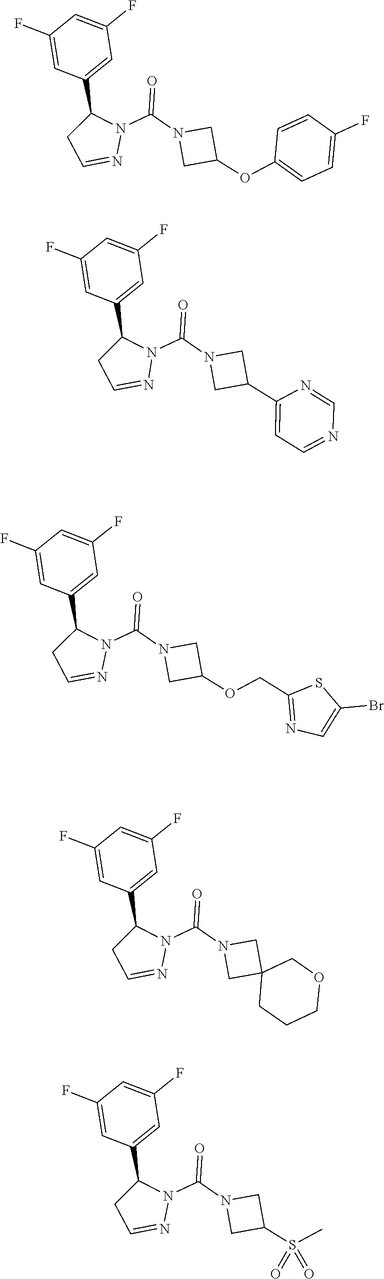 [SMILES: O=C(N1CC(Oc2ccc(F)cc2)C1)N1N=CC[C@H]1c1cc(F)cc(F)c1 ; O=C(N1CC(c2ccncn2)C1)N1N=CC[C@H]1c1cc(F)cc(F)c1 ; O=C(N1CC(OCc2ncc(Br)s2)C1)N1N=CC[C@H]1c1cc(F)cc(F)c1 ; O=C(N1CC2(CCCOC2)C1)N1N=CC[C@H]1c1cc(F)cc(F)c1 ; CS(=O)(=O)C1CN(C(=O)N2N=CC[C@H]2c2cc(F)cc(F)c2)C1]

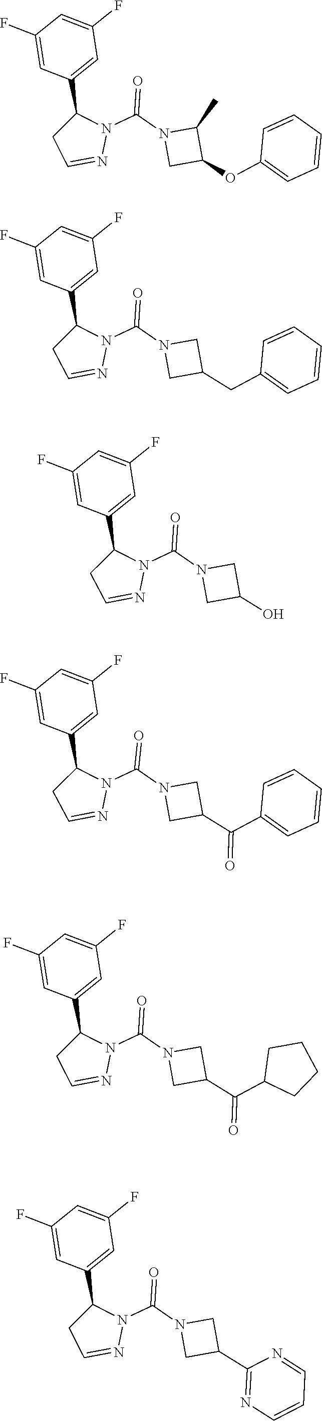 [SMILES: C[C@H]1[C@@H](Oc2ccccc2)CN1C(=O)N1N=CC[C@H]1c1cc(F)cc(F)c1 ; O=C(N1CC(Cc2ccccc2)C1)N1N=CC[C@H]1c1cc(F)cc(F)c1 ; O=C(N1CC(O)C1)N1N=CC[C@H]1c1cc(F)cc(F)c1 ; O=C(c1ccccc1)C1CN(C(=O)N2N=CC[C@H]2c2cc(F)cc(F)c2)C1 ; O=C(C1CCCC1)C1CN(C(=O)N2N=CC[C@H]2c2cc(F)cc(F)c2)C1 ; O=C(N1CC(c2ncccn2)C1)N1N=CC[C@H]1c1cc(F)cc(F)c1]

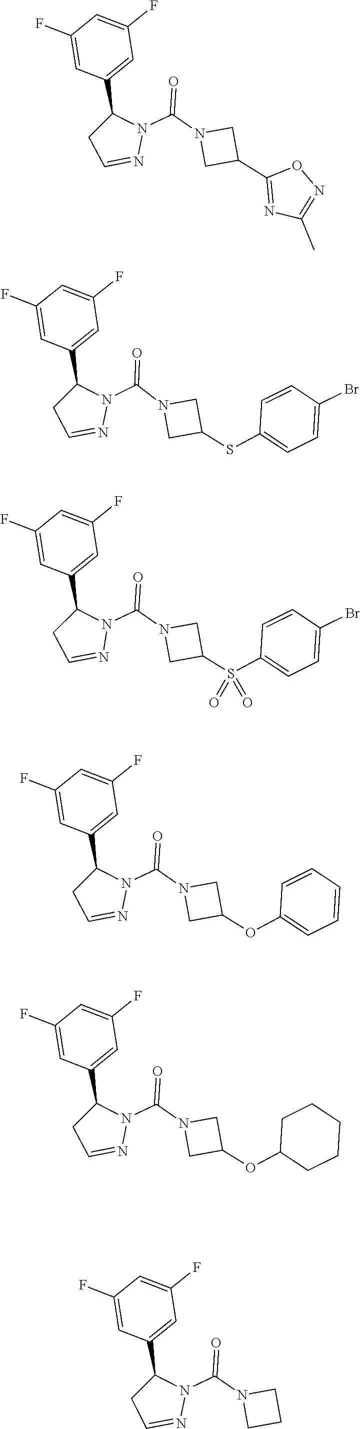 [SMILES: Cc1noc(C2CN(C(=O)N3N=CC[C@H]3c3cc(F)cc(F)c3)C2)n1 ; O=C(N1CC(Sc2ccc(Br)cc2)C1)N1N=CC[C@H]1c1cc(F)cc(F)c1 ; O=C(N1CC(S(=O)(=O)c2ccc(Br)cc2)C1)N1N=CC[C@H]1c1cc(F)cc(F)c1 ; O=C(N1CC(Oc2ccccc2)C1)N1N=CC[C@H]1c1cc(F)cc(F)c1 ; O=C(N1CC(OC2CCCCC2)C1)N1N=CC[C@H]1c1cc(F)cc(F)c1 ; O=C(N1CCC1)N1N=CC[C@H]1c1cc(F)cc(F)c1]

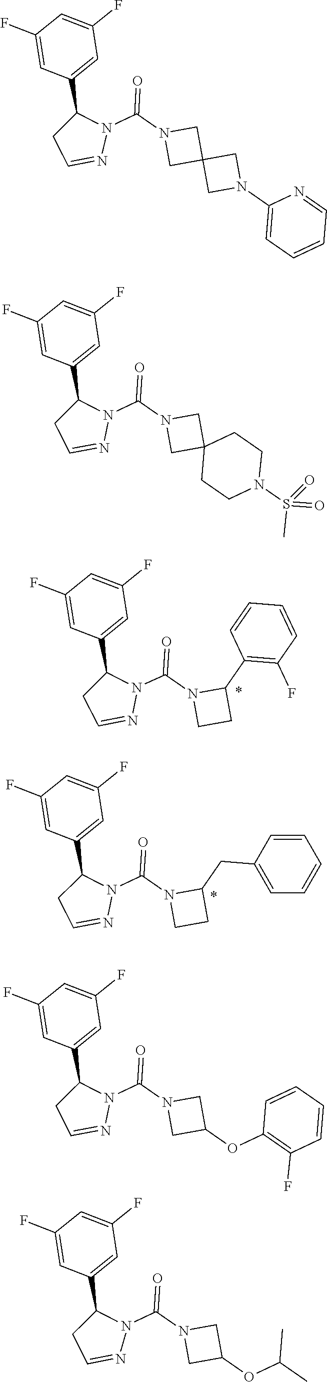 [SMILES: O=C(N1CC2(C1)CN(c1ccccn1)C2)N1N=CC[C@H]1c1cc(F)cc(F)c1 ; CS(=O)(=O)N1CCC2(CC1)CN(C(=O)N1N=CC[C@H]1c1cc(F)cc(F)c1)C2 ; O=C(N1CCC1c1ccccc1F)N1N=CC[C@H]1c1cc(F)cc(F)c1 ; O=C(N1CCC1Cc1ccccc1)N1N=CC[C@H]1c1cc(F)cc(F)c1 ; O=C(N1CC(Oc2ccccc2F)C1)N1N=CC[C@H]1c1cc(F)cc(F)c1 ; CC(C)OC1CN(C(=O)N2N=CC[C@H]2c2cc(F)cc(F)c2)C1]

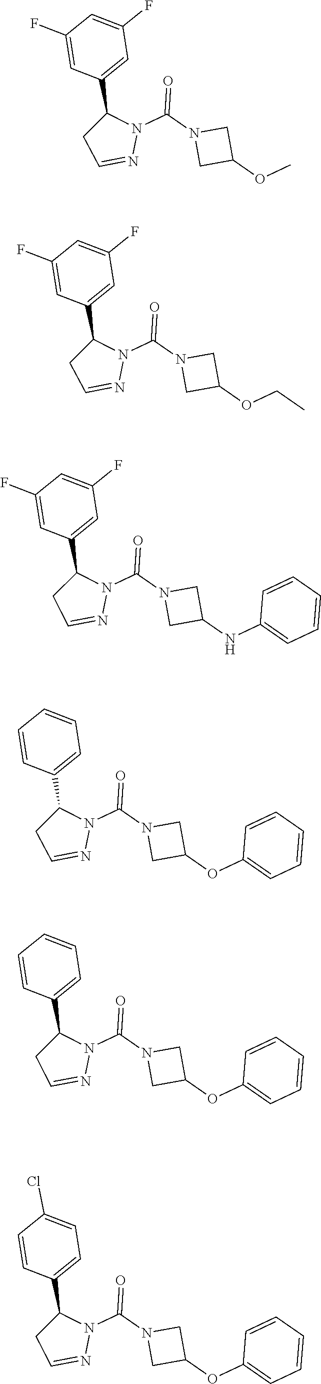 [SMILES: COC1CN(C(=O)N2N=CC[C@H]2c2cc(F)cc(F)c2)C1 ; CCOC1CN(C(=O)N2N=CC[C@H]2c2cc(F)cc(F)c2)C1 ; O=C(N1CC(Nc2ccccc2)C1)N1N=CC[C@H]1c1cc(F)cc(F)c1 ; O=C(N1CC(Oc2ccccc2)C1)N1N=CC[C@@H]1c1ccccc1 ; O=C(N1CC(Oc2ccccc2)C1)N1N=CC[C@H]1c1ccccc1 ; O=C(N1CC(Oc2ccccc2)C1)N1N=CC[C@H]1c1ccc(Cl)cc1]

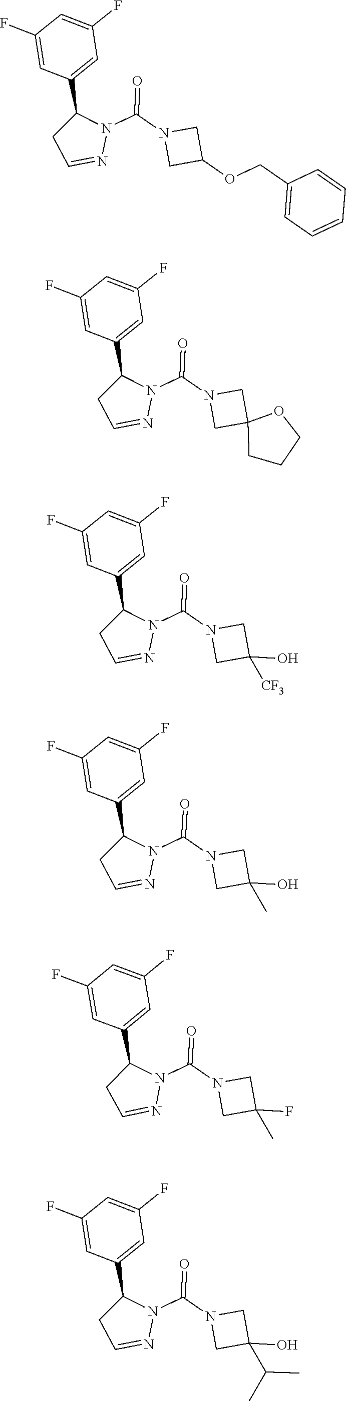 [SMILES: O=C(N1CC(OCc2ccccc2)C1)N1N=CC[C@H]1c1cc(F)cc(F)c1 ; O=C(N1CC2(CCCO2)C1)N1N=CC[C@H]1c1cc(F)cc(F)c1 ; O=C(N1CC(O)(C(F)(F)F)C1)N1N=CC[C@H]1c1cc(F)cc(F)c1 ; CC1(O)CN(C(=O)N2N=CC[C@H]2c2cc(F)cc(F)c2)C1 ; CC1(F)CN(C(=O)N2N=CC[C@H]2c2cc(F)cc(F)c2)C1 ; CC(C)C1(O)CN(C(=O)N2N=CC[C@H]2c2cc(F)cc(F)c2)C1]

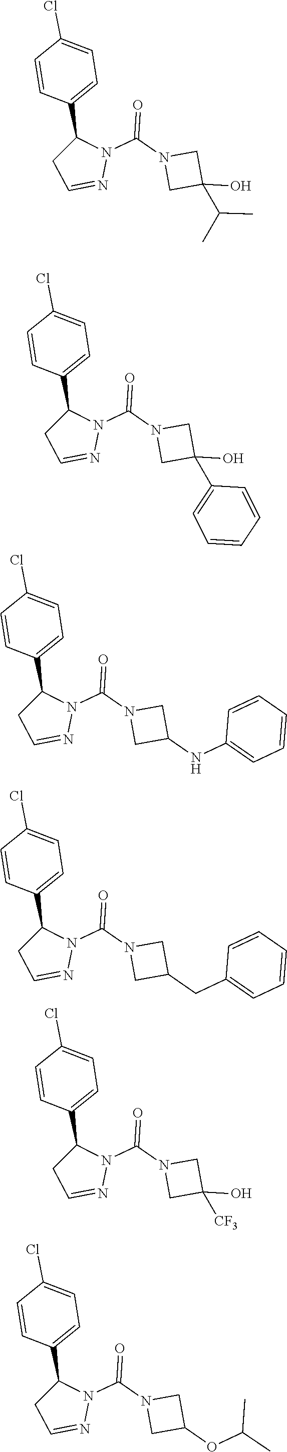

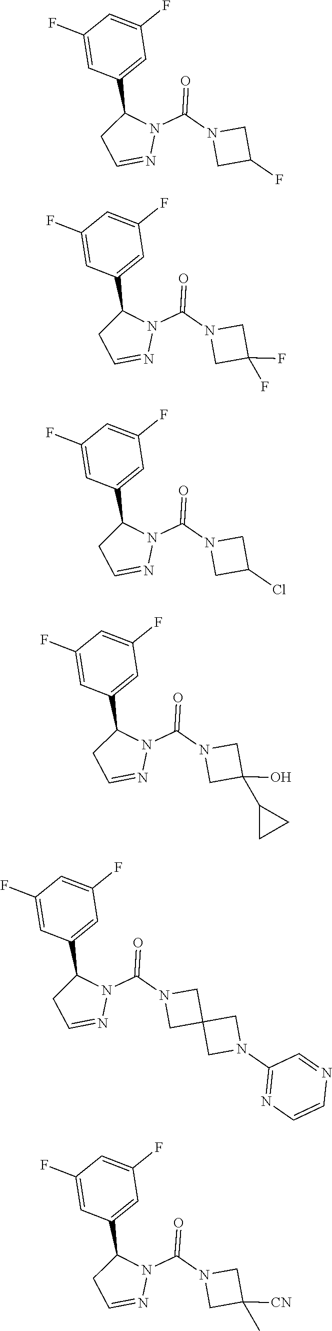

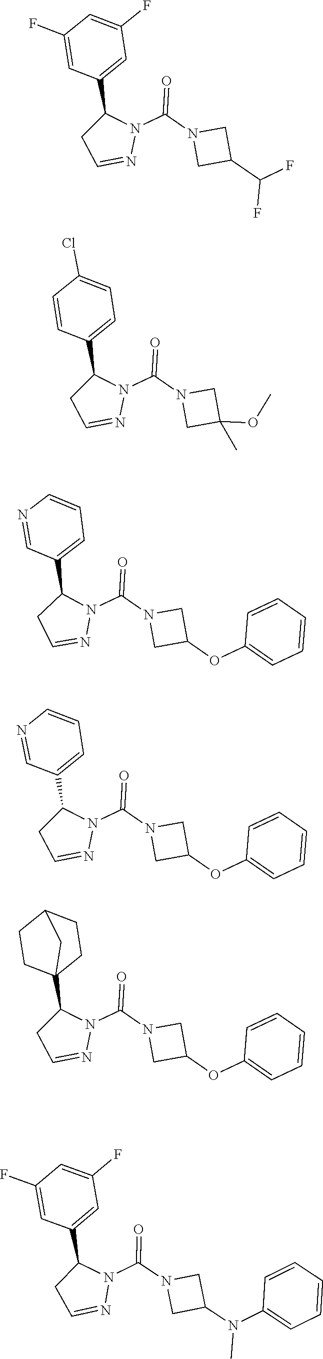 [SMILES: O=C(N1CC(C(F)F)C1)N1N=CC[C@H]1c1cc(F)cc(F)c1 ; COC1(C)CN(C(=O)N2N=CC[C@H]2c2ccc(Cl)cc2)C1 ; O=C(N1CC(Oc2ccccc2)C1)N1N=CC[C@H]1c1cccnc1 ; O=C(N1CC(Oc2ccccc2)C1)N1N=CC[C@@H]1c1cccnc1 ; O=C(N1CC(Oc2ccccc2)C1)N1N=CC[C@H]1C12CCC(CC1)C2 ; CN(c1ccccc1)C1CN(C(=O)N2N=CC[C@H]2c2cc(F)cc(F)c2)C1]

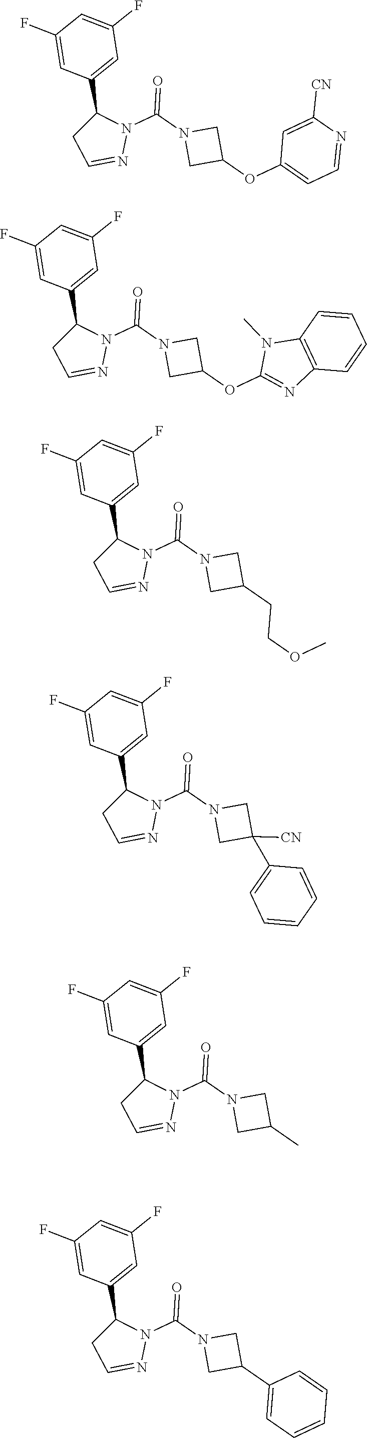 [SMILES: N#Cc1cc(OC2CN(C(=O)N3N=CC[C@H]3c3cc(F)cc(F)c3)C2)ccn1 ; Cn1c(OC2CN(C(=O)N3N=CC[C@H]3c3cc(F)cc(F)c3)C2)nc2ccccc21 ; COCCC1CN(C(=O)N2N=CC[C@H]2c2cc(F)cc(F)c2)C1 ; N#CC1(c2ccccc2)CN(C(=O)N2N=CC[C@H]2c2cc(F)cc(F)c2)C1 ; CC1CN(C(=O)N2N=CC[C@H]2c2cc(F)cc(F)c2)C1 ; O=C(N1CC(c2ccccc2)C1)N1N=CC[C@H]1c1cc(F)cc(F)c1]

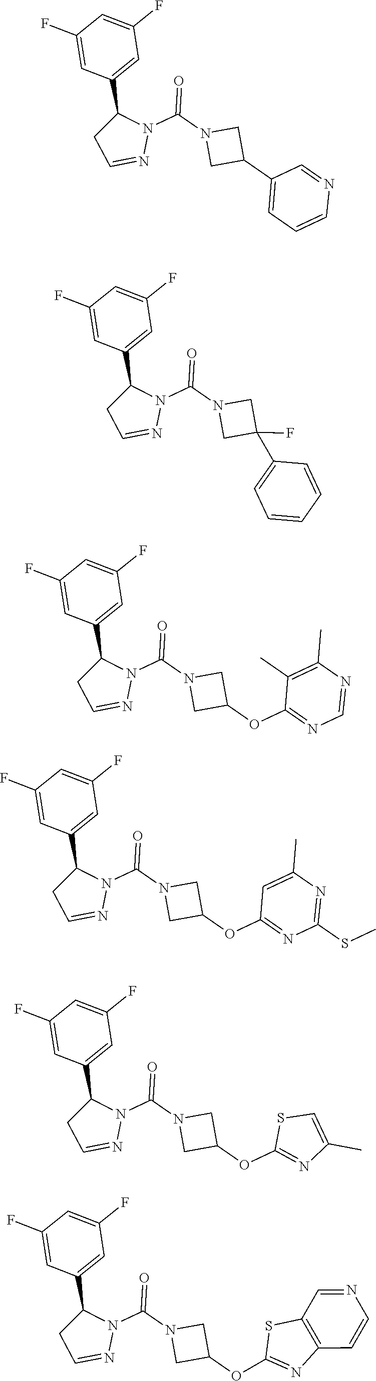 [SMILES: O=C(N1CC(c2cccnc2)C1)N1N=CC[C@H]1c1cc(F)cc(F)c1 ; O=C(N1CC(F)(c2ccccc2)C1)N1N=CC[C@H]1c1cc(F)cc(F)c1 ; Cc1ncnc(OC2CN(C(=O)N3N=CC[C@H]3c3cc(F)cc(F)c3)C2)c1C ; CSc1nc(C)cc(OC2CN(C(=O)N3N=CC[C@H]3c3cc(F)cc(F)c3)C2)n1 ; Cc1csc(OC2CN(C(=O)N3N=CC[C@H]3c3cc(F)cc(F)c3)C2)n1 ; O=C(N1CC(Oc2nc3ccncc3s2)C1)N1N=CC[C@H]1c1cc(F)cc(F)c1]

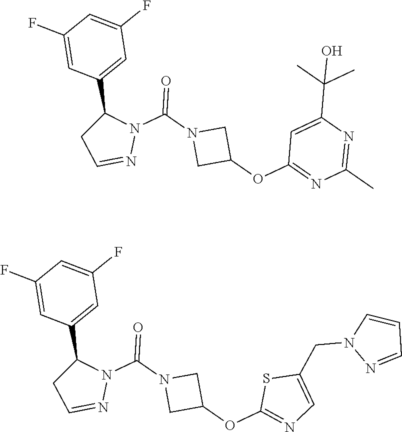 [SMILES: Cc1nc(OC2CN(C(=O)N3N=CC[C@H]3c3cc(F)cc(F)c3)C2)cc(C(C)(C)O)n1 ; O=C(N1CC(Oc2ncc(Cn3cccn3)s2)C1)N1N=CC[C@H]1c1cc(F)cc(F)c1]

Also described are the following compounds, or a pharmaceutically acceptable salt thereof:

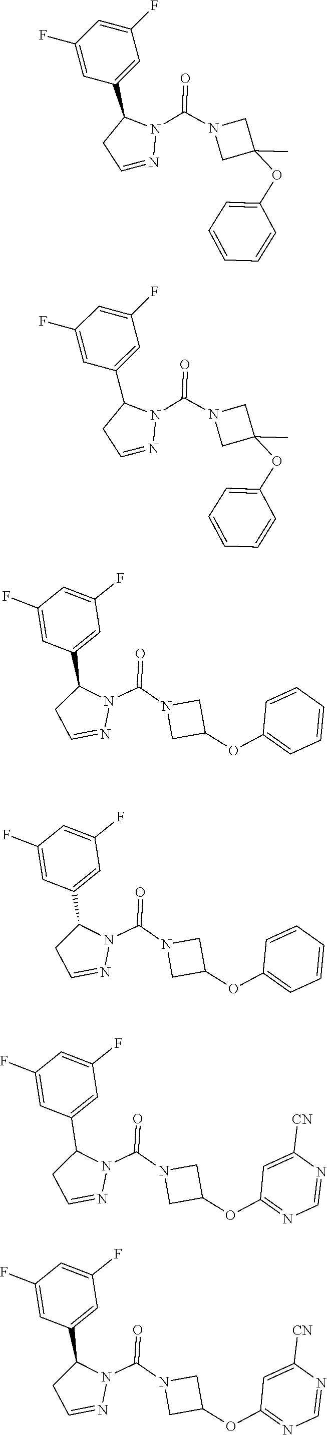 [SMILES: CC1(Oc2ccccc2)CN(C(=O)N2N=CC[C@H]2c2cc(F)cc(F)c2)C1 ; CC1(Oc2ccccc2)CN(C(=O)N2N=CCC2c2cc(F)cc(F)c2)C1 ; O=C(N1CC(Oc2ccccc2)C1)N1N=CC[C@H]1c1cc(F)cc(F)c1 ; O=C(N1CC(Oc2ccccc2)C1)N1N=CC[C@@H]1c1cc(F)cc(F)c1 ; N#Cc1cc(OC2CN(C(=O)N3N=CCC3c3cc(F)cc(F)c3)C2)ncn1 ; N#Cc1cc(OC2CN(C(=O)N3N=CC[C@H]3c3cc(F)cc(F)c3)C2)ncn1]

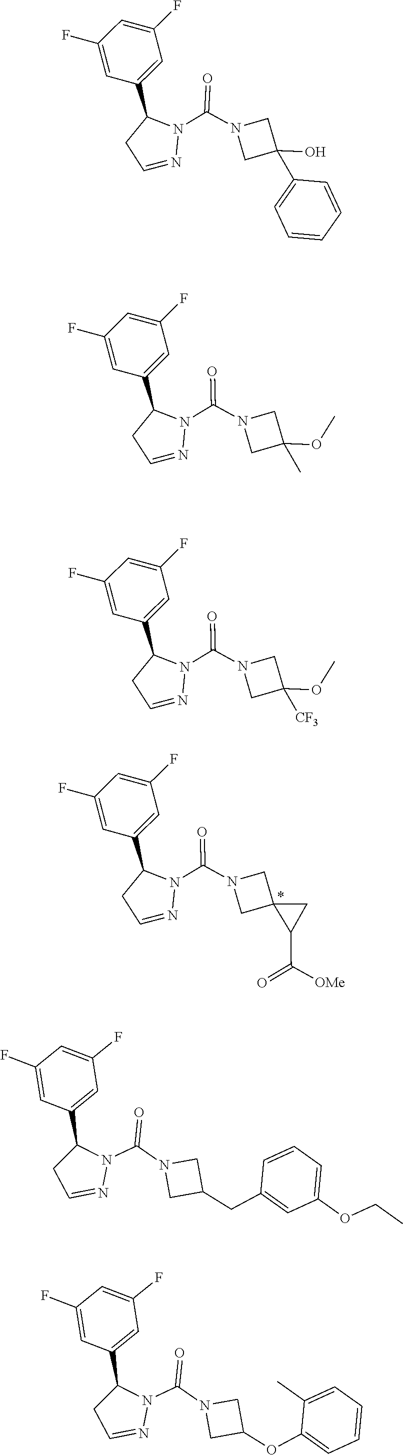 [SMILES: O=C(N1CC(O)(c2ccccc2)C1)N1N=CC[C@H]1c1cc(F)cc(F)c1 ; COC1(C)CN(C(=O)N2N=CC[C@H]2c2cc(F)cc(F)c2)C1 ; COC1(C(F)(F)F)CN(C(=O)N2N=CC[C@H]2c2cc(F)cc(F)c2)C1 ; COC(=O)C1CC12CN(C(=O)N1N=CC[C@H]1c1cc(F)cc(F)c1)C2 ; CCOc1cccc(CC2CN(C(=O)N3N=CC[C@H]3c3cc(F)cc(F)c3)C2)c1 ; Cc1ccccc1OC1CN(C(=O)N2N=CC[C@H]2c2cc(F)cc(F)c2)C1]

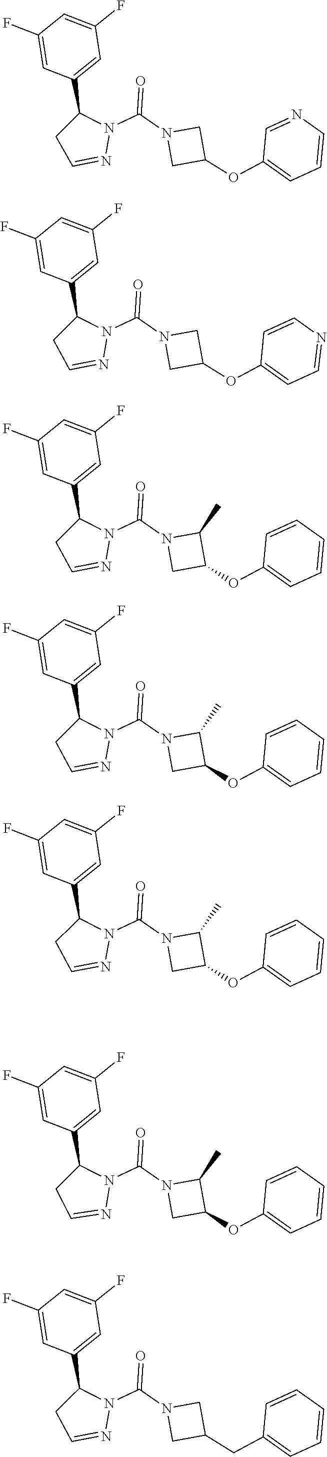

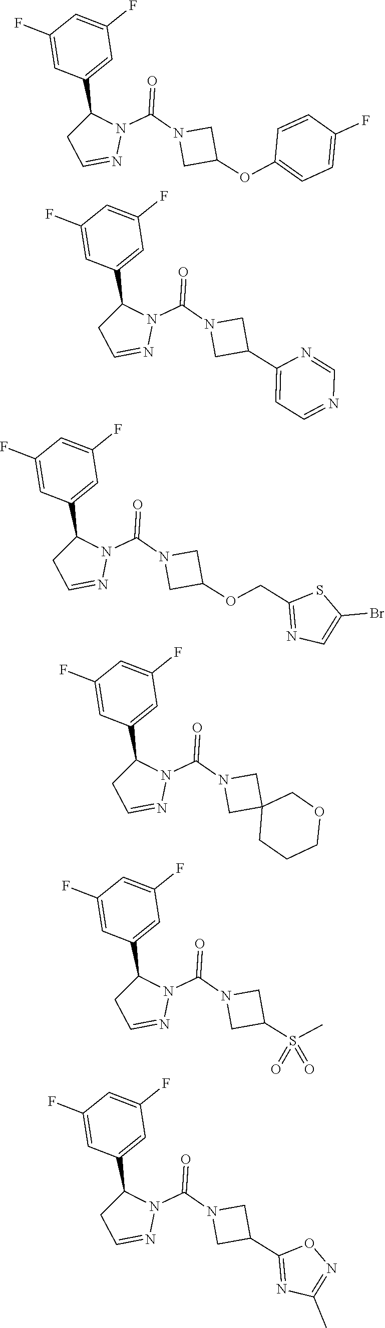

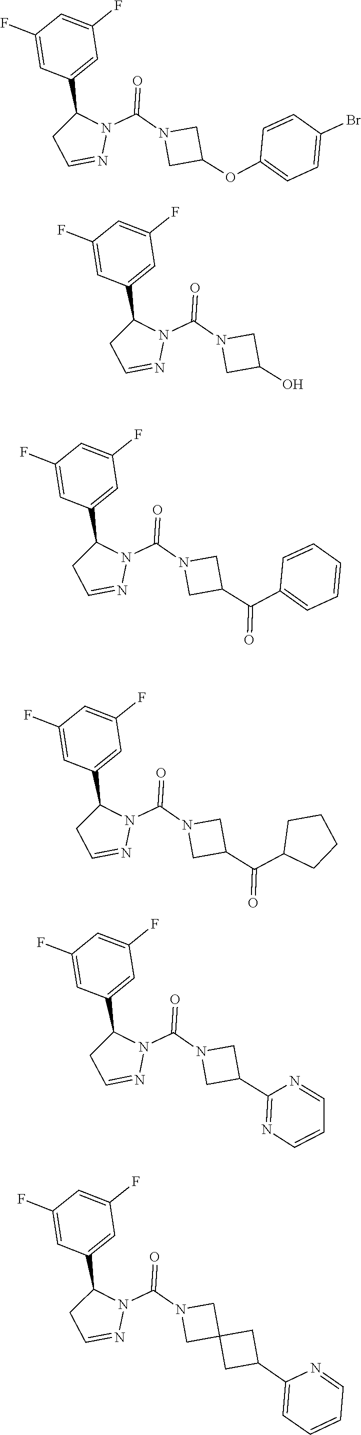 [SMILES: O=C(N1CC(Oc2ccc(Br)cc2)C1)N1N=CC[C@H]1c1cc(F)cc(F)c1 ; O=C(N1CC(O)C1)N1N=CC[C@H]1c1cc(F)cc(F)c1 ; O=C(c1ccccc1)C1CN(C(=O)N2N=CC[C@H]2c2cc(F)cc(F)c2)C1 ; O=C(C1CCCC1)C1CN(C(=O)N2N=CC[C@H]2c2cc(F)cc(F)c2)C1 ; O=C(N1CC(c2ncccn2)C1)N1N=CC[C@H]1c1cc(F)cc(F)c1 ; O=C(N1CC2(CC(c3ccccn3)C2)C1)N1N=CC[C@H]1c1cc(F)cc(F)c1]

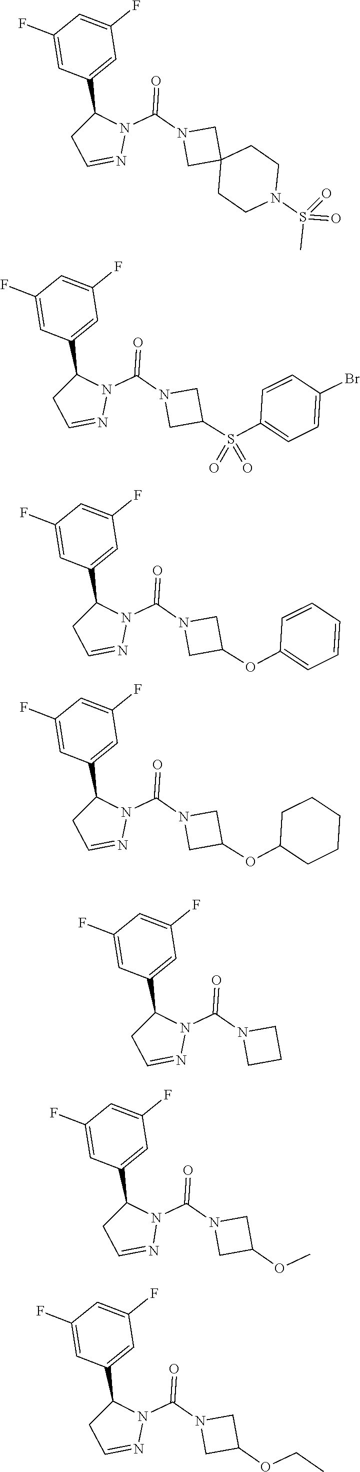

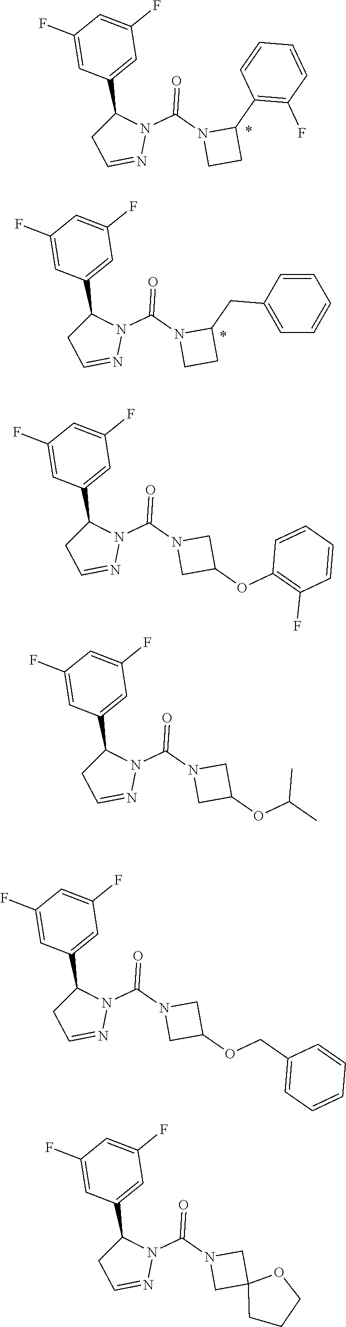

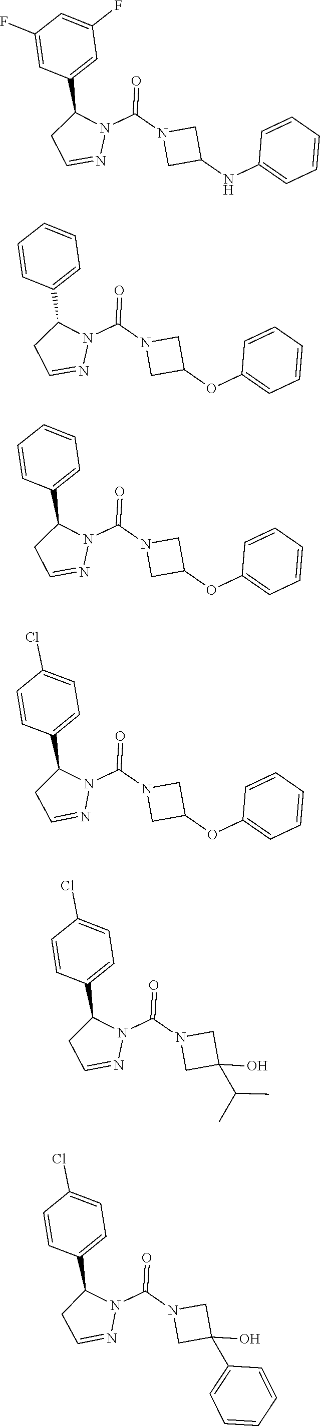 [SMILES: O=C(N1CC(Nc2ccccc2)C1)N1N=CC[C@H]1c1cc(F)cc(F)c1 ; O=C(N1CC(Oc2ccccc2)C1)N1N=CC[C@@H]1c1ccccc1 ; O=C(N1CC(Oc2ccccc2)C1)N1N=CC[C@H]1c1ccccc1 ; O=C(N1CC(Oc2ccccc2)C1)N1N=CC[C@H]1c1ccc(Cl)cc1 ; CC(C)C1(O)CN(C(=O)N2N=CC[C@H]2c2ccc(Cl)cc2)C1 ; O=C(N1CC(O)(c2ccccc2)C1)N1N=CC[C@H]1c1ccc(Cl)cc1]

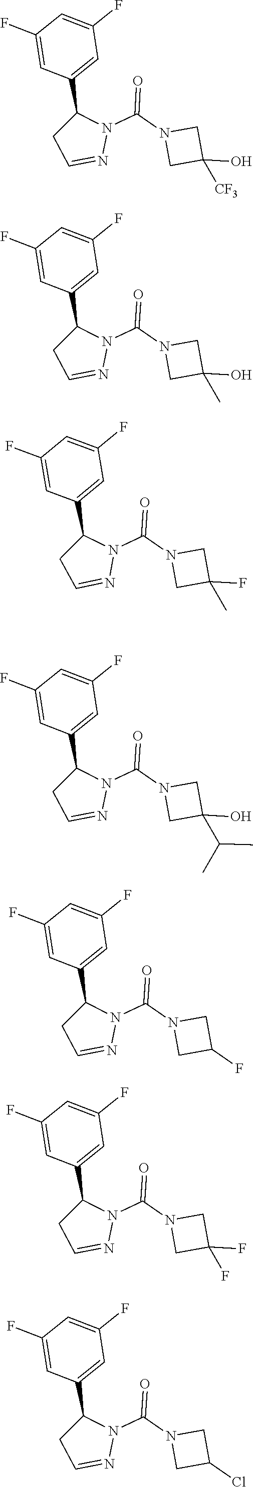

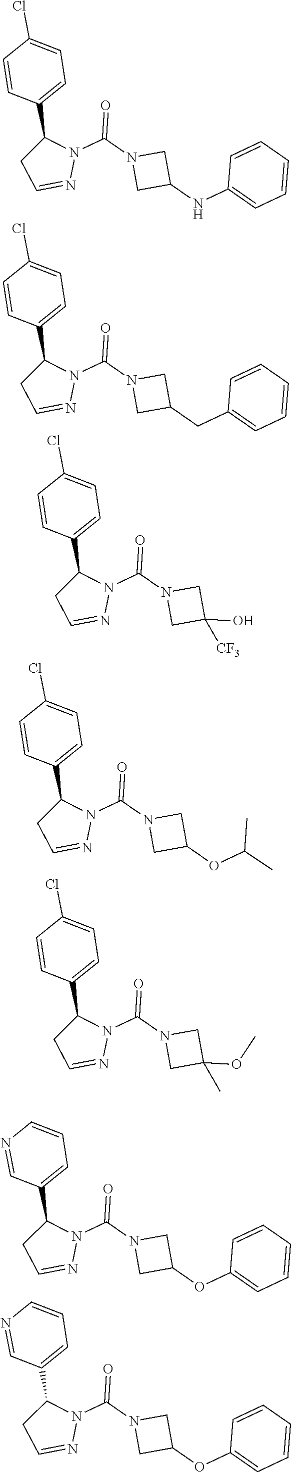

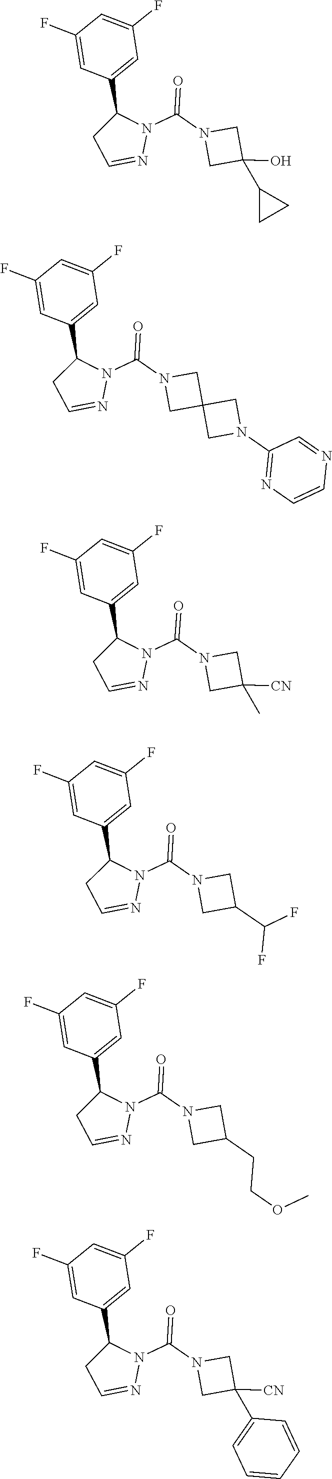 [SMILES: O=C(N1CC(O)(C2CC2)C1)N1N=CC[C@H]1c1cc(F)cc(F)c1 ; O=C(N1CC2(C1)CN(c1cnccn1)C2)N1N=CC[C@H]1c1cc(F)cc(F)c1 ; CC1(C#N)CN(C(=O)N2N=CC[C@H]2c2cc(F)cc(F)c2)C1 ; O=C(N1CC(C(F)F)C1)N1N=CC[C@H]1c1cc(F)cc(F)c1 ; COCCC1CN(C(=O)N2N=CC[C@H]2c2cc(F)cc(F)c2)C1 ; N#CC1(c2ccccc2)CN(C(=O)N2N=CC[C@H]2c2cc(F)cc(F)c2)C1]

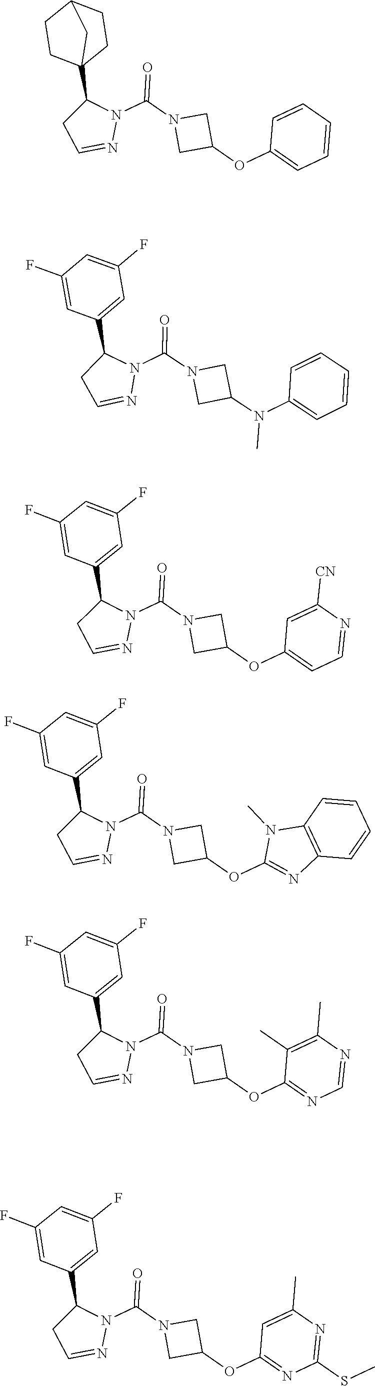 [SMILES: O=C(N1CC(Oc2ccccc2)C1)N1N=CC[C@H]1C12CCC(CC1)C2 ; CN(c1ccccc1)C1CN(C(=O)N2N=CC[C@H]2c2cc(F)cc(F)c2)C1 ; N#Cc1cc(OC2CN(C(=O)N3N=CC[C@H]3c3cc(F)cc(F)c3)C2)ccn1 ; Cn1c(OC2CN(C(=O)N3N=CC[C@H]3c3cc(F)cc(F)c3)C2)nc2ccccc21 ; Cc1ncnc(OC2CN(C(=O)N3N=CC[C@H]3c3cc(F)cc(F)c3)C2)c1C ; CSc1nc(C)cc(OC2CN(C(=O)N3N=CC[C@H]3c3cc(F)cc(F)c3)C2)n1]

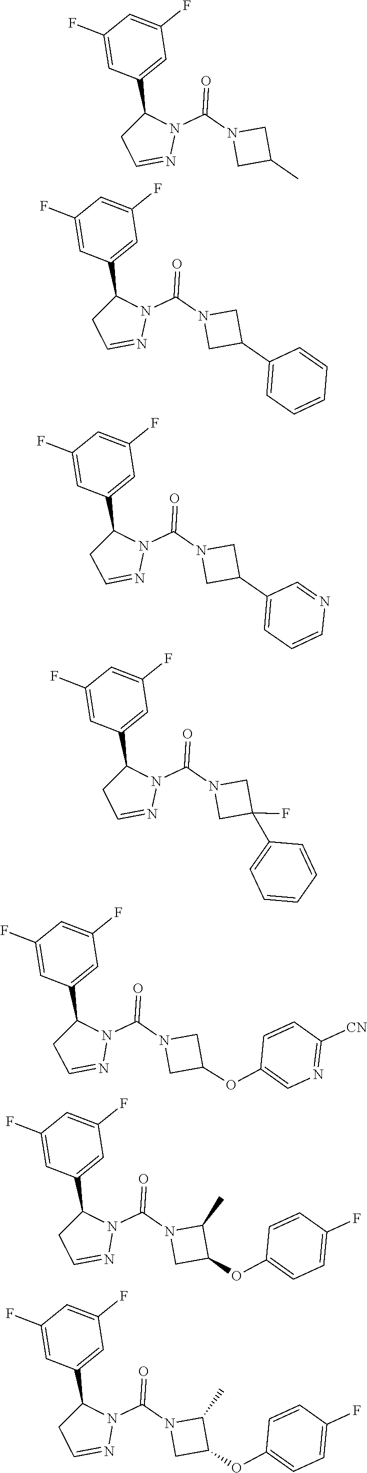

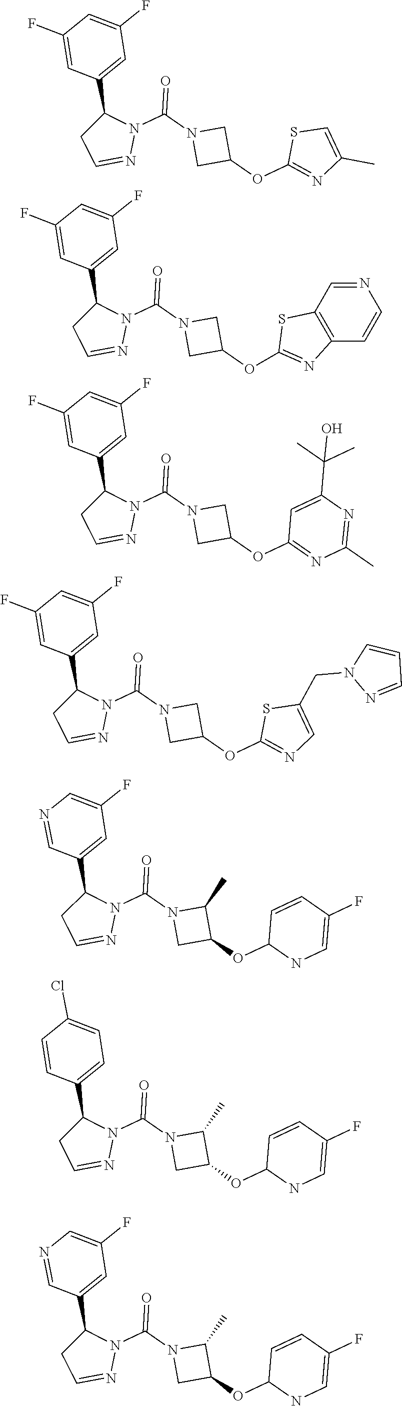

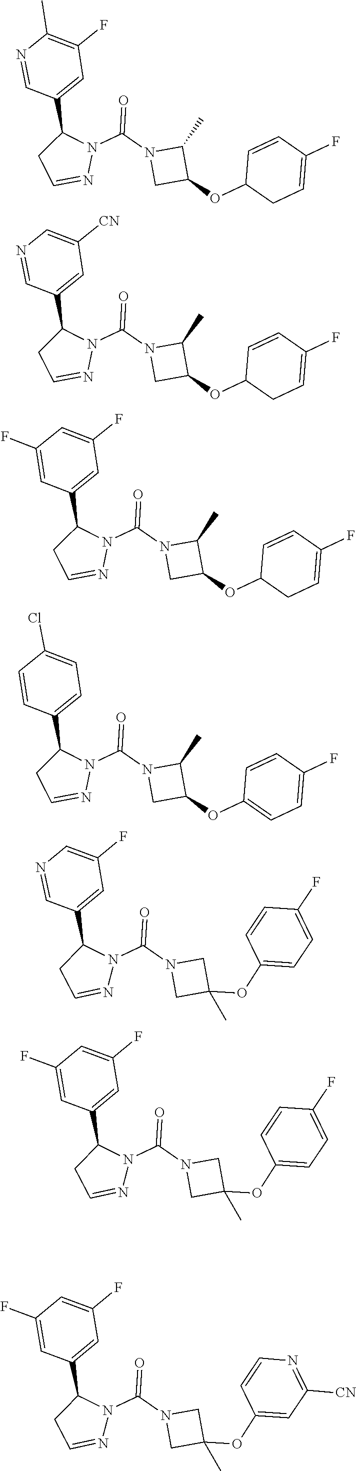

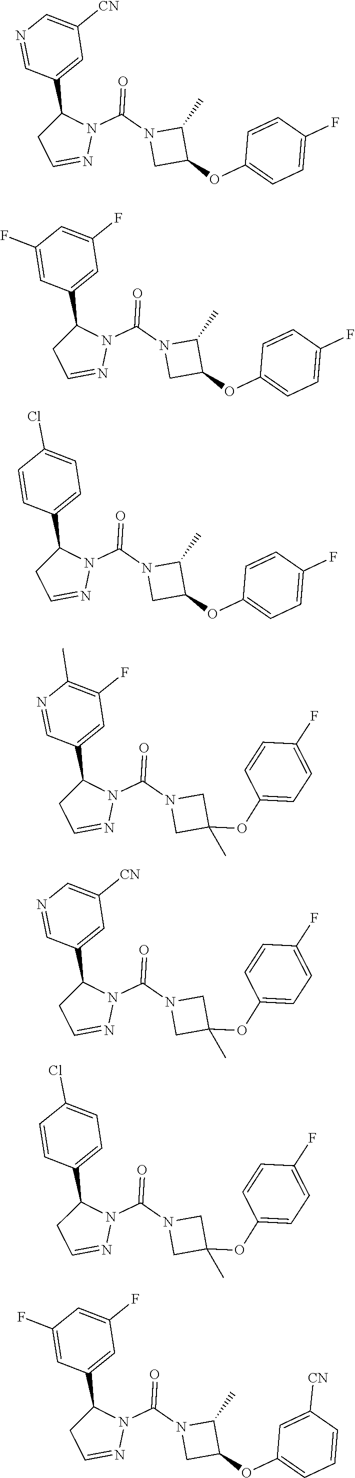

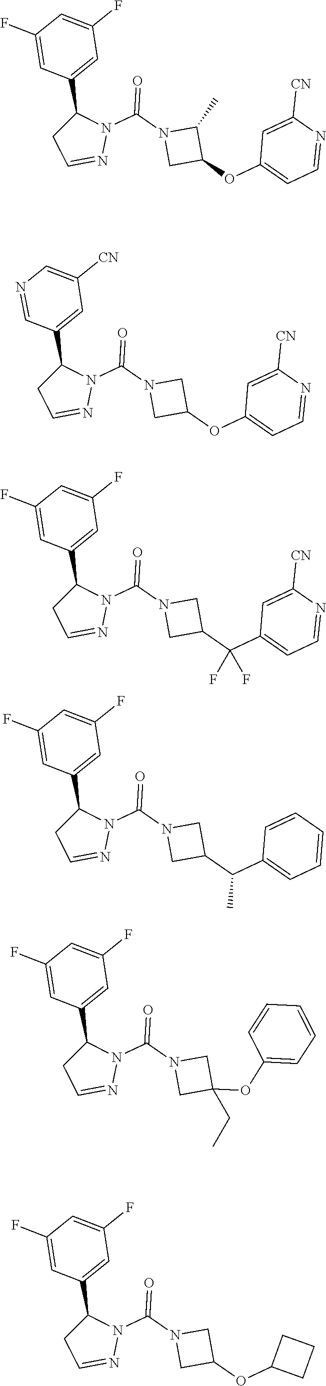 [SMILES: C[C@@H]1[C@@H](Oc2ccnc(C#N)c2)CN1C(=O)N1N=CC[C@H]1c1cc(F)cc(F)c1 ; N#Cc1cncc([C@@H]2CC=NN2C(=O)N2CC(Oc3ccnc(C#N)c3)C2)c1 ; N#Cc1cc(C(F)(F)C2CN(C(=O)N3N=CC[C@H]3c3cc(F)cc(F)c3)C2)ccn1 ; C[C@@H](c1ccccc1)C1CN(C(=O)N2N=CC[C@H]2c2cc(F)cc(F)c2)C1 ; CCC1(Oc2ccccc2)CN(C(=O)N2N=CC[C@H]2c2cc(F)cc(F)c2)C1 ; O=C(N1CC(OC2CCC2)C1)N1N=CC[C@H]1c1cc(F)cc(F)c1]

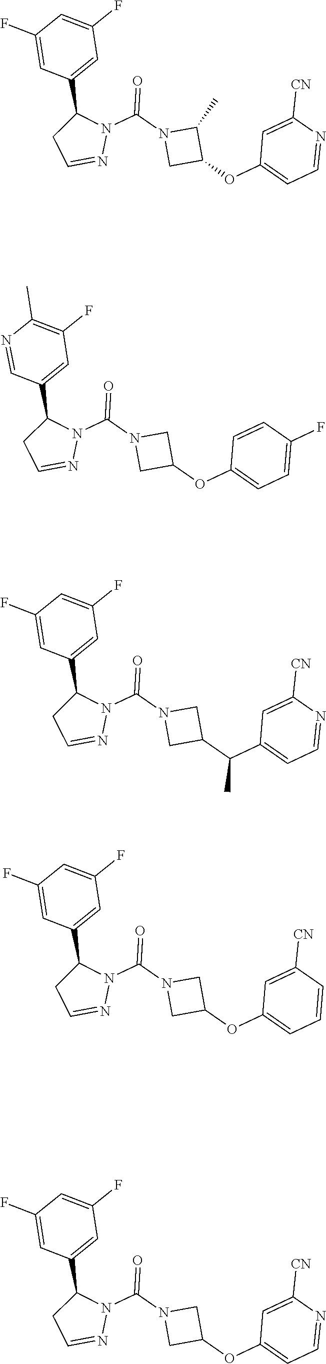 [SMILES: C[C@@H]1[C@H](Oc2ccnc(C#N)c2)CN1C(=O)N1N=CC[C@H]1c1cc(F)cc(F)c1 ; Cc1ncc([C@@H]2CC=NN2C(=O)N2CC(Oc3ccc(F)cc3)C2)cc1F ; C[C@H](c1ccnc(C#N)c1)C1CN(C(=O)N2N=CC[C@H]2c2cc(F)cc(F)c2)C1 ; N#Cc1cccc(OC2CN(C(=O)N3N=CC[C@H]3c3cc(F)cc(F)c3)C2)c1 ; N#Cc1cc(OC2CN(C(=O)N3N=CC[C@H]3c3cc(F)cc(F)c3)C2)ccn1]

Also described are the following compounds, or a pharmaceutically acceptable salt thereof:

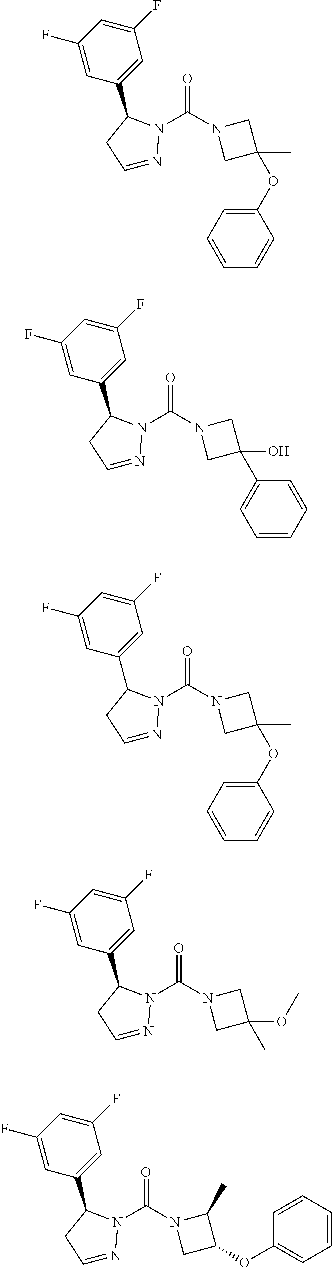 [SMILES: CC1(Oc2ccccc2)CN(C(=O)N2N=CC[C@H]2c2cc(F)cc(F)c2)C1 ; O=C(N1CC(O)(c2ccccc2)C1)N1N=CC[C@H]1c1cc(F)cc(F)c1 ; CC1(Oc2ccccc2)CN(C(=O)N2N=CCC2c2cc(F)cc(F)c2)C1 ; COC1(C)CN(C(=O)N2N=CC[C@H]2c2cc(F)cc(F)c2)C1 ; C[C@H]1[C@H](Oc2ccccc2)CN1C(=O)N1N=CC[C@H]1c1cc(F)cc(F)c1]

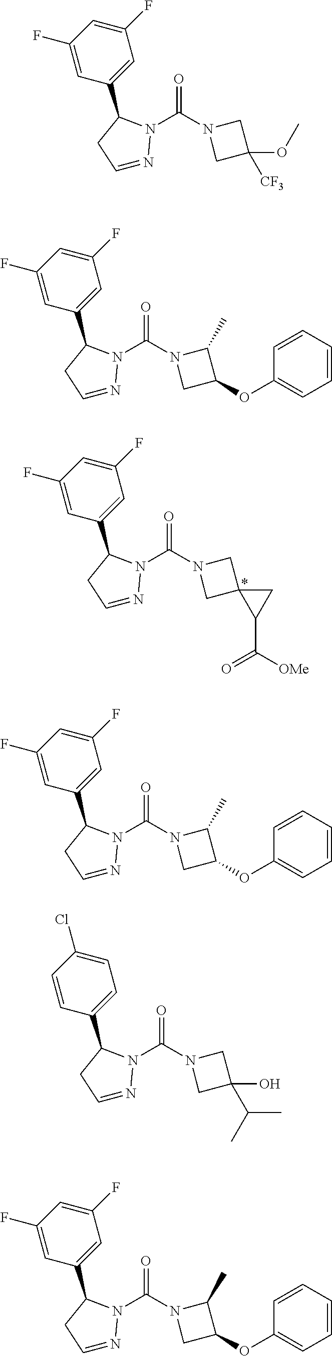

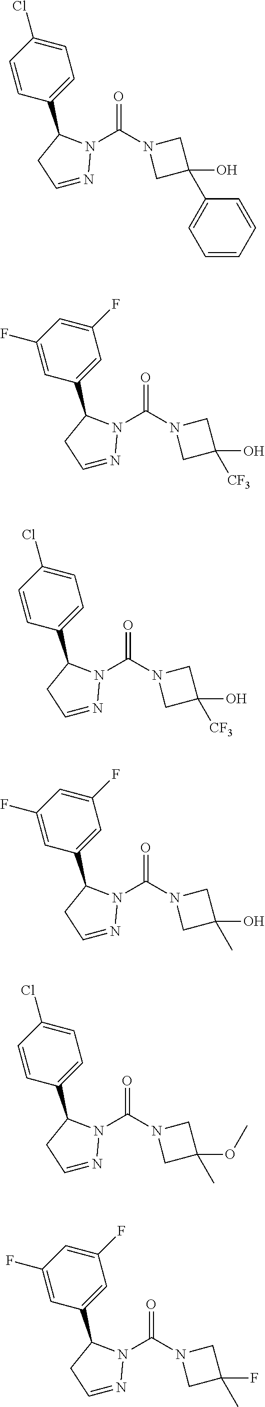 [SMILES: O=C(N1CC(O)(c2ccccc2)C1)N1N=CC[C@H]1c1ccc(Cl)cc1 ; O=C(N1CC(O)(C(F)(F)F)C1)N1N=CC[C@H]1c1cc(F)cc(F)c1 ; O=C(N1CC(O)(C(F)(F)F)C1)N1N=CC[C@H]1c1ccc(Cl)cc1 ; CC1(O)CN(C(=O)N2N=CC[C@H]2c2cc(F)cc(F)c2)C1 ; COC1(C)CN(C(=O)N2N=CC[C@H]2c2ccc(Cl)cc2)C1 ; CC1(F)CN(C(=O)N2N=CC[C@H]2c2cc(F)cc(F)c2)C1]

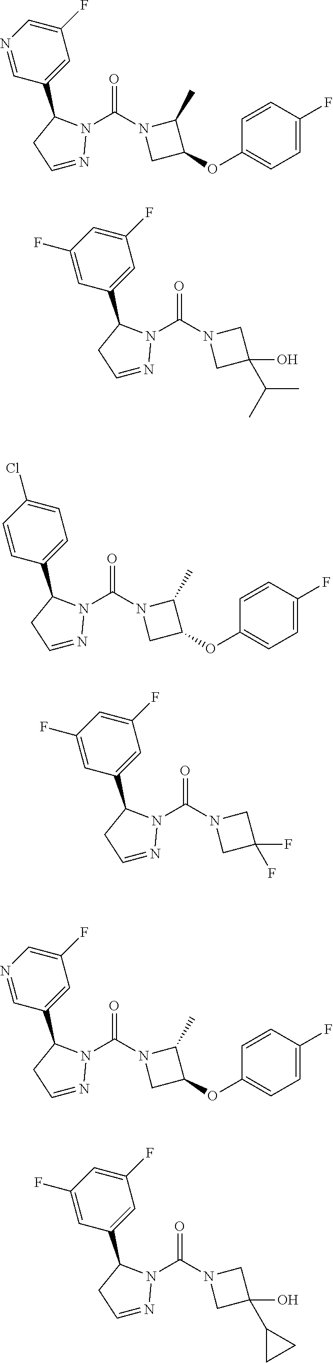 [SMILES: C[C@H]1[C@@H](Oc2ccc(F)cc2)CN1C(=O)N1N=CC[C@H]1c1cncc(F)c1 ; CC(C)C1(O)CN(C(=O)N2N=CC[C@H]2c2cc(F)cc(F)c2)C1 ; C[C@@H]1[C@H](Oc2ccc(F)cc2)CN1C(=O)N1N=CC[C@H]1c1ccc(Cl)cc1 ; O=C(N1CC(F)(F)C1)N1N=CC[C@H]1c1cc(F)cc(F)c1 ; C[C@@H]1[C@@H](Oc2ccc(F)cc2)CN1C(=O)N1N=CC[C@H]1c1cncc(F)c1 ; O=C(N1CC(O)(C2CC2)C1)N1N=CC[C@H]1c1cc(F)cc(F)c1]

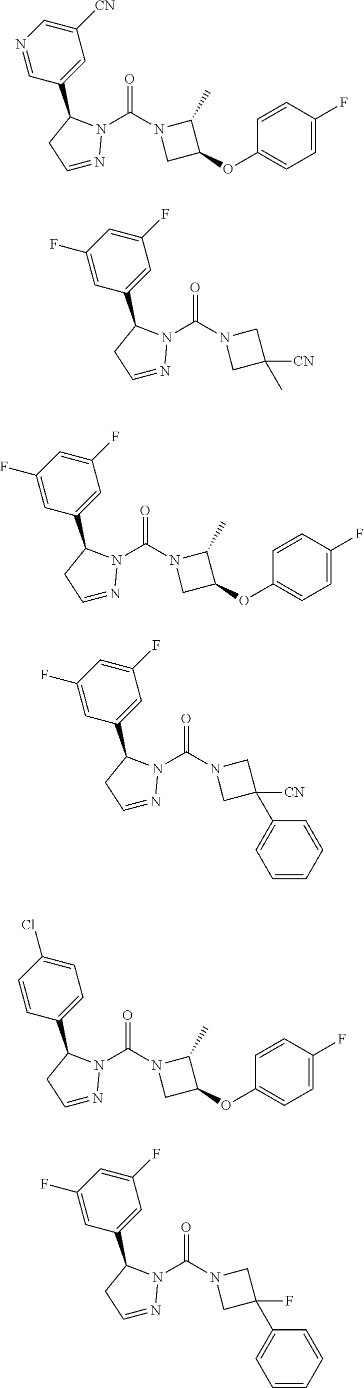 [SMILES: C[C@@H]1[C@@H](Oc2ccc(F)cc2)CN1C(=O)N1N=CC[C@H]1c1cncc(C#N)c1 ; CC1(C#N)CN(C(=O)N2N=CC[C@H]2c2cc(F)cc(F)c2)C1 ; C[C@@H]1[C@@H](Oc2ccc(F)cc2)CN1C(=O)N1N=CC[C@H]1c1cc(F)cc(F)c1 ; N#CC1(c2ccccc2)CN(C(=O)N2N=CC[C@H]2c2cc(F)cc(F)c2)C1 ; C[C@@H]1[C@@H](Oc2ccc(F)cc2)CN1C(=O)N1N=CC[C@H]1c1ccc(Cl)cc1 ; O=C(N1CC(F)(c2ccccc2)C1)N1N=CC[C@H]1c1cc(F)cc(F)c1]

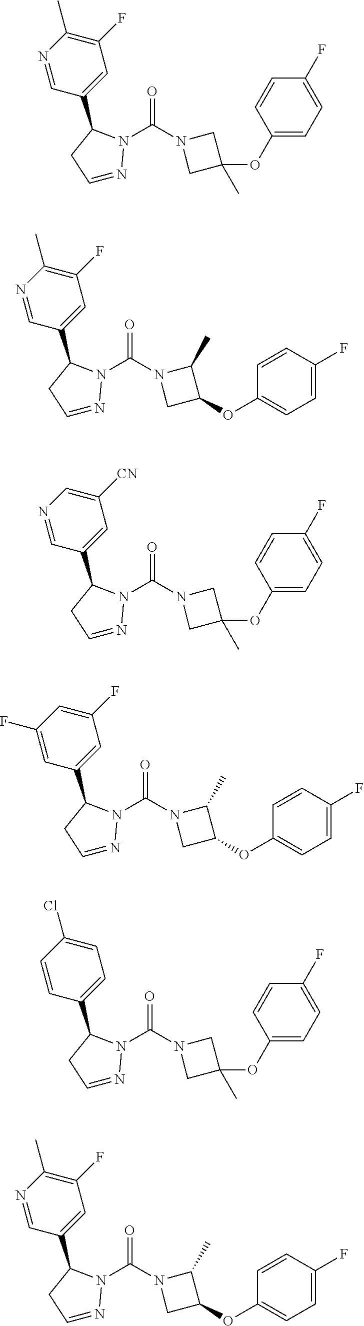 [SMILES: Cc1ncc([C@@H]2CC=NN2C(=O)N2CC(C)(Oc3ccc(F)cc3)C2)cc1F ; Cc1ncc([C@@H]2CC=NN2C(=O)N2C[C@H](Oc3ccc(F)cc3)[C@@H]2C)cc1F ; CC1(Oc2ccc(F)cc2)CN(C(=O)N2N=CC[C@H]2c2cncc(C#N)c2)C1 ; C[C@@H]1[C@H](Oc2ccc(F)cc2)CN1C(=O)N1N=CC[C@H]1c1cc(F)cc(F)c1 ; CC1(Oc2ccc(F)cc2)CN(C(=O)N2N=CC[C@H]2c2ccc(Cl)cc2)C1 ; Cc1ncc([C@@H]2CC=NN2C(=O)N2C[C@H](Oc3ccc(F)cc3)[C@H]2C)cc1F]

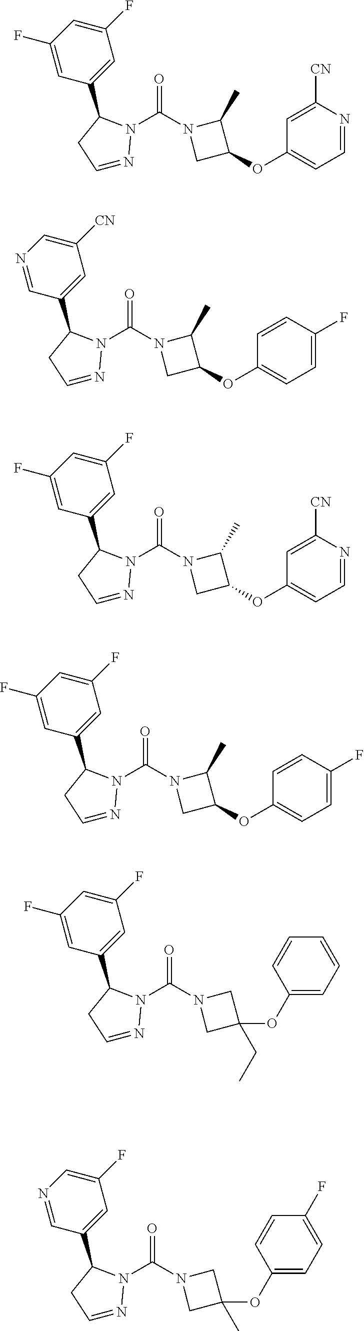 [SMILES: C[C@H]1[C@@H](Oc2ccnc(C#N)c2)CN1C(=O)N1N=CC[C@H]1c1cc(F)cc(F)c1 ; C[C@H]1[C@@H](Oc2ccc(F)cc2)CN1C(=O)N1N=CC[C@H]1c1cncc(C#N)c1 ; C[C@@H]1[C@H](Oc2ccnc(C#N)c2)CN1C(=O)N1N=CC[C@H]1c1cc(F)cc(F)c1 ; C[C@H]1[C@@H](Oc2ccc(F)cc2)CN1C(=O)N1N=CC[C@H]1c1cc(F)cc(F)c1 ; CCC1(Oc2ccccc2)CN(C(=O)N2N=CC[C@H]2c2cc(F)cc(F)c2)C1 ; CC1(Oc2ccc(F)cc2)CN(C(=O)N2N=CC[C@H]2c2cncc(F)c2)C1]

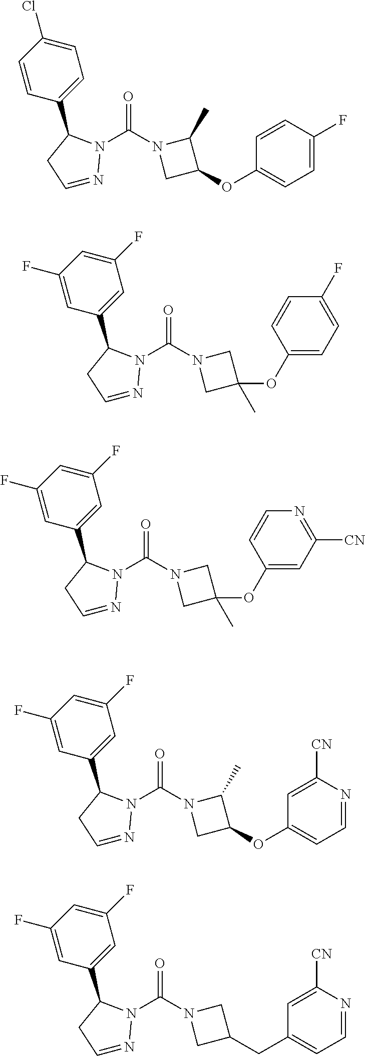 [SMILES: C[C@H]1[C@@H](Oc2ccc(F)cc2)CN1C(=O)N1N=CC[C@H]1c1ccc(Cl)cc1 ; CC1(Oc2ccc(F)cc2)CN(C(=O)N2N=CC[C@H]2c2cc(F)cc(F)c2)C1 ; CC1(Oc2ccnc(C#N)c2)CN(C(=O)N2N=CC[C@H]2c2cc(F)cc(F)c2)C1 ; C[C@@H]1[C@@H](Oc2ccnc(C#N)c2)CN1C(=O)N1N=CC[C@H]1c1cc(F)cc(F)c1 ; N#Cc1cc(CC2CN(C(=O)N3N=CC[C@H]3c3cc(F)cc(F)c3)C2)ccn1]

### Definitions

“Alkoxy” means an alkyl-O— group in which the alkyl group encompasses straight alkyl having a carbon number of 1 to 10 and branched alkyl having a carbon number of 3 to 10. Non-limiting examples of suitable alkoxy groups include methoxy, ethoxy, n-propoxy, isopropoxy and n-butoxy. The bond to the parent moiety is through the ether oxygen.

The term “halogen” includes fluorine, chlorine, bromine or iodine.

The term “C₁-C₆alkyl” encompasses straight alkyl having a carbon number of 1 to 6 and branched alkyl having a carbon number of 3 to 6. Specific examples thereof include methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl, 1-ethyl-1-methylpropyl, and the like.

The term “C₃-C₆cycloalkyl” encompasses bridged, saturated or unsaturated cycloalkyl groups having 3 to 6 carbons. Examples of cycloalkyl include cyclopropyl, cyclobutyl, cyclopentyl and cyclohexyl.

The term “C₃-C₁₀cycloalkyl” encompasses bridged, saturated or unsaturated cycloalkyl groups having 3 to 10 carbons. “Cycloalkyl” also includes non-aromatic rings as well as monocyclic, non-aromatic rings fused to a saturated cycloalkyl group. Examples of cycloalkyl include cyclopropyl, cyclobutyl, cyclopentyl, cyclohexyl, cycloheptyl, tetrahydronaphthyl, decahydronaphthyl, indanyl and the like. Examples described by structure include,

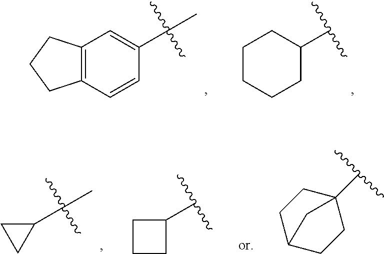

The term “heteroaryl” means a monocyclic or multicyclic, including bicyclic, aromatic cycloheteroalkyl that contains at least one ring heteroatom selected from O, S and N. Examples of heteroaryl groups include pyridyl (pyridinyl), oxazolyl, azabenzothiazole, benzothiazole, imidazolyl, triazolyl, furyl, triazinyl, thienyl, pyrimidyl, pyridazinyl, indolizinyl, cinnolinyl, phthalazinyl, quinazolinyl, naphthyridinyl, quinoxalinyl, purinyl, benzimidazolyl, quinolyl, isoquinolyl, and the like.

The term “cycloheteroalkyl” means mono- or bicyclic or bridged partially unsaturated and saturated rings containing at least one heteroatom selected from N, S and O, each of said rings having from 3 to 10 atoms in which the point of attachment may be carbon or nitrogen. Examples include azetidine, tetrahydropyranyl, tetrahydrofuranyl, pyrrolidinyl, piperidinyl, piperazinyl, dioxanyl, imidazolidinyl, 2,3-dihydrofuro(2,3-b)pyridyl, benzoxazinyl, benzoxazolinyl, 2-H-phthalazinyl, isoindolinyl, benzoxazepinyl, 5,6-dihydroimidazo[2,1-b]thiazolyl, tetrahydroquinolinyl, morpholinyl, tetrahydroisoquinolinyl, dihydroindolyl, and the like. The term also includes partially unsaturated monocyclic rings that are not aromatic, such as 2- or 4-pyridones attached through the nitrogen or n-substituted-(1H, 3H)-pyrimidine-2,4-diones (N-substituted uracils). The term also includes bridged rings such as 5-azabicyclo[2.2.1]heptyl, 2,5-diazabicyclo[2.2.1]heptyl, 2-azabicyclo[2.2.1]heptyl, 7-azabicyclo[2.2.1]heptyl, 2,5-diazabicyclo[2.2.2]octyl, 2-azabicyclo[2.2.2]octyl, and 3-azabicyclo[3.2.2]nonyl, and azabicyclo[2.2.1]heptanyl. Examples described by structure include,

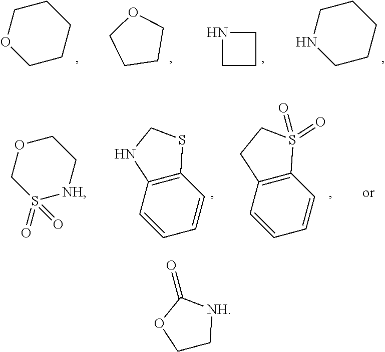

The term “pharmaceutically acceptable salt” refers to salts prepared from pharmaceutically acceptable non-toxic bases or acids including inorganic or organic bases and inorganic or organic acids. Salts of basic compounds encompassed within the term “pharmaceutically acceptable salt” refer to non-toxic salts of the compounds of this invention which are generally prepared by reacting the free base with a suitable organic or inorganic acid. Representative salts of basic compounds of the present invention include, but are not limited to, the following: acetate, benzenesulfonate, benzoate, bicarbonate, bisulfate, bitartrate, borate, bromide, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, n-methylglucamine ammonium salt, oleate, oxalate, pamoate (embonate), palmitate, pantothenate, phosphate/diphosphate, polygalacturonate, salicylate, stearate, sulfate, subacetate, succinate, tannate, tartrate, teoclate, tosylate, triethiodide and valerate. Furthermore, where the compounds of the invention carry an acidic moiety, suitable pharmaceutically acceptable salts thereof include, but are not limited to, salts derived from inorganic bases including aluminum, ammonium, calcium, copper, ferric, ferrous, lithium, magnesium, manganic, manganous, potassium, sodium, zinc, and the like. Particularly preferred are the ammonium, calcium, magnesium, potassium, and sodium salts. Salts derived from pharmaceutically acceptable organic non-toxic bases include salts of primary, secondary, and tertiary amines, cyclic amines, and basic ion-exchange resins, such as arginine, betaine, caffeine, choline, N,N-dibenzylethylenediamine, diethylamine, 2-diethylaminoethanol, 2-dimethylaminoethanol, ethanolamine, ethylenediamine, n-ethylmorpholine, n-ethylpiperidinyl, glucamine, glucosamine, histidine, hydrabamine, isopropylamine, lysine, methylglucamine, morpholine, piperazine, piperidinyl, polyamine resins, procaine, purines, theobromine, triethylamine, trimethylamine, tripropylamine, tromethamine, and the like.

The term “patient” refers to a mammalian patient, preferably a human patient, receiving or about to receive medical treatment.

The compounds of the present invention may contain one or more asymmetric centers and can thus occur as racemates, racemic mixtures, single enantiomers, diastereomeric mixtures, and individual diastereomers. The present invention is meant to comprehend all such isomeric forms of these compounds.

Some of the compounds described herein contain olefinic double bonds, and unless specified otherwise, are meant to include both E and Z geometric isomers.

Some of the compounds described herein contain substituted cycloalkanes having cis- and trans-isomers, and unless specified otherwise, are meant to include both cis- and trans-geometric isomers.

The independent syntheses of these diastereomers or their chromatographic separations may be achieved as known in the art by appropriate modification of the methodology disclosed herein. Their absolute stereochemistry may be determined by the X-ray crystallography of crystalline products or crystalline intermediates which are derivatized, if necessary, with a reagent containing an asymmetric center of known absolute configuration. If desired, racemic mixtures of the compounds may be separated so that the individual enantiomers are isolated. The separation can be carried out by methods well known in the art, such as the coupling of a racemic mixture of compounds to an enantiomerically pure compound to form a diastereomeric mixture, followed by separation of the individual diastereomers by standard methods, such as fractional crystallization or chromatography. The coupling reaction is often the formation of salts using an enantiomerically pure acid or base. The diastereomeric derivatives may then be converted to the pure enantiomers by cleavage of the added chiral residue. The racemic mixture of the compounds can also be separated directly by chromatographic methods utilizing chiral stationary phases, which methods are well known in the art.

Alternatively, any enantiomer of a compound may be obtained by stereoselective synthesis using optically pure starting materials or reagents of known configuration by methods well known in the art.

It will be understood that the present invention is meant to include the pharmaceutically acceptable salts, and also salts that are not pharmaceutically acceptable, of the compounds described herein, when they are used as precursors to the free compounds or their pharmaceutically acceptable salts or in other synthetic manipulations.

Solvates, and in particular, the hydrates of the compounds of the structural formulas described herein are included in the present invention as well.

Some of the compounds described herein may exist as tautomers, which have different points of attachment of hydrogen accompanied by one or more double bond shifts. For example, a ketone and its enol form are keto-enol tautomers. The individual tautomers as well as mixtures thereof are encompassed with compounds of the present invention.

In the compounds described herein, the atoms may exhibit their natural isotopic abundances, or one or more of the atoms may be artificially enriched in a particular isotope having the same atomic number, but an atomic mass or mass number different from the atomic mass or mass number predominantly found in nature. The present invention is meant to include all suitable isotopic variations of the compounds of the formulas described herein. For example, different isotopic forms of hydrogen (H) include protium (¹H) and deuterium (²H). Protium is the predominant hydrogen isotope found in nature. Enriching for deuterium may afford certain therapeutic advantages, such as increasing in vivo half-life or reducing dosage requirements, or may provide a compound useful as a standard for characterization of biological samples. A ³H, ¹¹C, ¹⁸F labeled compound may be used for PET or SPECT or other imaging studies. Isotopically-enriched compounds can be prepared without undue experimentation by conventional techniques well known to those skilled in the art or by processes analogous to those described in the Schemes and Examples herein using appropriate isotopically-enriched reagents or Intermediates.

It should be noted that chemically unstable compounds are excluded from the embodiments contained herein.

### Methods of Treatment

The compounds described herein may be particularly useful for the prevention, treatment or amelioration of RIPK1-mediated diseases or disorders. Such RIPK1-mediated diseases or disorders are likely to be regulated at least in part by programmed necrosis, apoptosis or the production of inflammatory cytokines, particularly inflammatory bowel disease (including Crohn's disease and ulcerative colitis), psoriasis, retinal detachment, retinal degeneration, retinitis pigmentosa, macular degeneration, age-related macular degeneration, pancreatitis, atopic dermatitis, arthritis (including rheumatoid arthritis, spondyloarthritis, gout, juvenile idiopathic arthritis (systemic onset juvenile idiopathic arthritis (SoJIA)), psoriatic arthritis), lupus, systemic lupus erythematosus (SLE), Sjogren's syndrome, systemic scleroderma, anti-phospholipid syndrome (APS), vasculitis, osteoarthritis, liver damage/diseases (non-alcohol steatohepatitis (NASH), alcohol steatohepatitis (ASH), autoimmune hepatitis, autoimmune hepatobiliary diseases, primary sclerosing cholangitis (PSC), acetaminophen toxicity, hepatotoxicity), non-alcohol steatohepatitis (NASH), alcohol steatohepatitis (ASH), autoimmune hepatitis, non-alcoholic fatty liver disease (NAFL D), kidney damage/injury (nephritis, renal transplant, surgery, administration of nephrotoxic drugs e.g. cisplatin, acute kidney injury (AKI)), Celiac disease, autoimmune idiopathic thrombocytopenic purpura (autoimmune ITP), transplant rejection (rejection of transplant organs, tissues and cells), ischemia reperfusion injury of solid organs, sepsis, systemic inflammatory response syndrome (SIRS), cerebrovascular accident (CV A, stroke), myocardial infarction (Ml), atherosclerosis, Huntington's disease, Alzheimer's disease, Parkinson's disease, amyotrophic lateral sclerosis (ALS), progressive supranuclear palsy (PSP), neonatal brain injury, neonatal hypoxic brain injury, ischemic brain injury, traumatic brain injury allergic diseases (including asthma and atopic dermatitis), peripheral nerve injury, burns, multiple sclerosis, type I diabetes, type II diabetes, obesity, Wegener's granulomatosis, pulmonary sarcoidosis, Behcet's disease, interleukin-I converting enzyme (ICE, also known as caspase-1) associated fever syndrome, chronic obstructive pulmonary disease (COPD), cigarette smoke-induced damage, cystic fibrosis, tumor necrosis factor receptor-associated periodic syndrome (TRAPS), a neoplastic tumor, peridontitis, NEMO-mutations (mutations of NF-kappa-B essential modulator gene (also known as IKK gamma or IKKG)), particularly, NEMO-deficiency syndrome, HOIL-1 deficiency (also known as RBCKI) heme-oxidized IRP 2 ubiquitin ligase-1 deficiency), linear ubiquitin chain assembly complex (LUBAC) deficiency syndrome, hematological and solid organ malignancies, bacterial infections and viral infections (such as influenza, staphylococcus, and mycobacterium (tuberculosis)), and Lysosomal storage diseases (particularly, Gaucher disease, and including GM2 gangliosidosis, alpha-mannosidosis, aspartylglucosaminuria, cholesteryl ester storage disease, chronic hexosaminidase A deficiency, cystinosis, Danon disease, Fabry disease, Farber disease, fucosidosis, galactosialidosis, GMl gangliosidosis, mucolipidosis, infantile free sialic acid storage disease, juvenile hexosaminidase A deficiency, Krabbe disease, lysosomal acid lipase deficiency, metachromatic leukodystrophy, mucopolysaccharidoses disorders, multiple sulfatase deficiency, Niemann-Pick disease, neuronal ceroid lipofuscinoses, Pompe disease, pycnodysostosis, Sandhoff disease, Schindler disease, sialic acid storage disease, Tay-Sachs, and Wolman disease), Stevens-Johnson syndrome, toxic epidermal necrolysis, glaucoma, spinal cord injury, fibrosis, complement-mediated cytotoxicity, pancreatic ductal adenocarcinoma, hepatocellular carcinoma, mesothelioma, melanoma, metastasis, breast cancer, non-small cell lung carcinoma (NSCLC), radiation induced necrosis, ischemic kidney damage, ophthalmologic ischemia, intracerebral hemorrhage, subarachnoid hemorrhage, acute liver failure and radiation protection/mitigation, auditory disorders such as noise-induced hearing loss and drugs associated with ototoxicity such as cisplatin, or for the treatment of cells ex vivo to preserve vitality and function.

The compounds of the Formulae described herein, or a pharmaceutically acceptable salt thereof, may be particularly useful for the treatment of the following RIPK1-mediated diseases or disorders: inflammatory bowel disease (including Crohn's disease and ulcerative colitis), psoriasis, retinal detachment, retinal degeneration, retinitis pigmentosa, macular degeneration, age-related macular degeneration, pancreatitis, atopic dermatitis, arthritis (including rheumatoid arthritis, spondyloarthritis, gout, systemic onset juvenile idiopathic arthritis (SoJIA), psoriatic arthritis), lupus, systemic lupus erythematosus (SLE), Sjogren's syndrome, systemic scleroderma, anti-phospholipid syndrome (APS), vasculitis, osteoarthritis, liver damage/diseases (non-alcohol steatohepatitis (NASH), alcohol steatohepatitis (ASH) autoimmune hepatitis, autoimmune hepatobiliary diseases, primary sclerosing cholangitis (PSC), acetaminophen toxicity, hepatotoxicity), non-alcoholic steatohepatitis (NASH), alcoholic steatohepatitis (ASH), autoimmune hepatitis, non-alcoholic fatty liver disease (NAFLD), kidney damage/injury (nephritis, renal transplant, surgery, administration of nephrotoxic drugs e.g. cisplatin, acute kidney injury (AKI)), Celiac disease, autoimmune idiopathic thrombocytopenic purpura (autoimmune ITP), transplant rejection (rejection of transplant organs, tissues and cells), ischemia reperfusion injury of solid organs, sepsis, systemic inflammatory response syndrome (SIRS), cerebrovascular accident (CVA, stroke), myocardial infarction (Ml), atherosclerosis, Huntington's disease, Alzheimer's disease, Parkinson's disease, amyotrophic lateral sclerosis (ALS), progressive supranuclear palsy (PSP), neonatal brain injury, neonatal hypoxic brain injury, traumatic brain injury, allergic diseases (including asthma and atopic dermatitis), peripheral nerve injury, burns, multiple sclerosis, type I diabetes, type II diabetes, obesity, Wegener's granulomatosis, pulmonary sarcoidosis, Behcet's disease, interleukin-I converting enzyme (ICE, also known as caspase-1) associated fever syndrome, chronic obstructive pulmonary disease (COPD), cigarette smoke-induced damage, cystic fibrosis, tumor necrosis factor receptor-associated periodic syndrome (TRAPS), a neoplastic tumor, melanoma, metastasis, breast cancer, non-small cell lung carcinoma (NSCLC), radiation induced necrosis, ischemic kidney damage, ophthalmologic ischemia, intracerebral hemorrhage, subarachnoid hemorrhage, peridontitis, NEMO-mutations (mutations of NF-kappa-B essential modulator gene (also known as IKK gamma or IKKG)), particularly, NEMO-deficiency syndrome, HOIL-1 deficiency ((also known as RBCK1) heme-oxidized IRP 2 ubiquitin ligase-1 deficiency), linear ubiquitin chain assembly complex (LUBAC) deficiency syndrome, hematological and solid organ malignancies, bacterial infections and viral infections (such as influenza, staphylococcus, and mycobacterium (tuberculosis)), and Lysosomal storage diseases (particularly, Gaucher disease, and including GM2 gangliosidosis, alpha-mannosidosis, aspartylglucosaminuria, cholesteryl ester storage disease, chronic hexosaminidase A deficiency, cystinosis, Danon disease, Fabry disease, Farber disease, fucosidosis, galactosialidosis, GMl gangliosidosis, mucolipidosis, infantile free sialic acid storage disease, juvenile hexosaminidase A deficiency, Krabbe disease, lysosomal acid lipase deficiency, metachromatic leukodystrophy, mucopolysaccharidoses disorders, multiple sulfatase deficiency, Niemann-Pick disease, neuronal ceroid lipofuscinoses, Pompe disease, pycnodysostosis, Sandhoff disease, Schindler disease, sialic acid storage disease, Tay-Sachs, and Wolman disease), spinal cord injury, Stevens-Johnson syndrome, fibrosis, complement-mediated cytotoxicity, toxic epidermal necrolysis, and/or for the treatment of cells ex vivo to preserve vitality and function.

The compounds of the Formulae described herein, or a pharmaceutically acceptable salt thereof, may be useful for the treatment of glaucoma.

The compounds of the Formulae described herein, or a pharmaceutically acceptable salt thereof, may be particularly useful for treatment of pancreatic ductal adenocarcinoma, hepatocellular carcinoma, mesothelioma, or melanoma.

The compounds of the Formulae described herein, or a pharmaceutically acceptable salt thereof, may be particularly useful for the treatment of the following RIPK11-mediated disease or disorder: rheumatoid arthritis, inflammatory bowel disease (including Crohn's disease and ulcerative colitis), and psoriasis.

The treatment of the above-noted diseases/disorders may concern, more specifically, the amelioration of organ injury or damage sustained as a result of the noted diseases/disorders. For example, the compounds of this invention may be particularly useful for amelioration of brain tissue injury or damage following ischemic brain injury or traumatic brain injury, or for amelioration of heart tissue injury or damage following myocardial infarction, or for amelioration of brain tissue injury or damage associated with Huntington's disease, Alzheimer's disease or Parkinson's disease, or for amelioration of liver tissue injury or damage associated with non-alcohol steatohepatitis, alcohol steatohepatitis, autoimmune hepatitis autoimmune hepatobiliary diseases, or primary sclerosing cholangitis, or overdose of acetaminophen.

The compounds of this invention may be particularly useful for the amelioration of organ injury or damage sustained as a result of radiation therapy, or amelioration of spinal tissue injury or damage following spinal cord injury or amelioration of liver tissue injury or damage associated acute liver failure. The compounds of this invention may be particularly useful for amelioration of auditory disorders, such as noise-induced hearing loss or auditory disorders following the administration of ototoxic drugs or substances e.g. cisplatin.

The compounds of this invention may be particularly useful for amelioration of solid organ tissue (particularly kidney, liver, and heart and/or lung) injury or damage following transplant or the administration of nephrotoxic drugs or substances e.g. cisplatin. It will be understood that amelioration of such tissue damage may be achieved where possible, by pre-treatment with a compound of the Formulae described herein, or a pharmaceutically acceptable salt thereof; for example, by pre-treatment of a patient prior to administration of cisplatin or pre-treatment of an organ or the organ recipient prior to transplant surgery.

Amelioration of such tissue damage may be achieved by treatment with a compound of the Formulae described herein, or a pharmaceutically acceptable salt thereof, during transplant surgery.

Amelioration of such tissue damage may also be achieved by short-term treatment of a patient with a compound of the Formulae described herein, or a pharmaceutically acceptable salt thereof, after transplant surgery.

In one embodiment, the compounds of the Formulae described herein, or a pharmaceutically acceptable salt thereof, may be useful for the treatment of retinal detachment, macular degeneration, and retinitis pigmentosa.

In another embodiment, the compounds of the Formulae described herein, or a pharmaceutically acceptable salt thereof, may be useful for the treatment of multiple sclerosis.

In one embodiment, the compounds of the Formulae described herein, or a pharmaceutically acceptable salt thereof, may be useful for the treatment of traumatic brain injury.

In another embodiment, the compounds of the Formulae described herein, or a pharmaceutically acceptable salt thereof, may be useful for the treatment of Huntington's Disease or Niemann-Pick disease.

In another embodiment, the compounds of the Formulae described herein, or a pharmaceutically acceptable salt thereof, may be useful for the treatment of amyotrophic lateral sclerosis (ALS), progressive supranuclear palsy (PSP), and Alzheimer's disease.

In another embodiment, the compounds of the Formulae described herein, or a pharmaceutically acceptable salt thereof, may be useful for the treatment of age-related macular degeneration.

The treatment of retinal detachment, macular degeneration, retinitis pigmentosa, multiple sclerosis, traumatic brain injury, Huntington's Disease, Alzheimer's Disease, amyotrophic lateral sclerosis, and Niemann-Pick disease may concern, more specifically, the amelioration of organ injury or damage sustained as a result of these diseases/disorders. For example, the compounds described herein may be particularly useful for amelioration of brain tissue injury or damage following traumatic brain injury, or for amelioration of brain tissue injury or damage associated of Huntington's Disease, Alzheimer's Disease, amyotrophic lateral sclerosis, and Niemann-Pick disease.

In another embodiment, the compounds of the Formulae described herein, or a pharmaceutically acceptable salt thereof, may be useful for the treatment of retinal detachment, macular degeneration, and retinitis pigmentosa, and the amelioration of brain tissue injury or damage as a result of multiple sclerosis, traumatic brain injury, Huntington's Disease, Alzheimer's Disease, amyotrophic lateral sclerosis, and Niemann-Pick disease.

In another embodiment, the compounds of the Formulae described herein, or a pharmaceutically acceptable salt thereof, may be useful for the treatment of Crohn's disease, ulcerative colitis, psoriasis, rheumatoid arthritis, spondyloarthritis, systemic onset juvenile idiopathic arthritis (SoJIA), and osteoarthritis.

In yet another embodiment, the compounds of the Formulae described herein, or a pharmaceutically acceptable salt thereof, may be useful for the treatment of psoriasis, rheumatoid arthritis, and ulcerative and colitis.

In another embodiment, the compounds of the Formulae described herein, or a pharmaceutically acceptable salt thereof, may be useful for the treatment of lupus, inflammatory bowel disease (IBD), Crohn's disease, and ulcerative colitis.

In another embodiment, the compounds of the Formulae described herein, or a pharmaceutically acceptable salt thereof, may be useful for the treatment of cerebrovascular accident (CVA, stroke), Huntington's disease, Alzheimer's disease, amyotrophic lateral sclerosis (ALS), traumatic brain injury, multiple sclerosis, Gaucher disease, Niemann-Pick disease, and spinal cord injury.

In another embodiment, the compounds of the Formulae described herein, or a pharmaceutically acceptable salt thereof, may be useful for the treatment of amyotrophic lateral sclerosis (ALS).

In another embodiment, the compounds of the Formulae described herein, or a pharmaceutically acceptable salt thereof, may be useful for the treatment of multiple sclerosis.

In another embodiment, the compounds of the Formulae described herein, or a pharmaceutically acceptable salt thereof, may be useful for the treatment of pancreatic ductal adenocarcinoma (PDAC), metastasis, melanoma, breast cancer, non-small cell lung carcinoma (NSCLC), and radiation induced necrosis.

In another embodiment, the compounds of the Formulae described herein, or a pharmaceutically acceptable salt thereof, may be useful for the treatment of pancreatic ductal adenocarcinoma (PDAC), metastasis, melanoma, breast cancer, and non-small cell lung carcinoma (NSCLC).

In another embodiment, the compounds of the Formulae described herein, or a pharmaceutically acceptable salt thereof, may be useful for the treatment of pancreatic ductal adenocarcinoma (PDAC).

In another embodiment, the compounds of the Formulae described herein, or a pharmaceutically acceptable salt thereof, may be useful for the treatment of intracerebral hemorrhage and subarachnoid hemorrhage.

In another embodiment, the compounds of the Formulae described herein, or a pharmaceutically acceptable salt thereof, may be useful for the treatment of type II diabetes and obesity.

In another embodiment, the compounds of the Formulae described herein, or a pharmaceutically acceptable salt thereof, may be useful for the treatment of atherosclerosis.

In another embodiment, the compounds of the Formulae described herein, or a pharmaceutically acceptable salt thereof, may be useful for the treatment of vasculitis.

In another embodiment, the compounds of the Formulae described herein, or a pharmaceutically acceptable salt thereof, may be useful for the treatment of dependent inflammation and cell death that occurs in inherited and sporadic diseases including Alzheimer's disease, amyotrophic lateral sclerosis, multiple sclerosis, Parkinson's disease, chronic traumatic encephalopathy, rheumatoid arthritis, ulcerative colitis, inflammatory bowel disease, psoriasis as well as acute tissue injury caused by stroke, traumatic brain injury, encephalitis.

In another embodiment, the compounds of the Formulae described herein, or pharmaceutically acceptable salt thereof, may be useful for the treatment of ischemic kidney damage, ophthalmologic ischemia, intracerebral hemorrhage, and subarachnoid hemorrhage.

In another embodiment, the compounds of the Formulae described herein, or pharmaceutically acceptable salt thereof, may be useful for the treatment of non-alcoholic steatohepatitis (NASH), alcoholic steatohepatitis (ASH), autoimmune hepatitis, and non-alcoholic fatty liver disease (NAFLD).

The compounds of the invention, particularly the compounds of the Formulae described herein, or a pharmaceutically acceptable salt thereof, may be particularly useful for the treatment of the RIPK1-mediated, cancer-related diseases or disorders. Gong et al., The role of necroptosis in cancer biology and therapy, Molecular Cancer (2019) 18:100. In one aspect the human has a solid tumor. In one aspect the tumor is selected from head and neck cancer, gastric cancer, melanoma, renal cell carcinoma (RCC), esophageal cancer, non-small cell lung carcinoma (NSCLC), prostate cancer, colorectal cancer, ovarian cancer, pancreatic cancer, and pancreatic ductal adenocarcinoma. In one aspect the human has one or more of the following: colorectal cancer (CRC), esophageal cancer, cervical, bladder, breast cancer, head and neck cancer, ovarian cancer, melanoma, renal cell carcinoma (RCC), EC squamous cell carcinoma, non-small cell lung carcinoma, mesothelioma, prostate cancer, and pancreatic ductal adenocarcinoma. In another aspect, the human has a liquid tumor such as diffuse large B cell lymphoma (DLBCL), multiple myeloma, chronic lyphomblastic leukemia (CLL), follicular lymphoma, acute myeloid leukemia and chronic myelogenous leukemia.

The present disclosure also relates to a method for treating or lessening the severity of a cancer selected from: brain (gliomas), glioblastomas, astrocytomas, Bannayan-Zonana syndrome, Cowden disease, Lhermitte-Duclos disease, breast cancer, triple negative breast cancer, inflammatory breast cancer, Wilm's tumor, Ewing's sarcoma, Rhabdomyosarcoma, ependymoma, medulloblastoma, colon cancer, head and neck cancer (including squamous cell carcinoma of head and neck), kidney cancer, lung cancer (including lung squamous cell carcinoma, lung adenocarcinoma, lung small cell carcinoma, and non-small cell lung carcinoma), liver cancer (including hepatocellular carcinoma), melanoma, ovarian cancer, pancreatic cancer (including squamous pancreatic cancer), prostate cancer, sarcoma, osteosarcoma, giant cell tumor of bone, thyroid cancer, lymphoblastic T-cell leukemia, chronic myelogenous leukemia, chronic lymphocytic leukemia, hairy-cell leukemia, acute lymphoblastic leukemia, acute myelogenous leukemia, chronic neutrophilic leukemia, acute lymphoblastic T-cell leukemia, plasmacytoma, immunoblastic large cell leukemia, mantle cell leukemia, multiple myeloma megakaryoblastic leukemia, multiple myeloma, acute megakaryocytic leukemia, promyelocytic leukemia, erythroleukemia, malignant lymphoma, Hodgkin's lymphoma, Non-Hodgkin's lymphoma, lymphoblastic T cell lymphoma, Burkitt's lymphoma, follicular lymphoma, neuroblastoma, bladder cancer, urothelial cancer, lung cancer, vulval cancer, cervical cancer, endometrial cancer, cancer of the uterus, renal cancer (including kidney clear cell cancer, kidney papillary cancer, renal cell carcinoma), mesothelioma, esophageal cancer, salivary gland cancer, hepatocellular cancer, gastric cancer, nasopharangeal cancer, buccal cancer, cancer of the mouth, GIST (gastrointestinal stromal tumor) and testicular cancer.

Specific examples of clinical conditions based on hematologic tumors include leukemias such as chronic myelocytic leukemia, acute myelocytic leukemia, chronic lymphocytic leukemia and acute lymphocytic leukemia; plasma cell malignancies such as multiple myeloma, MGUS and Waldenstrom's macroglobulinemia; lymphomas such as non-Hodgkin's lymphoma, Hodgkin's lymphoma; and the like.

The cancer may be any cancer in which an abnormal number of blast cells or unwanted cell proliferation is present or that is diagnosed as a hematological cancer, including both lymphoid and myeloid malignancies. Myeloid malignancies include, but are not limited to, acute myeloid (or myelocytic or myelogenous or myeloblastic) leukemia (undifferentiated or differentiated), acute promyeloid (or promyelocytic or promyelogenous or promyeloblastic) leukemia, acute myelomonocytic (or myelomonoblastic) leukemia, acute monocytic (or monoblastic) leukemia, erythroleukemia and megakaryocytic (or megakaryoblastic) leukemia. These leukemias may be referred together as acute myeloid (or myelocytic or myelogenous) leukemia (AML). Myeloid malignancies also include myeloproliferative disorders (MPD) which include, but are not limited to, chronic myelogenous (or myeloid) leukemia (CML), chronic myelomonocytic leukemia (CMML), essential thrombocythemia (or thrombocytosis), and polcythemia vera (PCV). Myeloid malignancies also include myelodysplasia (or myelodysplastic syndrome or MDS), which may be referred to as refractory anemia (RA), refractory anemia with excess blasts (RAEB), and refractory anemia with excess blasts in transformation (RAEBT); as well as myelofibrosis (MFS) with or without agnogenic myeloid metaplasia.

Specific examples of clinical conditions based on hematologic tumors include leukemias such as chronic myelocytic leukemia, acute myelocytic leukemia, chronic lymphocytic leukemia and acute lymphocytic leukemia; plasma cell malignancies such as multiple myeloma, MGUS and Waldenstrom's macroglobulinemia; lymphomas such as non-Hodgkin's lymphoma, Hodgkin's lymphoma; and the like. Hematopoietic cancers also include lymphoid malignancies, which may affect the lymph nodes, spleens, bone marrow, peripheral blood, and/or extranodal sites. Lymphoid cancers include B-cell malignancies, which include, but are not limited to, B-cell non-Hodgkin's lymphomas (B-NHLs). B-NHLs may be indolent (or low-grade), intermediate grade (or aggressive) or high-grade (very aggressive). Indolent B cell lymphomas include follicular lymphoma (FL); small lymphocytic lymphoma (SLL); marginal zone lymphoma (MZL) including nodal MZL, extranodal MZL, splenic MZL and splenic MZL with villous lymphocytes; lymphoplasmacytic lymphoma (LPL); and mucosa-associated-lymphoid tissue (MALT or extranodal marginal zone) lymphoma. Intermediate-grade B-NHLs include mantle cell lymphoma (MCL) with or without leukemic involvement, diffuse large cell lymphoma (DLBCL), follicular large cell (or grade 3 or grade 3B) lymphoma, and primary mediastinal lymphoma (PML). High-grade B-NHLs include Burkitt's lymphoma (BL), Burkitt-like lymphoma, small non-cleaved cell lymphoma (SNCCL) and lymphoblastic lymphoma. Other B-NHLs include immunoblastic lymphoma (or immunocytoma), primary effusion lymphoma, HIV associated (or AIDS related) lymphomas, and post-transplant lymphoproliferative disorder (PTLD) or lymphoma. B-cell malignancies also include, but are not limited to, chronic lymphocytic leukemia (CLL), prolymphocytic leukemia (PLL), Waldenstrom's macroglobulinemia (WM), hairy cell leukemia (HCL), large granular lymphocyte (LGL) leukemia, acute lymphoid (or lymphocytic or lymphoblastic) leukemia, and Castleman's disease. NHL may also include T-cell non-Hodgkin's lymphoma s(T-NHLs), which include, but are not limited to T-cell non-Hodgkin's lymphoma not otherwise specified (NOS), peripheral T-cell lymphoma (PTCL), anaplastic large cell lymphoma (ALCL), angioimmunoblastic lymphoid disorder (AILD), nasal natural killer (NK) cell/T-cell lymphoma, gamma/delta lymphoma, cutaneous T cell lymphoma, mycosis fungoides, and Sezary syndrome. Hematopoietic cancers also include Hodgkin's lymphoma (or disease) including classical Hodgkin's lymphoma, nodular sclerosing Hodgkin's lymphoma, mixed cellularity Hodgkin's lymphoma, lymphocyte predominant (LP) Hodgkin's lymphoma, nodular LP Hodgkin's lymphoma, and lymphocyte depleted Hodgkin's lymphoma. Hematopoietic cancers also include plasma cell diseases or cancers such as multiple myeloma (MM) including smoldering MM, monoclonal gammopathy of undetermined (or unknown or unclear) significance (MGUS), plasmacytoma (bone, extramedullary), lymphoplasmacytic lymphoma (LPL), Waldenstrom's Macroglobulinemia, plasma cell leukemia, and primary amyloidosis (AL). Hematopoietic cancers may also include other cancers of additional hematopoietic cells, including polymorphonuclear leukocytes (or neutrophils), basophils, eosinophils, dendritic cells, platelets, erythrocytes and natural killer cells. Tissues which include hematopoietic cells referred herein to as “hematopoietic cell tissues” include bone marrow; peripheral blood; thymus; and peripheral lymphoid tissues, such as spleen, lymph nodes, lymphoid tissues associated with mucosa (such as the gut-associated lymphoid tissues), tonsils, Peyer's patches and appendix, and lymphoid tissues associated with other mucosa, for example, the bronchial linings.

### Pharmaceutical Compositions

Compounds described herein may be administered orally or parenterally. As formulated into a dosage form suitable for administration, the compounds described herein can be used as a pharmaceutical composition for the prevention, treatment, or remedy of the above diseases.

In clinical use of the compounds described herein, usually, the compound is formulated into various preparations together with pharmaceutically acceptable additives according to the dosage form and may then be administered. By “pharmaceutically acceptable” it is meant the additive, carrier, diluent or excipient must be compatible with the other ingredients of the formulation and not deleterious to the recipient thereof. As such, various additives ordinarily used in the field of pharmaceutical preparations are usable. Specific examples thereof include gelatin, lactose, sucrose, titanium oxide, starch, crystalline cellulose, hydroxypropyl methylcellulose, carboxymethylcellulose, corn starch, microcrystalline wax, white petrolatum, magnesium metasilicate aluminate, anhydrous calcium phosphate, citric acid, trisodium citrate, hydroxypropylcellulose, sorbitol, sorbitan fatty acid ester, polysorbate, sucrose fatty acid ester, polyoxyethylene, hardened castor oil, polyvinylpyrrolidone, magnesium stearate, light silicic acid anhydride, talc, vegetable oil, benzyl alcohol, gum arabic, propylene glycol, polyalkylene glycol, cyclodextrin, hydroxypropyl cyclodextrin, and the like.

Preparations to be formed with those additives include, for example, solid preparations such as tablets, capsules, granules, powders and suppositories; and liquid preparations such as syrups, elixirs and injections. These may be formulated according to conventional methods known in the field of pharmaceutical preparations. The liquid preparations may also be in such a form that may be dissolved or suspended in water or in any other suitable medium in their use.

Especially for injections, if desired, the preparations may be dissolved or suspended in physiological saline or glucose liquid, and a buffer or a preservative may be optionally added thereto.

The pharmaceutical compositions may contain the compound of the invention in an amount of from 1 to 99.9% by weight, preferably from 1 to 60% by weight of the composition. The compositions may further contain any other therapeutically-effective compounds.

In case where the compounds of the invention are used for prevention or treatment for the above-mentioned diseases, the dose and the dosing frequency may be varied, depending on the sex, the age, the body weight and the disease condition of the patient and on the type and the range of the intended remedial effect. In general, when orally administered, the dose may be from 0.001 to 50 mg/kg of body weight/day, and it may be administered at a time or in several times. In specific embodiments, the dose is from about 0.01 to about 25 mg/kg/day, in particular embodiments, from about 0.05 to about 10 mg/kg/day. For oral administration, the compositions are preferably provided in the form of tablets or capsules containing from 0.01 mg to 1,000 mg. In specific embodiments, the dose is 0.01, 0.05, 0.1, 0.2, 0.5, 1.0, 2.5, 5, 10, 15, 20, 25, 30, 40, 50, 75, 100, 125, 150, 175, 200, 225, 250, 500, 750, 850 or 1,000 milligrams of a compound described herein. This dosage regimen may be adjusted to provide the optimal therapeutic response.

### Combination Therapy

The compounds of the present invention are further useful in methods for the prevention or treatment of the aforementioned diseases, disorders and conditions in combination with other therapeutic agents.

The compounds of the present invention may be used in combination with one or more other drugs in the treatment, prevention, suppression or amelioration of diseases or conditions for which compounds described herein or the other drugs may have utility, where the combination of the drugs together are safer or more effective than either drug alone. Such other drug(s) may be administered in an amount commonly used therefore, contemporaneously or sequentially with a compound described herein or a pharmaceutically acceptable salt thereof. When a compound described herein is used contemporaneously with one or more other drugs, the pharmaceutical composition may in specific embodiments contain such other drugs and the compound described herein or its pharmaceutically acceptable salt in unit dosage form. However, the combination therapy may also include therapies in which the compound described herein or its pharmaceutically acceptable salt and one or more other drugs are administered on different overlapping schedules. It is also contemplated that when used in combination with one or more other active ingredients, the compounds of the present invention and the other active ingredients may be used in lower doses than when each is used singly. Accordingly, the pharmaceutical compositions of the present invention include those that contain one or more other active ingredients, in addition to a compound described herein or a pharmaceutically acceptable salt thereof.

### EXAMPLES

The compounds of the present invention can be prepared according to the following schemes and examples, or modifications thereof, using available starting materials, reagents and conventional synthetic procedures. In these reactions, it is also possible to make use of variants which are themselves known to those of ordinary skill in this art but are not mentioned in detail. The general procedures for making the compounds claimed in this invention can be readily understood and appreciated by one skilled in the art from viewing the following Schemes and descriptions.

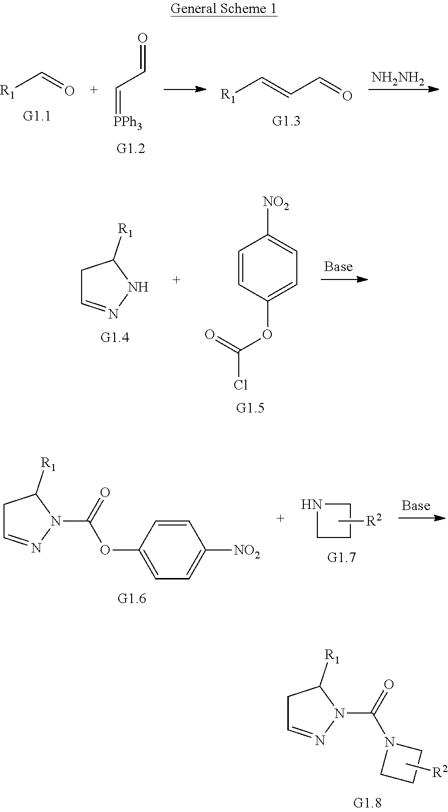

One general strategy for the synthesis of compounds of type G1.8 is via a four-step procedure shown in General Scheme 1, wherein R¹ and R² are as defined in Formula I. In the first step, aldehydes G1.1 can be combined with phosphorous ylide G1.2 in solvents such as THF to form unsaturated aldehyde intermediates of type G1.3. In the second step, intermediates of type G1.3 can be treated with hydrazine in solvents such as THF or t-BuOH to provide dihydropyrazole intermediates such as G1.4. In the third step, intermediates of type G1.4 can be combined with nitrophenyl carbonochloridate (G1.5) in the presence of a base to generate nitrophenyl carbamate intermediates G1.6. Bases such as potassium carbonate or triethylamine, and solvents such as THF or DCM, can be used. In the fourth step, intermediates of type G1.6 can be combined with substituted azetidines G1.7 in the presence of base to form urea products G1.8. Bases such as DIPEA, triethylamine, and cesium carbonate, and solvents such as DMF, DMSO, and DCM, can be used. Products of type G1.8 can be purified by silica gel chromatography or preparative reverse-phase HPLC.

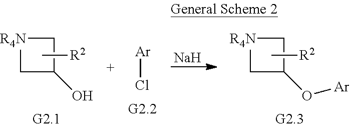

One general strategy for the synthesis of compounds of type G2.3 is via a one-step procedure shown in General Scheme 2, wherein R² is as defined in Formula I, Ar is an electron-deficient heteroaryl group, and R₄, taken together with the nitrogen to which it is attached, is a urea or carbamate group. Hydroxyazetidines G2.1 can be treated with sodium hydride, then combined with electron-deficient heteroaryl chlorides G2.2 in solvents such as THF to form heteroaryl ethers of type G2.3. Products of type G2.3 can be purified by silica gel chromatography or preparative reverse-phase HPLC.

Abbreviations used herein have the following meaning:

| | |
|---|---|
| 4ÅMS | 4 Ångstrom Molecular Sieves |
| ° C. | Degrees Celsius |
| μL | Microliter |
| μW | Microwave irradiation |
| Ac | Acetate |
| aq. | Aqueous |
| Boc | tert-butoxycarbonyl |
| Boc₂O | Di-tert-butyl dicarbonate |
| DAST | Diethylaminosulfur trifluoride |
| dba | Dibenzylideneacetone |
| DCE | Dichloroethane |
| DCM | Dichloromethane |
| DIPEA | N,N-Diisopropylethylamine |
| DMF | Dimethylacetamide |
| DMAP | 4-Dimethylaminopyridine |
| DME | Dimethoxyethane |
| DMF | Dimethylformamide |
| DMSO | Dimethyl Sulfoxide |
| DMSO-d₆ | Deuterated Dimethyl Sulfoxide |
| dppf | bis(diphenylphosphino)ferrocene |
| ESI | Electrospray Ionization |
| Et₃N | Triethylamine |
| EtOAc | Ethyl acetate |
| g | Grams |
| h | Hour/Hours |
| HPLC | High Performance Liquid Chromatography |
| i-PrOH | Isopropyl alcohol |
| M | Molar |
| MeCN | Acetonitrile |
| MeOH | Methanol |
| mg | Milligrams |
| MHz | Megahertz |
| min | Minutes |
| mL | Milliliters |
| mmol | Millimoles |
| MS | Mass Spectroscopy |
| MsCl | Methansulfonyl chloride |
| nM | Nanomolar |
| NMR | Nuclear Magnetic Resonance |
| PCC | Pyridinium chlorochromate |
| Pd/C | Palladium on Carbon |
| SFC | Supercritical Fluid (CO₂) Chromatography |
| sat. | Saturated |
| t-BuOH | tert-butanol |
| TBAI | Tetra-n-butylammonium iodide |
| TFA | Trifluoroacetic acid |
| THF | Tetrahydrofuran |

### General Experimental Information

Unless otherwise noted, all reactions were magnetically stirred and performed under an inert atmosphere such as nitrogen or argon.

Unless otherwise noted, “concentrated” means evaporating the solvent from a solution or mixture using a rotary evaporator or vacuum pump.

Unless otherwise noted, flash chromatography was carried out on an ISCO®, Analogix®, or Biotage® automated chromatography system using a commercially available cartridge as the column. Columns were usually filled with silica gel as the stationary phase. Reverse phase preparative HPLC conditions can be found at the end of the experimental section. Aqueous solutions were concentrated on a Genevac® evaporator or were lyophilized.

Unless otherwise noted, proton nuclear magnetic resonance (¹H NMR) spectra and proton-decoupled carbon nuclear magnetic resonance (¹³C{¹H} NMR) spectra were recorded on 400, 500, or 600 MHz Bruker or Varian NMR spectrometers at ambient temperature. All chemical shifts (δ) were reported in parts per million (ppm). Proton resonances were referenced to residual protium in the NMR solvent, which can include, but is not limited to, CDCl₃, DMSO-d₆, and MeOD-d₄. Carbon resonances are referenced to the carbon resonances of the NMR solvent. Data are represented as follows: chemical shift, multiplicity (br=broad, br s=broad singlet, s=singlet, d=doublet, dd=doublet of doublets, ddd=doublet of doublet of doublets, t=triplet, q=quartet, m=multiplet), coupling constants (J) in Hertz (Hz), integration.

### Preparation of Intermediate A.2, bicyclo[2.2.1]heptane-1-carbaldehyde

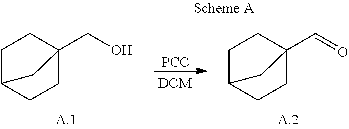

A stirring mixture of bicyclo[2.2.1]heptan-1-ylmethanol (830 mg, 6.25 mmol) in DCM (10 mL) was cooled to 0° C. and then treated with PCC (2.29 g, 10.6 mmol) and Celite® (700 mg). The resulting mixture was warmed to 20° C. and stirred for 16 h. After completion of the reaction, silica gel (1 g) was added, and the resulting mixture was filtered, rinsing with DCM (3×50 mL). The filtrate was directly concentrated at a temperature of 0° C. to provide bicyclo[2.2.1]heptane-1-carbaldehyde. ¹H NMR (400 MHz, CDCl₃) δ 9.87 (s, 1H), 2.47-2.40 (m, 1H), 1.94-1.87 (m, 2H), 1.77-1.70 (m, 2H), 1.67-1.53 (m, 2H), 1.39 (d, J=7.3 Hz, 4H).

### Preparation of Intermediate B.3, (E)-3-(3,5-difluorophenyl)acrylaldehyde

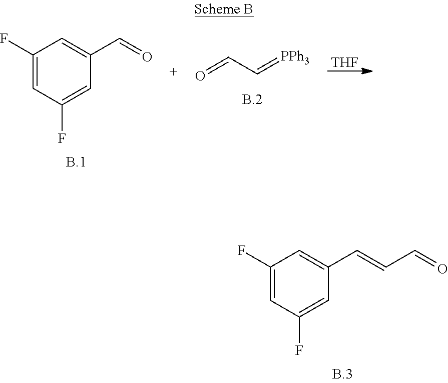

A stirring mixture of 3,5-difluorobenzaldehyde (3.0 g, 21.1 mmol) in THF (50 mL) was treated with 2-(triphenylphosphoranylidene)acetaldehyde (7.1 g, 23.2 mmol), and the resulting mixture was stirred at 80° C. for 15 h. The reaction mixture was then directly concentrated, and the crude residue was purified by silica gel chromatography (gradient elution: 0-50% EtOAc/hexanes) to provide (E)-3-(3,5-difluorophenyl)acrylaldehyde. MS (ESI) m/z calculated for C₉H₇F₂O [M+H]⁺ 169, found 169.

### Preparation of Intermediate C.2, (E)-3-(3,5-difluorophenyl)acrylaldehyde

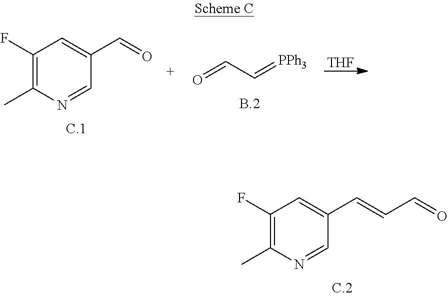

Intermediate C.2 was also synthesized according to the method shown in Scheme B, starting from C.1. MS (ESI) m/z calculated for C₉H₉NO [M+H]⁺166, found 166.

### Preparation of Intermediate D.2, (E)-3-(5-fluoro-6-methylpyridin-3-yl)acrylaldehyde

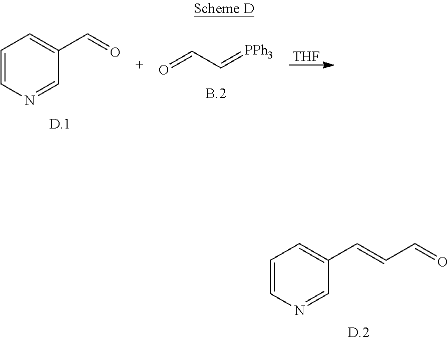

A stirring mixture of nicotinaldehyde (8.0 g, 74.7 mmol) in THF (50 mL) was treated with 2-(triphenylphosphoranylidene)acetaldehyde (25.0 g, 82.0 mmol), and the resulting mixture was stirred at 25° C. for 15 h. The reaction mixture was then directly concentrated, and the crude residue was purified by silica gel chromatography (elution: 9% EtOAc/petroleum ether) to provide (E)-3-(pyridin-3-yl)acrylaldehyde. ¹H NMR (400 MHz, CDCl₃) δ 9.54 (d, J=8.1 Hz, 1 H), 7.12 (d, J=15.7 Hz, 1H), 6.14 (dd, J=7.8, 15.9 Hz, 1H), 2.41-2.35 (m, 1H), 1.65 (br s, 2 H), 1.60-1.55 (m, 2H), 1.52-1.46 (m, 2H), 1.42 (s, 4H).

Compounds in Table 1 were prepared according to Scheme D and General Scheme 1, starting from the appropriate commercially available aldehyde intermediate. A slightly modified procedure was used wherein THF was replaced by DCM. Compounds in Table 1 were purified by silica gel chromatography.

TABLE 1
Intermediate Compounds Prepared According to General
Scheme 1 and Scheme D

| | Structure | Observed m/z |
|---|---|---|
| Entry | Name | [M + H]⁺ |
| D.3 | 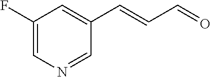 [SMILES: O=C/C=C/c1cncc(F)c1] | 152 |
| | (E)-3-(5-fluoropyridin-3-yl)acrylaldehyde | |
| D.4 | 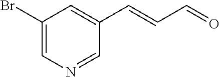 [SMILES: O=C/C=C/c1cncc(Br)c1] | 212, 214 |
| | (E)-3-(5-bromopyridin-3-yl)acrylaldehyde | |

### Preparation of Intermediate E.1, (E)-3-(bicyclo[2.2.1]heptan-1-yl)acrylaldehyde

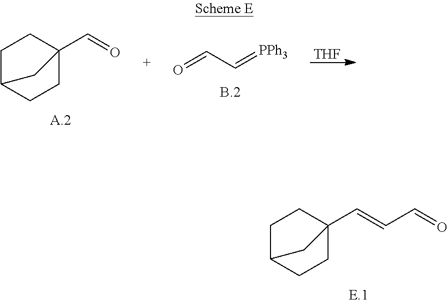

A stirring mixture of bicyclo[2.2.1]heptane-1-carbaldehyde (1.0 g, 7.25 mmol) in THF (10 mL) was treated with 2-(triphenylphosphoranylidene)acetaldehyde (1.99 g, 6.52 mmol), and the resulting mixture was stirred at 40° C. for 72 h. The reaction mixture was then directly concentrated, and the crude residue was purified by silica gel chromatography (gradient elution: 0-2% EtOAc/petroleum ether) to provide (E)-3-(bicyclo[2.2.1]heptan-1-yl)acrylaldehyde. ¹H NMR (400 MHz, CDCl₃) δ 9.54 (d, J=8.1 Hz, 1H), 7.12 (d, J=15.7 Hz, 1H), 6.14 (dd, J=15.9, 7.8 Hz, 1H), 2.41-2.35 (m, 1H), 1.65 (br s, 2H), 1.60-1.55 (m, 2H), 1.52-1.46 (m, 2H), 1.42 (s, 4H).

### Preparation of Intermediate F.1, 5-(3,5-difluorophenyl)-4,5-dihydro-1H-pyrazole

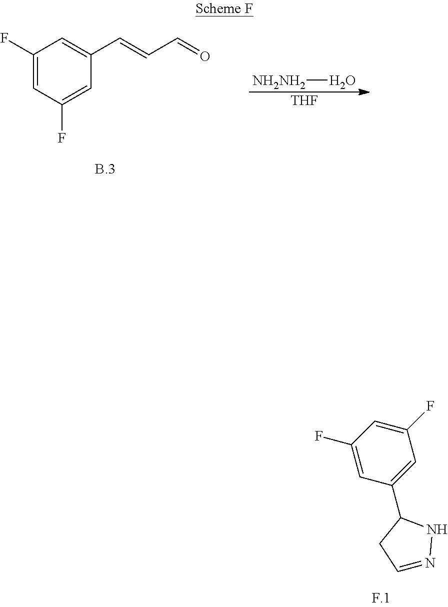

A 100 mL round bottom flask was charged with hydrazine hydrate (2.88 ml, 59.5 mmol) and THF (6 mL). A solution of (E)-3-(3,5-difluorophenyl)acrylaldehyde (1.0 g, 5.95 mmol) in THF (17.8 mL) was then added dropwise over the course of 3 min. The reaction mixture was stirred at 25° C. for 30 min. The reaction mixture was then directly concentrated in vacuo at a water bath temperature of 40° C. The crude residue was dried under vacuum for 10 min to provide 5-(3,5-difluorophenyl)-4,5-dihydro-1H-pyrazole, which was used directly in the subsequent reaction. MS (ESI) m/z calculated for C₉H₉F₂N₂ [M+H]⁺183, found 183.

Compounds in Table 2 were prepared according to Scheme F and General Scheme 1, starting from the appropriate commercially available acrylaldehyde intermediate or C.2, D.2, D.3, or D.4. Some compounds in Table 2 were isolated with slightly modified procedures from Scheme F. For Intermediates F.2 through F.4, the reaction was directly concentrated and the crude residue purified by silica gel chromatography. For Intermediates F.5 and F.6, the reactions were extracted with DCM, and the crude residue was used directly in the subsequent reaction.

TABLE 2
Intermediate Compounds Prepared According to General
Scheme 1 and Scheme F

| | | Observed |
|---|---|---|
| | Structure | m/z |
| Entry | Name | [M + H]⁺ |
| F.2 | 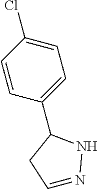 [SMILES: Clc1ccc(C2CC=NN2)cc1] | 181 |
| | 5-(4-chlorophenyl)-4,5-dihydro-1H-pyrazole | |
| F.3 | 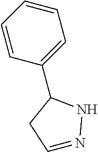 [SMILES: C1=NNC(c2ccccc2)C1] | 147 |
| | 5-phenyl-4,5-dihydro-1H-pyrazole | |
| F.4 | 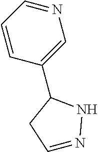 [SMILES: C1=NNC(c2cccnc2)C1] | 148 |
| | 3-(4,5-dihydro-1H-pyrazol-5-yl)pyridine | |
| F.5 | 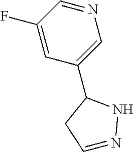 [SMILES: Fc1cncc(C2CC=NN2)c1] | 166 |
| | 3-(4,5-dihydro-1H-pyrazol-5-yl)-5-fluoropyridine | |
| F.6 | 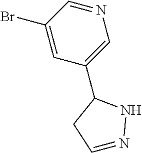 [SMILES: Brc1cncc(C2CC=NN2)c1] | 226, 228 |
| | 3-bromo-5-(4,5-dihydro-1H-pyrazol-5-yl)pyridine | |
| F.7 | 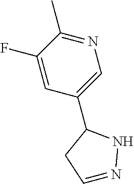 | 180 |
| | 5-(4,5-dihydro-1H-pyrazol-5-yl)-3-fluoro-2-methylpyridine | |

### Preparation of Intermediate G.1, 5-(bicyclo[2.2.1]heptan-1-yl)-4,5-dihydro-1H-pyrazole

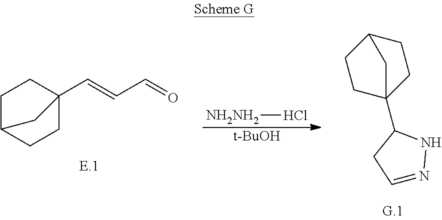

A stirring solution of (E)-3-(bicyclo[2.2.1]heptan-1-yl)acrylaldehyde (10 mg, 0.067 mmol) in t-BuOH (2 mL) was treated with hydrazine hydrochloride (9.1 mg, 0.133 mmol). The resulting mixture was then heated to 90° C. and stirred for 16 h. After cooling, the reaction mixture was then directly concentrated to provide 5-(bicyclo[2.2.1]heptan-1-yl)-4,5-dihydro-1H-pyrazole, which was used directly in the subsequent reaction. MS (ESI) m/z calculated for C₁₀H₁₇N₂ [M+H]⁺ 165, found 165.

### Preparation of Intermediates H.2 and H.3, tert-butyl(R)-5-(4-chlorophenyl)-4,5-dihydro-1H-pyrazole-1-carboxylate and tert-butyl(S)-5-(4-chlorophenyl)-4,5-dihydro-1H-pyrazole-1-carboxylate

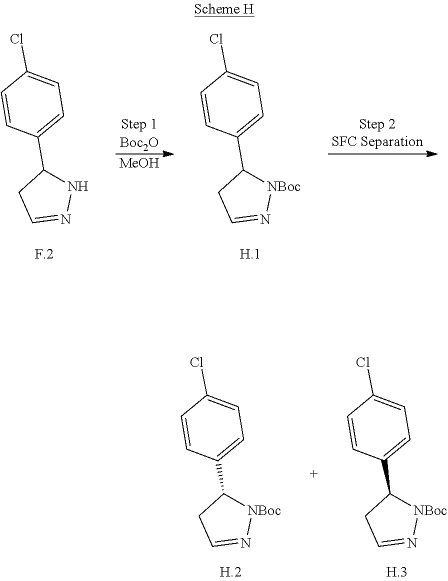 [SMILES: "Boc2O" ; "MeOH" ; Clc1ccc(C2CC=NN2)cc1 ; CC(C)(C)OC(=O)N1N=CCC1c1ccc(Cl)cc1 ; CC(C)(C)OC(=O)N1N=CC[C@@H]1c1ccc(Cl)cc1 ; CC(C)(C)OC(=O)N1N=CC[C@H]1c1ccc(Cl)cc1]

### Step 1—Synthesis of Intermediate H.1, tert-butyl 5-(4-chlorophenyl)-4,5-dihydro-1H-pyrazole-1-carboxylate

A stirring solution of 5-(4-chlorophenyl)-4,5-dihydro-1H-pyrazole (7.50 g, 41.5 mmol) in MeOH (200 mL) was treated with di-tert-butyl dicarbonate (23.9 mL, 104 mmol). The resulting mixture was stirred at 25° C. for 15 h. The reaction mixture was then directly concentrated and the crude residue was purified by silica gel chromatography (gradient elution: 0-15% EtOAc/petroleum ether) to provide tert-butyl 5-(4-chlorophenyl)-4,5-dihydro-1H-pyrazole-1-carboxylate. MS (ESI) m/z calculated for C₁₀H₁₀ClN₂O₂ [M+H−(C₄H₈)]⁺225, found 225.

### Step 2—SFC Separation of tert-butyl 5-(4-chlorophenyl)-4,5-dihydro-1H-pyrazole-1-carboxylate

Tert-butyl 5-(4-chlorophenyl)-4,5-dihydro-1H-pyrazole-1-carboxylate was purified by CHIRAL-Prep SFC [Column: Phenomenex Synergi C18, 150×30 mm: Gradient elution: 27-47% (0.1% TFA in MeCN)/CO₂ over 9 min; Flow rate: 25 mL/min; Column temp: 40° C.; 220 nm; First Eluting Peak (H.2); Second Eluting Peak (H.3)]. This provided tert-butyl(R)-5-(4-chlorophenyl)-4,5-dihydro-1H-pyrazole-1-carboxylate (H.2) and tert-butyl(S)-5-(4-chlorophenyl)-4,5-dihydro-1H-pyrazole-1-carboxylate (H.3).

Compounds in Table 3 were prepared according to Scheme H, starting from intermediate F.4. SFC conditions are provided following the table.

TABLE 3
Intermediate Compounds Prepared According to Scheme H

| | | Observed |
|---|---|---|
| | Structure | m/z |
| Entry | Name | [M + H]⁺ |
| H.4 | 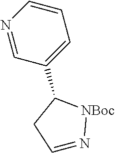 [SMILES: CC(C)(C)OC(=O)N1N=CC[C@@H]1c1cccnc1] | 248 |
| | tert-butyl (R)-5-(pyridin-3-yl)-4,5-dihydro-1H-pyrazole-1- | |
| | carboxylate | |
| H.5 | 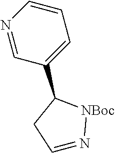 [SMILES: CC(C)(C)OC(=O)N1N=CC[C@H]1c1cccnc1] | 248 |
| | tert-butyl (S)-5-(pyridin-3-yl)-4,5-dihydro-1H-pyrazole-1- | |
| | carboxylate | |

### Intermediates H.4/H.5

Tert-butyl 5-(pyridin-3-yl)-4,5-dihydro-1H-pyrazole-1-carboxylate was purified by CHIRAL-Prep SFC [Column: Chiralpak AD-3, 150×4.6 mm; Gradient elution: 5-40% (0.05% diethylamine in EtOH)/CO₂ over 5 min, followed by 40-5% (0.05% diethylamine in EtOH)/CO₂ for 0.5 min, then 5% (0.05% diethylamine in EtOH)/CO₂ for 1.5 min; Flow rate: 2.5 mL/min; Column temp: 35° C.; First Eluting Peak (H.4); Second Eluting Peak (H.5)].

### Preparation of Intermediate 1.2, tert-butyl 5-(5-cyanopyridin-3-yl)-4,5-dihydro-1H-pyrazole-1-carboxylate

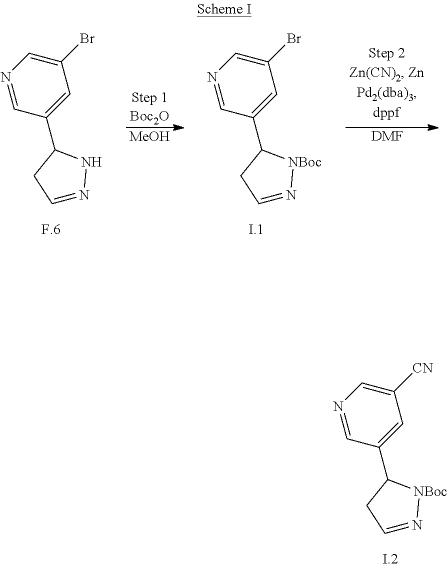

### Step 1—Synthesis of Intermediate 1.1, tert-butyl 5-(5-bromopyridin-3-yl)-4,5-dihydro-1H-pyrazole-1-carboxylate

A stirring solution of 4-bromo-2-(4,5-dihydro-1H-pyrazol-5-yl)pyridine (2.5 g, 11.1 mmol) in MeOH (80 mL) was treated with di-tert-butyl dicarbonate (12.7 ml, 55.3 mmol). The resulting mixture was stirred at 25° C. for 15 h. The reaction mixture was then directly concentrated and the crude residue was purified by preparative TLC (silica gel, eluent: 50% EtOAc/petroleum ether) to provide tert-butyl 5-(5-bromopyridin-3-yl)-4,5-dihydro-1H-pyrazole-1-carboxylate. MS (ESI) m/z calculated for C₁₃H₁₇BrN₃O₂ [M+H]⁺ 326, found 326, 328.

### Step 2—Preparation of Intermediate 1.2, tert-butyl 5-(5-cyanopyridin-3-yl)-4,5-dihydro-1H-pyrazole-1-carboxylate

A solution of tert-butyl 5-(4-bromopyridin-2-yl)-4,5-dihydro-1H-pyrazole-1-carboxylate (2.0 g, 6.13 mmol) in DMF (40 mL) was treated with dicyanozinc (3.32 g, 28.3 mmol), zinc (80 mg, 1.23 mmol), dppf (680 mg, 1.23 mmol) and Pd₂(dba)₃ (561 mg, 0.613 mmol) under an atmosphere of nitrogen. The resulting mixture was stirred at 110° C. for 12 h. The reaction mixture was then directly purified by preparative TLC (silica gel, eluent: 50% EtOAc/petroleum ether) to provide tert-butyl 5-(4-cyanopyridin-2-yl)-4,5-dihydro-1H-pyrazole-1-carboxylate. MS (ESI) m/z calculated for C₁₄H₁₇N₄O₂ [M+H]⁺ 273, found 273.

### Preparation of Intermediate J.1, (S)-5-(4-chlorophenyl)-4,5-dihydro-1H-pyrazole, TFA salt

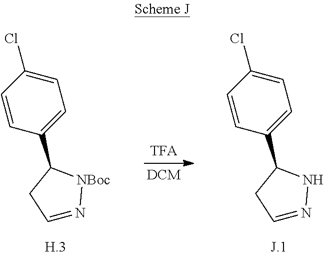 [SMILES: CC(C)(C)OC(=O)N1N=CC[C@H]1c1ccc(Cl)cc1 ; Clc1ccc([C@@H]2CC=NN2)cc1]

A solution of tert-butyl(S)-5-(4-chlorophenyl)-4,5-dihydro-H-pyrazole-1-carboxylate (1.40 g, 4.99 mmol) in DCM (15 mL) and TFA (15 mL) was stirred at 20° C. for 1 h. The reaction mixture was then directly concentrated to provide (S)-5-(4-chlorophenyl)-4,5-dihydro-1H-pyrazole, which was used in the subsequent reaction without further purification. MS (ESI) m/z calculated for C₉H₁₀ClN₂ [M+H]⁺ 181, found 181.

Compounds in Table 4 were prepared according to Scheme J, starting from intermediates H.4 H.5, or 1.2. In the case of J.2 and J.3, the reaction was run in a 4 M solution of HCl in dioxane, instead of a mixture of DCM and TFA.

TABLE 4
Intermediate Compounds Prepared According to Scheme J

| | | Observed |
|---|---|---|
| | Structure | m/z |
| Entry | Name | [M + H]⁺ |
| J.2 | 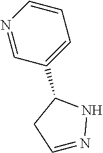 [SMILES: C1=NN[C@@H](c2cccnc2)C1] | 148 |
| | (R)-3-(4,5-dihydro-1H-pyrazol-5-yl)pyridine hydrochloride | |
| J.3 | 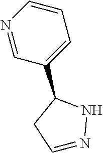 [SMILES: C1=NN[C@H](c2cccnc2)C1] | 148 |
| | (S)-3-(4,5-dihydro-1H-pyrazol-5-yl)pyridine hydrochloride | |
| J.4 | 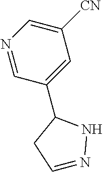 [SMILES: N#Cc1cncc(C2CC=NN2)c1] | 173 |
| | 5-(4,5-dihydro-1H-pyrazol-5-yl)nicotinonitrile | |

### Preparation of Intermediate K.2, 4-nitrophenyl(S)-5-(4-chlorophenyl)-4,5-dihydro-1H-pyrazole-1-carboxylate

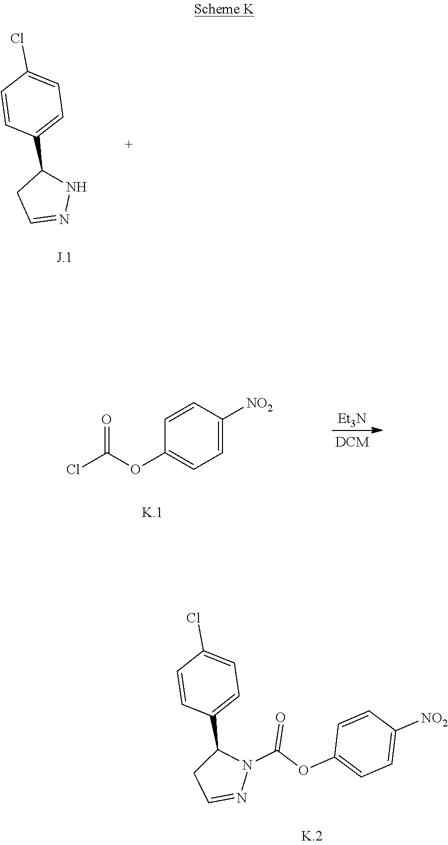 [SMILES: "Et3N" ; Clc1ccc([C@@H]2CC=NN2)cc1 ; O=C(Cl)Oc1ccc([N+](=O)[O-])cc1 ; O=C(Oc1ccc([N+](=O)[O-])cc1)N1N=CC[C@H]1c1ccc(Cl)cc1]

A solution of (S)-5-(4-chlorophenyl)-4,5-dihydro-1H-pyrazole (901 mg, 4.99 mmol) in DCM (20 mL) was treated with 4-nitrophenyl carbonochloridate (1.51 g, 7.48 mmol) and triethylamine (2.07 mL, 15.0 mmol). The resulting mixture was stirred at 20° C. for 16 h. The reaction was quenched with water (20 mL), then the layers were separated, and the aq. layer was extracted with DCM (3×20 mL). The combined organic layers were dried over anhydrous Na₂SO₄ and concentrated. The crude residue was then purified by silica gel chromatography (gradient elution: 0-10% EtOAc/petroleum ether) to provide 4-nitrophenyl(S)-5-(4-chlorophenyl)-4,5-dihydro-1H-pyrazole-1-carboxylate. MS (ESI) m/z calculated for C₁₆H₁₃ClN₃₀₄ [M+H]⁺346, found 346.

Compounds in Table 5 were prepared according to Scheme K and General Scheme 1, starting from intermediates F.3, J.2, J.3, G.1, F.5, J.4, or F.7. For K.7 through K.9, a slightly modified procedure was used wherein Et₃N was replaced by DIPEA, and the reaction mixture was directly concentrated prior to silica gel chromatography. K.6 and K.8 were separated from their enantiomers by SFC following purification with silica gel chromatography. SFC conditions are provided following the table.

TABLE 5
Intermediate Compounds Prepared According to General
Scheme 1 and Scheme K

| | | Observed |
|---|---|---|
| | Structure | m/z |
| Entry | Name | [M + H]⁺ |
| K.3 | 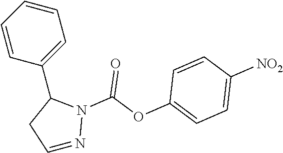 [SMILES: O=C(Oc1ccc([N+](=O)[O-])cc1)N1N=CCC1c1ccccc1] | 312 |
| | 4-nitrophenyl 5-phenyl-4,5-dihydro-1H-pyrazole-1- | |
| | carboxylate | |
| K.4 | 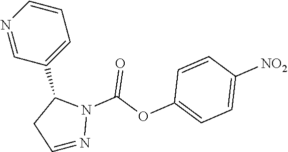 [SMILES: O=C(Oc1ccc([N+](=O)[O-])cc1)N1N=CC[C@@H]1c1cccnc1] | 313 |
| | 4-nitrophenyl (R)-5-(pyridin-3-yl)-4,5-dihydro-1H- | |
| | pyrazole-1-carboxylate | |
| K.5 | 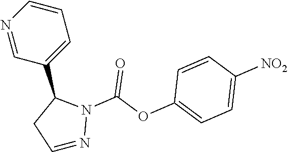 [SMILES: O=C(Oc1ccc([N+](=O)[O-])cc1)N1N=CC[C@H]1c1cccnc1] | 313 |
| | 4-nitrophenyl (S)-5-(pyridin-3-yl)-4,5-dihydro-1H- | |
| | pyrazole-1-carboxylate | |
| K.6 | 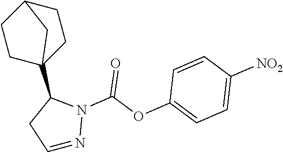 [SMILES: O=C(Oc1ccc([N+](=O)[O-])cc1)N1N=CC[C@H]1C12CCC(CC1)C2] | 330 |
| | 4-nitrophenyl (5S)-5-(bicyclo[2.2.1]heptan-1-yl)-4,5- | |
| | dihydro-1H-pyrazole-1-carboxylate | |
| K.7 | 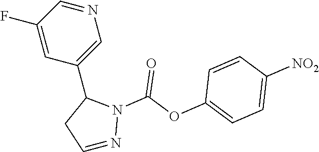 [SMILES: O=C(Oc1ccc([N+](=O)[O-])cc1)N1N=CCC1c1cncc(F)c1] | 331 |
| | 4-nitrophenyl 5-(5-fluoropyridin-3-yl)-4,5-dihydro-1H- | |
| | pyrazole-1-carboxylate | |
| K.8 | 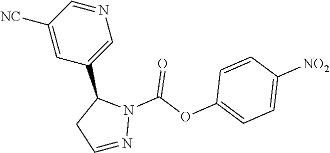 [SMILES: N#Cc1cncc([C@@H]2CC=NN2C(=O)Oc2ccc([N+](=O)[O-])cc2)c1] | 338 |
| | 4-nitrophenyl (S)-5-(5-cyanopyridin-3-yl)-4,5-dihydro- | |
| | 1H-pyrazole-1-carboxylate | |
| K.9 | 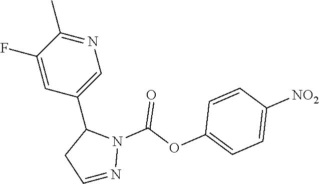 [SMILES: Cc1ncc(C2CC=NN2C(=O)Oc2ccc([N+](=O)[O-])cc2)cc1F] | 345 |
| | 4-nitrophenyl 5-(5-fluoro-6-methylpyridin-3-yl)-4,5- | |
| | dihydro-1H-pyrazole-1-carboxylate | |

### Intermediate K.6

4-nitrophenyl 5-(bicyclo[2.2.1]heptan-1-yl)-4,5-dihydro-1H-pyrazole-1-carboxylate was purified by CHIRAL-Prep SFC [Column: Daicel Chiralcel OJ-H, 250×30 mm; 30% i-PrOH/CO₂; Flow rate: 60 mL/min; First Eluting Peak (enantiomer of K.6); Second Eluting Peak (K.6)].

### Intermediate K.8

4-nitrophenyl 5-(5-cyanopyridin-3-yl)-4,5-dihydro-1H-pyrazole-1-carboxylate was purified by CHIRAL-Prep SFC [Column: Daicel Chiralpak AD, 250×30 mm; 60% EtOH/CO₂; Flow rate: 80 mL/min; First Eluting Peak (enantiomer of K.8); Second Eluting Peak (K.8)].

### Preparation of Intermediate L.1, 4-nitrophenyl 5-(3,5-difluorophenyl)-4,5-dihydro-1H-pyrazole-1-carboxylate

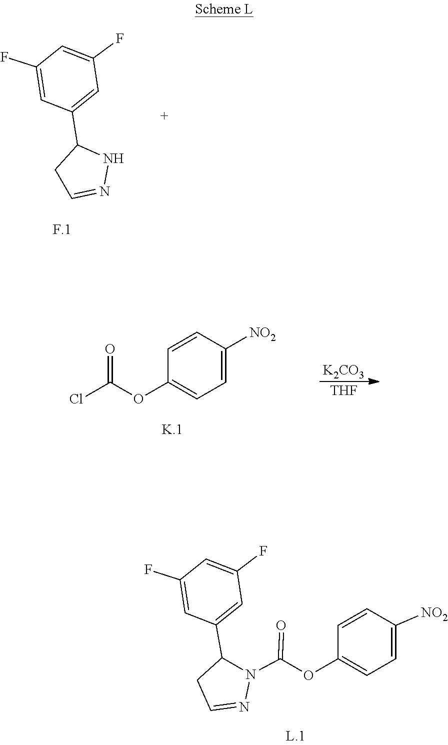

A 500 mL round bottom flask containing 5-(3,5-difluorophenyl)-4,5-dihydro-1H-pyrazole (1.08 g, 5.93 mmol) was charged with potassium carbonate (1.23 g, 8.89 mmol) and THF (148 mL). 4-nitrophenyl carbonochloridate (3.58 g, 17.79 mmol) was then added, and the resulting mixture was stirred at 25° C. for 2 h. Sat. aq. NaHCO₃(75 mL) and DCM (200 mL) were then added and the layers were separated. The aq. phase was extracted with DCM (2×100 mL), and the combined organic layer was dried over MgSO₄ and concentrated. The crude residue was then purified by silica gel chromatography (gradient elution: 0-100% EtOAc/hexanes) to provide 4-nitrophenyl 5-(3,5-difluorophenyl)-4,5-dihydro-1H-pyrazole-1-carboxylate. MS (ESI) m/z calculated for C₁₆H₁₂F₂N₃O₄ [M+H]⁺348, found 348.

### SFC Separation of 4-nitrophenyl 5-(3,5-difluorophenyl)-4,5-dihydro-1H-pyrazole-1-carboxylate

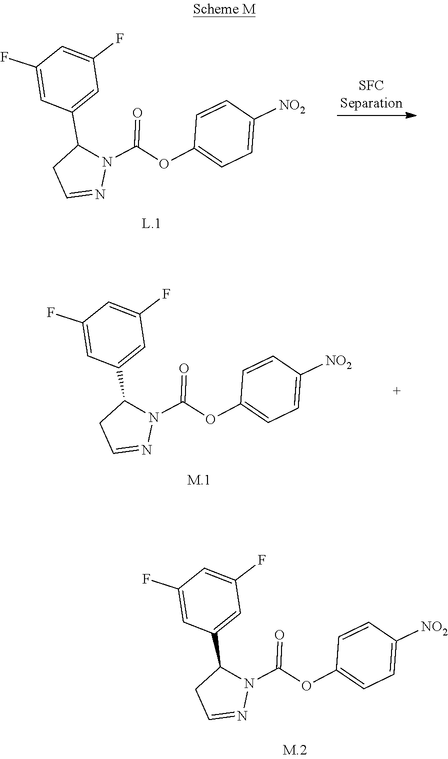 [SMILES: O=C(Oc1ccc([N+](=O)[O-])cc1)N1N=CCC1c1cc(F)cc(F)c1 ; O=C(Oc1ccc([N+](=O)[O-])cc1)N1N=CC[C@@H]1c1cc(F)cc(F)c1 ; O=C(Oc1ccc([N+](=O)[O-])cc1)N1N=CC[C@H]1c1cc(F)cc(F)c1]

4-nitrophenyl 5-(3,5-difluorophenyl)-4,5-dihydro-1H-pyrazole-1-carboxylate was purified by CHIRAL-Prep SFC [Column: AD-H, 21×250 mm: 25% [MeOH w/0.1% NaOH]/CO₂; Flow rate: 70 mL/min; 220 nm; First Eluting Peak (M.1); Second Eluting Peak (M.2)]. This provided 4-nitrophenyl(R)-5-(3,5-difluorophenyl)-4,5-dihydro-1H-pyrazole-1-carboxylate (M.1) and 4-nitrophenyl(S)-5-(3,5-difluorophenyl)-4,5-dihydro-1H-pyrazole-1-carboxylate (M.2).

Compounds in Table 6 were separated similarly to Scheme M, starting from intermediates K.7 or K.9. SFC conditions are provided following the table.

TABLE 6
Intermediate Compounds Prepared According to Scheme M

| | | Observed |
|---|---|---|
| | Structure | m/z |
| Entry | Name | [M + H]⁺ |
| M.3 | 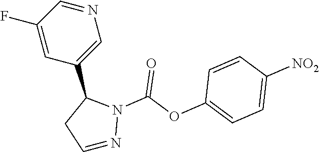 [SMILES: O=C(Oc1ccc([N+](=O)[O-])cc1)N1N=CC[C@H]1c1cncc(F)c1] | 331 |
| | 4-nitrophenyl (S)-5-(5-fluoropyridin-3-yl)-4,5-dihydro-1H- | |
| | pyrazole-1-carboxylate | |
| M.4 | 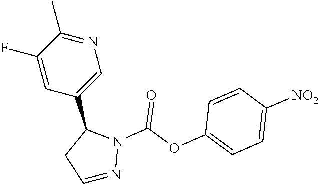 [SMILES: Cc1ncc([C@@H]2CC=NN2C(=O)Oc2ccc([N+](=O)[O-])cc2)cc1F] | 313 |
| | 4-nitrophenyl (S)-5-(5-fluoro-6-methylpyridin-3-yl)-4,5- | |
| | dihydro-1H-pyrazole-1-carboxylate | |

### Intermediate M.3

4-nitrophenyl 5-(5-fluoropyridin-3-yl)-4,5-dihydro-1H-pyrazole-1-carboxylate was purified by CHIRAL-Prep SFC [Column: Daicel Chiralpak IC, 250×50 mm; 55% EtOH/CO₂; Flow rate: 200 mL/min; First Eluting Peak (M.3); Second Eluting Peak (Enantiomer of M.3)].

### Intermediate M.4

4-nitrophenyl 5-(5-fluoro-6-methylpyridin-3-yl)-4,5-dihydro-1H-pyrazole-1-carboxylate was purified by CHIRAL-Prep SFC [Column: Daicel Chiralpak OJ, 250×30 mm: 20% [0.1% NH₄OH in EtOH]/CO₂; Flow rate: 60 mL/min; First Eluting Peak (Enantiomer of M.4); Second Eluting Peak (M.4)].

### Preparation of Intermediates N.3 and N.4, tert-butyl (2S,3R and 2R,3S)-2-methyl-3-phenoxyazetidine-1-carboxylate and tert-butyl (2S,3S and 2R,3R)-2-methyl-3-phenoxyazetidine-1-carboxylate

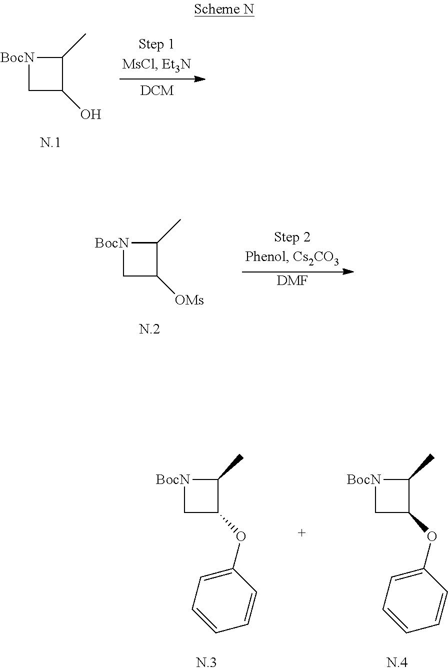

### Step 1—Synthesis of Intermediate N.2, tert-butyl 2-methyl-3-((methylsulfonyl)oxy)azetidine-1-carboxylate

Triethylamine (0.502 mL, 3.61 mmol) and MsCl (0.156 mL, 2.00 mmol) were added to a stirred solution of tert-butyl 3-hydroxy-2-methylazetidine-1-carboxylate (300 mg, 1.60 mmol) in DCM (6.4 mL) that had been cooled to 0° C. The reaction mixture was stirred at 0° C. for 10 min, then warmed to 25° C. and stirred for 18 h. Water (8 mL) and DCM (2 mL) were then added, and the layers sere separated. The aq. layer was extracted with DCM (2×8 mL), and the combined organic layers were dried over MgSO₄ and concentrated to provide tert-butyl 2-methyl-3-((methylsulfonyl)oxy)azetidine-1-carboxylate, which was used directly in the subsequent reaction. MS (ESI) calculated for C₆H₁₂NO₅S [M+H−(C₄H₈)]⁺210, found 210.

### Step 2—Preparation of Intermediates N.3 and N.4, tert-butyl (2S,3R and 2R,3S)-2-methyl-3-phenoxyazetidine-1-carboxylate and tert-butyl (2S,3S and 2R,3R)-2-methyl-3-phenoxyazetidine-1-carboxylate

A 40 mL scintillation vial was charged with tert-butyl 2-methyl-3-((methylsulfonyl)oxy)azetidine-1-carboxylate (430 mg, 1.62 mmol), phenol (198 mg, 2.11 mmol), and cesium carbonate (792 mg, 2.43 mmol). DMF (8.1 mL) was then added, and the resulting mixture was stirred at 120° C. for 18 h. After cooling, the reaction mixture was poured into Et₂O (50 mL) and water (50 mL). The layers were then separated, and the aq. layer was extracted with Et₂O (2×50 mL). The combined organic layers were washed with water (2×20 mL) and brine (15 mL), dried over MgSO₄, and concentrated. The crude residue was then purified by silica gel chromatography (gradient elution: 0-100% EtOAc/hexanes) to provide tert-butyl (2S,3R and 2R,3S)-2-methyl-3-phenoxyazetidine-1-carboxylate as the first eluting peak, and tert-butyl (2S,3S and 2R,3R)-2-methyl-3-phenoxyazetidine-1-carboxylate as the second eluting peak. MS (ESI) m/z calculated for C₁₁H₁₄NO₃ [M+H−(C₄H₈)]⁺208, found 208.

### SFC Separation of tert-butyl (2S,3R and 2R,3S)-2-methyl-3-phenoxyazetidine-1-carboxylate

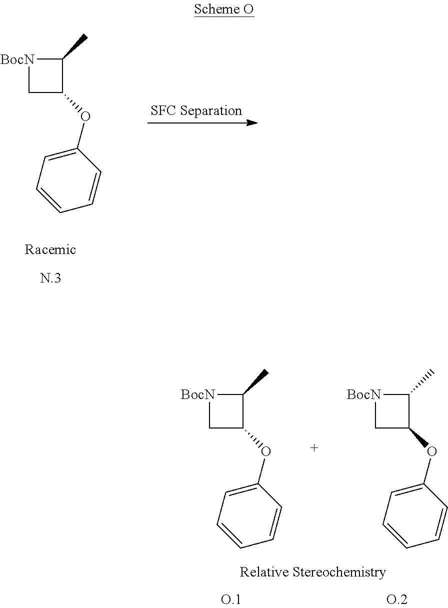 [SMILES: C[C@H]1[C@H](Oc2ccccc2)CN1C(=O)OC(C)(C)C ; C[C@H]1[C@H](Oc2ccccc2)CN1C(=O)OC(C)(C)C ; C[C@@H]1[C@@H](Oc2ccccc2)CN1C(=O)OC(C)(C)C]

Tert-butyl (2S,3R and 2R,3S)-2-methyl-3-phenoxyazetidine-1-carboxylate was purified by CHIRAL-Prep SFC [Column: Lux-2, 21×250 mm: 5% [MeOH w/0.1% NaOH]/CO₂; Flow rate: 70 mL/min; 220 nm; First Eluting Peak (O.1); Second Eluting Peak (O.2)]. This provided tert-butyl (2S,3R or 2R,3S)-2-methyl-3-phenoxyazetidine-1-carboxylate (O.1) and tert-butyl (2R,3S or 2S,3R)-2-methyl-3-phenoxyazetidine-1-carboxylate (0.2).

### Preparation of Intermediate P.3, tert-butyl (2R,3R)-3-(4-fluorophenoxy)-2-methylazetidine-1-carboxylate

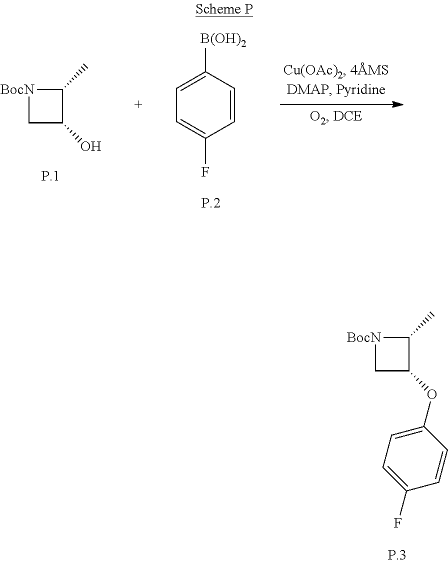 [SMILES: C[C@@H]1[C@H](O)CN1C(=O)OC(C)(C)C ; OB(O)c1ccc(F)cc1 ; C[C@@H]1[C@H](Oc2ccc(F)cc2)CN1C(=O)OC(C)(C)C]

A solution of tert-butyl (2R,3R)-3-hydroxy-2-methylazetidine-1-carboxylate (50 mg, 0.267 mmol) in DCE (5 mL) was treated with (4-fluorophenyl)boronic acid (299 mg, 2.14 mmol), DMAP (6.5 mg, 0.053 mmol), pyridine (63 mg, 0.801 mmol) and 4 Å molecular sieves (1 g). The resulting mixture was stirred 20° C. for 10 min, then Cu(OAc)₂ (48.5 mg, 0.267 mmol) was added. The reaction mixture was then stirred at 90° C. under an atmosphere of oxygen for 24 h. The reaction mixture was then directly filtered, rinsing with DCM (2×20 mL). Water (30 mL) was added to the filtrate and the resulting mixture was extracted with DCM (3×30 mL), washed with brine (20 mL), dried over Na₂SO₄, and concentrated. The crude residue was purified by silica gel chromatography (gradient elution: 0-18% EtOAc/petroleum ether) to provide tert-butyl (2R,3R)-3-(4-fluorophenoxy)-2-methylazetidine-1-carboxylate. MS (ESI) m/z calculated for C₁₁H₁₀FNO₃ [M+H−(C₄H₈)]⁺226, found 226.

Compounds in Table 7 were prepared according to Scheme P, starting from commercially available azetidinols and (4-fluorophenyl)boronic acid.

TABLE 7
Intermediate Compounds Prepared According to Scheme P

| | Structure | Observed m/z |
|---|---|---|
| Entry | Name | [M + H − C₄H₈]⁺ |
| P.4 | 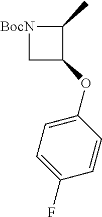 [SMILES: C[C@H]1[C@@H](Oc2ccc(F)cc2)CN1C(=O)OC(C)(C)C] | 226 |
| | tert-butyl (2S,3S)-3-(4-fluorophenoxy)-2- | |
| | methylazetidine-1-carboxylate | |
| P.5 | 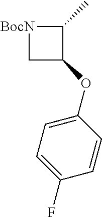 [SMILES: C[C@@H]1[C@@H](Oc2ccc(F)cc2)CN1C(=O)OC(C)(C)C] | 226 |
| | tert-butyl (2R,3S)-3-(4-fluorophenoxy)-2- | |
| | methylazetidine-1-carboxylate | |
| P.6 | 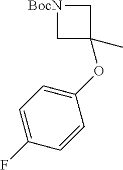 [SMILES: CC(C)(C)OC(=O)N1CC(C)(Oc2ccc(F)cc2)C1] | 226 |
| | tert-butyl 3-(4-fluorophenoxy)-3-methylazetidine-1- | |
| | carboxylate | |

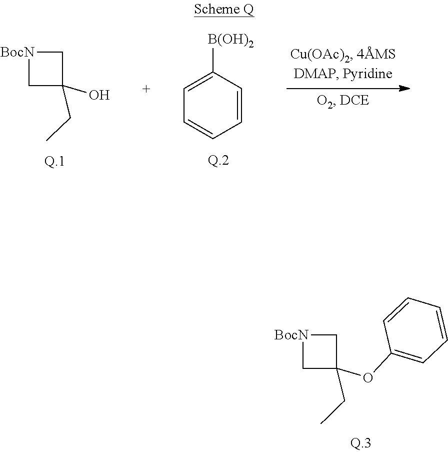 [SMILES: CCC1(O)CN(C(=O)OC(C)(C)C)C1 ; OB(O)c1ccccc1 ; CCC1(Oc2ccccc2)CN(C(=O)OC(C)(C)C)C1]

Intermediate Q.3 was also synthesized according to the method shown in Scheme P, starting from Q.1 and phenylboronic acid. ¹H NMR (400 MHz, CDCl₃) δ 7.29 (s, 1H), 7.25 (s, 1 H), 7.02-6.94 (m, 1H), 6.75-6.68 (m, 2H), 4.16 (d, J 9.6 Hz, 2H), 3.97 (d, J 9.6 Hz, 2H), 2.06 (q, J 7.6 Hz, 2H), 1.45 (s, 9H), 0.96-0.88 (m, 3H).

### Preparation of Intermediate R.3, tert-butyl 3-((6-cyanopyrimidin-4-yl)oxy)azetidine-1-carboxylate

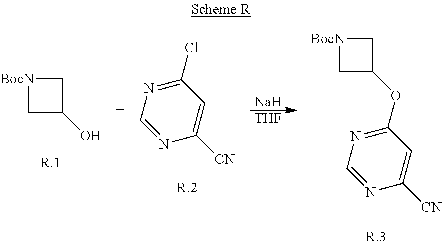

Sodium hydride was added to stirred solution of tert-butyl 3-hydroxyazetidine-1-carboxylate (tert-butyl 3-hydroxyazetidine-1-carboxylate) (500 mg, 2.89 mmol) in THF (29 mL) that had been cooled to 0° C. The resulting cloudy suspension was stirred for 15 min at 0° C., then a solution of 6-chloropyrimidine-4-carbonitrile (403 mg, 2.89 mmol) in THF (1 mL) was added. The reaction was stirred at 0° C. for 10 min, then warmed to 25° C. and stirred for 2 h. Water (5 mL) and DCM (15 mL) were then added, and the resulting biphasic mixture was stirred at 25° C. for 5 min. The layers were then separated, and the aq. layer was extracted with DCM (15 mL). The combined organic layers were dried over Na₂SO₄ and concentrated. The crude residue was purified by silica gel chromatography (gradient elution: 0-100% EtOAc/hexanes) to provide tert-butyl 3-((6-cyanopyrimidin-4-yl)oxy)azetidine-1-carboxylate. MS (ESI) m/z calculated for C₁₃H₁₇N₄O₃ [M+H]⁺ 277, found 277.

Compounds in Table 8 were prepared using a similar procedure to Scheme R, starting from the appropriate commercially available azetidinol and either 4-chloropicolinonitrile or bromocyclobutane. A slight modification was employed, replacing THF with DMF as the solvent. For R.4, R.5, and R.7, the reaction was run at 40° C. following the addition of 4-chloropicolinonitrile. Compounds in Table 8 were purified by silica gel chromatography or preparative TLC (silica gel).

TABLE 8
Intermediate Compounds Prepared According to Scheme R

| | | Observed |
|---|---|---|
| | Structure | m/z |
| Entry | Name | [M + H]⁺ |
| R.4 | 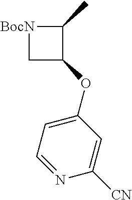 | 290 |
| | tert-butyl (2S,3S)-3-((2-cyanopyridin-4-yl)oxy)-2- | |
| | methylazetidine-1-carboxylate | |
| R.5 | 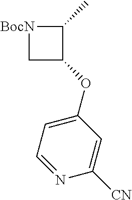 | 290 |
| | tert-butyl (2R,3R)-3-((2-cyanopyridin-4-yl)oxy)-2- | |
| | methylazetidine-1-carboxylate | |
| R.6 | 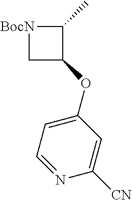 | 290 |
| | tert-butyl (2R,3S)-3-((2-cyanopyridin-4-yl)oxy)-2- | |
| | methylazetidine-1-carboxylate | |
| R.7 | 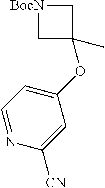 | 290 |
| | tert-butyl 3-((2-cyanopyridin-4-yl)oxy)-3-methylazetidine- | |
| | 1-carboxylate | |
| R.8 | 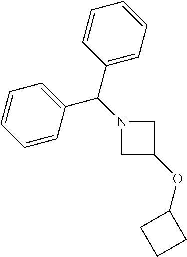 | 294 |
| | 1-benzhydryl-3-cyclobutoxyazetidine | |

### Preparation of Intermediate S.2, tert-butyl 3-(cyclopentyloxy)azetidine-1-carboxylate

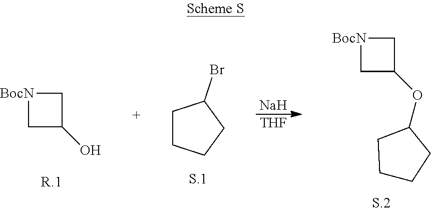

Sodium hydride (277 mg, 6.93 mmol) was added to stirred solution of tert-butyl 3-hydroxyazetidine-1-carboxylate (150 mg, 0.866 mmol) in THF (18 mL) that had been cooled to 0° C. The resulting cloudy suspension was stirred for 30 min at 0° C., and then bromocyclopentane (0.70 mL, 6.93 mmol) was added. The reaction mixture was then stirred at 0° C. for an additional 10 min, then heated at 75° C. for 3 days. After cooling, the reaction was then poured into water (30 mL) and DCM (40 mL), and the resulting biphasic mixture was stirred at 25° C. for 5 min. The layers were then separated, and the aq. layer was extracted with DCM (2×30 mL). The combined organic layers were dried over MgSO₄ and concentrated. The crude residue was then purified by silica gel chromatography (gradient elution: 0-100% [25% EtOH in EtOAc]/hexanes) to provide tert-butyl 3-(cyclopentyloxy)azetidine-1-carboxylate. MS (ESI) m/z calculated for C₉H₁₆NO₃ [M+H−(C₄H₈)]⁺186, found 186.

### Preparation of Intermediate T.3, tert-butyl 3-((6-cyanopyridin-3-yl)oxy)azetidine-1-carboxylate

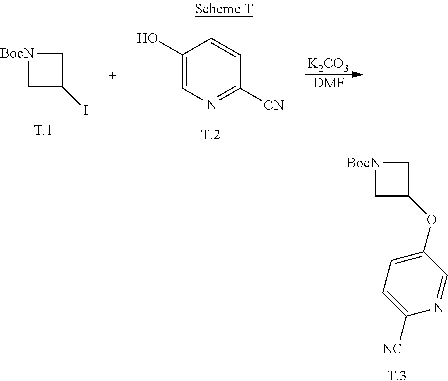

A stirring mixture of 5-hydroxypicolinonitrile (100 mg, 0.833 mmol) and K₂CO₃ (230 mg, 1.67 mmol) in DMF (0.5 mL) was cooled to 0° C. Tert-butyl 3-iodoazetidine-1-carboxylate (0.290 mL, 1.67 mmol) was then added, and the reaction mixture was heated to 80° C. and stirred for 3 days. After cooling, water (5 mL) and Et₂O (6 mL) were added. The layers were then separated, and the aq. layer was extracted with Et₂O (2×10 mL). The combined organic layers were washed with water (2×5 mL) and brine (5 mL), dried over MgSO₄ and concentrated. The crude residue was then purified by silica gel chromatography (gradient elution: 0-20% MeOH in DCM) to provide tert-butyl 3-((6-cyanopyridin-3-yl)oxy)azetidine-1-carboxylate. MS (ESI) m/z calculated for C₁₀H₁₀N₃O₃ [M+H−(C₄H₈)]⁺220, found 220.

### Preparation of Intermediate U.2, tert-butyl 3-(3-cyanophenoxy)azetidine-1-carboxylate

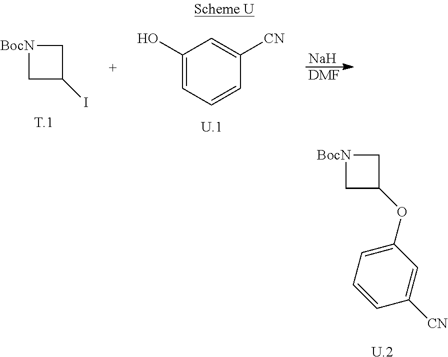

A stirring mixture of 3-hydroxybenzonitrile (500 mg, 4.20 mmol) in DMF (10 mL) was cooled to 0° C. and treated with sodium hydride (201 mg, 5.04 mmol). The resulting mixture was stirred at 20° C. for 30 min, then tert-butyl 3-iodoazetidine-1-carboxylate (1.55 g, 5.46 mmol) was added. The reaction mixture was then stirred at 40° C. for 2 h. After cooling, water (20 mL) and EtOAc (20 mL) were added and the layers were separated. The aq. layer was extracted with EtOAc (2×10 mL), and the combined organic layers were washed with brine (2×30 mL), dried over Na₂SO₄ and concentrated. The crude residue was purified by preparative TLC (silica gel, eluent: 17% EtOAc/petroleum ether) to provide tert-butyl 3-(3-cyanophenoxy)azetidine-1-carboxylate. MS (ESI) m/z calculated for C₁₁H₁₁N₂O₃ [M+H−(C₄H₈)]⁺219, found 219.

### Preparation of Intermediate V.5, tert-butyl 3-((2-cyanopyridin-4-yl)difluoromethyl)azetidine-1-carboxylate

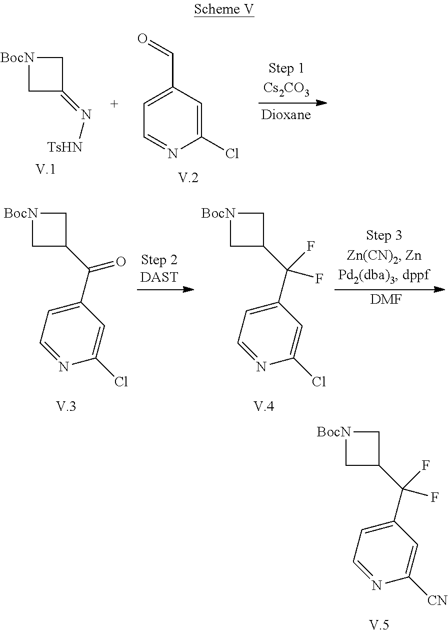

### Step 1—Synthesis of Intermediate V.3, tert-butyl 3-(2-chloroisonicotinoyl)azetidine-1-carboxylate

A mixture of tert-butyl 3-(2-tosylhydrazineylidene) azetidine-1-carboxylate (1.0 g, 2.95 mmol), Cs₂CO₃ (1.44 g, 4.42 mmol) and 2-chloroisonicotinaldehyde (417 mg, 2.95 mmol) in dioxane (20 mL) was heated to 110° C. and stirred for 6 h. The reaction was then quenched with sat. aq. NH₄Cl (30 mL), extracted with EtOAc (3×40 mL), dried over Na₂SO₄, and concentrated. The crude residue was then purified by silica gel chromatography (gradient elution: 0-30% EtOAc/petroleum ether) to provide tert-butyl 3-(2-chloroisonicotinoyl)azetidine-1-carboxylate. MS (ESI) calculated for C₁₀H₁₀ClN₂O₃ [M+H−(C₄H₈)]⁺241, found 241.

### Step 2—Synthesis of Intermediate V.4, tert-butyl 3-((2-chloropyridin-4-yl)difluoromethyl)azetidine-1-carboxylate

Tert-butyl 3-(2-chloroisonicotinoyl)azetidine-1-carboxylate (150 mg, 0.505 mmol) was treated with DAST (7 mL), and the resulting mixture was stirred at 50° C. for 12 h. The reaction was then quenched with sat. aq. NaHCO₃(40 mL) and extracted with DCM (3×50 mL). The combined organic layers were washed with brine (2×50 mL), dried over Na₂SO₄ and concentrated. The crude residue was then purified by preparative TLC (silica gel, eluent: 25% EtOAc/petroleum ether) to provide tert-butyl 3-((2-chloropyridin-4-yl)difluoromethyl)azetidine-1-carboxylate. MS (ESI) calculated for C₁₀H₁₀ClF₂N₂O₂ [M+H−(C₄H₈)]⁺263, found 263.

### Step 3—Preparation of Intermediate V.5, tert-butyl 3-((2-cyanopyridin-4-yl)difluoromethyl)azetidine-1-carboxylate

A solution of tert-butyl 3-((2-chloropyridin-4-yl)difluoromethyl)azetidine-1-carboxylate (50 mg, 0.094 mmol) in DMF (1 mL) was treated with dicyanozinc (33.2 mg, 0.282 mmol), zinc (2.5 mg, 0.038 mmol), dppf (21 mg, 0.038 mmol) and Pd₂(dba)₃ (17 mg, 0.019 mmol) under an atmosphere of nitrogen. The resulting mixture was stirred at 110° C. for 12 h. The reaction mixture was then diluted with water (10 mL) and extracted with EtOAc (3×10 mL). The combined organic layers were dried over Na₂SO₄ and concentrated. The crude residue was then purified by preparative TLC (silica gel, eluent: 25% EtOAc/petroleum ether) to provide tert-butyl 3-((2-cyanopyridin-4-yl)difluoromethyl)azetidine-1-carboxylate. MS (ESI) m/z calculated for C₁₁H₁₀F₂N₃O₂ [M+H−(C₄H₈)]⁺254, found 254.

### Preparation of Intermediate W.4, tert-butyl 3-(1-phenylethyl)azetidine-1-carboxylate

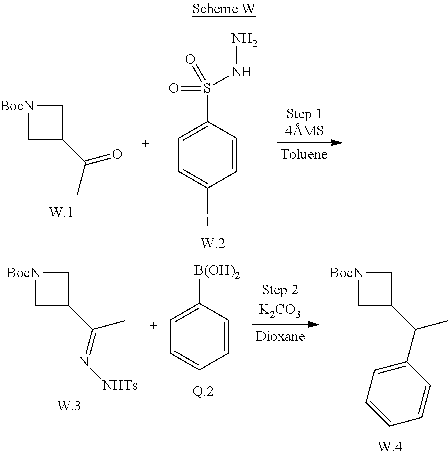

### Step 1—Synthesis of Intermediate W.3, tert-butyl 3-(1-(2-tosylhydrazono)ethyl)azetidine-1-carboxylate

A solution of tert-butyl 3-acetylazetidine-1-carboxylate (200 mg, 1.00 mmol) in toluene (2 ml) was treated with 4-methylbenzenesulfonohydrazide (374 mg, 2.01 mmol) and 4 Å molecular sieves. The resulting reaction mixture was stirred at 120° C. for 8 h. The reaction was then filtered and concentrated to provide tert-butyl 3-(1-(2-tosylhydrazono)ethyl)azetidine-1-carboxylate, which was used directly in the subsequent reaction. ¹H NMR (400 MHz, CDCl₃) δ 7.83 (d, J=8.4 Hz, 2H), 7.50 (s, 1H), 7.30 (d, J=8.0, 2H), 4.02-3.95 (m, 2H), 3.90-3.84 (m, 2H), 3.32-2.23 (m, 1H), 2.41 (s, 3H), 1.79 (s, 3H), 1.41 (s, 9H).

### Step 2—Preparation of Intermediate W.4, Preparation of Intermediate P.5, tert-butyl 3-(1-phenylethyl)azetidine-1-carboxylate

A mixture of tert-butyl 3-(1-(2-tosylhydrazono)ethyl)azetidine-1-carboxylate (30 mg, 0.082 mmol), K₂CO₃ (33.8 mg, 0.245 mmol) and phenylboronic acid (14.9 mg, 0.122 mmol) in dioxane (1.5 mL) was heated to 110° C. and stirred for 5 h. The reaction was then directly filtered and concentrated. The crude residue was purified by preparative TLC (silica gel, eluent: EtOAc) to provide tert-butyl 3-(1-phenylethyl)azetidine-1-carboxylate. ¹H NMR (400 MHz, CDCl₃) δ 7.29-7.23 (m, 2H), 7.21-7.18 (m, 1H), 7.15-7.11 (m, 2H), 4.08-4.02 (m, 1H), 3.74-3.66 (m, 2H), 3.47-3.43 (m, 1H), 2.87-2.82 (m, 1H), 2.70-2.65 (m, 1H), 1.40 (s, 9H), 1.18 (d, J=6.8 Hz, 3H).

### Preparation of Intermediate X.3, tert-butyl 3-((2-cyanopyridin-4-yl)methyl)azetidine-1-carboxylate

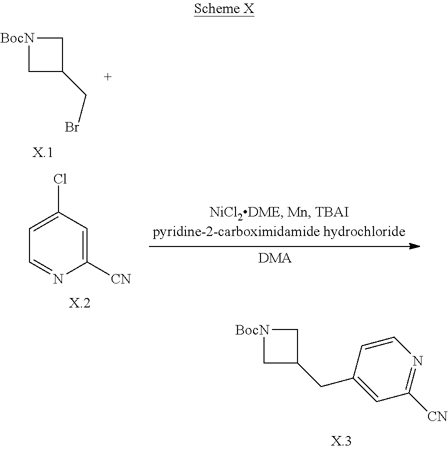

A stirred mixture of 4-chloropyridine-2-carbonitrile (78 mg, 0.560 mmol), tert-butyl 3-(bromomethyl)azetidine-1-carboxylate (100 mg, 0.400 mmol), tetrabutylammonium iodide (73.8 mg, 0.200 mmol), nickel(II) chloride ethylene glycol dimethyl ether complex (35.1 mg, 0.160 mmol), and pyridine-2-carboximidamide hydrochloride (31.5 mg, 0.200 mmol) in DMA (3 mL) was stirred under nitrogen atmosphere at 20° C. Manganese (7.5 μL, 0.999 mmol) was then added and the reaction mixture was stirred at 75° C. for 16 h. After cooling, the reaction was quenched with water (20 mL) and extracted with EtOAc (3×15 mL). The combined organic layers were washed with brine (30 mL), dried over Na₂SO₄ and concentrated. The crude residue was purified by preparative TLC (silica gel, eluent: 33% EtOAc/petroleum ether) to provide tert-butyl 3-((2-cyanopyridin-4-yl)methyl)azetidine-1-carboxylate. MS (ESI) m/z calculated for C₁₁H₁₂N₃O₂ [M+H−(C₄H₈)]⁺218, found 218.

### Preparation of Intermediate Y.1, tert-butyl 3-cyclobutoxyazetidine-1-carboxylate

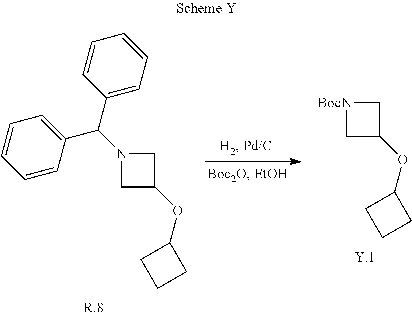 [SMILES: c1ccc(C(c2ccccc2)N2CC(OC3CCC3)C2)cc1 ; CC(C)(C)OC(=O)N1CC(OC2CCC2)C1]

A solution of 1-benzhydryl-3-cyclobutoxyazetidine (20 mg, 0.068 mmol) in EtOH (1 mL) was treated with Boc₂O (0.032 mL, 0.136 mmol) and Pd/C (0.73 mg, 6.8 μmol). The resulting mixture was stirred at 20° C. for 1 h under hydrogen atmosphere. The reaction mixture was then filtered and concentrated to provide tert-butyl 3-cyclobutoxyazetidine-1-carboxylate, which was used directly in the subsequent reaction. MS (ESI) m/z calculated for C₈H₁₄NO₃ [M+H−(C₄H₈)]⁺172, found 172.

### Preparation of Intermediate Z.1, 6-(azetidin-3-yloxy)pyrimidine-4-carbonitrile hydrochloride

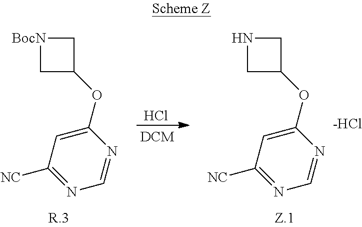

HCl (4 M in dioxane, 0.353 mL, 1.41 mmol) was added to a stirred solution of tert-butyl 3-((6-cyanopyrimidin-4-yl)oxy)azetidine-1-carboxylate (78 mg, 0.282 mmol) in DCM (2.8 mL). The resulting mixture was stirred at 25° C. for 18 h. The solid precipitate that formed during the reaction was then filtered, and the precipitate rinsed with 5 mL DCM. This provided 6-(azetidin-3-yloxy)pyrimidine-4-carbonitrile hydrochloride. MS (ESI) m/z calculated for C₈H₉N₄₀ [M+H]⁺ 177, found 177.

Compounds in Table 9 were prepared according to Scheme Z, starting from tert-butyl 3-benzoylazetidine-1-carboxylate or intermediate T.3.

TABLE 9
Intermediate Compounds Prepared According to Scheme Z

| | | Observed m/z |
|---|---|---|
| Entry | Structure | [M + H]⁺ |
| Z.2 | 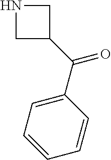 [SMILES: O=C(c1ccccc1)C1CNC1] | 162 |
| | azetidin-3-yl(phenyl)methanone hydrochloride | |
| Z.3 | 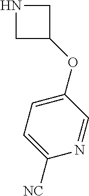 [SMILES: N#Cc1ccc(OC2CNC2)cn1] | 176 |
| | 5-(azetidin-3-yloxy)picolinonitrile hydrochloride | |

### Preparation of Intermediate AA.1, (2S,3S and 2R,3R)-2-methyl-3-phenoxyazetidine

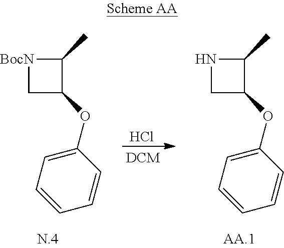 [SMILES: C[C@H]1[C@@H](Oc2ccccc2)CN1C(=O)OC(C)(C)C ; Cl ; C[C@@H]1NC[C@@H]1Oc1ccccc1]

HCl (4 M in dioxane, 0.266 mL, 1.06 mmol) was added to a stirred solution of tert-butyl (2S,3R and 2R,3S)-2-methyl-3-phenoxyazetidine-1-carboxylate (56 mg, 0.213 mmol) in DCM (2.1 mL). The resulting mixture was stirred at 25° C. for 18 h. Sat. aq. NaHCO₃(10 mL) and DCM (5 mL) were then added, and the resulting biphasic mixture was stirred for 5 min. The layers were then separated, and the aq. layer was extracted with DCM (3×12 mL). The combined organic layers were dried over MgSO₄ and concentrated to provide (2S,3S and 2R,3R)-2-methyl-3-phenoxyazetidine, which was used directly in the subsequent reaction. MS (ESI) m/z calculated for C₁₀H₁₄NO [M+H]⁺164, found 164.

Compounds in Table 10 were prepared according to Scheme AA, starting from intermediates O.1, O.2 or S.2.

TABLE 10
Intermediate Compounds Prepared According to Scheme AA

| | Structure | Observed m/z |
|---|---|---|
| Entry | Name | [M + H]⁺ |
| AA.2 | 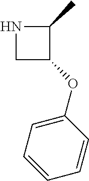 | 164 |
| | (2S,3R or 2R,3S)-2-methyl-3-phenoxyazetidine | |
| AA.3 | 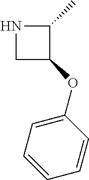 | 164 |
| | (2R,3S or 2S,3R)-2-methyl-3-phenoxyazetidine | |
| AA.4 | 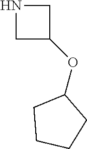 | 142 |
| | 3-(cyclopentyloxy)azetidine | |

### Preparation of Intermediate AB.2, 3-((4-bromophenyl)sulfonyl)azetidine hydrochloride

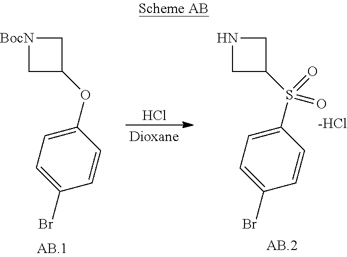

HCl (4 M in dioxane, 0.199 mL, 0.797 mmol) was added to a stirred solution of tert-butyl 3-((4-bromophenyl)sulfonyl)azetidine-1-carboxylate (60 mg, 0.159 mmol) in dioxane (1 mL). The resulting mixture was stirred at 25° C. for 4 h, then directly concentrated to provide 3-((4-bromophenyl)sulfonyl)azetidine hydrochloride, which was used in the subsequent reaction without further purification. MS (ESI) m/z calculated for C₉H₁₁BrNO₂S [M+H]⁺ 276, found 276, 278.

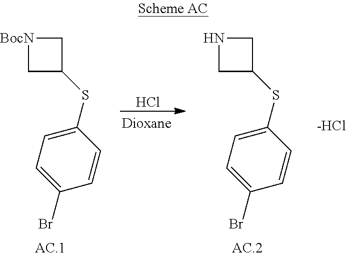

Intermediate AC.2 was also synthesized according to the method shown in Scheme AB, starting from AC.1. MS (ESI) m/z calculated for C₉H₁₁BrNS [M+H]⁺ 244, found 244, 246.

### Preparation of Intermediate AD.1, (2R,3R)-3-(4-fluorophenoxy)-2-methylazetidine, TFA salt

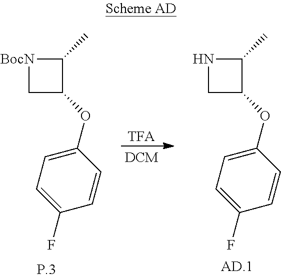 [SMILES: C[C@@H]1[C@H](Oc2ccc(F)cc2)CN1C(=O)OC(C)(C)C ; C[C@H]1NC[C@H]1Oc1ccc(F)cc1 ; "TFA"]

A solution of tert-butyl (2R,3R)-3-(4-fluorophenoxy)-2-methylazetidine-1-carboxylate (35 mg, 0.124 mmol) in DCM (2 mL) and TFA (1 mL) was stirred at 30° C. for 1 h. The reaction mixture was then directly concentrated to provide (2R,3R)-3-(4-fluorophenoxy)-2-methylazetidine, TFA salt, which was used directly in the subsequent reaction. MS (ESI) m/z calculated for C₁₀H₁₃FNO [M+H]⁺ 182, found 182.

Compounds in Table 11 were prepared according to Scheme AD, starting from intermediates P.4, P.5, P.6, R.4, R.5, R.6, R.7, V.5, W.4, U.2, Q.3, X.3, or Y.1.

TABLE 11
Intermediate Compounds Prepared According to Scheme AD

| | | Observed |
|---|---|---|
| | Structure | m/z |
| Entry | Name | [M + H]⁺ |
| AD.2 | 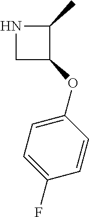 | 182 |
| | (2S,3S)-3-(4-fluorophenoxy)-2-methylazetidine, | |
| | TFA salt | |
| AD.3 | 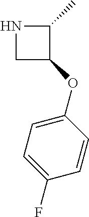 | 182 |
| | (2R,3S)-3-(4-fluorophenoxy)-2-methylazetidine, | |
| | TFA salt | |
| AD.4 | 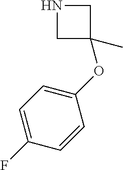 | 182 |
| | 3-(4-fluorophenoxy)-3-methylazetidine, TFA salt | |
| AD.5 | 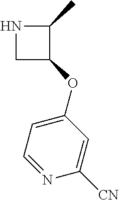 | 190 |
| | 4-(((25,3S)-2-methylazetidin-3-yl)oxy)picolinonitrile, | |
| | TFA salt | |
| AD.6 | 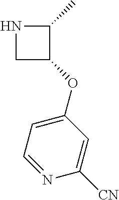 | 190 |
| | 4-(((2R,3R)-2-methylazetidin-3-yl)oxy)picolinonitrile, | |
| | TFA salt | |
| AD.7 | 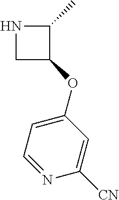 | 190 |
| | 4-(((2R,3S)-2-methylazetidin-3-yl)oxy)picolinonitrile, | |
| | TFA salt | |
| AD.8 | 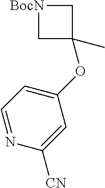 [SMILES: CC(C)(C)OC(=O)N1CC(C)(Oc2ccnc(C#N)c2)C1] | 190 |
| | 4-((3-methylazetidin-3-yl)oxy)picolinonitrile, TFA salt | |
| AD.9 | 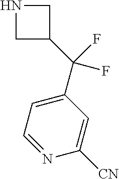 [SMILES: N#Cc1cc(C(F)(F)C2CNC2)ccn1] | 210 |
| | 4-(azetidin-3-yldifluoromethyl)picolinonitrile, TFA salt | |
| AD.10 | 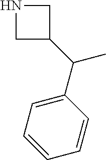 | 162 |
| | 3-(1-phenylethyl)azetidine, TFA salt | |
| AD.11 | 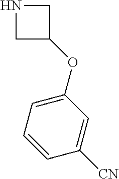 [SMILES: N#Cc1cccc(OC2CNC2)c1] | 175 |
| | 3-(azetidin-3-yloxy)benzonitrile, TFA salt | |
| AD.12 | 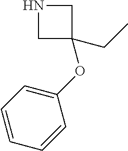 | 178 |
| | 3-ethyl-3-phenoxyazetidine, TFA salt | |
| AD.13 | 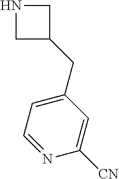 [SMILES: N#Cc1cc(CC2CNC2)ccn1] | 174 |
| | 4-(azetidin-3-ylmethyl)picolinonitrile, TFA salt | |
| AD.14 | 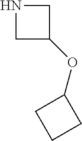 | 128 |
| | 3-cyclobutoxyazetidine, TFA salt | |

### Preparation of Example 1.1, (S)-(5-(3,5-difluorophenyl)-4,5-dihydro-1H-pyrazol-1-yl)(3-methyl-3-phenoxyazetidin-1-yl)methanone, TFA salt

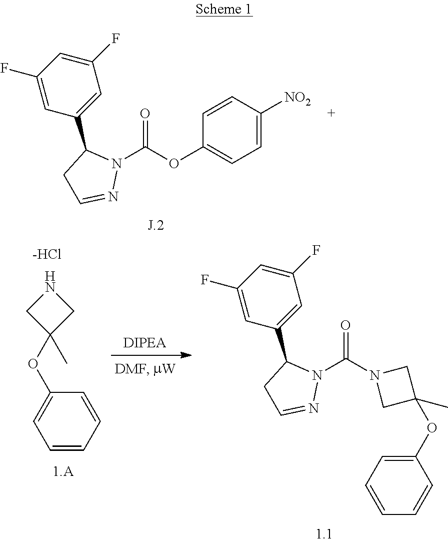

A 2 mL Biotage® microwave vial was charged with a 0.37 M DMSO solution of 4-nitrophenyl(S)-5-(3,5-difluorophenyl)-4,5-dihydro-1H-pyrazole-1-carboxylate (0.29 mL, 0.107 mmol), 3-methyl-3-phenoxyazetidine, HCl (21.4 mg, 0.107 mmol) and DIPEA (18.7 μl, 0.107 mmol), and the vial was evacuated and backfilled with nitrogen (3×). DMF (1.6 mL) was added, and the vial was heated under microwave irradiation at 150° C. for 30 min. After completion, the reaction was filtered and purified via reversed phase HPLC [Method A]. This provided (S)-(5-(3,5-difluorophenyl)-4,5-dihydro-1H-pyrazol-1-yl)(3-methyl-3-phenoxyazetidin-1-yl)methanone, TFA salt. MS (ESI) m/z calculated for C₂₀H₂₀F₂N₃O₂ [M+H]⁺372, found 372. ¹H NMR (600 MHz, DMSO-d₆) δ 7.31-7.27 (m, 2H), 7.12-7.07 (m, 1H), 7.02 (br s, 1H), 6.96 (t, J=7.3 Hz, 1H), 6.92-6.87 (m, 2H), 6.78 (d, J=7.9 Hz, 2H), 5.23 (dd, J-12.1, 6.5 Hz, 1H), 4.24-4.09 (m, 4H), 3.38 (ddd, J=18.7, 12.2, 1.5 Hz, 1H), 2.64 (ddd, J=18.7, 6.5, 1.6 Hz, 1 H), 1.62 (s, 3H). RIPK1 EC₅₀ 2.5 nM.

The following examples in Table 12 were prepared according to Scheme 1 and General Scheme 1 above, using intermediates L.1, M.2, or M.1, and the appropriate azetidine or azetidine hydrochloride coupling partner, either commercially available or intermediates Z.1, AA.2, AA.3, AA.1, Z.2, AA.4, AC.2, or AB.2. For Example 1.18 through Example 1.55, a slightly modified procedure was used wherein DIPEA was replaced by cesium carbonate, and the reaction was run at 100° C. for 16 h using conventional heating. The compounds were generally purified by reversed phase HPLC and SFC. Where isomers were separated by SFC conditions are provided, following the table.

TABLE 12
Examples Prepared According to General Scheme 1 and Scheme 1

| | Structure | Observed m/z | RIPK1 EC₅₀ |
|---|---|---|---|
| Example | Name | [M + H]⁺ | (nM) |
| 1.2 | 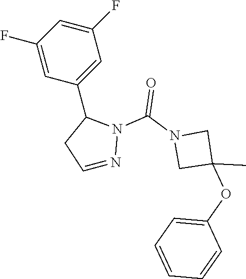 [SMILES: CC1(Oc2ccccc2)CN(C(=O)N2N=CCC2c2cc(F)cc(F)c2)C1] | 372 | 9.7 |
| | (R and S)-(5-(3,5-difluorophenyl)-4,5- | | |
| | dihydro-1H-pyrazol-1-yl)(3-methyl-3- | | |
| | phenoxyazetidin-1-yl)methanone, TFA salt. | | |
| 1.3 | 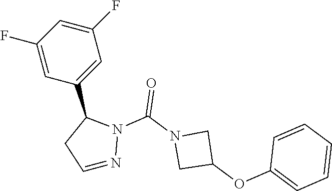 [SMILES: O=C(N1CC(Oc2ccccc2)C1)N1N=CC[C@H]1c1cc(F)cc(F)c1] | 358 | 3.8 |
| | (S)-(5-(3,5-difluorophenyl)-4,5-dihydro-1H- | | |
| | pyrazol-1-yl)(3-phenoxyazetidin-1- | | |
| | yl)methanone, TFA salt. | | |
| 1.4 | 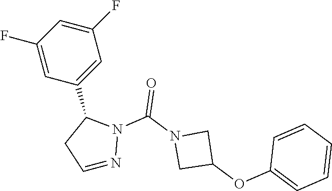 [SMILES: O=C(N1CC(Oc2ccccc2)C1)N1N=CC[C@@H]1c1cc(F)cc(F)c1] | 358 | 600.2 |
| | (R)-(5-(3,5-difluorophenyl)-4,5-dihydro-1H- | | |
| | pyrazol-1-yl)(3-phenoxyazetidin-1- | | |
| | yl)methanone, TFA salt. | | |
| 1.5 | 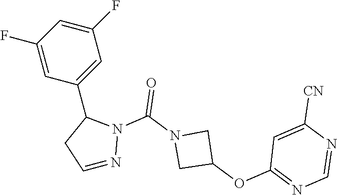 [SMILES: N#Cc1cc(OC2CN(C(=O)N3N=CCC3c3cc(F)cc(F)c3)C2)ncn1] | 385 | 140.2 |
| | (R and S)-6-((1-(5-(3,5-difluorophenyl)-4,5- | | |
| | dihydro-1H-pyrazole-1-carbonyl)azetidin-3- | | |
| | yl)oxy)pyrimidine-4-carbonitrile, TFA salt | | |
| 1.6 | 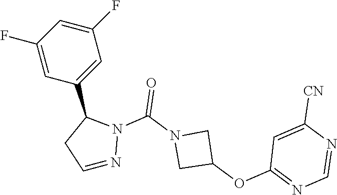 [SMILES: N#Cc1cc(OC2CN(C(=O)N3N=CC[C@H]3c3cc(F)cc(F)c3)C2)ncn1] | 385 | 83.0 |
| | (S)-6-((1-(5-(3,5-difluorophenyl)-4,5- | | |
| | dihydro-1H-pyrazole-1-carbonyl)azetidin-3- | | |
| | yl)oxy)pyrimidine-4-carbonitrile | | |
| 1.7 | 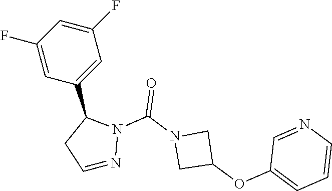 [SMILES: O=C(N1CC(Oc2cccnc2)C1)N1N=CC[C@H]1c1cc(F)cc(F)c1] | 359 | 12.8 |
| | (S)-(5-(3,5-difluorophenyl)-4,5-dihydro-1H- | | |
| | pyrazol-1-yl)(3-(pyridin-3-yloxy)azetidin-1- | | |
| | yl)methanone, TFA salt | | |
| 1.8 | 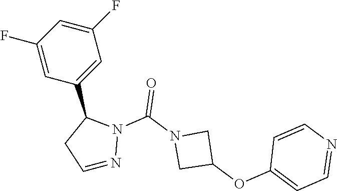 [SMILES: O=C(N1CC(Oc2ccncc2)C1)N1N=CC[C@H]1c1cc(F)cc(F)c1] | 359 | 8.7 |
| | (S)-(5-(3,5-difluorophenyl)-4,5-dihydro-1H- | | |
| | pyrazol-1-yl)(3-(pyridin-4-yloxy)azetidin-1- | | |
| | yl)methanone, TFA salt | | |
| 1.9 | 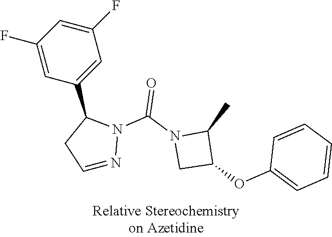 [SMILES: C[C@H]1[C@H](Oc2ccccc2)CN1C(=O)N1N=CC[C@H]1c1cc(F)cc(F)c1] | 372 | 104.1 |
| | ((S)-5-(3,5-difluorophenyl)-4,5-dihydro-1H- | | |
| | pyrazol-1-yl)((2S,3R or 2R,3S)-2-methyl-3- | | |
| | phenoxyazetidin-1-yl)methanone, TFA salt | | |
| 1.10 | 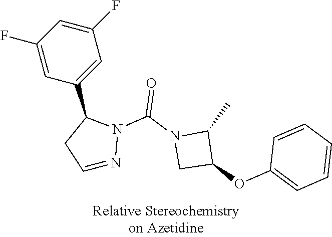 [SMILES: C[C@@H]1[C@@H](Oc2ccccc2)CN1C(=O)N1N=CC[C@H]1c1cc(F)cc(F)c1] | 372 | 10.5 |
| | ((S)-5-(3,5-difluorophenyl)-4,5-dihydro-1H- | | |
| | pyrazol-1-yl)((2R,3S or 2S,3R)-2-methyl-3- | | |
| | phenoxyazetidin-1-yl)methanone, TFA salt | | |
| 1.11 | 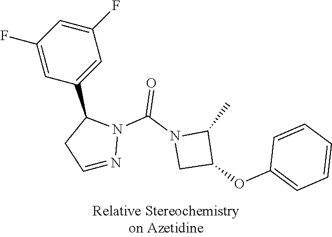 [SMILES: C[C@@H]1[C@H](Oc2ccccc2)CN1C(=O)N1N=CC[C@H]1c1cc(F)cc(F)c1] | 372 | 11.8 |
| | ((S)-5-(3,5-difluorophenyl)-4,5-dihydro-1H- | | |
| | pyrazol-1-yl)((2R,3R and 2S,3S)-2-methyl-3- | | |
| | phenoxyazetidin-1-yl)methanone, TFA salt | | |
| 1.12 | 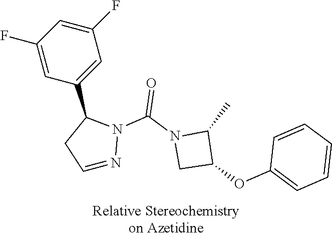 [SMILES: C[C@@H]1[C@H](Oc2ccccc2)CN1C(=O)N1N=CC[C@H]1c1cc(F)cc(F)c1] | 372 | 8.8 |
| | ((S)-5-(3,5-difluorophenyl)-4,5-dihydro-1H- | | |
| | pyrazol-1-yl)((2R,3R or 2S,3S)-2-methyl-3- | | |
| | phenoxyazetidin-1-yl)methanone, TFA salt | | |
| 1.13 | 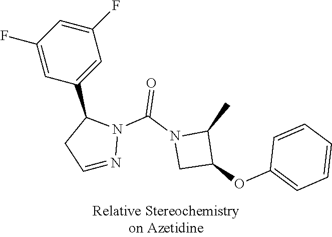 [SMILES: C[C@H]1[C@@H](Oc2ccccc2)CN1C(=O)N1N=CC[C@H]1c1cc(F)cc(F)c1] | 372 | 7.6 |
| | ((S)-5-(3,5-difluorophenyl)-4,5-dihydro-1H- | | |
| | pyrazol-1-yl)((2S,3S or 2R,3R)-2-methyl-3- | | |
| | phenoxyazetidin-1-yl)methanone, TFA salt | | |
| 1.14 | 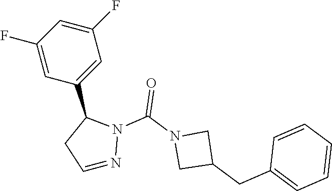 [SMILES: O=C(N1CC(Cc2ccccc2)C1)N1N=CC[C@H]1c1cc(F)cc(F)c1] | 356 | 6.1 |
| | (S)-(3-benzylazetidin-1-yl)(5-(3,5- | | |
| | difluorophenyl)-4,5-dihydro-1H-pyrazol-1- | | |
| | yl)methanone, TFA salt | | |
| 1.15 | 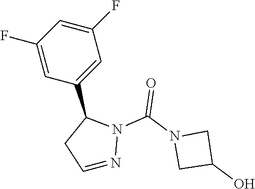 [SMILES: O=C(N1CC(O)C1)N1N=CC[C@H]1c1cc(F)cc(F)c1] | 282 | 3678 |
| | (S)-(5-(3,5-difluorophenyl)-4,5-dihydro-1H- | | |
| | pyrazol-1-yl)(3-hydroxyazetidin-1- | | |
| | yl)methanone | | |
| 1.16 | 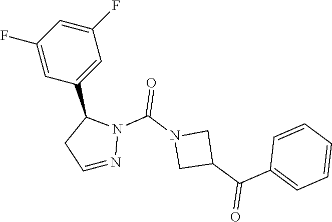 [SMILES: O=C(c1ccccc1)C1CN(C(=O)N2N=CC[C@H]2c2cc(F)cc(F)c2)C1] | 370 | 5.6 |
| | (S)-(3-benzoylazetidin-1-yl)(5-(3,5- | | |
| | difluorophenyl)-4,5-dihydro-1H-pyrazol-1- | | |
| | yl)methanone | | |
| 1.17 | 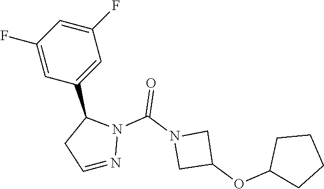 [SMILES: O=C(N1CC(OC2CCCC2)C1)N1N=CC[C@H]1c1cc(F)cc(F)c1] | 350 | 11.5 |
| | (S)-(3-(cyclopentyloxy)azetidin-1-yl)(5-(3,5- | | |
| | difluorophenyl)-4,5-dihydro-1H-pyrazol-1- | | |
| | yl)methanone, TFA salt | | |
| 1.18 | 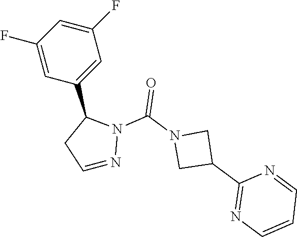 [SMILES: O=C(N1CC(c2ncccn2)C1)N1N=CC[C@H]1c1cc(F)cc(F)c1] | 344 | 324.6 |
| | (S)-(5-(3,5-difluorophenyl)-4,5-dihydro-1H- | | |
| | pyrazol-1-yl)(3-(pyrimidin-2-yl)azetidin-1- | | |
| | yl)methanone | | |
| 1.19 | 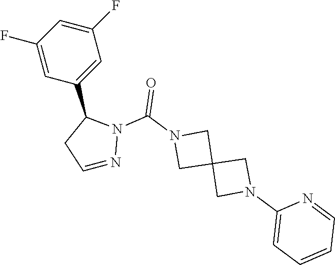 [SMILES: O=C(N1CC2(C1)CN(c1ccccn1)C2)N1N=CC[C@H]1c1cc(F)cc(F)c1] | 384 | 3446 |
| | (S)-(5-(3,5-difluorophenyl)-4,5-dihydro-1H- | | |
| | pyrazol-1-yl)(6-(pyridin-2-yl)-2,6- | | |
| | diazaspiro[3.3]heptan-2-yl)methanone, TFA | | |
| | salt | | |
| 1.20 | 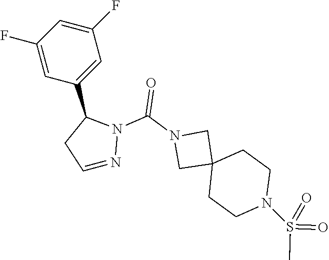 [SMILES: CS(=O)(=O)N1CCC2(CC1)CN(C(=O)N1N=CC[C@H]1c1cc(F)cc(F)c1)C2] | 413 | 1319 |
| | (S)-(5-(3,5-difluorophenyl)-4,5-dihydro-1H- | | |
| | pyrazol-1-yl)(7-(methylsulfonyl)-2,7- | | |
| | diazaspiro[3.5]nonan-2-yl)methanone | | |
| 1.21 | 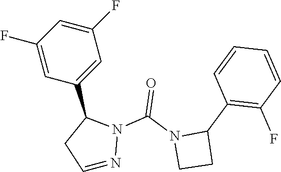 [SMILES: O=C(N1CCC1c1ccccc1F)N1N=CC[C@H]1c1cc(F)cc(F)c1] | 360 | 27.6 |
| | ((S)-5-(3,5-difluorophenyl)-4,5-dihydro-1H- | | |
| | pyrazol-1-yl)((R and S)-2-(2- | | |
| | fluorophenyl)azetidin-1-yl)methanone | | |
| 1.22 | 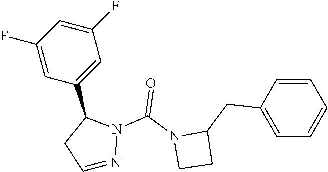 [SMILES: O=C(N1CCC1Cc1ccccc1)N1N=CC[C@H]1c1cc(F)cc(F)c1] | 356 | 73.6 |
| | ((S)-2-benzylazetidin-1-yl)((R and S)-5-(3,5- | | |
| | difluorophenyl)-4,5-dihydro-1H-pyrazol-1- | | |
| | yl)methanone | | |
| 1.23 | 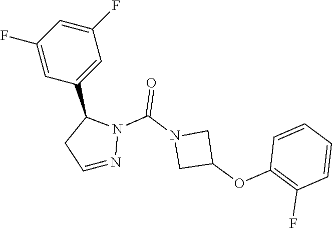 [SMILES: O=C(N1CC(Oc2ccccc2F)C1)N1N=CC[C@H]1c1cc(F)cc(F)c1] | 376 | 3.1 |
| | (S)-(5-(3,5-difluorophenyl)-4,5-dihydro-1H- | | |
| | pyrazol-1-yl)(3-(2-fluorophenoxy)azetidin-1- | | |
| | yl)methanone | | |
| 1.24 | 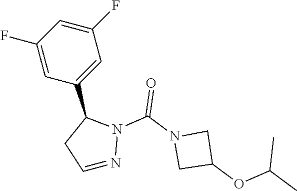 [SMILES: CC(C)OC1CN(C(=O)N2N=CC[C@H]2c2cc(F)cc(F)c2)C1] | 324 | 29.9 |
| | (S)-(5-(3,5-difluorophenyl)-4,5-dihydro-1H- | | |
| | pyrazol-1-yl)(3-isopropoxyazetidin-1- | | |
| | yl)methanone | | |
| 1.25 | 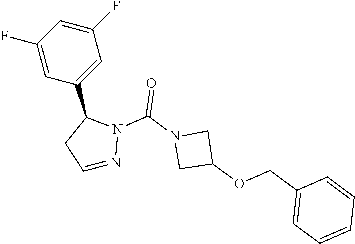 [SMILES: O=C(N1CC(OCc2ccccc2)C1)N1N=CC[C@H]1c1cc(F)cc(F)c1] | 372 | 11.8 |
| | (S)-(3-(benzyloxy)azetidin-1-yl)(5-(3,5- | | |
| | difluorophenyl)-4,5-dihydro-1H-pyrazol-1- | | |
| | yl)methanone | | |
| 1.26 | 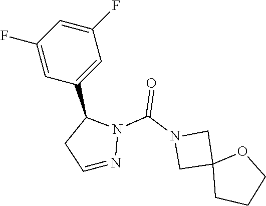 [SMILES: O=C(N1CC2(CCCO2)C1)N1N=CC[C@H]1c1cc(F)cc(F)c1] | 322 | 47.4 |
| | (S)-(5-(3,5-difluorophenyl)-4,5-dihydro-1H- | | |
| | pyrazol-1-yl)(5-oxa-2-azaspiro[3.4]octan-2- | | |
| | yl)methanone | | |
| 1.27 | 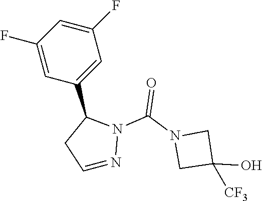 [SMILES: O=C(N1CC(O)(C(F)(F)F)C1)N1N=CC[C@H]1c1cc(F)cc(F)c1] | 350 | 988.4 |
| | (S)-(5-(3,5-difluorophenyl)-4,5-dihydro-1H- | | |
| | pyrazol-1-yl)(3-hydroxy-3- | | |
| | (trifluoromethyl)azetidin-1-yl)methanone | | |
| 1.28 | 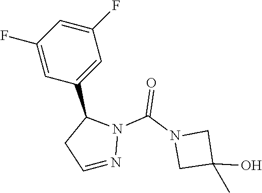 [SMILES: CC1(O)CN(C(=O)N2N=CC[C@H]2c2cc(F)cc(F)c2)C1] | 296 | 3591 |
| | (S)-(5-(3,5-difluorophenyl)-4,5-dihydro-1H- | | |
| | pyrazol-1-yl)(3-hydroxy-3-methylazetidin-1- | | |
| | yl)methanone | | |
| 1.29 | 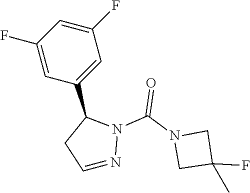 [SMILES: CC1(F)CN(C(=O)N2N=CC[C@H]2c2cc(F)cc(F)c2)C1] | 298 | 61.7 |
| | (S)-(5-(3,5-difluorophenyl)-4,5-dihydro-1H- | | |
| | pyrazol-1-yl)(3-fluoro-3-methylazetidin-1- | | |
| | yl)methanone | | |
| 1.30 | 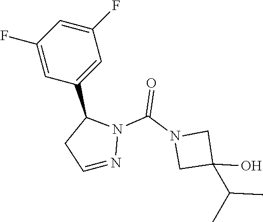 [SMILES: CC(C)C1(O)CN(C(=O)N2N=CC[C@H]2c2cc(F)cc(F)c2)C1] | 324 | 2292 |
| | (S)-(5-(3,5-difluorophenyl)-4,5-dihydro-1H- | | |
| | pyrazol-1-yl)(3-hydroxy-3-isopropylazetidin- | | |
| | 1-yl)methanone | | |
| 1.31 | 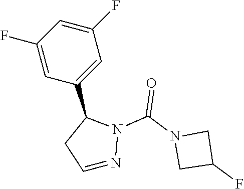 [SMILES: O=C(N1CC(F)C1)N1N=CC[C@H]1c1cc(F)cc(F)c1] | 284 | 38.4 |
| | (S)-(5-(3,5-difluorophenyl)-4,5-dihydro-1H- | | |
| | pyrazol-1-yl)(3-fluoroazetidin-1- | | |
| | yl)methanone | | |
| 1.32 | 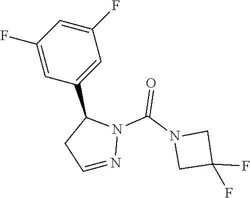 [SMILES: O=C(N1CC(F)(F)C1)N1N=CC[C@H]1c1cc(F)cc(F)c1] | 302 | 17.2 |
| | (S)-(3,3-difluoroazetidin-1-yl)(5-(3,5- | | |
| | difluorophenyl)-4,5-dihydro-1H-pyrazol-1- | | |
| | yl)methanone | | |
| 1.33 | 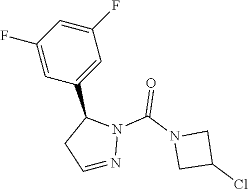 [SMILES: O=C(N1CC(Cl)C1)N1N=CC[C@H]1c1cc(F)cc(F)c1] | 300 | 18.3 |
| | (S)-(3-chloroazetidin-1-yl)(5-(3,5- | | |
| | difluorophenyl)-4,5-dihydro-1H-pyrazol-1- | | |
| | yl)methanone | | |
| 1.34 | 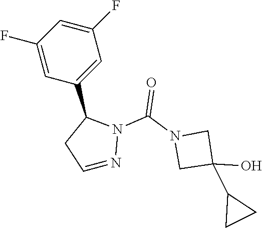 [SMILES: O=C(N1CC(O)(C2CC2)C1)N1N=CC[C@H]1c1cc(F)cc(F)c1] | 322 | 2464 |
| | (S)-(3-cyclopropyl-3-hydroxyazetidin-1- | | |
| | yl)(5-(3,5-difluorophenyl)-4,5-dihydro-1H- | | |
| | pyrazol-1-yl)methanone | | |
| 1.35 | 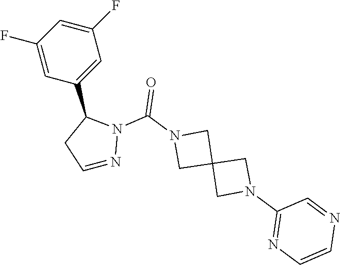 [SMILES: O=C(N1CC2(C1)CN(c1cnccn1)C2)N1N=CC[C@H]1c1cc(F)cc(F)c1] | 385 | 5149 |
| | (S)-(5-(3,5-difluorophenyl)-4,5-dihydro-1H- | | |
| | pyrazol-1-yl)(6-(pyrazin-2-yl)-2,6- | | |
| | diazaspiro[3.3]heptan-2-yl)methanone | | |
| 1.36 | 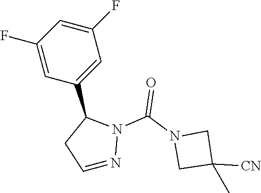 [SMILES: CC1(C#N)CN(C(=O)N2N=CC[C@H]2c2cc(F)cc(F)c2)C1] | 305 | 62.3 |
| | (S)-1-(5-(3,5-difluorophenyl)-4,5-dihydro- | | |
| | 1H-pyrazole-1-carbonyl)-3-methylazetidine- | | |
| | 3-carbonitrile | | |
| 1.37 | 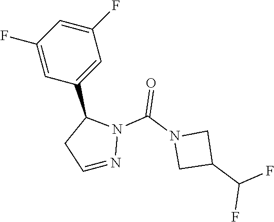 [SMILES: O=C(N1CC(C(F)F)C1)N1N=CC[C@H]1c1cc(F)cc(F)c1] | 316 | 22.6 |
| | (S)-(3-(difluoromethyl)azetidin-1-yl)(5-(3,5- | | |
| | difluorophenyl)-4,5-dihydro-1H-pyrazol-1- | | |
| | yl)methanone | | |
| 1.38 | 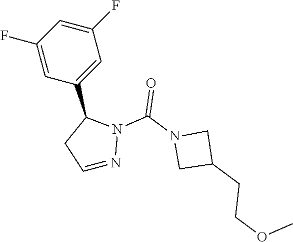 [SMILES: COCCC1CN(C(=O)N2N=CC[C@H]2c2cc(F)cc(F)c2)C1] | 324 | 105.1 |
| | (S)-(5-(3,5-difluorophenyl)-4,5-dihydro-1H- | | |
| | pyrazol-1-yl)(3-(2-methoxyethyl)azetidin-1- | | |
| | yl)methanone | | |
| 1.39 | 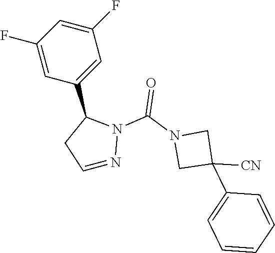 [SMILES: N#CC1(c2ccccc2)CN(C(=O)N2N=CC[C@H]2c2cc(F)cc(F)c2)C1] | 367 | 647.5 |
| | (S)-1-(5-(3,5-difluorophenyl)-4,5-dihydro- | | |
| | 1H-pyrazole-1-carbonyl)-3-phenylazetidine- | | |
| | 3-carbonitrile | | |
| 1.40 | 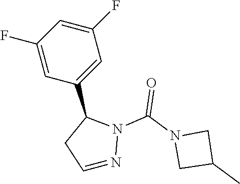 [SMILES: CC1CN(C(=O)N2N=CC[C@H]2c2cc(F)cc(F)c2)C1] | 280 | 26.0 |
| | (S)-(5-(3,5-difluorophenyl)-4,5-dihydro-1H- | | |
| | pyrazol-1-yl)(3-methylazetidin-1- | | |
| | yl)methanone | | |
| 1.41 | 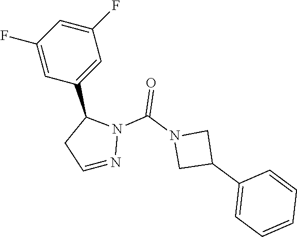 [SMILES: O=C(N1CC(c2ccccc2)C1)N1N=CC[C@H]1c1cc(F)cc(F)c1] | 342 | 6.6 |
| | (S)-(5-(3,5-difluorophenyl)-4,5-dihydro-1H- | | |
| | pyrazol-1-yl)(3-phenylazetidin-1- | | |
| | yl)methanone | | |
| 1.42 | 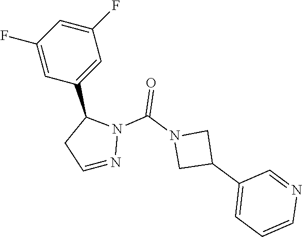 [SMILES: O=C(N1CC(c2cccnc2)C1)N1N=CC[C@H]1c1cc(F)cc(F)c1] | 343 | 28.6 |
| | (S)-(5-(3,5-difluorophenyl)-4,5-dihydro-1H- | | |
| | pyrazol-1-yl)(3-(pyridin-3-yl)azetidin-1- | | |
| | yl)methanone, TFA salt | | |
| 1.43 | 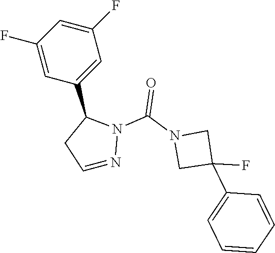 [SMILES: O=C(N1CC(F)(c2ccccc2)C1)N1N=CC[C@H]1c1cc(F)cc(F)c1] | 360 | 775.3 |
| | (S)-(5-(3,5-difluorophenyl)-4,5-dihydro-1H- | | |
| | pyrazol-1-yl)(3-fluoro-3-phenylazetidin-1- | | |
| | yl)methanone | | |
| 1.44 | 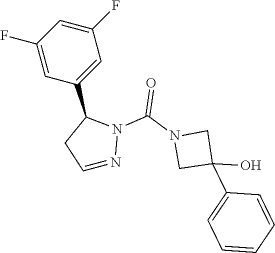 [SMILES: O=C(N1CC(O)(c2ccccc2)C1)N1N=CC[C@H]1c1cc(F)cc(F)c1] | 358 | 811.0 |
| | (S)-(5-(3,5-difluorophenyl)-4,5-dihydro-1H- | | |
| | pyrazol-1-yl)(3-hydroxy-3-phenylazetidin-1- | | |
| | yl)methanone | | |
| 1.45 | 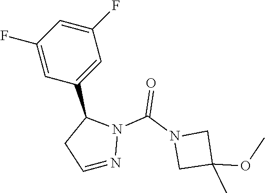 [SMILES: COC1(C)CN(C(=O)N2N=CC[C@H]2c2cc(F)cc(F)c2)C1] | 310 | 98.5 |
| | (S)-(5-(3,5-difluorophenyl)-4,5-dihydro-1H- | | |
| | pyrazol-1-yl)(3-methoxy-3-methylazetidin-1- | | |
| | yl)methanone | | |
| 1.46 | 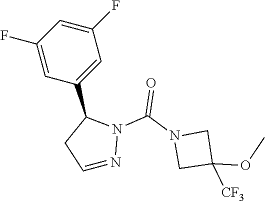 [SMILES: COC1(C(F)(F)F)CN(C(=O)N2N=CC[C@H]2c2cc(F)cc(F)c2)C1] | 364 | 232.9 |
| | (S)-(5-(3,5-difluorophenyl)-4,5-dihydro-1H- | | |
| | pyrazol-1-yl)(3-methoxy-3- | | |
| | (trifluoromethyl)azetidin-1-yl)methanone | | |
| 1.47 | 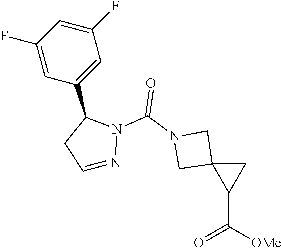 [SMILES: COC(=O)C1CC12CN(C(=O)N1N=CC[C@H]1c1cc(F)cc(F)c1)C2] | 350 | 195.4 |
| | methyl (R and S)-5-((S)-5-(3,5- | | |
| | difluorophenyl)-4,5-dihydro-1H-pyrazole-1- | | |
| | carbonyl)-5-azaspiro[2.3]hexane-1- | | |
| | carboxylate | | |
| 1.48 | 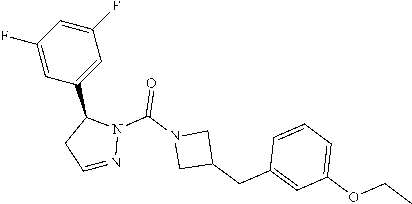 [SMILES: CCOc1cccc(CC2CN(C(=O)N3N=CC[C@H]3c3cc(F)cc(F)c3)C2)c1] | 400 | 17.1 |
| | (S)-(5-(3,5-difluorophenyl)-4,5-dihydro-1H- | | |
| | pyrazol-1-yl)(3-(3-ethoxybenzyl)azetidin-1- | | |
| | yl)methanone | | |
| 1.49 | 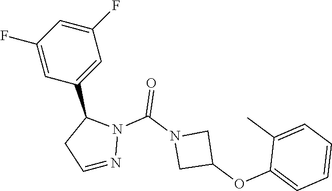 [SMILES: Cc1ccccc1OC1CN(C(=O)N2N=CC[C@H]2c2cc(F)cc(F)c2)C1] | 372 | 11.7 |
| | (S)-(5-(3,5-difluorophenyl)-4,5-dihydro-1H- | | |
| | pyrazol-1-yl)(3-(o-tolyloxy)azetidin-1- | | |
| | yl)methanone | | |
| 1.50 | 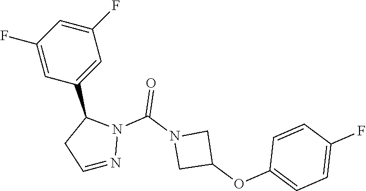 [SMILES: O=C(N1CC(Oc2ccc(F)cc2)C1)N1N=CC[C@H]1c1cc(F)cc(F)c1] | 376 | 7.0 |
| | (S)-(5-(3,5-difluorophenyl)-4,5-dihydro-1H- | | |
| | pyrazol-1-yl)(3-(4-fluorophenoxy)azetidin-1- | | |
| | yl)methanone | | |
| 1.51 | 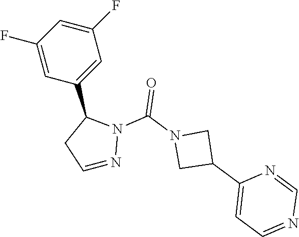 [SMILES: O=C(N1CC(c2ccncn2)C1)N1N=CC[C@H]1c1cc(F)cc(F)c1] | 344 | 1114 |
| | (S)-(5-(3,5-difluorophenyl)-4,5-dihydro-1H- | | |
| | pyrazol-1-yl)(3-(pyrimidin-4-yl)azetidin-1- | | |
| | yl)methanone | | |
| 1.52 | 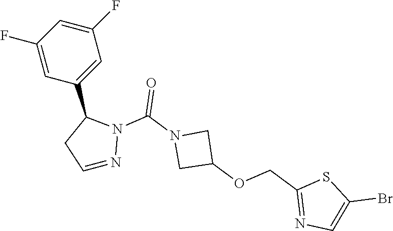 [SMILES: O=C(N1CC(OCc2ncc(Br)s2)C1)N1N=CC[C@H]1c1cc(F)cc(F)c1] | 457, 459 | 23.9 |
| | (S)-(3-((5-bromothiazol-2- | | |
| | yl)methoxy)azetidin-1-yl)(5-(3,5- | | |
| | difluorophenyl)-4,5-dihydro-1H-pyrazol-1- | | |
| | yl)methanone | | |
| 1.53 | 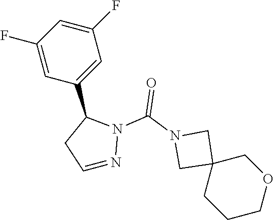 [SMILES: O=C(N1CC2(CCCOC2)C1)N1N=CC[C@H]1c1cc(F)cc(F)c1] | 336 | 530.2 |
| | (S)-(5-(3,5-difluorophenyl)-4,5-dihydro-1H- | | |
| | pyrazol-1-yl)(6-oxa-2-azaspiro[3.5]nonan-2- | | |
| | yl)methanone | | |
| 1.54 | 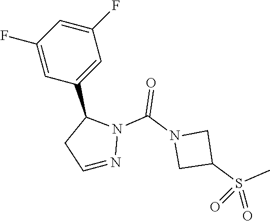 [SMILES: CS(=O)(=O)C1CN(C(=O)N2N=CC[C@H]2c2cc(F)cc(F)c2)C1] | 344 | 4232 |
| | (S)-(5-(3,5-difluorophenyl)-4,5-dihydro-1H- | | |
| | pyrazol-1-yl)(3-(methylsulfonyl)azetidin-1- | | |
| | yl)methanone | | |
| 1.55 | 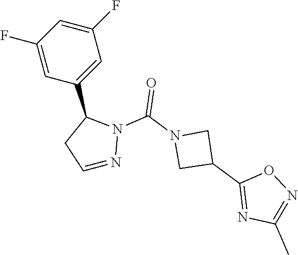 [SMILES: Cc1noc(C2CN(C(=O)N3N=CC[C@H]3c3cc(F)cc(F)c3)C2)n1] | 348 | 174.7 |
| | (S)-(5-(3,5-difluorophenyl)-4,5-dihydro-1H- | | |
| | pyrazol-1-yl)(3-(3-methyl-1,2,4-oxadiazol-5- | | |
| | yl)azetidin-1-yl)methanone | | |
| 1.56 | 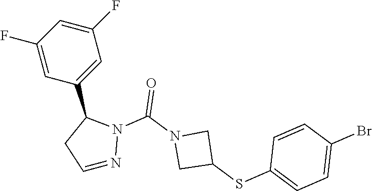 [SMILES: O=C(N1CC(Sc2ccc(Br)cc2)C1)N1N=CC[C@H]1c1cc(F)cc(F)c1] | 452, 454 | 5.9 |
| | (S)-(3-((4-bromophenyl)thio)azetidin-1- | | |
| | yl)(5-(3,5-difluorophenyl)-4,5-dihydro-1H- | | |
| | pyrazol-1-yl)methanone | | |
| 1.57 | 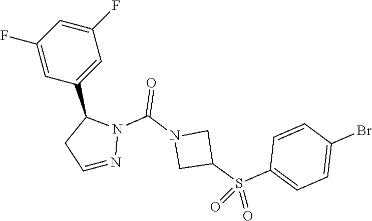 [SMILES: O=C(N1CC(S(=O)(=O)c2ccc(Br)cc2)C1)N1N=CC[C@H]1c1cc(F)cc(F)c1] | 484, 486 | 12.4 |
| | (S)-(3-((4-bromophenyl)sulfonyl)azetidin-1- | | |
| | yl)(5-(3,5-difluorophenyl)-4,5-dihydro-1H- | | |
| | pyrazol-1-yl)methanone | | |
| 1.58 | 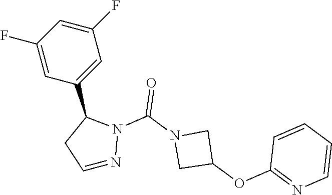 [SMILES: O=C(N1CC(Oc2ccccn2)C1)N1N=CC[C@H]1c1cc(F)cc(F)c1] | 359 | 1379 |
| | (S)-(5-(3,5-difluorophenyl)-4,5-dihydro-1H- | | |
| | pyrazol-1-yl)(3-(pyridin-2-yloxy)azetidin-1- | | |
| | yl)methanone | | |
| 1.59 | 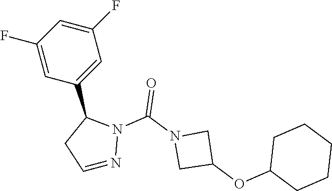 [SMILES: O=C(N1CC(OC2CCCCC2)C1)N1N=CC[C@H]1c1cc(F)cc(F)c1] | 364 | 244.9 |
| | (S)-(3-(cyclohexyloxy)azetidin-1-yl)(5-(3,5- | | |
| | difluorophenyl)-4,5-dihydro-1H-pyrazol-1- | | |
| | yl)methanone | | |
| 1.60 | 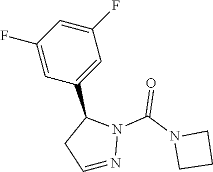 [SMILES: O=C(N1CCC1)N1N=CC[C@H]1c1cc(F)cc(F)c1] | 266 | 64.5 |
| | (S)-azetidin-1-yl(5-(3,5-difluorophenyl)-4,5- | | |
| | dihydro-1H-pyrazol-1-yl)methanone | | |
| 1.61 | 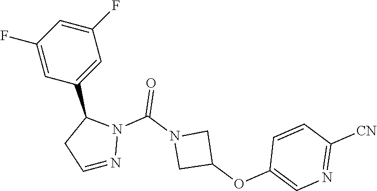 [SMILES: N#Cc1ccc(OC2CN(C(=O)N3N=CC[C@H]3c3cc(F)cc(F)c3)C2)cn1] | 384 | 26.3 |
| | (S)-5-((1-(5-(3,5-difluorophenyl)-4,5- | | |
| | dihydro-1H-pyrazole-1-carbonyl)azetidin-3- | | |
| | yl)oxy)picolinonitrile | | |

### Example 1.6

(R and S)-6-((1-(5-(3,5-difluorophenyl)-4,5-dihydro-1H-pyrazole-1-carbonyl)azetidin-3-yl)oxy)pyrimidine-4-carbonitrile, TFA salt was purified by CHIRAL-Prep SFC [Column: AS-H, 21×250 mm: 20% [MeOH w/0.1% NaOH]/CO₂; Flow rate: 70 mL/min; 220 nm; Second Eluting Peak (1.6); the first eluting peak was the enantiomer of 1.6].

### Example 1.12/1.13

((S)-5-(3,5-difluorophenyl)-4,5-dihydro-1H-pyrazol-1-yl)((2R,3R and 2S,3S)-2-methyl-3-phenoxyazetidin-1-yl)methanone, TFA salt was purified by CHIRAL-Prep SFC [Column: Lux-2, 21×250 mm; 20% [MeOH w/0.1% NaOH]/CO₂; Flow rate: 70 mL/min; 220 nm; First Eluting Peak (1.12); Second Eluting Peak (1.13)].

### Preparation of Example 2.1, (S)-(5-(3,5-difluorophenyl)-4,5-dihydro-1H-pyrazol-1-yl)(3-methoxyazetidin-1-yl)methanone

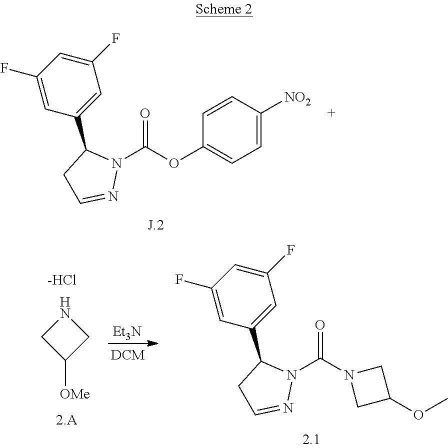

A stirring solution of (S)-4-nitrophenyl 5-(3,5-difluorophenyl)-4,5-dihydro-1H-pyrazole-1-carboxylate (60 mg, 0.173 mmol) in DCM (1 mL) was treated with 3-methoxyazetidine hydrochloride (57 mg, 0.464 mmol) and triethylamine (96 μL, 0.691 mmol). The resulting mixture was stirred at 30° C. for 2 h, then directly concentrated and purified via reverse-phase HPLC [Method A]. This provided (S)-(5-(3,5-difluorophenyl)-4,5-dihydro-1H-pyrazol-1-yl)(3-methoxyazetidin-1-yl)methanone. MS (ESI) m/z calculated for C₁₄H₁₆F₂N₃O₂ [M+H]⁺ 296, found 296. ¹H NMR (500 MHz, CDCl₃) δ 6.71-6.80 (m, 3H), 6.67 (tt, J=9.0, 2.5 Hz, 1H), 5.26 (dd, J=12.0, 6.5 Hz, 1H), 4.24-4.39 (m, 2H), 4.15-4.21 (m, 1H), 3.95-4.11 (m, 2H), 3.31-3.37 (m, 1H), 3.29 (s, 3H), 2.67 (ddd, J=18.5, 6.5, 1.5 Hz, 1H). RIPK1 EC₅₀ 11.0 nM.

The following examples in Table 13 were prepared according to Scheme 2 and General Scheme 1 above, using intermediates M.2, K.3, K.2, K.5, K.4, K.6, M.3, M.4, K.8, K.7, or K.9, and the appropriate azetidine or azetidine salt coupling partner, either commercially available or intermediates AD.2, AD.1, AD.3, AD.4, AD.8, AD.5, AD.7, AD.6, AD.9, AD.10, AD.11, AD.12, AD.13, or AD.14. For Example 2.18 through Example 2.47, a slightly modified procedure was used wherein Et₃N was replaced by DIPEA. The compounds were generally purified by reversed phase HPLC and SFC. Where isomers were separated by SFC conditions are provided, following the table.

TABLE 13
Examples Prepared According to General Scheme 1 and Scheme 2

| | Structure | Observed m/z | RIPK1 EC₅₀ |
|---|---|---|---|
| Example | Name | [M + H]⁺ | (nM) |
| 2.2 | 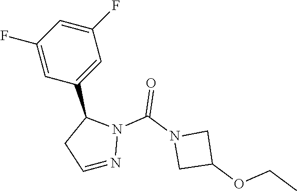 [SMILES: CCOC1CN(C(=O)N2N=CC[C@H]2c2cc(F)cc(F)c2)C1] | 310 | 27.2 |
| | (S)-(5-(3,5-difluorophenyl)-4,5-dihydro-1H- | | |
| | pyrazol-1-yl)(3-ethoxyazetidin-1- | | |
| | yl)methanone | | |
| 2.3 | 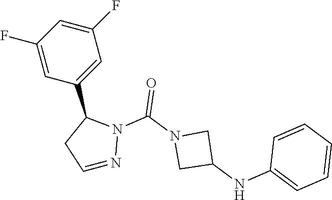 [SMILES: O=C(N1CC(Nc2ccccc2)C1)N1N=CC[C@H]1c1cc(F)cc(F)c1] | 357 | 6.7 |
| | (S)-(5-(3,5-difluorophenyl)-4,5-dihydro-1H- | | |
| | pyrazol-1-yl)(3-(phenylamino)azetidin-1- | | |
| | yl)methanone | | |
| 2.4 | 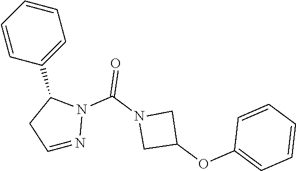 [SMILES: O=C(N1CC(Oc2ccccc2)C1)N1N=CC[C@@H]1c1ccccc1] | 322 | 5001 |
| | (R)-(3-phenoxyazetidin-1-yl)(5-phenyl-4,5- | | |
| | dihydro-1H-pyrazol-1-yl)methanone | | |
| 2.5 | 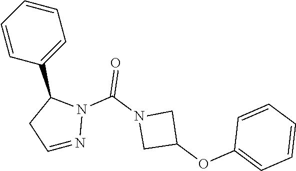 [SMILES: O=C(N1CC(Oc2ccccc2)C1)N1N=CC[C@H]1c1ccccc1] | 322 | 0.5 |
| | (S)-(3-phenoxyazetidin-1-yl)(5-phenyl-4,5- | | |
| | dihydro-1H-pyrazol-1-yl)methanone | | |
| 2.6 | 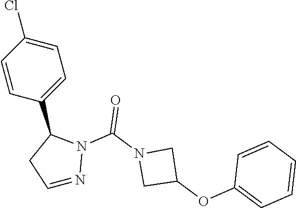 [SMILES: O=C(N1CC(Oc2ccccc2)C1)N1N=CC[C@H]1c1ccc(Cl)cc1] | 356 | 9.6 |
| | (S)-(5-(4-chlorophenyl)-4,5-dihydro-1H- | | |
| | pyrazol-1-yl)(3-phenoxyazetidin-1- | | |
| | yl)methanone | | |
| 2.7 | 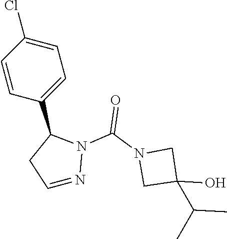 [SMILES: CC(C)C1(O)CN(C(=O)N2N=CC[C@H]2c2ccc(Cl)cc2)C1] | 322 | 5163 |
| | (S)-(5-(4-chlorophenyl)-4,5-dihydro-1H- | | |
| | pyrazol-1-yl)(3-hydroxy-3-isopropylazetidin- | | |
| | 1-yl)methanone | | |
| 2.8 | 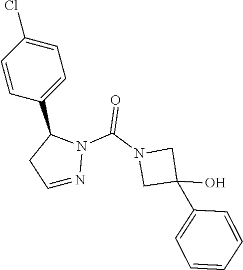 [SMILES: O=C(N1CC(O)(c2ccccc2)C1)N1N=CC[C@H]1c1ccc(Cl)cc1] | 356 | 2428 |
| | (S)-(5-(4-chlorophenyl)-4,5-dihydro-1H- | | |
| | pyrazol-1-yl)(3-hydroxy-3-phenylazetidin-1- | | |
| | yl)methanone | | |
| 2.9 | 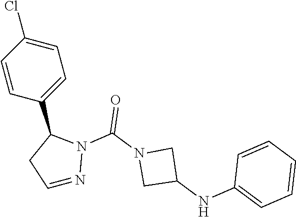 [SMILES: O=C(N1CC(Nc2ccccc2)C1)N1N=CC[C@H]1c1ccc(Cl)cc1] | 355 | 22.7 |
| | (S)-(5-(4-chlorophenyl)-4,5-dihydro-1H- | | |
| | pyrazol-1-yl)(3-(phenylamino)azetidin-1- | | |
| | yl)methanone | | |
| 2.10 | 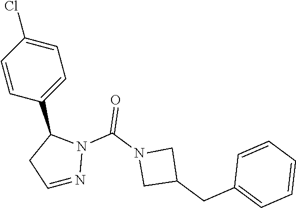 [SMILES: O=C(N1CC(Cc2ccccc2)C1)N1N=CC[C@H]1c1ccc(Cl)cc1] | 354 | 11.4 |
| | (S)-(3-benzylazetidin-1-yl)(5-(4- | | |
| | chlorophenyl)-4,5-dihydro-1H-pyrazol-1- | | |
| | yl)methanone | | |
| 2.11 | 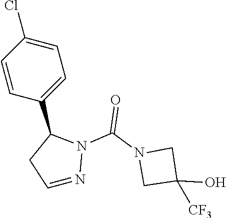 [SMILES: O=C(N1CC(O)(C(F)(F)F)C1)N1N=CC[C@H]1c1ccc(Cl)cc1] | 348 | 578.0 |
| | (S)-(5-(4-chlorophenyl)-4,5-dihydro-1H- | | |
| | pyrazol-1-yl)(3-hydroxy-3- | | |
| | (trifluoromethyl)azetidin-1-yl)methanone | | |
| 2.12 | 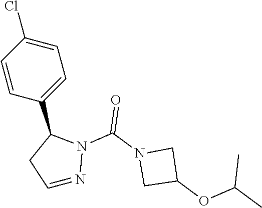 [SMILES: CC(C)OC1CN(C(=O)N2N=CC[C@H]2c2ccc(Cl)cc2)C1] | 322 | 10.6 |
| | (S)-(5-(4-chlorophenyl)-4,5-dihydro-1H- | | |
| | pyrazol-1-yl)(3-isopropoxyazetidin-1- | | |
| | yl)methanone | | |
| 2.13 | 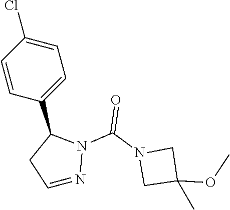 [SMILES: COC1(C)CN(C(=O)N2N=CC[C@H]2c2ccc(Cl)cc2)C1] | 308 | 64.9 |
| | (S)-(5-(4-chlorophenyl)-4,5-dihydro-1H- | | |
| | pyrazol-1-yl)(3-methoxy-3-methylazetidin-1- | | |
| | yl)methanone | | |
| 2.14 | 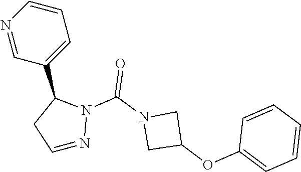 [SMILES: O=C(N1CC(Oc2ccccc2)C1)N1N=CC[C@H]1c1cccnc1] | 323 | 20.5 |
| | (S)-(3-phenoxyazetidin-1-yl)(5-(pyridin-3- | | |
| | yl)-4,5-dihydro-1H-pyrazol-1-yl)methanone | | |
| 2.15 | 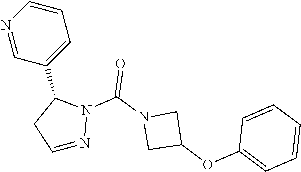 [SMILES: O=C(N1CC(Oc2ccccc2)C1)N1N=CC[C@@H]1c1cccnc1] | 323 | 95.6 |
| | (R)-(3-phenoxyazetidin-1-yl)(5-(pyridin-3- | | |
| | yl)-4,5-dihydro-1H-pyrazol-1-yl)methanone | | |
| 2.16 | 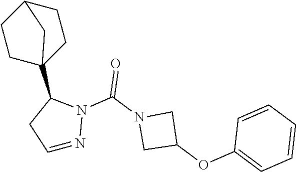 [SMILES: O=C(N1CC(Oc2ccccc2)C1)N1N=CC[C@H]1C12CCC(CC1)C2] | 340 | 92.7 |
| | ((5S)-5-(bicyclo[2.2.1]heptan-1-yl)-4,5- | | |
| | dihydro-1H-pyrazol-1-yl)(3- | | |
| | phenoxyazetidin-1-yl)methanone | | |
| 2.17 | 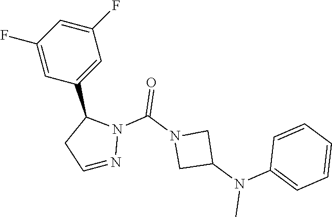 [SMILES: CN(c1ccccc1)C1CN(C(=O)N2N=CC[C@H]2c2cc(F)cc(F)c2)C1] | 371 | 7.1 |
| | (S)-(5-(3,5-difluorophenyl)-4,5-dihydro-1H- | | |
| | pyrazol-1-yl)(3- | | |
| | (methyl(phenyl)amino)azetidin-1- | | |
| | yl)methanone, TFA salt | | |
| 2.18 | 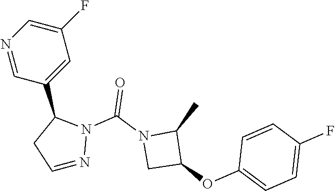 [SMILES: C[C@H]1[C@@H](Oc2ccc(F)cc2)CN1C(=O)N1N=CC[C@H]1c1cncc(F)c1] | 373 | 10.0 |
| | ((2S,3S)-3-(4-fluorophenoxy)-2- | | |
| | methylazetidin-1-yl)((S)-5-(5-fluoropyridin- | | |
| | 3-yl)-4,5-dihydro-1H-pyrazol-1- | | |
| | yl)methanone | | |
| 2.19 | 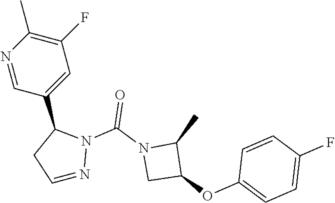 [SMILES: Cc1ncc([C@@H]2CC=NN2C(=O)N2C[C@H](Oc3ccc(F)cc3)[C@@H]2C)cc1F] | 387 | 12.3 |
| | ((S)-5-(5-fluoro-6-methylpyridin-3-yl)-4,5- | | |
| | dihydro-1H-pyrazol-1-yl)((2S,3S)-3-(4- | | |
| | fluorophenoxy)-2-methylazetidin-1- | | |
| | yl)methanone | | |
| 2.20 | 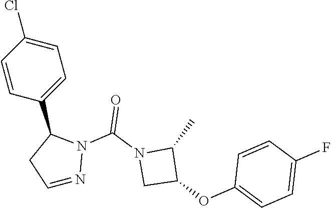 [SMILES: C[C@@H]1[C@H](Oc2ccc(F)cc2)CN1C(=O)N1N=CC[C@H]1c1ccc(Cl)cc1] | 388 | 25.7 |
| | ((S)-5-(4-chlorophenyl)-4,5-dihydro-1H- | | |
| | pyrazol-1-yl)((2R,3R)-3-(4-fluorophenoxy)- | | |
| | 2-methylazetidin-1-yl)methanone | | |
| 2.21 | 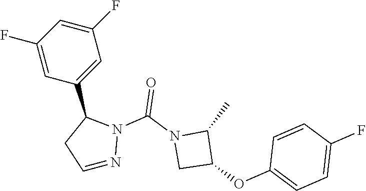 [SMILES: C[C@@H]1[C@H](Oc2ccc(F)cc2)CN1C(=O)N1N=CC[C@H]1c1cc(F)cc(F)c1] | 390 | 39.1 |
| | ((S)-5-(3,5-difluorophenyl)-4,5-dihydro-1H- | | |
| | pyrazol-1-yl)((2R,3R)-3-(4-fluorophenoxy)- | | |
| | 2-methylazetidin-1-yl)methanone | | |
| 2.22 | 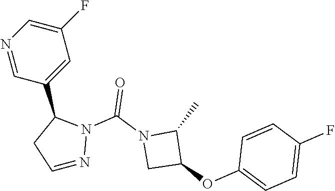 [SMILES: C[C@@H]1[C@@H](Oc2ccc(F)cc2)CN1C(=O)N1N=CC[C@H]1c1cncc(F)c1] | 373 | 7.6 |
| | ((2R,3S)-3-(4-fluorophenoxy)-2- | | |
| | methylazetidin-1-yl)((S)-5-(5-fluoropyridin- | | |
| | 3-yl)-4,5-dihydro-1H-pyrazol-1- | | |
| | yl)methanone | | |
| 2.23 | 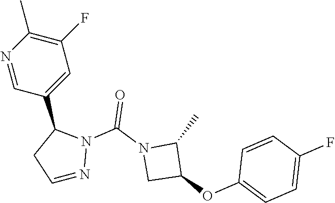 [SMILES: Cc1ncc([C@@H]2CC=NN2C(=O)N2C[C@H](Oc3ccc(F)cc3)[C@H]2C)cc1F] | 387 | 10.1 |
| | ((S)-5-(5-fluoro-6-methylpyridin-3-yl)-4,5- | | |
| | dihydro-1H-pyrazol-1-yl)((2R,3S)-3-(4- | | |
| | fluorophenoxy)-2-methylazetidin-1- | | |
| | yl)methanone | | |
| 2.24 | 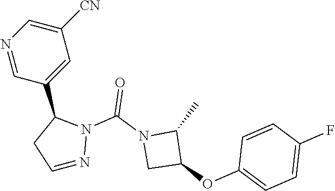 [SMILES: C[C@@H]1[C@@H](Oc2ccc(F)cc2)CN1C(=O)N1N=CC[C@H]1c1cncc(C#N)c1] | 380 | 11.4 |
| | 5-((S)-1-((2R,3S)-3-(4-fluorophenoxy)-2- | | |
| | methylazetidine-1-carbonyl)-4,5-dihydro- | | |
| | 1H-pyrazol-5-yl)nicotinonitrile | | |
| 2.25 | 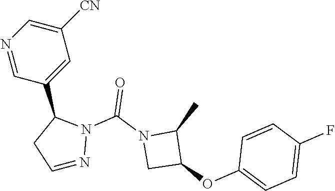 [SMILES: C[C@H]1[C@@H](Oc2ccc(F)cc2)CN1C(=O)N1N=CC[C@H]1c1cncc(C#N)c1] | 380 | 12.8 |
| | 5-((S)-1-((2S,3S)-3-(4-fluorophenoxy)-2- | | |
| | methylazetidine-1-carbonyl)-4,5-dihydro- | | |
| | 1H-pyrazol-5-yl)nicotinonitrile | | |
| 2.26 | 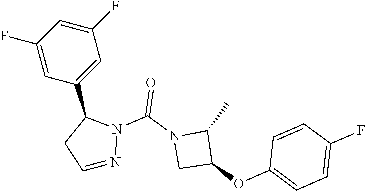 [SMILES: C[C@@H]1[C@@H](Oc2ccc(F)cc2)CN1C(=O)N1N=CC[C@H]1c1cc(F)cc(F)c1] | 390 | 7.8 |
| | ((S)-5-(3,5-difluorophenyl)-4,5-dihydro-1H- | | |
| | pyrazol-1-yl)((2R,3S)-3-(4-fluorophenoxy)- | | |
| | 2-methylazetidin-1-yl)methanone | | |
| 2.27 | 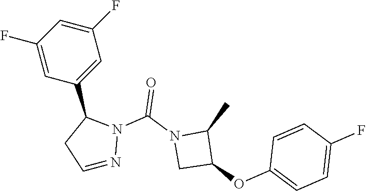 [SMILES: C[C@H]1[C@@H](Oc2ccc(F)cc2)CN1C(=O)N1N=CC[C@H]1c1cc(F)cc(F)c1] | 390 | 6.8 |
| | ((S)-5-(3,5-difluorophenyl)-4,5-dihydro-1H- | | |
| | pyrazol-1-yl)((2S,3S)-3-(4-fluorophenoxy)-2- | | |
| | methylazetidin-1-yl)methanone | | |
| 2.28 | 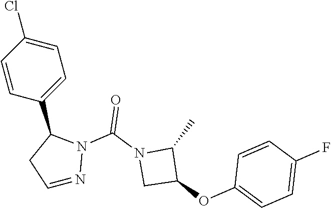 [SMILES: C[C@@H]1[C@@H](Oc2ccc(F)cc2)CN1C(=O)N1N=CC[C@H]1c1ccc(Cl)cc1] | 388 | 5.9 |
| | ((S)-5-(4-chlorophenyl)-4,5-dihydro-1H- | | |
| | pyrazol-1-yl)((2R,3S)-3-(4-fluorophenoxy)- | | |
| | 2-methylazetidin-1-yl)methanone | | |
| 2.29 | 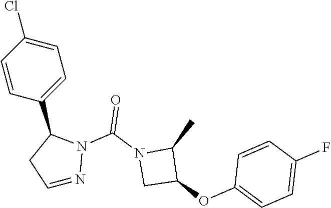 [SMILES: C[C@H]1[C@@H](Oc2ccc(F)cc2)CN1C(=O)N1N=CC[C@H]1c1ccc(Cl)cc1] | 388 | <5.1 |
| | ((S)-5-(4-chlorophenyl)-4,5-dihydro-1H- | | |
| | pyrazol-1-yl)((2S,3S)-3-(4-fluorophenoxy)-2- | | |
| | methylazetidin-1-yl)methanone | | |
| 2.30 | 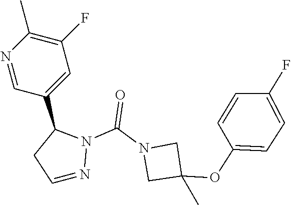 [SMILES: Cc1ncc([C@@H]2CC=NN2C(=O)N2CC(C)(Oc3ccc(F)cc3)C2)cc1F] | 387 | 14.2 |
| | (S)-(5-(5-fluoro-6-methylpyridin-3-yl)-4,5- | | |
| | dihydro-1H-pyrazol-1-yl)(3-(4- | | |
| | fluorophenoxy)-3-methylazetidin-1- | | |
| | yl)methanone | | |
| 2.31 | 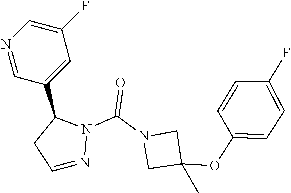 [SMILES: CC1(Oc2ccc(F)cc2)CN(C(=O)N2N=CC[C@H]2c2cncc(F)c2)C1] | 373 | 17.1 |
| | (S)-(3-(4-fluorophenoxy)-3-methylazetidin- | | |
| | 1-yl)(5-(5-fluoropyridin-3-yl)-4,5-dihydro- | | |
| | 1H-pyrazol-1-yl)methanone | | |
| 2.32 | 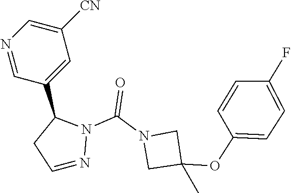 [SMILES: CC1(Oc2ccc(F)cc2)CN(C(=O)N2N=CC[C@H]2c2cncc(C#N)c2)C1] | 380 | 25.5 |
| | (S)-5-(1-(3-(4-fluorophenoxy)-3- | | |
| | methylazetidine-1-carbonyl)-4,5-dihydro- | | |
| | 1H-pyrazol-5-yl)nicotinonitrile | | |
| 2.33 | 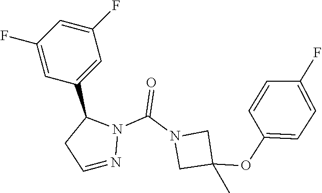 [SMILES: CC1(Oc2ccc(F)cc2)CN(C(=O)N2N=CC[C@H]2c2cc(F)cc(F)c2)C1] | 390 | 19.8 |
| | (S)-(5-(3,5-difluorophenyl)-4,5-dihydro-1H- | | |
| | pyrazol-1-yl)(3-(4-fluorophenoxy)-3- | | |
| | methylazetidin-1-yl)methanone | | |
| 2.34 | 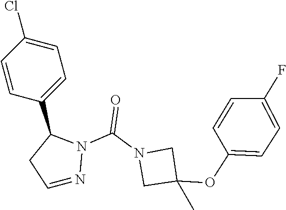 [SMILES: CC1(Oc2ccc(F)cc2)CN(C(=O)N2N=CC[C@H]2c2ccc(Cl)cc2)C1] | 388 | 12.7 |
| | (S)-(5-(4-chlorophenyl)-4,5-dihydro-1H- | | |
| | pyrazol-1-yl)(3-(4-fluorophenoxy)-3- | | |
| | methylazetidin-1-yl)methanone | | |
| 2.35 | 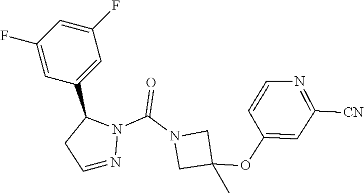 [SMILES: CC1(Oc2ccnc(C#N)c2)CN(C(=O)N2N=CC[C@H]2c2cc(F)cc(F)c2)C1] | 398 | 24.2 |
| | (S)-4-((1-(5-(3,5-difluorophenyl)-4,5- | | |
| | dihydro-1H-pyrazole-1-carbonyl)-3- | | |
| | methylazetidin-3-yl)oxy)picolinonitrile | | |
| 2.36 | 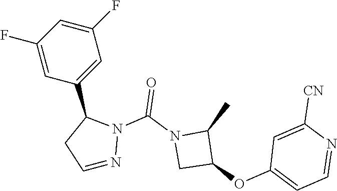 [SMILES: C[C@H]1[C@@H](Oc2ccnc(C#N)c2)CN1C(=O)N1N=CC[C@H]1c1cc(F)cc(F)c1] | 398 | 20.5 |
| | 4-(((25,3S)-1-((S)-5-(3,5-difluorophenyl)- | | |
| | 4,5-dihydro-1H-pyrazole-1-carbonyl)-2- | | |
| | methylazetidin-3-yl)oxy)picolinonitrile | | |
| 2.37 | 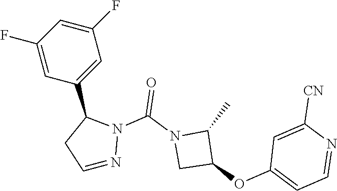 [SMILES: C[C@@H]1[C@@H](Oc2ccnc(C#N)c2)CN1C(=O)N1N=CC[C@H]1c1cc(F)cc(F)c1] | 398 | 6.6 |
| | 4-(((2R,3S)-1-((S)-5-(3,5-difluorophenyl)- | | |
| | 4,5-dihydro-1H-pyrazole-1-carbonyl)-2- | | |
| | methylazetidin-3-yl)oxy)picolinonitrile | | |
| 2.38 | 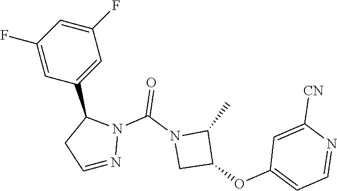 [SMILES: C[C@@H]1[C@H](Oc2ccnc(C#N)c2)CN1C(=O)N1N=CC[C@H]1c1cc(F)cc(F)c1] | 398 | 85.9 |
| | 4-(((2R,3R)-1-((S)-5-(3,5-difluorophenyl)- | | |
| | 4,5-dihydro-1H-pyrazole-1-carbonyl)-2- | | |
| | methylazetidin-3-yl)oxy)picolinonitrile | | |
| 2.39 | 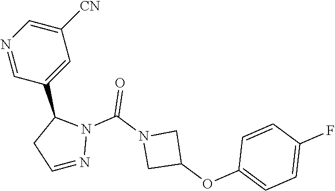 [SMILES: N#Cc1cncc([C@@H]2CC=NN2C(=O)N2CC(Oc3ccc(F)cc3)C2)c1] | 366 | 33.1 |
| | (S)-5-(1-(3-(4-fluorophenoxy)azetidine-1- | | |
| | carbonyl)-4,5-dihydro-1H-pyrazol-5- | | |
| | yl)nicotinonitrile | | |
| 2.40 | 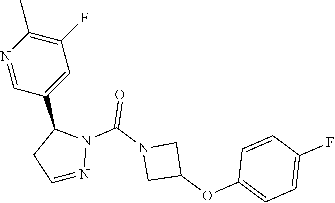 [SMILES: Cc1ncc([C@@H]2CC=NN2C(=O)N2CC(Oc3ccc(F)cc3)C2)cc1F] | 373 | 14.7 |
| | (S)-(5-(5-fluoro-6-methylpyridin-3-yl)-4,5- | | |
| | dihydro-1H-pyrazol-1-yl)(3-(4- | | |
| | fluorophenoxy)azetidin-1-yl)methanone | | |
| 2.41 | 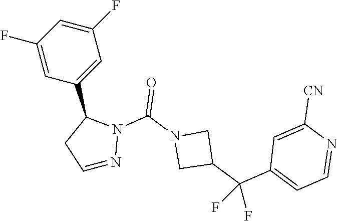 [SMILES: N#Cc1cc(C(F)(F)C2CN(C(=O)N3N=CC[C@H]3c3cc(F)cc(F)c3)C2)ccn1] | 418 | 22.9 |
| | (S)-4-((1-(5-(3,5-difluorophenyl)-4,5- | | |
| | dihydro-1H-pyrazole-1-carbonyl)azetidin-3- | | |
| | yl)difluoromethyl)picolinonitrile | | |
| 2.42 | 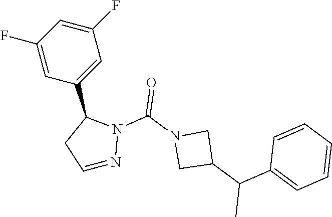 [SMILES: CC(c1ccccc1)C1CN(C(=O)N2N=CC[C@H]2c2cc(F)cc(F)c2)C1] | 370 | 18.6 |
| | ((S)-5-(3,5-difluorophenyl)-4,5-dihydro-1H- | | |
| | pyrazol-1-yl)(3-((R or S)-1- | | |
| | phenylethyl)azetidin-1-yl)methanone | | |
| 2.43 | 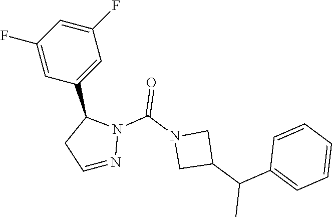 [SMILES: CC(c1ccccc1)C1CN(C(=O)N2N=CC[C@H]2c2cc(F)cc(F)c2)C1] | 370 | 46.7 |
| | ((S)-5-(3,5-difluorophenyl)-4,5-dihydro-1H- | | |
| | pyrazol-1-yl)(3-((S or R)-1- | | |
| | phenylethyl)azetidin-1-yl)methanone | | |
| 2.44 | 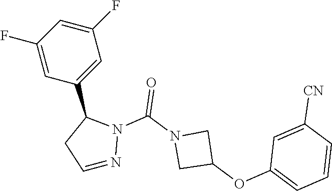 [SMILES: N#Cc1cccc(OC2CN(C(=O)N3N=CC[C@H]3c3cc(F)cc(F)c3)C2)c1] | 383 | 11.9 |
| | (S)-3-((1-(5-(3,5-difluorophenyl)-4,5- | | |
| | dihydro-1H-pyrazole-1-carbonyl)azetidin-3- | | |
| | yl)oxy)benzonitrile | | |
| 2.45 | 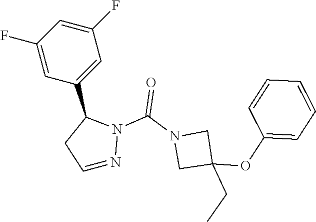 [SMILES: CCC1(Oc2ccccc2)CN(C(=O)N2N=CC[C@H]2c2cc(F)cc(F)c2)C1] | 386 | 57.4 |
| | (S)-(5-(3,5-difluorophenyl)-4,5-dihydro-1H- | | |
| | pyrazol-1-yl)(3-ethyl-3-phenoxyazetidin-1- | | |
| | yl)methanone | | |
| 2.46 | 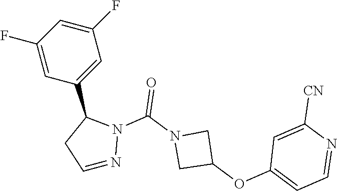 [SMILES: N#Cc1cc(OC2CN(C(=O)N3N=CC[C@H]3c3cc(F)cc(F)c3)C2)ccn1] | 382 | 46.3 |
| | (S)-4-((1-(5-(3,5-difluorophenyl)-4,5- | | |
| | dihydro-1H-pyrazole-1-carbonyl)azetidin-3- | | |
| | yl)methyl)picolinonitrile | | |
| 2.47 | 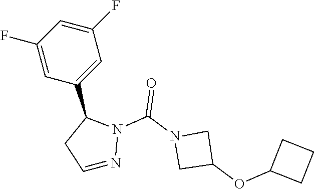 [SMILES: O=C(N1CC(OC2CCC2)C1)N1N=CC[C@H]1c1cc(F)cc(F)c1] | 336 | 92.7 |
| | (S)-(3-cyclobutoxyazetidin-1-yl)(5-(3,5- | | |
| | difluorophenyl)-4,5-dihydro-1H-pyrazol-1- | | |
| | yl)methanone | | |

### Example 2.4/2.5

(3-phenoxyazetidin-1-yl)(5-phenyl-4,5-dihydro-1H-pyrazol-1-yl)methanone was purified by CHIRAL-Prep SFC [Column: DAICEL CHIRALPAK AD, 250×30 mm: 40% [0.1% NH₄OH in i-PrOH]/CO₂; Flow rate: 70 mL/min; First Eluting Peak (2.4); Second Eluting Peak (2.5)].

### Example 2.31

(R and S)-(5-(5-fluoro-6-methylpyridin-3-yl)-4,5-dihydro-1H-pyrazol-1-yl)(3-(4-fluorophenoxy)azetidin-1-yl)methanone, TFA salt was purified by CHIRAL-Prep SFC [Column: Daicel Chiralpak AD, 250×30 mm: 55% [EtOH w/0.1% NH₄OH]/CO₂; Flow rate: 80 mL/min; Second Eluting Peak (2.31); the first eluting peak was the enantiomer of 2.31].

### Example 2.40

(R and S)-(3-(4-fluorophenoxy)-3-methylazetidin-1-yl)(5-(5-fluoropyridin-3-yl)-4,5-dihydro-1H-pyrazol-1-yl)methanone, TFA salt was purified by CHIRAL-Prep SFC [Column: Daicel Chiralpak AD, 250×30 mm: 40% [i-PrOH w/0.1% NH₄OH]/CO₂; Flow rate: 70 mL/min; Second Eluting Peak (2.40); the first eluting peak was the enantiomer of 2.40].

### Example 2.42/2.43

((S)-5-(3,5-difluorophenyl)-4,5-dihydro-1H-pyrazol-1-yl)(3-((R and S)-1-phenylethyl)azetidin-1-yl)methanone was purified by CHIRAL-Prep SFC [Column: DAICEL CHIRALPAK AD, 250×30 mm: 35% [0.1% NH₄OH in EtOH]/CO₂; Flow rate: 70 mL/min; First Eluting Peak (2.42); Second Eluting Peak (2.43)]

### Preparation of Example 3.1, (S)-4-((1-(5-(3,5-difluorophenyl)-4,5-dihydro-1H-pyrazole-1-carbonyl)azetidin-3-yl)oxy)picolinonitrile, TFA Salt

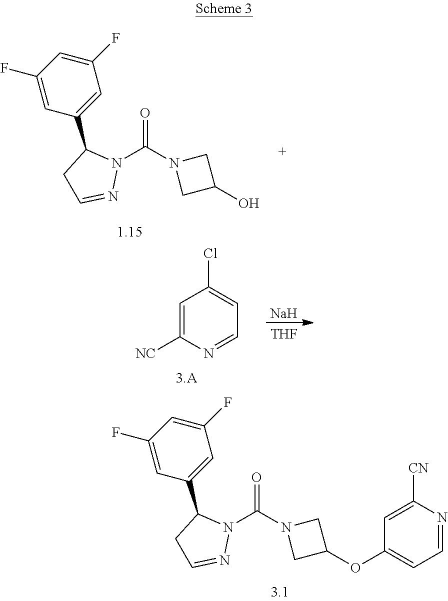

A stirring solution of (S)-(5-(3,5-difluorophenyl)-4,5-dihydro-1H-pyrazol-1-yl)(3-hydroxyazetidin-1-yl)methanone (28.1 mg, 0.100 mmol) in THF (0.75 mL) was cooled to 0° C. and treated with NaH (60% in mineral oil, 6.0 mg, 0.150 mmol). The resulting mixture was stirred at 0° C. for 20 min, and then it was transferred to a 5 mL Biotage® microwave vial containing 4-chloropicolinonitrile (22.7 mg, 0.164 mmol) and a stir bar. The reaction mixture was then stirred at 25° C. for 2.5 h, then quenched with sat. aq. NH₄Cl (2 mL) and DCM (2 mL). The layers were separated, and the aq. layer was extracted with DCM (2×3 mL). The combined organic layers were concentrated, and the crude residue was taken up in DMSO (2 mL), filtered and purified via reversed phase HPLC [Method A] This provided (S)-4-((1-(5-(3,5-difluorophenyl)-4,5-dihydro-1H-pyrazole-1-carbonyl)azetidin-3-yl)oxy)picolinonitrile, TFA salt. MS (ESI) m/z calculated for C₁₉H₁₆F₂N₅O₂ [M+H]⁺ 384, found 384. ¹H NMR (500 MHz, DMSO-d₆) δ 8.58-8.53 (m, 1H), 7.64 (s, 1H), 7.26-7.20 (m, 1H), 7.15-7.05 (m, 1H), 7.02 (s, 1 H), 6.94-6.86 (m, 2H), 5.27-5.19 (m, 1H), 5.17 (br s, 1H), 4.53 (br s, 2H), 3.98 (br s, 2H), 3.46-3.36 (m, 1H), 2.68-2.50 (in, 1H). RIPK1 EC₅₀ 6.6 nM.

The following examples in Table 14 were prepared according to Scheme 3 and General Scheme 2 above, using Example 1.15 and the appropriate commercial heteroaryl halide coupling partner.

TABLE 14
Examples Prepared According to General Scheme 2 and Scheme 3

| | Structure | Observed m/z | RIPK1 EC₅₀ |
|---|---|---|---|
| Example | Name | [M + H]⁺ | (nM) |
| 3.2 | 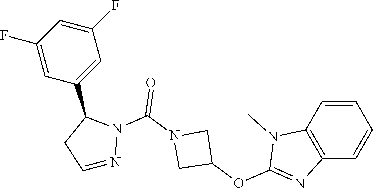 [SMILES: Cn1c(OC2CN(C(=O)N3N=CC[C@H]3c3cc(F)cc(F)c3)C2)nc2ccccc21] | 412 | 97.0 |
| | (S)-(5-(3,5-difluorophenyl)-4,5-dihydro-1H- | | |
| | pyrazol-1-yl)(3-((1-methyl-1H- | | |
| | benzo[d]imidazol-2-yl)oxy)azetidin-1- | | |
| | yl)methanone, TFA salt | | |
| 3.3 | 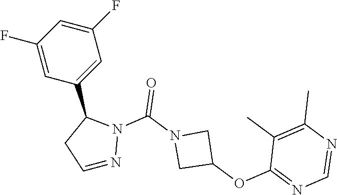 [SMILES: Cc1ncnc(OC2CN(C(=O)N3N=CC[C@H]3c3cc(F)cc(F)c3)C2)c1C] | 388 | 75.9 |
| | (S)-(5-(3,5-difluorophenyl)-4,5-dihydro-1H- | | |
| | pyrazol-1-yl)(3-((5,6-dimethylpyrimidin-4- | | |
| | yl)oxy)azetidin-1-yl)methanone, TFA salt | | |
| 3.4 | 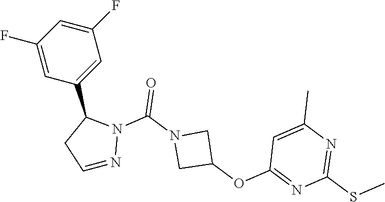 [SMILES: CSc1nc(C)cc(OC2CN(C(=O)N3N=CC[C@H]3c3cc(F)cc(F)c3)C2)n1] | 420 | 38.1 |
| | (S)-(5-(3,5-difluorophenyl)-4,5-dihydro-1H- | | |
| | pyrazol-1-yl)(3-((6-methyl-2- | | |
| | (methylthio)pyrimidin-4-yl)oxy)azetidin-1- | | |
| | yl)methanone, TFA salt | | |
| 3.5 | 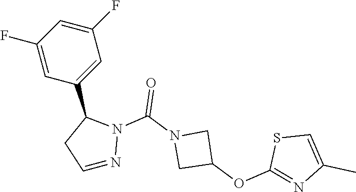 [SMILES: Cc1csc(OC2CN(C(=O)N3N=CC[C@H]3c3cc(F)cc(F)c3)C2)n1] | 379 | 8.9 |
| | (S)-(5-(3,5-difluorophenyl)-4,5-dihydro-1H- | | |
| | pyrazol-1-yl)(3-((4-methylthiazol-2- | | |
| | yl)oxy)azetidin-1-yl)methanone, TFA salt | | |
| 3.6 | 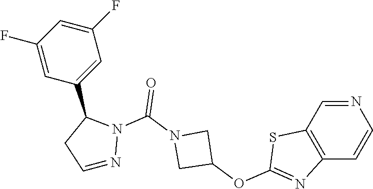 [SMILES: O=C(N1CC(Oc2nc3ccncc3s2)C1)N1N=CC[C@H]1c1cc(F)cc(F)c1] | 416 | 18.2 |
| | (S)-(5-(3,5-difluorophenyl)-4,5-dihydro-1H- | | |
| | pyrazol-1-yl)(3-(thiazolo[5,4-c]pyridin-2- | | |
| | yloxy)azetidin-1-yl)methanone, TFA salt | | |
| 3.7 | 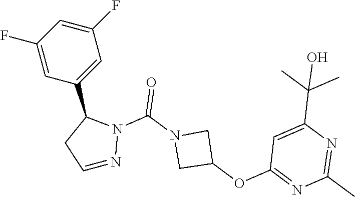 [SMILES: Cc1nc(OC2CN(C(=O)N3N=CC[C@H]3c3cc(F)cc(F)c3)C2)cc(C(C)(C)O)n1] | 432 | 1488 |
| | (S)-(5-(3,5-difluorophenyl)-4,5-dihydro-1H- | | |
| | pyrazol-1-yl)(3-((6-(2-hydroxypropan-2-yl)- | | |
| | 2-methylpyrimidin-4-yl)oxy)azetidin-1- | | |
| | yl)methanone, TFA salt | | |
| 3.8 | 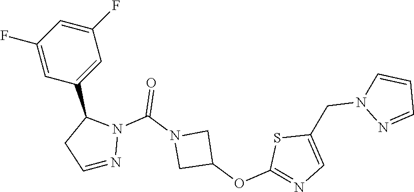 [SMILES: O=C(N1CC(Oc2ncc(Cn3cccn3)s2)C1)N1N=CC[C@H]1c1cc(F)cc(F)c1] | 445 | 105.2 |
| | (S)-(3-((5-((1H-pyrazol-1-yl)methyl)thiazol- | | |
| | 2-yl)oxy)azetidin-1-yl)(5-(3,5- | | |
| | difluorophenyl)-4,5-dihydro-1H-pyrazol-1- | | |
| | yl)methanone, TFA salt | | |

### Reverse Phase Prep-HPLC Methods:

Method a—TFA Modifier

C18 reverse-phase Prep-HPLC (gradient elution, MeCN/H₂O/0.1% TFA). Electrospray (ESI) Mass-triggered fraction collection was employed using positive ion polarity scanning to monitor for the target mass.

### Method B—Basic Modifier

C18 reverse-phase Prep-HPLC (gradient elution, MeCN/H₂O/basic modifier—either 0.1% NH₄OH or 0.05% NH₄HCO₃). Electrospray (ESI) Mass-triggered fraction collection was employed using positive ion polarity scanning to monitor for the target mass.

### RIPK1-ADP-Glo Enzymatic Assay

The enzymatic activity of RIPK1 is measured using an assay derived from ADP-Glo kit (Promega™), which provides a luminescent-based ADP detection system. Specifically, the ADP generated by RIPK1 kinase is proportionally detected as luminescent signals in a homogenous fashion. In this context, the assessment of the inhibitory effect of small molecules (EC₅₀) is measured by the effectiveness of the compounds to inhibit the ATP to ADP conversion by RIPK1.

In this assay, the potency (EC₅₀) of each compound was determined from a ten-point (1:3 serial dilution; top compound concentration of 100000 nM) titration curve using the following outlined procedure. To each well of a white ProxiPlus 384 well-plate, 30 nL of compound (1% DMSO in final assay volume of 3 μL) was dispensed, followed by the addition of 2 μL of 1×assay buffer (25 mM Hepes 7.3, 20 mM MgCl₂, 50 mM NaCl, 1 mM DTT, 0.005% Tween20, and 0.02% BSA) containing 37.5 nM of GST-RIPK1 (recombinant GST-RIPK1 kinase domain (residues 1-327) enzyme produced from baculovirus-transfected Sf21 cells: MW=62 kDa). Plates were placed in an ambient temperature humidified chamber for a 30 minutes pre-incubation with compound. Subsequently, each reaction was initiated by the addition of 1 μL 1×assay buffer containing 900 μM ATP and 3 μM dephosphorylated-MBP substrate. The final reaction in each well of 3 μL consists of 25 nM of GST-RIPK1, 300 μM ATP, and 3 μM dephosphorylated-MBP. Kinase reactions were allowed to proceed for 150 minutes prior to adding ADP-Glo reagents per Promega's outlined kit protocol. Dose-response curves were generated by plotting percent effect (% product conversion; Y-axis) vs. Log₁₀ compound concentrations (X-axis). EC₅₀ values were calculated using a non-linear regression, four-parameters sigmoidal dose-response model.

What is claimed is:

1. A compound of Formula I: 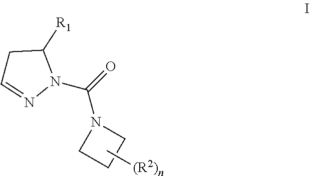
or a pharmaceutically acceptable salt thereof, wherein:
R¹ is aryl, C₃-C₁₀cycloalkyl or heteroaryl, wherein the aryl, C₃-C₁₀cycloalkyl or heteroaryl is unsubstituted or substituted with one to three substituents selected from the group consisting of halogen, C₁-C₆alkyl, CN, OH, alkoxy, —N(R³)₂, —SC₁-C₆alkyl and C₃-C₆cycloalkyl;
Each occurrence of R² is selected from the group consisting of
hydrogen,
OH,
C₁-C₆alkylOH,
CN,
C₁-C₆alkylCN,
C₁-C₆alkyl,
haloC₁-C₆alkyl,
halogen,
alkoxy,
C₁-C₆alkylOC₁-C₆alkyl,
aryl,
heteroaryl,
cycloheteroalkyl,
C₃-C₁₀cycloalkyl,
—O-aryl,
—O-heteroaryl,
—O-cycloheteroalkyl,
—OC₃-C₁₀cycloalkyl,
C₁-C₆alkylaryl,
C₁-C₆alkylheteroaryl,
C₁-C₆alkyl-cycloheteroalkyl,
C₁-C₆alkylC₃-C₁₀cycloalkyl,
haloC₁-C₆alkylaryl,
haloC₁-C₆alkylheteroaryl,
haloC₁-C₆alkyl-cycloheteroalkyl,
haloC₁-C₆alkylC₃-C₁₀cycloalkyl,
—CO-aryl,
—OC₁-C₆alkylaryl,
—OC₁-C₆alkylheteroaryl,
—OC₁-C₆alkyl-cycloheteroalkyl,
—OC₁-C₆alkylC₃-C₆cycloalkyl,
—SO₂C₁-C₆alkyl,
—SO₂aryl,
—S-aryl,
—SC₁-C₆alkyl,
—N(R³)₂, and
C₁-C₆alkylN(R³)₂,
wherein any aryl, heteroaryl, cycloalkyl or cycloheteroalkyl is unsubstituted or substituted with one to three substituents selected from the group consisting of OH, haloC₁-C₆alkyl, aryl, heteroaryl, N(R³)₂, SO₂C₁-C₆alkyl, alkoxy, CN, halogen, C₁-C₆alkyl, —SC₁-C₆alkyl, C₁-C₆alkyl-cycloheteroalkyl, C₁-C₆alkylheteroaryl and C₁-C₆alkylOH;
R³ is hydrogen, C₁-C₆alkyl, aryl or heteroaryl, wherein the aryl or heteroaryl is unsubstituted or substituted with 1-3 substituents selected from the group consisting of CN, C₁-C₆alkyl, haloC₁-C₆alkyl and alkoxy; and
n is 1, 2, 3, 4, 5 or 6, wherein, when n is 2, 3, 4, 5 or 6, two R² substituents can be taken together to form a cycloheteroalkyl or C₃-C₁₀cycloalkyl, wherein the cycloheteroalkyl or C₃-C₁₀cycloalkyl is unsubstituted or substituted with one to four substituents selected from the group consisting of halogen, C₁-C₆alkyl, aryl, alkoxy, —O-aryl, —O-heteroaryl, N(R³)₂, CN, —SO₂C₁-C₆alkyl, C₁-C₆alkylOH, heteroaryl, C(O)OC₁-C₆alkyl, and C₁-C₆alkyl-cycloheteroalkyl.

2. A compound of claim 1, wherein n is 2 or 3.

3. A compound of claim 1, wherein each occurrence of R² is independently selected from the group consisting of methyl, OH, fluorine, CN, methoxy, chlorine, ethoxy, difluoromethyl, trifluoromethyl, —SO₂CH₃, isopropyl, cyclopropyl,

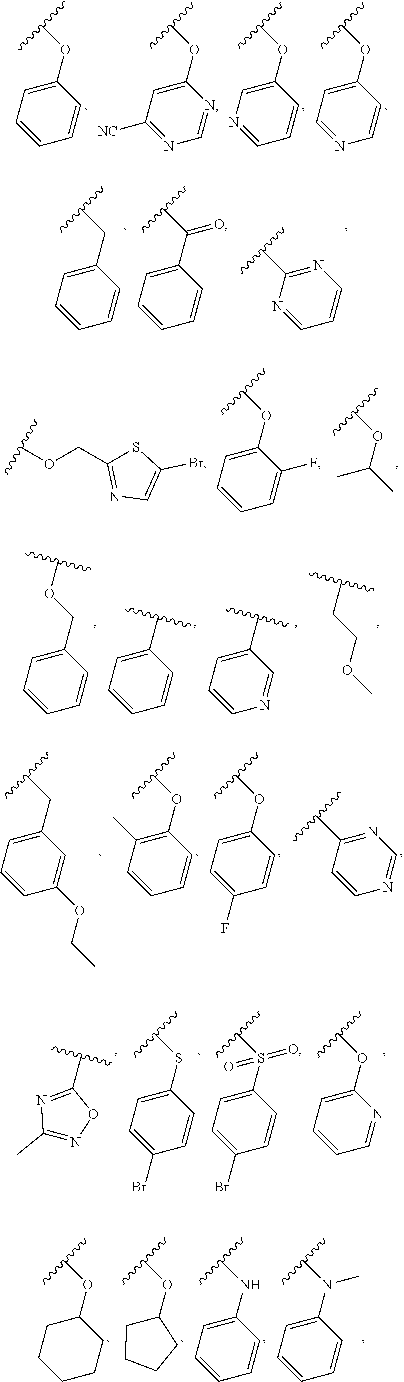

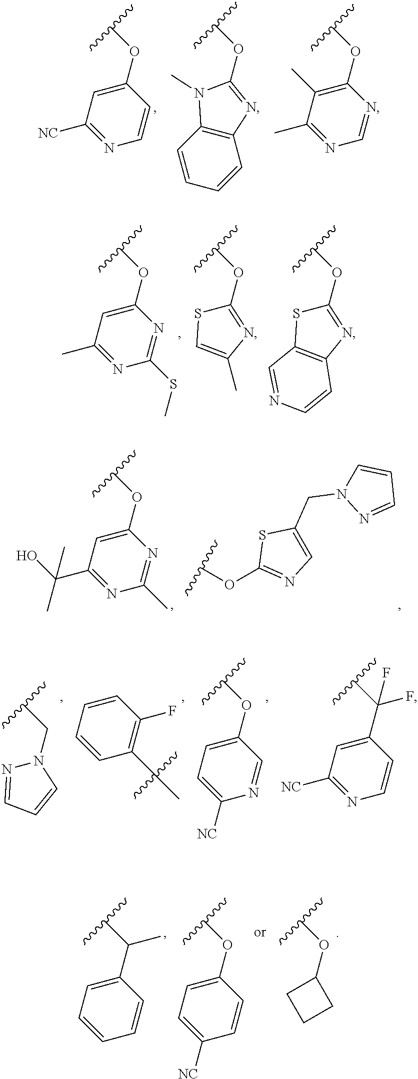

4. The compound of claim 1, or a pharmaceutically acceptable salt thereof, wherein n is at least 2, and wherein the two R² substituents can be taken together to form:

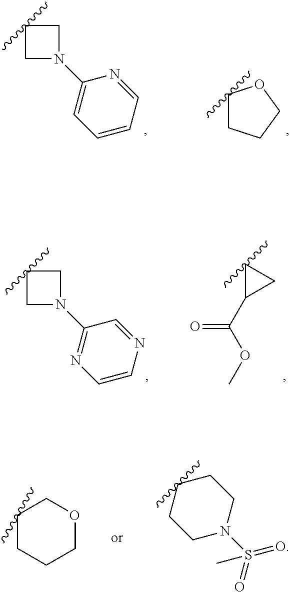

5. A compound of Formula II: 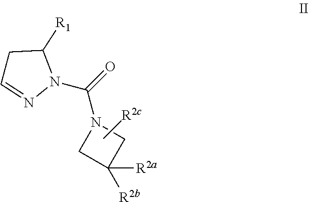
or a pharmaceutically acceptable salt thereof, wherein:
R¹ is aryl, C₃-C₁₀cycloalkyl or heteroaryl, wherein the aryl, C₃-C₁₀cycloalkyl or heteroaryl is unsubstituted or substituted with one to three substituents selected from the group consisting of halogen, C₁-C₆alkyl, CN, OH, alkoxy, —N(R³)₂, —SC₁-C₆alkyl and C₃-C₆cycloalkyl;
R^{2a} is hydrogen, halogen, CN, OH, C₁-C₆alkyl, alkoxy, C₃-C₆cycloalkyl, C₁-C₆alkylOH, C₁-C₆alkylOC₁-C₆alkyl, N(R³)₂ or haloC₁-C₆alkyl, or taken with R^{2b} forms a cycloheteroalkyl or C₃-C₁₀cycloalkyl, wherein the cycloheteroalkyl or C₃-C₁₀cycloalkyl is unsubstituted or substituted with one to four substituents selected from the group consisting —SO₂C₁-C₆alkyl, C₁-C₆alkylOH, heteroaryl, C(O)OC₁-C₆alkyl, halogen, C₁-C₆alkyl, aryl, alkoxy, —O-aryl, —O-heteroaryl, N(R³)₂, CN and C₁-C₆alkyl-cycloheteroalkyl;
R^{2b} is hydrogen, OH, C₁-C₆alkylOH, CN, C₁-C₆alkylCN, C₁-C₆alkyl, haloC₁-C₆alkyl, halogen, alkoxy, C₁-C₆alkylOC₁-C₆alkyl, aryl, heteroaryl, cycloheteroalkyl, C₃-C₁₀cycloalkyl, —O-aryl, —O-heteroaryl, —O-cycloheteroalkyl, —OC₃-C₁₀cycloalkyl, C₁-C₆alkylaryl, C₁-C₆alkylheteroaryl, C₁-C₆alkyl-cycloheteroalkyl, C₁-C₆alkylC₃-C₁₀cycloalkyl, haloC₁-C₆alkylaryl, haloC₁-C₆alkylheteroaryl, haloC₁-C₆alkyl-cycloheteroalkyl, haloC₁-C₆alkylC₃-C₁₀cycloalkyl, —CO-aryl, —OC₁-C₆alkylaryl, —OC₁-C₆alkylheteroaryl, —OC₁-C₆alkyl-cycloheteroalkyl, —OC₁-C₆alkylC₃-C₆cycloalkyl, —SO₂C₁-C₆alkyl, —SO₂aryl, —S-aryl, —SC₁-C₆alkyl, —N(R³)₂ or C₁-C₆alkylN(R³)₂, wherein any aryl, heteroaryl, cycloalkyl or cycloheteroalkyl is unsubstituted or substituted with one to three substituents selected from the group consisting of OH, haloC₁-C₆alkyl, aryl, heteroaryl, N(R³)₂, SO₂C₁-C₆alkyl, alkoxy, CN, halogen, C₁-C₆alkyl, —SC₁-C₆alkyl, C₁-C₆alkyl-cycloheteroalkyl, C₁-C₆alkylheteroaryl and C₁-C₆alkylOH or, when taken with R^{2a} forms a cycloheteroalkyl or C₃-C₁₀cycloalkyl, wherein the cycloheteroalkyl or C₃-C₁₀cycloalkyl is unsubstituted or substituted with one to four substituents selected from the group consisting halogen, C₁-C₆alkyl, aryl, alkoxy, —O-aryl, —O-heteroaryl, N(R³)₂, CN, —SO₂C₁-C₆alkyl, C₁-C₆alkylOH, heteroaryl, C(O)OC₁-C₆alkyl, and C₁-C₆alkyl-cycloheteroalkyl;
R^{2c} is hydrogen, C₁-C₆alkyl, C₁-C₆alkylOH, C₃-C₁₀cycloalkyl, heteroaryl, C₁-C₆alkylOC₁-C₆alkyl, C₁-C₆alkylO-heteroaryl, C₁-C₆alkylheteroaryl, aryl or C₁-C₆alkylaryl, wherein the aryl, heteroaryl, C₃-C₆cycloalkyl, C₁-C₆alkylheteroaryl or C₁-C₆alkylaryl is unsubstituted or substituted with one to four substituents selected from the group consisting of halogen C₁-C₆alkyl, CN, OH and alkoxy; and
R³ is hydrogen, C₁-C₆alkyl, aryl or heteroaryl, wherein the aryl and heteroaryl is unsubstituted or substituted with 1-3 substituents selected from the group consisting of CN, C₁-C₆alkyl, haloC₁-C₆alkyl and alkoxy.

6. The compound of claim 1, or a pharmaceutically acceptable salt thereof, wherein R¹ is aryl.

7. The compound of claim 1, or a pharmaceutically acceptable salt thereof, wherein R¹ is phenyl.

8. The compound of claim 1, or a pharmaceutically acceptable salt thereof, wherein R¹ is unsubstituted or substituted with one or two halogens selected from the group consisting of fluorine and chlorine.

9. The compound of claim 1, or a pharmaceutically acceptable salt thereof, wherein R¹ is C₃-C₁₀cycloalkyl.

10. The compound of claim 1, or a pharmaceutically acceptable salt thereof, wherein R¹ is:

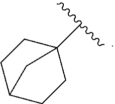 [SMILES: *C12CCC(CC1)C2]

11. The compound of claim 1, or a pharmaceutically acceptable salt thereof, wherein R¹ is heteroaryl.

12. The compound of claim 1, or a pharmaceutically acceptable salt thereof, wherein R¹ is 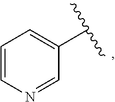
wherein R¹ is unsubstituted or substituted with one or two substituents selected from the group consisting of methyl, fluorine, and CN.

13. The compound of claim 5, or a pharmaceutically acceptable salt thereof, wherein R^{2c} is hydrogen, methyl,

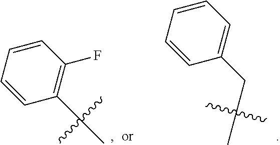 [SMILES: *c1ccccc1F ; *Cc1ccccc1]

14. The compound of claim 5, or a pharmaceutically acceptable salt thereof, wherein R^{2a} is hydrogen, methyl, OH, fluorine, CN, or methoxy.

15. The compound of claim 5, or a pharmaceutically acceptable salt thereof, wherein R^{2a} is taken with R^{2b} and forms:

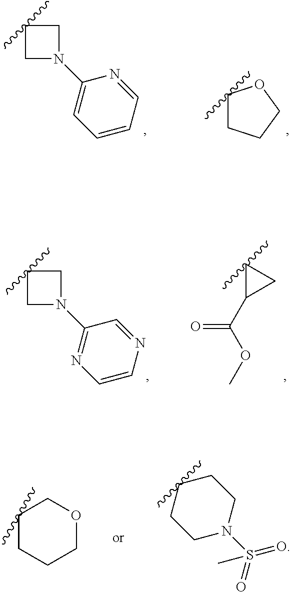

16. The compound of claim 5, or a pharmaceutically acceptable salt thereof, wherein R^{2b} is hydrogen, OH, chlorine, fluorine, CN, methoxy, ethoxy, methyl, difluoromethyl, trifluoromethyl, —SO₂CH₃, isopropyl, cyclopropyl,

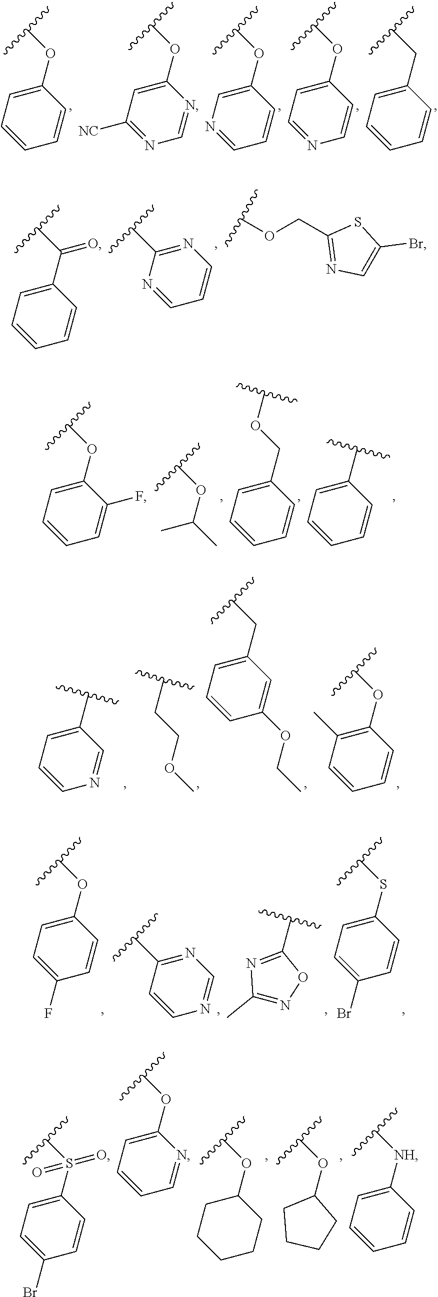

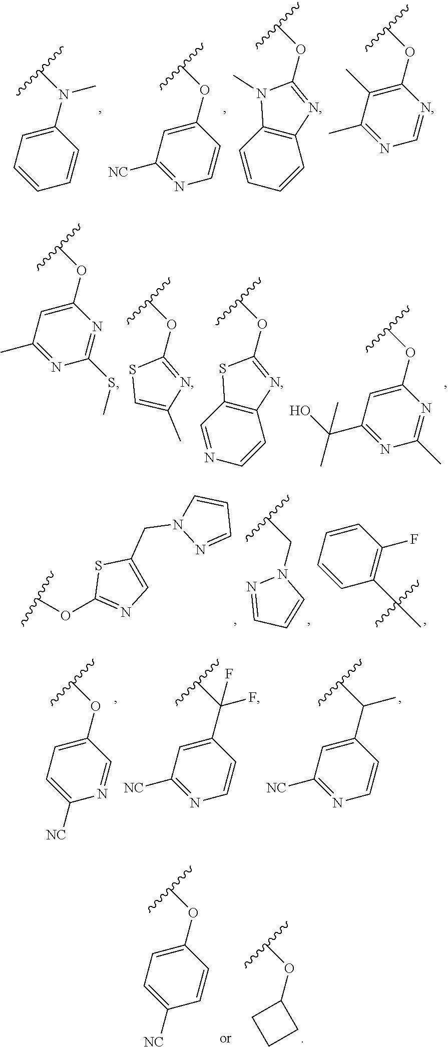

17. The compound of claim 1, or a pharmaceutically acceptable salt thereof, and R³ is hydrogen, methyl, or phenyl.

18. A compound of Formula III: 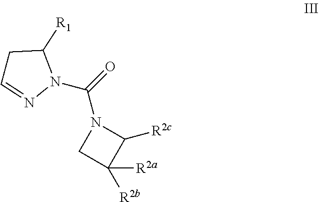
or a pharmaceutically acceptable salt thereof, wherein:
R¹ is 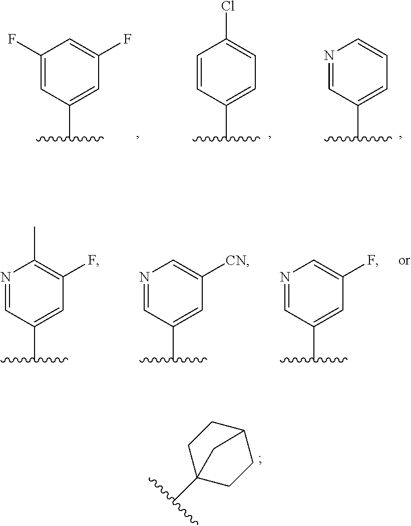
R^{2a} is hydrogen, halogen, CN, OH, C₁-C₆alkyl, alkoxy or haloC₁-C₆alkyl, or taken with R^{2b} forms a cycloheteroalkyl or C₃-C₁₀cycloalkyl, wherein the cycloheteroalkyl or C₃-C₁₀cycloalkyl is unsubstituted or substituted with one to four substituents selected from the group consisting —SO₂C₁-C₆alkyl, C₁-C₆alkylOH, heteroaryl, C(O)OC₁-C₆alkyl, and C₁-C₆alkyl-cycloheteroalkyl;
R^{2b} is hydrogen, —O-aryl, —O-heteroaryl, C₁-C₆alkylaryl, OH, —CO-aryl, —OC₃-C₁₀cycloalkyl, heteroaryl, alkoxy, —OC₁-C₆alkylaryl, haloC₁-C₆alkyl, C₁-C₆alkyl, halogen, C₃-C₁₀cycloalkyl, C₁-C₆alkylOC₁-C₆alkyl, aryl, —OC₁-C₆alkyl-cycloheteroalkyl, —SO₂C₁-C₆alkyl, cycloheteroalkyl, —S-aryl, —SO₂aryl, —N(R³)₂, C₁-C₆alkylheteroaryl, haloC₁-C₆alkylheteroaryl, C₁-C₆alkylC₃-C₁₀cycloalkyl, —O-cycloheteroalkyl, C₁-C₆alkyl-cycloheteroalkyl, —OC₁-C₆alkylheteroaryl or CN, wherein the —O-heteroaryl, —O-aryl, C₁-C₆alkylaryl, —OC₁-C₆alkyl-cycloheteroalkyl, cycloheteroalkyl, —S-aryl, —SO₂aryl, heteroaryl, —OC₁-C₆alkylheteroaryl or —O-cycloheteroalkyl is unsubstituted or substituted with one to three substituents selected from the group consisting of alkoxy, CN, halogen, C₁-C₆alkyl, —SC₁-C₆alkyl, C₁-C₆alkyl-cycloheteroalkyl, C₁-C₆alkylheteroaryl and C₁-C₆alkylOH or, when taken with R^{2a} forms a cycloheteroalkyl or C₃-C₁₀cycloalkyl, wherein the cycloheteroalkyl or C₃-C₁₀cycloalkyl is unsubstituted or substituted with one to four substituents selected from the group consisting —SO₂C₁-C₆alkyl, C₁-C₆alkylOH, heteroaryl, C(O)OC₁-C₆alkyl, and C₁-C₆alkyl-cycloheteroalkyl;
R^{2c} is hydrogen, C₁-C₆alkyl, aryl or C₁-C₆alkylaryl, wherein the aryl or C₁-C₆alkylaryl is unsubstituted or substituted with one to four halogen substituents; and
R³ is hydrogen, C₁-C₆alkyl or aryl.

19. A compound of Formula I, or a pharmaceutically acceptable salt thereof, having the structure:

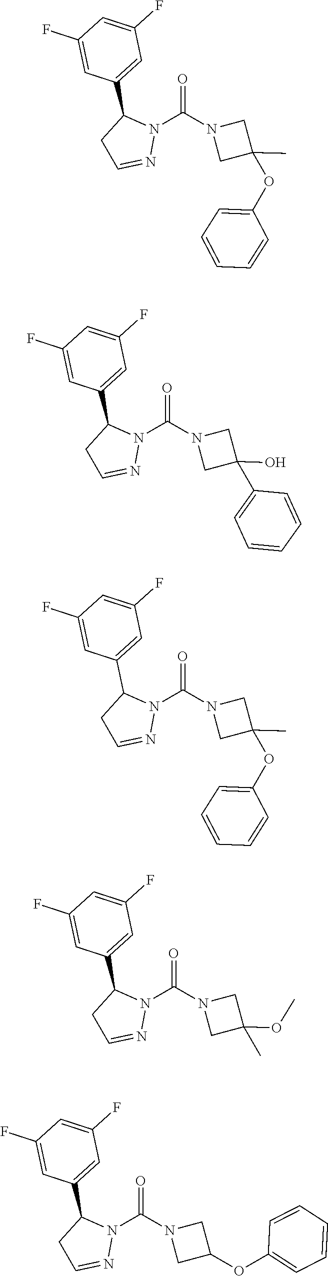 [SMILES: CC1(Oc2ccccc2)CN(C(=O)N2N=CC[C@H]2c2cc(F)cc(F)c2)C1 ; O=C(N1CC(O)(c2ccccc2)C1)N1N=CC[C@H]1c1cc(F)cc(F)c1 ; CC1(Oc2ccccc2)CN(C(=O)N2N=CCC2c2cc(F)cc(F)c2)C1 ; COC1(C)CN(C(=O)N2N=CC[C@H]2c2cc(F)cc(F)c2)C1 ; O=C(N1CC(Oc2ccccc2)C1)N1N=CC[C@H]1c1cc(F)cc(F)c1]

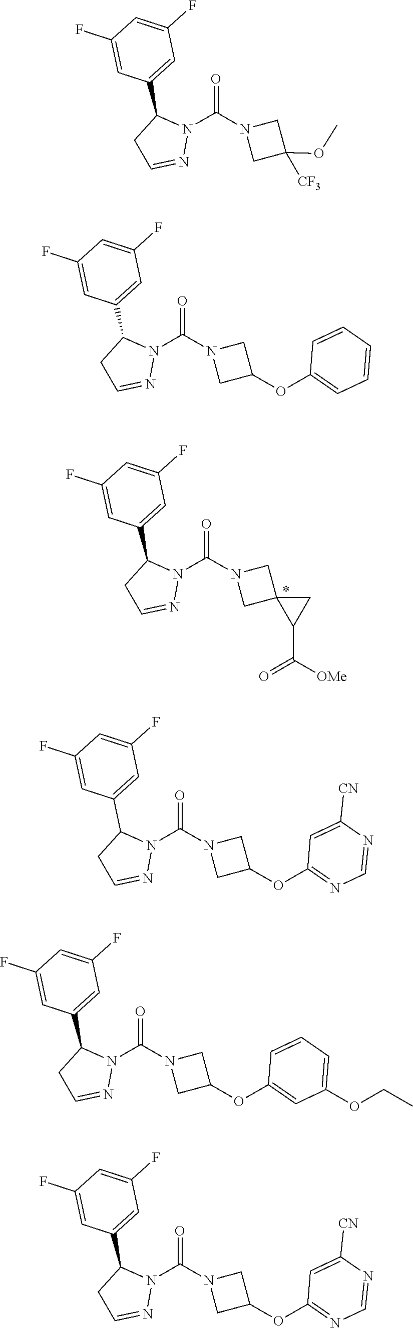 [SMILES: COC1(C(F)(F)F)CN(C(=O)N2N=CC[C@H]2c2cc(F)cc(F)c2)C1 ; O=C(N1CC(Oc2ccccc2)C1)N1N=CC[C@@H]1c1cc(F)cc(F)c1 ; COC(=O)C1CC12CN(C(=O)N1N=CC[C@H]1c1cc(F)cc(F)c1)C2 ; N#Cc1cc(OC2CN(C(=O)N3N=CCC3c3cc(F)cc(F)c3)C2)ncn1 ; CCOc1cccc(OC2CN(C(=O)N3N=CC[C@H]3c3cc(F)cc(F)c3)C2)c1 ; N#Cc1cc(OC2CN(C(=O)N3N=CC[C@H]3c3cc(F)cc(F)c3)C2)ncn1]

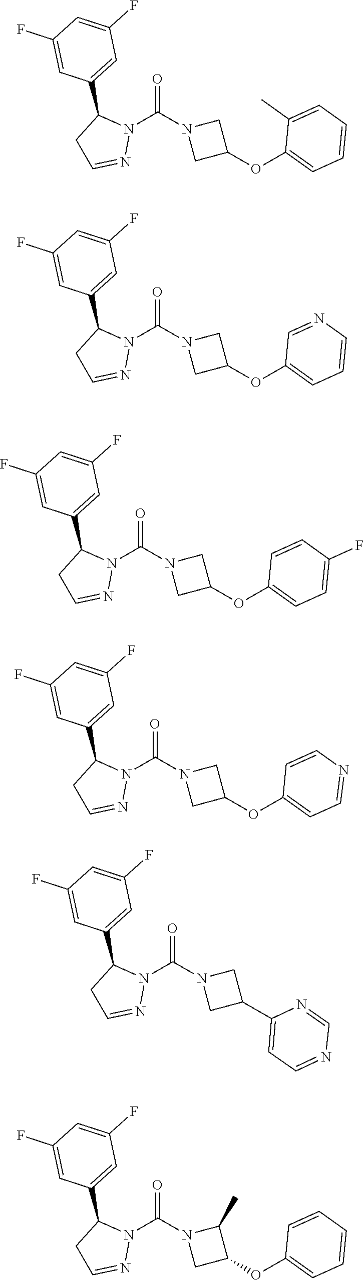 [SMILES: Cc1ccccc1OC1CN(C(=O)N2N=CC[C@H]2c2cc(F)cc(F)c2)C1 ; O=C(N1CC(Oc2cccnc2)C1)N1N=CC[C@H]1c1cc(F)cc(F)c1 ; O=C(N1CC(Oc2ccc(F)cc2)C1)N1N=CC[C@H]1c1cc(F)cc(F)c1 ; O=C(N1CC(Oc2ccncc2)C1)N1N=CC[C@H]1c1cc(F)cc(F)c1 ; O=C(N1CC(c2ccncn2)C1)N1N=CC[C@H]1c1cc(F)cc(F)c1 ; C[C@H]1[C@H](Oc2ccccc2)CN1C(=O)N1N=CC[C@H]1c1cc(F)cc(F)c1]

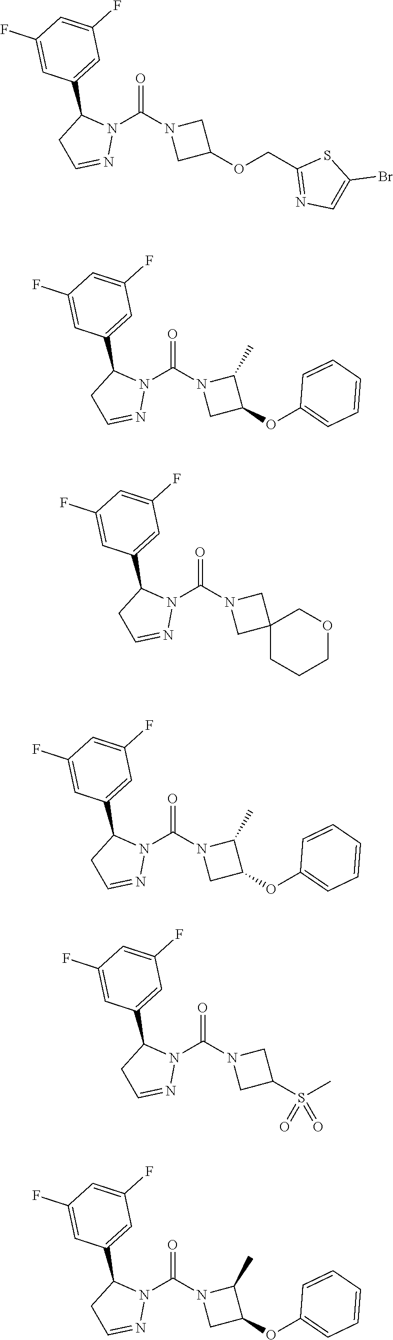 [SMILES: O=C(N1CC(OCc2ncc(Br)s2)C1)N1N=CC[C@H]1c1cc(F)cc(F)c1 ; C[C@@H]1[C@@H](Oc2ccccc2)CN1C(=O)N1N=CC[C@H]1c1cc(F)cc(F)c1 ; O=C(N1CC2(CCCOC2)C1)N1N=CC[C@H]1c1cc(F)cc(F)c1 ; C[C@@H]1[C@H](Oc2ccccc2)CN1C(=O)N1N=CC[C@H]1c1cc(F)cc(F)c1 ; CS(=O)(=O)C1CN(C(=O)N2N=CC[C@H]2c2cc(F)cc(F)c2)C1 ; C[C@H]1[C@@H](Oc2ccccc2)CN1C(=O)N1N=CC[C@H]1c1cc(F)cc(F)c1]

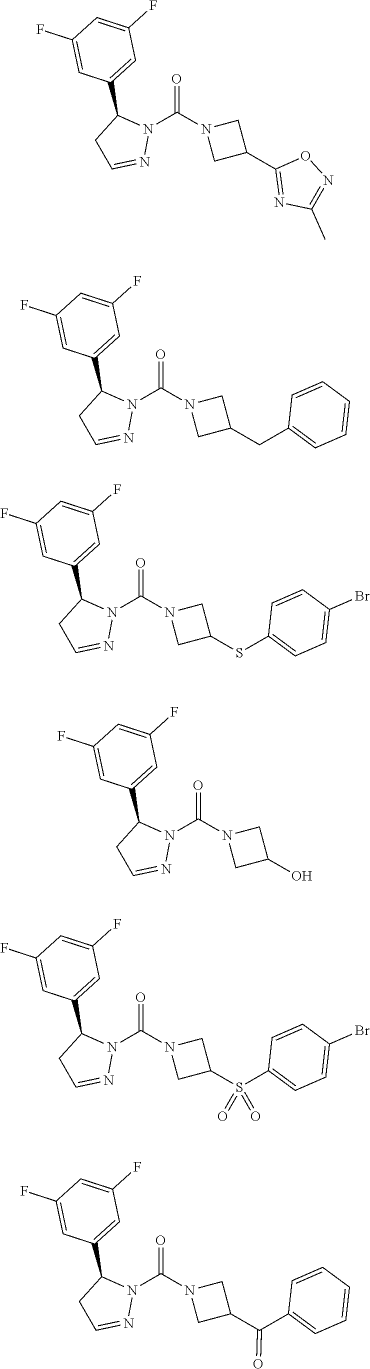 [SMILES: Cc1noc(C2CN(C(=O)N3N=CC[C@H]3c3cc(F)cc(F)c3)C2)n1 ; O=C(N1CC(Cc2ccccc2)C1)N1N=CC[C@H]1c1cc(F)cc(F)c1 ; O=C(N1CC(Sc2ccc(Br)cc2)C1)N1N=CC[C@H]1c1cc(F)cc(F)c1 ; O=C(N1CC(O)C1)N1N=CC[C@H]1c1cc(F)cc(F)c1 ; O=C(N1CC(S(=O)(=O)c2ccc(Br)cc2)C1)N1N=CC[C@H]1c1cc(F)cc(F)c1 ; O=C(c1ccccc1)C1CN(C(=O)N2N=CC[C@H]2c2cc(F)cc(F)c2)C1]

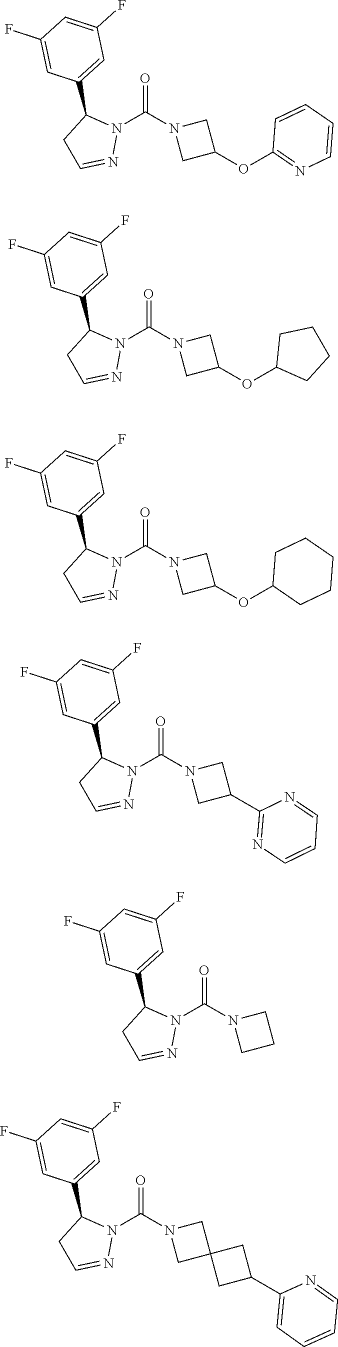 [SMILES: O=C(N1CC(Oc2ccccn2)C1)N1N=CC[C@H]1c1cc(F)cc(F)c1 ; O=C(N1CC(OC2CCCC2)C1)N1N=CC[C@H]1c1cc(F)cc(F)c1 ; O=C(N1CC(OC2CCCCC2)C1)N1N=CC[C@H]1c1cc(F)cc(F)c1 ; O=C(N1CC(c2ncccn2)C1)N1N=CC[C@H]1c1cc(F)cc(F)c1 ; O=C(N1CCC1)N1N=CC[C@H]1c1cc(F)cc(F)c1 ; O=C(N1CC2(CC(c3ccccn3)C2)C1)N1N=CC[C@H]1c1cc(F)cc(F)c1]

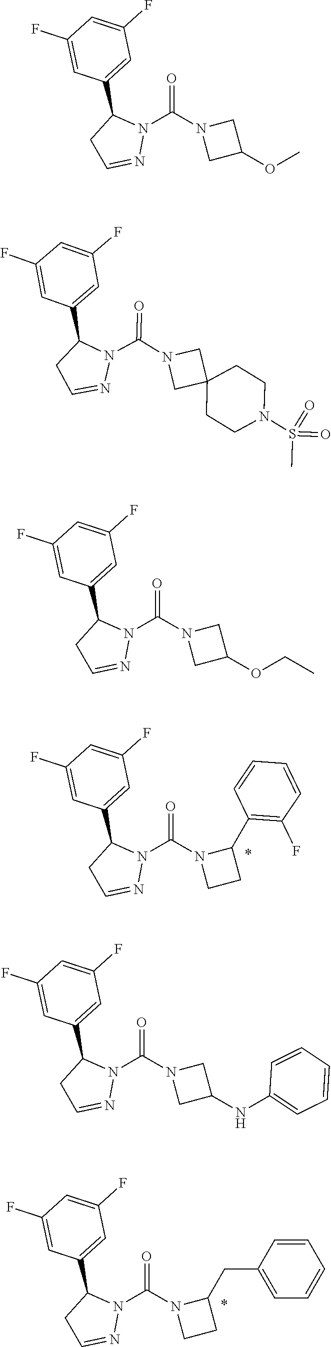 [SMILES: COC1CN(C(=O)N2N=CC[C@H]2c2cc(F)cc(F)c2)C1 ; CS(=O)(=O)N1CCC2(CC1)CN(C(=O)N1N=CC[C@H]1c1cc(F)cc(F)c1)C2 ; CCOC1CN(C(=O)N2N=CC[C@H]2c2cc(F)cc(F)c2)C1 ; O=C(N1CCC1c1ccccc1F)N1N=CC[C@H]1c1cc(F)cc(F)c1 ; O=C(N1CC(Nc2ccccc2)C1)N1N=CC[C@H]1c1cc(F)cc(F)c1 ; O=C(N1CCC1Cc1ccccc1)N1N=CC[C@H]1c1cc(F)cc(F)c1]

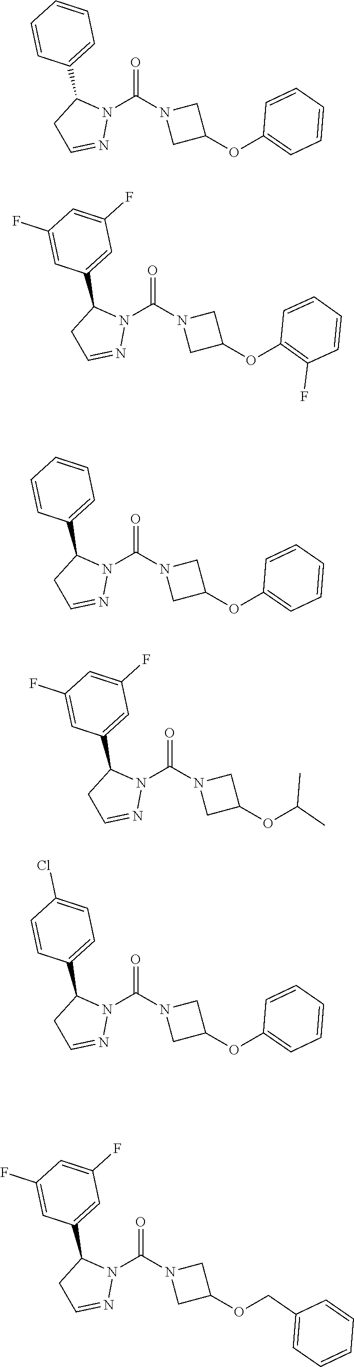 [SMILES: O=C(N1CC(Oc2ccccc2)C1)N1N=CC[C@@H]1c1ccccc1 ; O=C(N1CC(Oc2ccccc2F)C1)N1N=CC[C@H]1c1cc(F)cc(F)c1 ; O=C(N1CC(Oc2ccccc2)C1)N1N=CC[C@H]1c1ccccc1 ; CC(C)OC1CN(C(=O)N2N=CC[C@H]2c2cc(F)cc(F)c2)C1 ; O=C(N1CC(Oc2ccccc2)C1)N1N=CC[C@H]1c1ccc(Cl)cc1 ; O=C(N1CC(OCc2ccccc2)C1)N1N=CC[C@H]1c1cc(F)cc(F)c1]

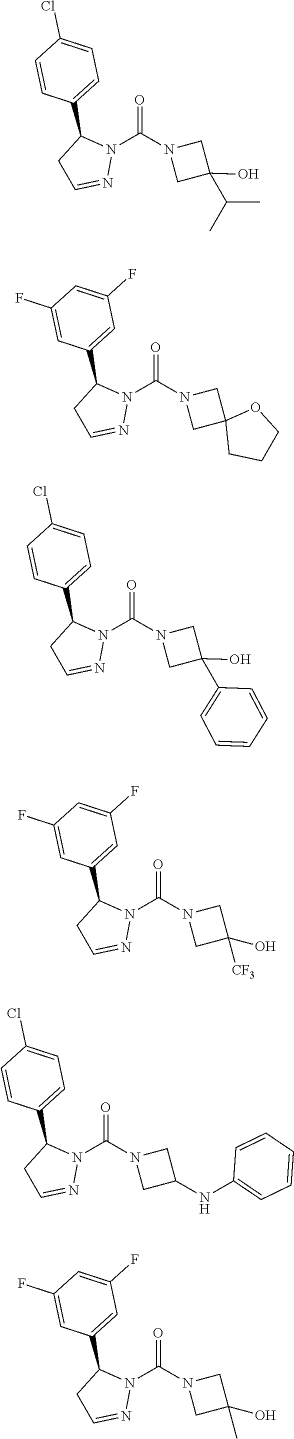 [SMILES: CC(C)C1(O)CN(C(=O)N2N=CC[C@H]2c2ccc(Cl)cc2)C1 ; O=C(N1CC2(CCCO2)C1)N1N=CC[C@H]1c1cc(F)cc(F)c1 ; O=C(N1CC(O)(c2ccccc2)C1)N1N=CC[C@H]1c1ccc(Cl)cc1 ; O=C(N1CC(O)(C(F)(F)F)C1)N1N=CC[C@H]1c1cc(F)cc(F)c1 ; O=C(N1CC(Nc2ccccc2)C1)N1N=CC[C@H]1c1ccc(Cl)cc1 ; CC1(O)CN(C(=O)N2N=CC[C@H]2c2cc(F)cc(F)c2)C1]

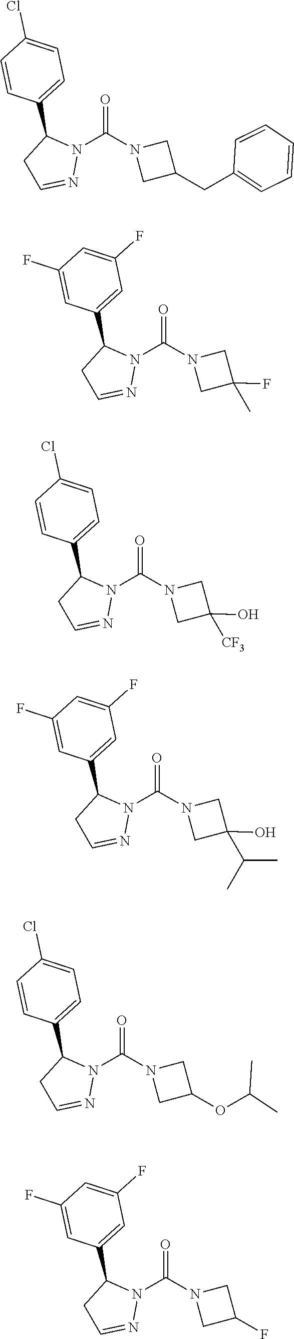 [SMILES: O=C(N1CC(Cc2ccccc2)C1)N1N=CC[C@H]1c1ccc(Cl)cc1 ; CC1(F)CN(C(=O)N2N=CC[C@H]2c2cc(F)cc(F)c2)C1 ; O=C(N1CC(O)(C(F)(F)F)C1)N1N=CC[C@H]1c1ccc(Cl)cc1 ; CC(C)C1(O)CN(C(=O)N2N=CC[C@H]2c2cc(F)cc(F)c2)C1 ; CC(C)OC1CN(C(=O)N2N=CC[C@H]2c2ccc(Cl)cc2)C1 ; O=C(N1CC(F)C1)N1N=CC[C@H]1c1cc(F)cc(F)c1]

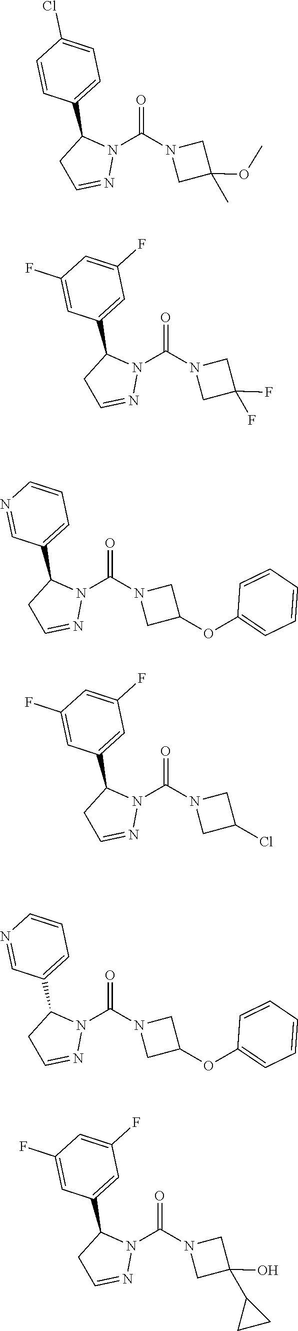 [SMILES: COC1(C)CN(C(=O)N2N=CC[C@H]2c2ccc(Cl)cc2)C1 ; O=C(N1CC(F)(F)C1)N1N=CC[C@H]1c1cc(F)cc(F)c1 ; O=C(N1CC(Oc2ccccc2)C1)N1N=CC[C@H]1c1cccnc1 ; O=C(N1CC(Cl)C1)N1N=CC[C@H]1c1cc(F)cc(F)c1 ; O=C(N1CC(Oc2ccccc2)C1)N1N=CC[C@@H]1c1cccnc1 ; O=C(N1CC(O)(C2CC2)C1)N1N=CC[C@H]1c1cc(F)cc(F)c1]

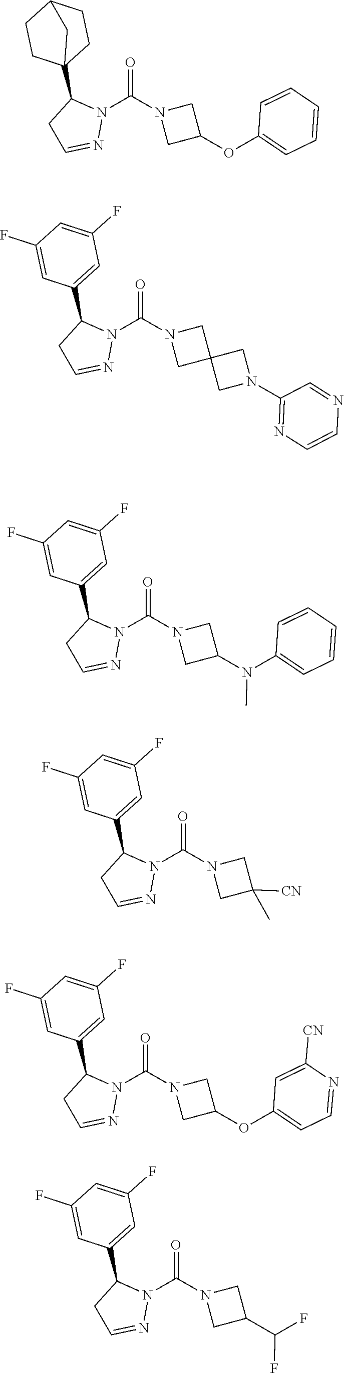 [SMILES: O=C(N1CC(Oc2ccccc2)C1)N1N=CC[C@H]1C12CCC(CC1)C2 ; O=C(N1CC2(C1)CN(c1cnccn1)C2)N1N=CC[C@H]1c1cc(F)cc(F)c1 ; CN(c1ccccc1)C1CN(C(=O)N2N=CC[C@H]2c2cc(F)cc(F)c2)C1 ; CC1(C#N)CN(C(=O)N2N=CC[C@H]2c2cc(F)cc(F)c2)C1 ; N#Cc1cc(OC2CN(C(=O)N3N=CC[C@H]3c3cc(F)cc(F)c3)C2)ccn1 ; O=C(N1CC(C(F)F)C1)N1N=CC[C@H]1c1cc(F)cc(F)c1]

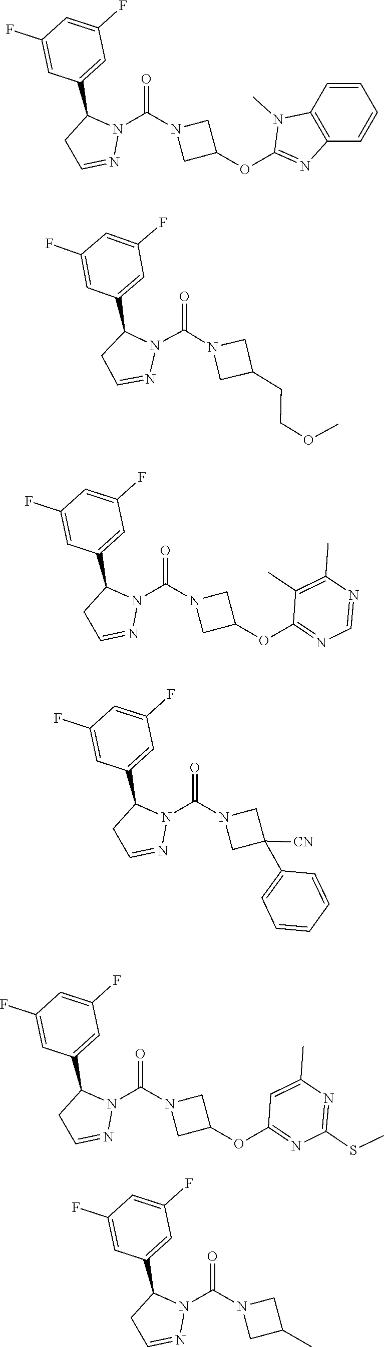 [SMILES: Cn1c(OC2CN(C(=O)N3N=CC[C@H]3c3cc(F)cc(F)c3)C2)nc2ccccc21 ; COCCC1CN(C(=O)N2N=CC[C@H]2c2cc(F)cc(F)c2)C1 ; Cc1ncnc(OC2CN(C(=O)N3N=CC[C@H]3c3cc(F)cc(F)c3)C2)c1C ; N#CC1(c2ccccc2)CN(C(=O)N2N=CC[C@H]2c2cc(F)cc(F)c2)C1 ; CSc1nc(C)cc(OC2CN(C(=O)N3N=CC[C@H]3c3cc(F)cc(F)c3)C2)n1 ; CC1CN(C(=O)N2N=CC[C@H]2c2cc(F)cc(F)c2)C1]

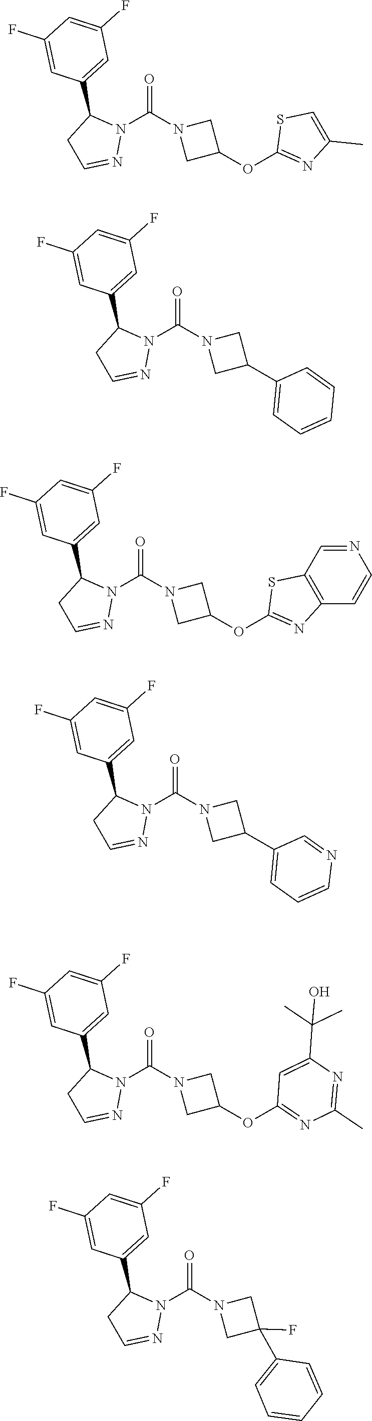 [SMILES: Cc1csc(OC2CN(C(=O)N3N=CC[C@H]3c3cc(F)cc(F)c3)C2)n1 ; O=C(N1CC(c2ccccc2)C1)N1N=CC[C@H]1c1cc(F)cc(F)c1 ; O=C(N1CC(Oc2nc3ccncc3s2)C1)N1N=CC[C@H]1c1cc(F)cc(F)c1 ; O=C(N1CC(c2cccnc2)C1)N1N=CC[C@H]1c1cc(F)cc(F)c1 ; Cc1nc(OC2CN(C(=O)N3N=CC[C@H]3c3cc(F)cc(F)c3)C2)cc(C(C)(C)O)n1 ; O=C(N1CC(F)(c2ccccc2)C1)N1N=CC[C@H]1c1cc(F)cc(F)c1]

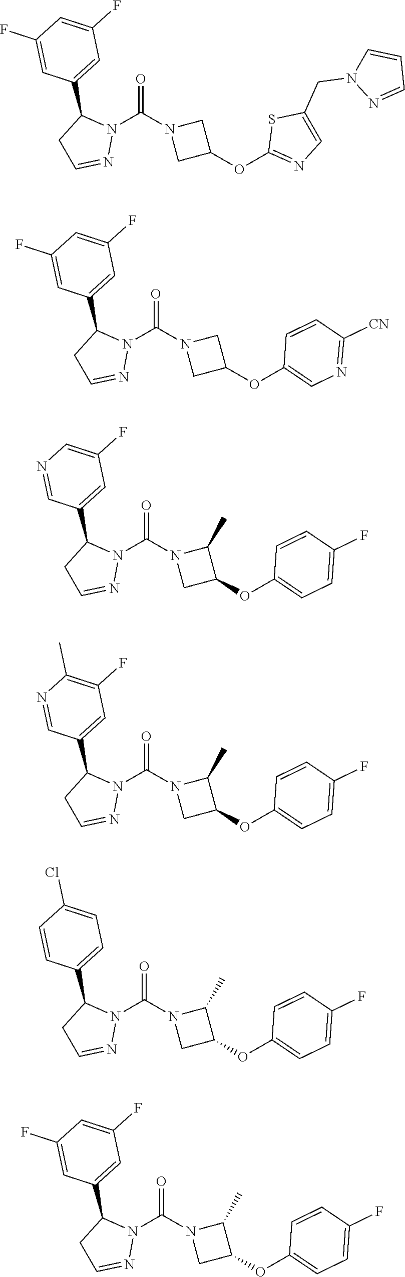 [SMILES: O=C(N1CC(Oc2ncc(Cn3cccn3)s2)C1)N1N=CC[C@H]1c1cc(F)cc(F)c1 ; N#Cc1ccc(OC2CN(C(=O)N3N=CC[C@H]3c3cc(F)cc(F)c3)C2)cn1 ; C[C@H]1[C@@H](Oc2ccc(F)cc2)CN1C(=O)N1N=CC[C@H]1c1cncc(F)c1 ; Cc1ncc([C@@H]2CC=NN2C(=O)N2C[C@H](Oc3ccc(F)cc3)[C@@H]2C)cc1F ; C[C@@H]1[C@H](Oc2ccc(F)cc2)CN1C(=O)N1N=CC[C@H]1c1ccc(Cl)cc1 ; C[C@@H]1[C@H](Oc2ccc(F)cc2)CN1C(=O)N1N=CC[C@H]1c1cc(F)cc(F)c1]

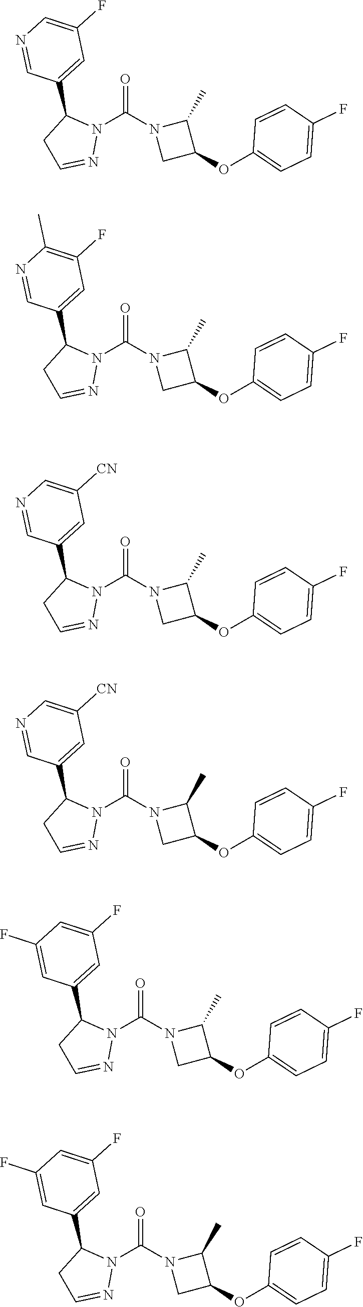 [SMILES: C[C@@H]1[C@@H](Oc2ccc(F)cc2)CN1C(=O)N1N=CC[C@H]1c1cncc(F)c1 ; Cc1ncc([C@@H]2CC=NN2C(=O)N2C[C@H](Oc3ccc(F)cc3)[C@H]2C)cc1F ; C[C@@H]1[C@@H](Oc2ccc(F)cc2)CN1C(=O)N1N=CC[C@H]1c1cncc(C#N)c1 ; C[C@H]1[C@@H](Oc2ccc(F)cc2)CN1C(=O)N1N=CC[C@H]1c1cncc(C#N)c1 ; C[C@@H]1[C@@H](Oc2ccc(F)cc2)CN1C(=O)N1N=CC[C@H]1c1cc(F)cc(F)c1 ; C[C@H]1[C@@H](Oc2ccc(F)cc2)CN1C(=O)N1N=CC[C@H]1c1cc(F)cc(F)c1]

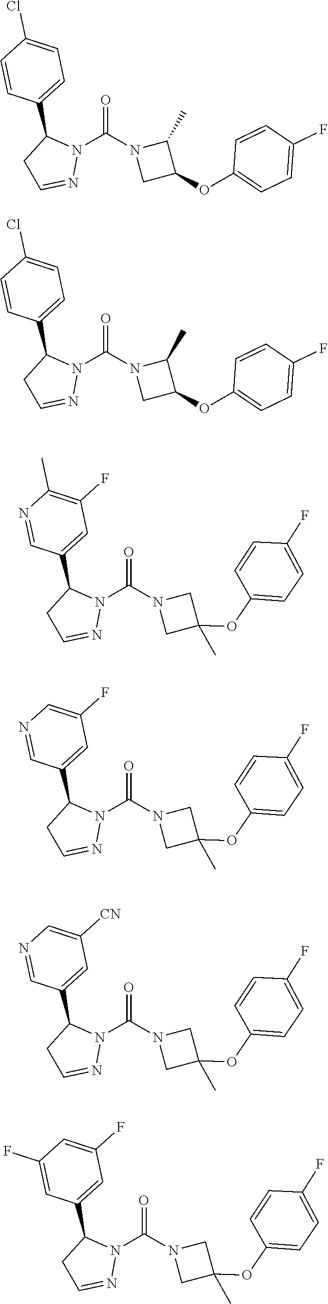

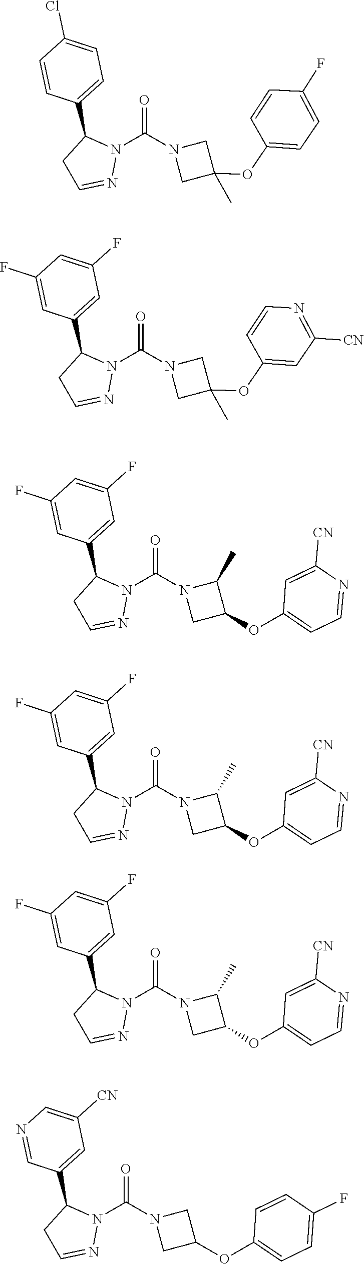

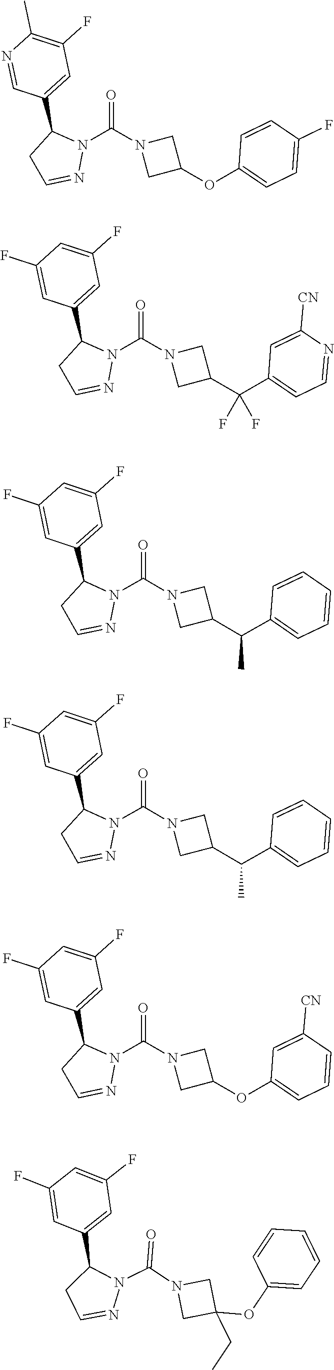 [SMILES: Cc1ncc([C@@H]2CC=NN2C(=O)N2CC(Oc3ccc(F)cc3)C2)cc1F ; N#Cc1cc(C(F)(F)C2CN(C(=O)N3N=CC[C@H]3c3cc(F)cc(F)c3)C2)ccn1 ; C[C@H](c1ccccc1)C1CN(C(=O)N2N=CC[C@H]2c2cc(F)cc(F)c2)C1 ; C[C@@H](c1ccccc1)C1CN(C(=O)N2N=CC[C@H]2c2cc(F)cc(F)c2)C1 ; N#Cc1cccc(OC2CN(C(=O)N3N=CC[C@H]3c3cc(F)cc(F)c3)C2)c1 ; CCC1(Oc2ccccc2)CN(C(=O)N2N=CC[C@H]2c2cc(F)cc(F)c2)C1]

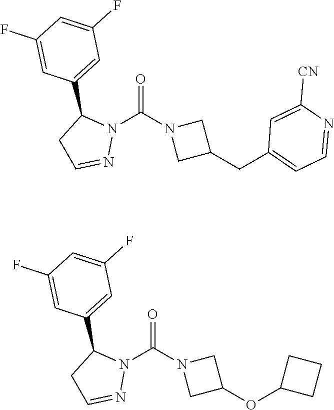 [SMILES: N#Cc1cc(CC2CN(C(=O)N3N=CC[C@H]3c3cc(F)cc(F)c3)C2)ccn1 ; O=C(N1CC(OC2CCC2)C1)N1N=CC[C@H]1c1cc(F)cc(F)c1]

20. A method for treating RIPK1 dependent inflammation and cell death that occurs in inherited and sporadic diseases including Alzheimer's disease, amyotrophic lateral sclerosis, multiple sclerosis, Parkinson's disease, chronic traumatic encephalopathy, rheumatoid arthritis, ulcerative colitis, inflammatory bowel disease, psoriasis as well as acute tissue injury caused by stroke, traumatic brain injury, encephalitis comprising administering to a patient in need thereof a compound, or pharmaceutically acceptable salt thereof, of claim 1.

21. A method of treating amyotrophic lateral sclerosis comprising administering to a patient in need thereof a compound, or pharmaceutically acceptable salt thereof, of claim 1.

22. (canceled)

23. A pharmaceutical composition comprising a compound of 1, or a pharmaceutically acceptable salt thereof and a pharmaceutically acceptable carrier.

24. A pharmaceutical composition comprising a compound of claim 1, and a pharmaceutically acceptable carrier.
